(19) United States
(12) Patent Application Publication
(10) Pub. No.: US 2023/0058054 A1
(43) Pub. Date: Feb. 23, 2023

(54) CRISPR/CAS SYSTEM AND USES THEREOF

(71) Applicant: HuiGene Therapeutics Co., Ltd., Shanghai (CN)

(72) Inventor: Xing Wang, Shanghai (CN)

(21) Appl. No.: 17/851,030

(22) Filed: Jun. 28, 2022

(30) Foreign Application Priority Data
Jun. 29, 2021 (CN) PCT/CN2021/103326

(51) Int. Cl. C12N 15/90; C12N 15/11; C12N 9/22

(52) U.S. Cl. CPC C12N 15/907; C12N 15/11; C12N 9/22; C12N 2310/20; C07K 2319/00; A61K 38/00

(57) ABSTRACT

The invention provides novel CRISPR/Cas compositions and uses thereof for targeting nucleic acids. In particular, the invention provides non-naturally occurring or engineered RNA-targeting systems comprising a novel RNA-targeting Cas13c, Cas13d, Cas13e, or Cas 13f effector protein, and at least one targeting nucleic acid component such as a guide RNA (gRNA) or crRNA. The novel Cas effector proteins are among the smallest of the known Cas effector proteins, at about 800-900 amino acids in size, and are thus uniquely suitable for delivery using vectors of small capacity, such as an AAV vector.

### REFERENCE TO RELATED APPLICATION

The application claims foreign priority under 35 U.S.C. 365(a) to International Patent Application No. PCT/CN2021/103326, filed on Jun. 29, 2021, the entire contents of which, including any drawings and sequence listing thereof, are incorporated herein by reference.

### SEQUENCE LISTING

The instant application contains a Sequence Listing which has been submitted electronically in ASCII format and is hereby incorporated by reference in its entirety. Said ASCII copy, created on Jun. 9, 2022, is named 132045-00701_SL.txt and is 264,795 bytes in size.

### BACKGROUND OF THE INVENTION

CRISPR (clustered regularly interspaced short palindromic repeats) is a family of DNA sequences found within the genomes of prokaryotic organisms such as bacteria and archaea. These sequences are understood to be derived from DNA fragments of bacteriophages that have previously infected the prokaryote, and are used to detect and destroy DNA from similar bacterialphages during subsequent infections of the prokaryotes.

CRISPR-associated systems is a set of homologous genes, or Cas genes, some of which encode Cas protein having helicase and nuclease activities. The Cas proteins are enzymes that utilize RNA derived from the CRISPR sequences (crRNA) as guide sequences to recognize and cleave specific strands of polynucleotide (e.g., DNA) that are complementary to the crRNA.

Together, the CRISPR-Cas system constitutes a primitive prokaryotic “immune system” that confers resistance or acquired immunity to foreign pathogenic genetic elements, such as those present within extrachromosomal DNA (e.g., plasmids) and bacterialphages, or foreign RNA encoded by foreign DNA.

In nature, the CRISPR/Cas system appears to be a widespread prokaryotic defense mechanism against foreign genetic materials, and is found in approximately 50% of sequenced bacterial genomes and nearly 90% of sequenced archaea. This prokaryotic system has since been developed to form the basis of a technology known as CRISPR-Cas that found extensive use in numerous eukaryotic organisms including human, in a wide variety of applications including basic biological research, development of biotechnology products, and disease treatment.

The prokaryotic CRISPR-Cas systems comprise an extremely diverse group of proteins effectors, non-coding elements, as well as loci architectures, some examples of which have been engineered and adapted to produce important biotechnologies.

The CRISPR locus structure has been studied in many systems. In these systems, the CRISPR array in the genomic DNA typically comprises an AT-rich leader sequence, followed by short DR sequences separated by unique spacer sequences. These CRISPR DR sequences typically range in size from 28 to 37 bps, though the range can be 23-55 bps. Some DR sequences show dyad symmetry, implying the formation of a secondary structure such as a stem-loop (“hairpin”) in the RNA, while others appear unstructured. The size of spacers in different CRISPR arrays is typically 32-38 bps (with a range of 21-72 bps). There are usually fewer than 50 units of the repeat-spacer sequence in a CRISPR array.

Small clusters of cas genes are often found next to such CRISPR repeat-spacer arrays. So far, the 93 identified cas genes have been grouped into 35 families, based on sequence similarity of their encoded proteins. Eleven of the 35 families form the so-called cas core, which includes the protein families Cas1 through Cas9. A complete CRISPR-Cas locus has at least one gene belonging to the cas core.

CRISPR-Cas systems can be broadly divided into two classes—Class 1 systems use a complex of multiple Cas proteins to degrade foreign nucleic acids, while Class 2 systems use a single large Cas protein for the same purpose. The single-subunit effector compositions of the Class 2 systems provide a simpler component set for engineering and application translation, and has thus far been important sources of discovery, engineering, and optimization of novel powerful programmable technologies for genome engineering and beyond.

Class 1 system is further divided into types I, III, and IV; and Class 2 system is divided into types II, V, and VI. These 6 system types are additionally divided into 19 subtypes. Classification is also based on the complement of cas genes that are present. Most CRISPR-Cas systems have a Cas1 protein. Many prokaryotes contain multiple CRISPR-Cas systems, suggesting that they are compatible and may share components.

One of the first and best characterized Cas proteins—Cas9—is a prototypical member of Class 2, type II, and originates from Streptococcus pyogenes (SpCas9). Cas9 is a DNA endonuclease activated by a small crRNA molecule that complements a target DNA sequence, and a separate trans-activating CRISPR RNA (tracrRNA). The crRNA consists of a direct repeat (DR) sequence responsible for protein binding to the crRNA and a spacer sequence, which may be engineered to be complementary to any desired nucleic acid target sequence. In this way, CRISPR systems can be programmed to target DNA or RNA targets by modifying the spacer sequence of the crRNA. The crRNA and tracrRNA have been fused to form a single guide RNA (sgRNA) for better practical utility. When combined with Cas9, sgRNA hybridizes with its target DNA, and guides Cas9 to cut the target DNA. Other Cas9 effector protein from other species have also been identified and used similarly, including Cas9 from the S. thermophilus CRISPR system. These CRISPR/Cas9 systems have been widely used in numerous eukaryotic organisms, including baker's yeast (Saccharomyces cerevisiae), the opportunistic pathogen Candida albicans, zebrafish (Danio rerio), fruit flies (Drosophila melanogaster), ants (Harpegnathos saltator and Ooceraea biroi), mosquitoes (Aedes aegypti), nematodes (Caenorhabditis elegans), plants, mice, monkeys, and human embryos.

Another recently characterized Cas effector protein is Cas12a (formerly known as Cpf1). Cas12a, together with C2c1 and C2c3, are members belonging to Class 2, type V Cas proteins that lack HNH nuclease, but have RuvC nuclease activity. Cas12a which was initially characterized in the CRISPR/Cpf1 system of the bacterium Francisella novicida. Its original name reflects the prevalence of its CRISPR-Cas subtype in the Prevotella and Francisella lineages. Cas12a showed several key differences from Cas9, including: causing a “staggered” cut in double stranded DNA as opposed to the “blunt” cut produced by Cas9, relying on a “T rich” PAM sequence (which provides alternative targeting sites to Cas9) and requiring only a CRISPR RNA (crRNA) and no tracrRNA for successful targeting. Cas12a's small crRNAs are better suited than Cas9 for multiplexed genome editing, as more of them can be packaged in one vector than can Cas9's sgRNAs. Further, the sticky 5′ overhangs left by Cas12a can be used for DNA assembly that is much more target-specific than traditional Restriction Enzyme cloning. Finally, Cas12a cleaves DNA 18-23 base pairs downstream from its PAM site, which means no disruption to the nuclease recognition sequence after DNA repair following the creation of double stranded break (DSB) by the NHEJ system, thus Cas enables multiple rounds of DNA cleavage, as opposed to the likely one round after Cas9 cleavage because the Cas9 cleavage sequence is only 3 base pairs upstream of the PAM site, and the NHEJ pathway typically results in indel mutations which destroy the recognition sequence, thereby preventing further rounds of cutting. In theory, repeated rounds of DNA cleavage is associated with an increased chance for the desired genomic editing to occur.

More recently, several Class 2, type VI Cas proteins, including Cas13 (also known as C2c2), Cas13b, Cas13c, and Cas13d have been identified, each is an RNA-guided RNase (i.e., these Cas proteins use their crRNA to recognize target RNA sequences, rather than target DNA sequences in Cas9 and Cas12a). Overall, the CRISPR/Cas13 systems can achieve higher RNA digestion efficiency compared to the traditional RNAi and CRISPRi technologies, while simultaneously exhibiting much less off-target cleavage compared to RNAi.

One drawback from these currently identified Cas13 proteins is their relatively large size. Each of Cas13a, Cas13b, and Cas13c has more than 1100 amino acid residues. Thus it is difficult, if possible at all, to package their coding sequence (about 3.3 kb) and sgRNA, plus any required promoter sequences and translation regulatory sequences, into certain small capacity gene therapy vectors, such as the current most efficient and safest gene therapy vector based on adeno associated virus (AAV), which has a package capacity of about 4.7 kb. Although Cas13d, the smallest Cas13 protein so far, only has about 920 amino acids (i.e., about 2.8 kb coding sequence), and can in theory be packaged into the AAV vector, it has limited use for single-base editing-based gene therapy that depends on using Cas13d-based fusion proteins with single-base editing functions, such as dCas13d-ADAR2DD (which has a coding sequence of about 3.9 kb).

### SUMMARY OF THE INVENTION

One aspect of the invention provides a Clustered Regularly Interspaced Short Palindromic Repeat (CRISPR)-Cas complex, comprising: (1) an RNA guide sequence comprising a spacer sequence capable of hybridizing to a target RNA, and a direct repeat (DR) sequence 3′ to the spacer sequence; and, (2) a CRISPR-associated protein (Cas) having an amino acid sequence of any one of SEQ ID NOs: 2-7 and 9-17, or a derivative (such as one with at least about 75%, 80%, 85%, 90%, 95%, 96%, 97%, 98%, 99%, 99.2%, 99.5%, 99.7%, 99.9% amino acid sequence identity with the wild-type Cas, or one with at least about 1, 2, 3, 4, 5, 6, 7, 8, 9, 10, 15, 20, 25, 30, 35, 40, 45, 50, 55, 60, 65, 70, 75, 80, 85, or 90 amino acid substitutions, such as conserved substitutions, but no more than 150, 140, 130, 120, 110, or 100 substitutions, such as conserved substitutions) or functional fragment of said Cas (such as N- and/or C-terminal deletions, each independently with at least about 4, 10, 15, 20, 25, 30, 35, 40, 45, 50, 55, 60, 65, 70, 75, 80, 85, 90, 100, 110, 120, 130, 140, 150, 160, 170, 180, 190, 200, 210, 220 residue deletions); wherein the Cas, the derivative, and the functional fragment of said Cas, are capable of (i) binding to the RNA guide sequence, and/or (ii) targeting the target RNA, with the proviso that the spacer sequence is not 100% complementary to a naturally-occurring bacterialphage nucleic acid when the complex comprises the Cas of any one of SEQ ID NOs: 2-7 and 9-17 or wherein the target RNA is encoded by a eukaryotic DNA.

In certain embodiments, the DR sequence has substantially the same secondary structure as the secondary structure of any one of SEQ ID NOs: 19-24 and 26-34.

In certain embodiments, the DR sequence is encoded by any one of SEQ ID NOs: 19-24 and 26-34, or contains no more than 1, 2, 3, 4, 5, 6, 7, 8, 9, or 10 substitutions, deletions, or additions of any one of SEQ ID NOs: 19-24 and 26-34.

In certain embodiments, the target RNA is encoded by a eukaryotic DNA.

In certain embodiments, the eukaryotic DNA is a non-human mammalian DNA, a non-human primate DNA, a human DNA, a plant DNA, an insect DNA, a bird DNA, a reptile DNA, a rodent DNA, a fish DNA, a worm/nematode DNA, a yeast DNA.

In certain embodiments, the target RNA is an mRNA.

In certain embodiments, the spacer sequence is between 15-55 nucleotides, between 25-35 nucleotides, or about 30 nucleotides.

In certain embodiments, the spacer sequence is 90-100% complementary to the target RNA.

In certain embodiments, the derivative is at least about 90%, 95%, 96%, 97%, 98%, 99% identical to any one of SEQ ID NOs: 2-7 and 9-17, or comprises conserved amino acid substitutions of one or more residues of any one of SEQ ID NOs: 2-7 and 9-17.

In certain embodiments, the derivative comprises only conserved amino acid substitutions.

In certain embodiments, the derivative has identical sequence to wild-type Cas of any one of SEQ ID NOs: 2-7 and 9-17 in the HEPN domain or the RXXXXH motif.

In certain embodiments, the derivative is capable of binding to the RNA guide sequence hybridized to the target RNA, but has no RNase catalytic activity due to a mutation in the RNase catalytic site of the Cas.

In certain embodiments, the derivative has an N-terminal deletion of no more than 210 residues, and/or a C-terminal deletion of no more than 180 residues.

In certain embodiments, the derivative has an N-terminal deletion of about 180 residues, and/or a C-terminal deletion of about 150 residues.

In certain embodiments, the derivative further comprises an RNA base-editing domain.

In certain embodiments, the RNA base-editing domain is an adenosine deaminase, such as a double-stranded RNA-specific adenosine deaminase (e.g., ADAR1 or ADAR2); apolipoprotein B mRNA editing enzyme; catalytic polypeptide-like (APOBEC); or activation-induced cytidine deaminase (AID).

In certain embodiments, the ADAR has E488Q/T375G double mutation or is ADAR2DD.

In certain embodiments, the base-editing domain is further fused to an RNA-binding domain, such as MS2.

In certain embodiments, the derivative further comprises an RNA methyltransferase, a RNA demethylase, an RNA splicing modifier, a localization factor, or a translation modification factor.

In certain embodiments, the Cas, the derivative, or the functional fragment comprises a nuclear localization signal (NLS) sequence or a nuclear export signal (NES).

In certain embodiments, targeting of the target RNA results in a modification of the target RNA.

In certain embodiments, the modification of the target RNA is a cleavage of the target RNA.

In certain embodiments, the modification of the target RNA is deamination of an adenosine (A) to an inosine (I).

In certain embodiments, the CRISPR-Cas complex of the invention further comprises a target RNA comprising a sequence capable of hybridizing to the spacer sequence.

Another aspect of the invention provides a fusion protein, comprising (1) the Cas, the derivative thereof, or the functional fragment thereof, of the invention, and (2) a heterologous functional domain.

In certain embodiments, the heterologous functional domain comprises: a nuclear localization signal (NLS), a reporter protein or a detection label (e.g., GST, HRP, CAT, GFP, HcRed, DsRed, CFP, YFP, BFP), a localization signal, a protein targeting moiety, a DNA binding domain (e.g., MBP, Lex A DBD, Gal4 DBD), an epitope tag (e.g., His, myc, V5, FLAG, HA, VSV-G, Trx, etc), a transcription activation domain (e.g., VP64 or VPR), a transcription inhibition domain (e.g., KRAB moiety or SID moiety), a nuclease (e.g., Fold), a deamination domain (e.g., ADAR1, ADAR2, APOBEC, AID, or TAD), a methylase, a demethylase, a transcription release factor, an HDAC, a polypeptide having ssRNA cleavage activity, a polypeptide having dsRNA cleavage activity, a polypeptide having ssDNA cleavage activity, a polypeptide having dsDNA cleavage activity, a DNA or RNA ligase, or any combination thereof.

In certain embodiments, the heterologous functional domain is fused N-terminally, C-terminally, or internally in the fusion protein.

Another aspect of the invention provides a conjugate, comprising (1) the Cas, the derivative thereof, or the functional fragment thereof, of the invention, conjugated to (2) a heterologous functional moiety.

In certain embodiments, the heterologous functional moiety comprises: a nuclear localization signal (NLS), a reporter protein or a detection label (e.g., GST, HRP, CAT, GFP, HcRed, DsRed, CFP, YFP, BFP), a localization signal, a protein targeting moiety, a DNA binding domain (e.g., MBP, Lex A DBD, Gal4 DBD), an epitope tag (e.g., His, myc, V5, FLAG, HA, VSV-G, Trx, etc), a transcription activation domain (e.g., VP64 or VPR), a transcription inhibition domain (e.g., KRAB moiety or SID moiety), a nuclease (e.g., Fold), a deamination domain (e.g., ADAR1, ADAR2, APOBEC, AID, or TAD), a methylase, a demethylase, a transcription release factor, an HDAC, a polypeptide having ssRNA cleavage activity, a polypeptide having dsRNA cleavage activity, a polypeptide having ssDNA cleavage activity, a polypeptide having dsDNA cleavage activity, a DNA or RNA ligase, or any combination thereof.

In certain embodiments, the heterologous functional moiety is conjugated N-terminally, C-terminally, or internally with respect to the Cas, the derivative thereof, or the functional fragment thereof.

Another aspect of the invention provides a polynucleotide encoding any one of SEQ ID NOs: 2-7 and 9-17, or a derivative polynucleotide thereof (such as a polynucleotide at least about 60%, 65%, 70%, 75%, 80%, 85%, 90%, 95%, 96%, 97%, 98%, 99%, 99.2%, 99.5%, 99.7%, 99.9% identical to the wild-type polynucleotide encoding any one of SEQ ID NOs: 2-7 and 9-17), or a polynucleotide encoding a derivative of any one of SEQ ID NOs: 2-7 and 9-17, or a functional fragment of any one of SEQ ID NOs: 2-7 and 9-17 (see above), or a fusion protein of any one of SEQ ID NOs: 2-7 and 9-17, or a polynucleotide at least about 70%, 80%, 85%, 90%, 95%, 96%, 97%, 98%, 99% identical thereto, provided that the polynucleotide is not any one of SEQ ID NOs:1 and 8.

In certain embodiments, the polynucleotide is codon-optimized for expression in a cell.

In certain embodiments, the cell is a eukaryotic cell.

Another aspect of the invention provides a non-naturally occurring polynucleotide comprising a derivative of any one of SEQ ID NOs: 19-24 and 26-34, wherein said derivative (i) has one or more (e.g., 1, 2, 3, 4, 5, 6, 7, 8, 9 or 10) nucleotides additions, deletions, or substitutions compared to any one of SEQ ID NOs: 19-24 and 26-34; (ii) has at least 20%, 30%, 40%, 50%, 60%, 70%, 80%, 90%, 95%, or 97% sequence identity to any one of SEQ ID NOs: 19-24 and 26-34; (iii) hybridize under stringent conditions with any one of SEQ ID NOs: 19-24 and 26-34 or any of (i) and (ii); or (iv) is a complement of any of (i)-(iii), provided that the derivative is not any one of SEQ ID NOs: 19-24 and 26-34, and that the derivative encodes an RNA (or is an RNA) that has maintained substantially the same secondary structure as any of the RNA encoded by SEQ ID NOs: 19-24 and 26-34.

In certain embodiments, the derivative functions as a DR sequence for any one of the Cas, the derivative thereof, or the functional fragment thereof, of the invention.

Another aspect of the invention provides a vector comprising the polynucleotide of the invention.

In certain embodiments, the polynucleotide is operably linked to a promoter and optionally an enhancer.

In certain embodiments, the promoter is a constitutive promoter, an inducible promoter, a ubiquitous promoter, or a tissue specific promoter.

In certain embodiments, the vector is a plasmid.

In certain embodiments, the vector is a retroviral vector, a phage vector, an adenoviral vector, a herpes simplex viral (HSV) vector, an AAV vector, or a lentiviral vector.

In certain embodiments, the AAV vector is a recombinant AAV vector of the serotype AAV1, AAV2, AAV4, AAV5, AAV6, AAV7, AAVrh74, AAV8, AAV9, AAV10, AAV 11, AAV 12, or AAV 13.

Another aspect of the invention provides a delivery system comprising (1) a delivery vehicle, and (2) the CRISPR-Cas complex of the invention, the fusion protein of the invention, the conjugate of the invention, the polynucleotide of the invention, or the vector of the invention.

In certain embodiments, the delivery vehicle is a nanoparticle, a liposome, an exosome, a microvesicle, or a gene-gun.

Another aspect of the invention provides a cell or a progeny thereof, comprising the CRISPR-Cas complex of the invention, the fusion protein of the invention, the conjugate of the invention, the polynucleotide of the invention, or the vector of the invention.

In certain embodiments, the cell or progeny thereof is a eukaryotic cell (e.g., a non-human mammalian cell, a human cell, or a plant cell) or a prokaryotic cell (e.g., a bacteria cell).

Another aspect of the invention provides a non-human multicellular eukaryote comprising the cell of the invention.

In certain embodiments, the non-human multicellular eukaryote is an animal (e.g., rodent or primate) model for a human genetic disorder.

Another aspect of the invention provides a method of modifying a target RNA, the method comprising contacting the target RNA with the CRISPR-Cas complex of the invention, wherein the spacer sequence is complementary to at least 15 nucleotides of the target RNA; wherein the Cas, the derivative, or the functional fragment associates with the RNA guide sequence to form the complex; wherein the complex binds to the target RNA; and wherein upon binding of the complex to the target RNA, the Cas, the derivative, or the functional fragment modifies the target RNA.

In certain embodiments, the target RNA is modified by cleavage by the Cas.

In certain embodiments, the target RNA is modified by deamination by a derivative comprising a Double-stranded RNA-specific adenosine deaminase.

In certain embodiments, the target RNA is an mRNA, a tRNA, an rRNA, a non-coding RNA, an lncRNA, or a nuclear RNA.

In certain embodiments, upon binding of the complex to the target RNA, the Cas, the derivative, and the functional fragment does not exhibit substantial (or detectable) collateral RNase activity.

In certain embodiments, the target RNA is within a cell.

In certain embodiments, the cell is a cancer cell.

In certain embodiments, the cell is infected with an infectious agent.

In certain embodiments, the infectious agent is a virus, a prion, a protozoan, a fungus, or a parasite.

In certain embodiments, the CRISPR-Cas complex is encoded by a first polynucleotide encoding any one of SEQ ID NOs: 2-7 and 9-17, or a derivative or functional fragment thereof, and a second polynucleotide comprising any one of SEQ ID NOs: 19-24 and 26-34 and a sequence encoding a spacer RNA capable of binding to the target RNA, wherein the first and the second polynucleotides are introduced into the cell.

In certain embodiments, the first and the second polynucleotides are introduced into the cell by the same vector.

In certain embodiments, the method causes one or more of: (i) in vitro or in vivo induction of cellular senescence; (ii) in vitro or in vivo cell cycle arrest; (iii) in vitro or in vivo cell growth inhibition and/or cell growth inhibition; (iv) in vitro or in vitro induction of anergy; (v) in vitro or in vitro induction of apoptosis; and (vi) in vitro or in vitro induction of necrosis.

Another aspect of the invention provides a method of treating a condition or disease in a subject in need thereof, the method comprising administering to the subject a composition comprising the CRISPR-Cas complex of the invention or a polynucleotide encoding the same; wherein the spacer sequence is complementary to at least 15 nucleotides of a target RNA associated with the condition or disease; wherein the Cas, the derivative, or the functional fragment associates with the RNA guide sequence to form the complex; wherein the complex binds to the target RNA; and wherein upon binding of the complex to the target RNA, the Cas, the derivative or the functional fragment cleaves the target RNA, thereby treating the condition or disease in the subject.

In certain embodiments, the condition or disease is a cancer or an infectious disease. In certain embodiments, the cancer is Wilms' tumor, Ewing sarcoma, a neuroendocrine tumor, a glioblastoma, a neuroblastoma, a melanoma, skin cancer, breast cancer, colon cancer, rectal cancer, prostate cancer, liver cancer, renal cancer, pancreatic cancer, lung cancer, biliary cancer, cervical cancer, endometrial cancer, esophageal cancer, gastric cancer, head and neck cancer, medullary thyroid carcinoma, ovarian cancer, glioma, lymphoma, leukemia, myeloma, acute lymphoblastic leukemia, acute myelogenous leukemia, chronic lymphocytic leukemia, chronic myelogenous leukemia, Hodgkin's lymphoma, non-Hodgkin's lymphoma, or urinary bladder cancer.

In certain embodiments, the method is an in vitro method, an in vivo method, or an ex vivo method.

Another aspect of the invention provides a cell or a progeny thereof, obtained by the method of the invention, wherein the cell and the progeny comprises a non-naturally existing modification (e.g., a non-naturally existing modification in a transcribed RNA of the cell/progeny).

Another aspect of the invention provides a method to detect the presence of a target RNA, the method comprising contacting the target RNA with a composition comprising a fusion protein of the invention, or a conjugate of the invention, or a polynucleotide encoding the fusion protein, wherein the fusion protein or the conjugate comprises a detectable label (e.g., one that can be detected by fluorescence, Northern blot, or FISH) and a complexed spacer sequence capable of binding to the target RNA.

Another aspect of the invention provides a eukaryotic cell comprising a Clustered Regularly Interspaced Short Palindromic Repeat (CRISPR)-Cas complex, said CRISPR-Cas complex comprising: (1) an RNA guide sequence comprising a spacer sequence capable of hybridizing to a target RNA, and a direct repeat (DR) sequence 3′ to the spacer sequence; and, (2) a CRISPR-associated protein (Cas) having an amino acid sequence of any one of SEQ ID NOs: 2-7 and 9-17, or a derivative or functional fragment of said Cas; wherein the Cas, the derivative, and the functional fragment of said Cas, are capable of (i) binding to the RNA guide sequence and (ii) targeting the target RNA.

It should be understood that any one embodiment of the invention described herein, including those described only in the examples or claims, or only in one aspects/sections below, can be combined with any other one or more embodiments of the invention, unless explicitly disclaimed or improper.

### BRIEF DESCRIPTION OF THE DRAWINGS

FIG. 1 is a schematic drawing showing that the three plasmids, encoding (1) a Cas13e effector protein, (2) a coding sequence for the guide RNA (gRNA) which can produce the guide RNA that is complementary to the mCherry mRNA and that can form a complex with the Cas13e effector protein, and (3) the mCherry reporter gene, respectively, can be transfected to a cell to express their respective gene products, resulting in the degradation of the reporter mCherry mRNA.

FIG. 2 shows putative secondary structures of the DR sequences associated with the respective Cas13e, Cas13f, Cas13d, and Cas13c proteins. Their coding sequences, from left to right and from top to bottom, are represented by SEQ ID NOs: 106-120, respectively, in order of appearance.

FIG. 3 shows the cleavage activity of two subject Cas13c proteins, Cas13c.1 and Cas13c.2, using three different single-guide RNA's (sgRNA) (i.e., mCherry-sg1, mCherry-sg2, and mCherry-sg3) targeting the reporter transcript mCherry in mammalian HEK293T cells. An sgRNA targeting LacZ (LacZ-sg) was included as a negative control.

FIG. 4 shows the cleavage activity of five subject Cas13d proteins, Cas13d.1, Cas13d.2, Cas13d.3, Cas13d.4, and Cas13d.5, using three different single-guide RNA's (sgRNA) (i.e., mCherry-sg1, mCherry-sg2, and mCherry-sg3) targeting the reporter transcript mCherry in mammalian HEK293T cells. An sgRNA targeting LacZ (LacZ-sg) was included as a negative control.

FIG. 5 shows the cleavage activity of a subject Cas13e protein, Cas13e.3, using three different single-guide RNA's (sgRNA) (i.e., mCherry-sg1, mCherry-sg2, and mCherry-sg3) targeting the reporter transcript mCherry in mammalian HEK293T cells. An sgRNA targeting LacZ (LacZ-sg) was included as a negative control. The previously discovered Cas13e.1 was used as a positive control.

FIG. 6 shows the cleavage activity of two subject Cas13f proteins, Cas13f.6 and Cas13f.7, using three different single-guide RNA's (sgRNA) (i.e., mCherry-sg1, mCherry-sg2, and mCherry-sg3) targeting the reporter transcript mCherry in mammalian HEK293T cells. An sgRNA targeting LacZ (LacZ-sg) was included as a negative control.

FIG. 7 shows the cleavage activity of Cas13e.4, Cas13e.5, Cas13e.6, Cas13e.7, and Cas13e.8, using three different single-guide RNA's (sgRNA) (i.e., mCherry-sg1, mCherry-sg2, and mCherry-sg3) targeting the reporter transcript mCherry in mammalian HEK293T cells. An sgRNA targeting LacZ (LacZ-sg) was included as a negative control.

### DETAILED DESCRIPTION OF THE INVENTION

### 1. Overview

The invention described herein provides novel Class 2, type VI Cas effector proteins, sometimes referred herein as Cas13c, Cas13d, Cas13e and Cas13f (collectively “Cas13” herein). The novel Cas13 proteins of the invention are much smaller (e.g., about 800-900 residues) than the previously discovered Cas13 effector proteins (Cas13a-Cas13d), such that they can be easily packaged with their crRNA coding sequences into small capacity gene therapy vectors, such as the AAV vectors. Further, at least some of the newly discovered Cas13 effector proteins are more potent in knocking down RNA target sequences, and more efficient in RNA single base editing, as compared to the known Cas13a, Cas13b, and Cas13d effector proteins, while exhibiting negligible non-specific/collateral RNase activity upon activation by crRNA-based target recognition, except when the spacer sequence is within a specific narrow range (e.g., about 30 nucleotide). Thus these new Cas proteins are ideally suited for gene therapy.

Thus in the first aspect, the invention provides Cas13c, Cas13d, Cas13e, and Cas13f effector proteins, such as those with amino acid sequences of SEQ ID NOs: 2-7 and 9-17, or orthologs, homologs, the various derivatives (described herein below), functional fragments thereof (described herein bellow), wherein said orthologs, homologs, derivatives and functional fragments have maintained at least one function of any one of the proteins of SEQ ID NOs: 2-7 and 9-17. Such functions include, but are not limited to, the ability to bind a guide RNA/crRNA of the invention (described herein below) to form a complex, the RNase activity, and the ability to bind to and cleave a target RNA at a specific site, under the guidance of the crRNA that is at least partially complementary to the target RNA.

In certain embodiments, the Cas13e or Cas13f effector proteins of the invention can be: (i) any one of SEQ ID NOs: 2-7 and 9-17; (ii) a derivative having one or more amino acids (e.g., 1, 2, 3, 4, 5, 6, 7, 8, 9, or 10 residues) of addition, deletion, and/or substitution (e.g., conserved substitution) of any one of SEQ ID NOs: 2-7 and 9-17 (such as one with at least about 75%, 80%, 85%, 90%, 95%, 96%, 97%, 98%, 99%, 99.2%, 99.5%, 99.7%, 99.9% amino acid sequence identity with the wild-type Cas, or one with at least about 1, 2, 3, 4, 5, 6, 7, 8, 9, 10, 15, 20, 25, 30, 35, 40, 45, 50, 55, 60, 65, 70, 75, 80, 85, or 90 amino acid substitutions, such as conserved substitutions, but no more than 150, 140, 130, 120, 110, or 100 substitutions, such as conserved substitutions); or (iii) a derivative having amino acid sequence identity of at least about 80%, 85%, 90%, 91%, 92%, 93%, 94%, 95%, 96%, 97%, 98%, or 99% compared to any one of SEQ ID NOs: 2-7 and 9-17.

In certain embodiments, the Cas13c, Cas13d, Cas13e, and Cas13f effector proteins, orthologs, homologs, derivatives and functional fragments thereof are not naturally existing, e.g., having at least one amino acid difference compared to a naturally existing sequence.

In a related aspect, the invention provides additional derivatives Cas13c, Cas13d, Cas13e, and Cas13f effector proteins based on any one of SEQ ID NOs: 2-7 and 9-17, or the above orthologs, homologs, derivatives and functional fragments thereof, which comprises another covalently or non-covalently linked protein or polypeptide or other molecules (such as detection reagents or drug/chemical moieties). Such other proteins/polypeptides/other molecules can be linked through, for example, chemical coupling, gene fusion, or other non-covalent linkage (such as biotin-streptavidin binding). Such derived proteins do not affect the function of the original protein, such as the ability to bind a guide RNA/crRNA of the invention (described herein below) to form a complex, the RNase activity, and the ability to bind to and cleave a target RNA at a specific site, under the guidance of the crRNA that is at least partially complementary to the target RNA.

Such derivation may be used, for example, to add a nuclear localization signal (NLS, such as SV40 large T antigen NLS) to enhance the ability of the subject Cas13c, Cas13d, Cas13e, and Cas13f effector proteins to enter cell nucleus. Such derivation can also be used to add a targeting molecule or moiety to direct the subject Cas13c, Cas13d, Cas13e, and Cas13f effector proteins to specific cellular or subcellular locations. Such derivation can also be used to add a detectable label to facilitate the detection, monitoring, or purification of the subject Cas13c, Cas13d, Cas13e, and Cas13f effector proteins. Such derivation can further be used to add a deamination enzyme moiety (such as one with adenine or cytosine deamination activity) to facilitate RNA base editing.

The derivation can be through adding any of the additional moieties at the N- or C-terminal of the subject Cas13c, Cas13d, Cas13e, and Cas13f effector proteins, or internally (e.g., internal fusion or linkage through side chains of internal amino acids).

In a related second aspect, the invention provides conjugates of the subject Cas13c, Cas13d, Cas13e, and Cas13f effector proteins based on any one of SEQ ID NOs: 2-7 and 9-17, or the above orthologs, homologs, derivatives and functional fragments thereof, which are conjugated with moieties such as other proteins or polypeptides, detectable labels, or combinations thereof. Such conjugated moieties may include, without limitation, localization signals, reporter genes (e.g., GST, HRP, CAT, GFP, HcRed, DsRed, CFP, YFP, BFP), labels (e.g., fluorescent dye such as FITC, or DAPI), NLS, targeting moieties, DNA binding domains (e.g., MBP, Lex A DBD, Gal4 DBD), epitope tags (e.g., His, myc, V5, FLAG, HA, VSV-G, Trx, etc), transcription activation domains (e.g., VP64 or VPR), transcription inhibition domains (e.g., KRAB moiety or SID moiety), nucleases (e.g., Fold), deamination domain (e.g., ADAR1, ADAR2, APOBEC, AID, or TAD), methylase, demethylase, transcription release factor, HDAC, ssRNA cleavage activity, dsRNA cleavage activity, ssDNA cleavage activity, dsDNA cleavage activity, DNA or RNA ligase, any combination thereof, etc.

For example, the conjugate may include one or more NLSs, which can be located at or near N-terminal, C-terminal, internally, or combination thereof. The linkage can be through amino acids (such as D or E, or S or T), amino acid derivatives (such as Ahx, β-Ala, GABA or Ava), or PEG linkage.

In certain embodiments, conjugations do not affect the function of the original protein, such as the ability to bind a guide RNA/crRNA of the invention (described herein below) to form a complex, the RNase activity, and the ability to bind to and cleave a target RNA at a specific site, under the guidance of the crRNA that is at least partially complementary to the target RNA.

In a related third aspect, the invention provides fusions of the subject Cas13c, Cas13d, Cas13e, and Cas13f effector proteins based on any one of SEQ ID NOs: 2-7 and 9-17, or the above orthologs, homologs, derivatives and functional fragments thereof, which fusions are with moieties such as localization signals, reporter genes (e.g., GST, HRP, CAT, GFP, HcRed, DsRed, CFP, YFP, BFP), NLS, protein targeting moieties, DNA binding domains (e.g., MBP, Lex A DBD, Gal4 DBD), epitope tags (e.g., His, myc, V5, FLAG, HA, VSV-G, Trx, etc), transcription activation domains (e.g., VP64 or VPR), transcription inhibition domains (e.g., KRAB moiety or SID moiety), nucleases (e.g., FokI), deamination domain (e.g., ADAR1, ADAR2, APOBEC, AID, or TAD), methylase, demethylase, transcription release factor, HDAC, ssRNA cleavage activity, dsRNA cleavage activity, ssDNA cleavage activity, dsDNA cleavage activity, DNA or RNA ligase, any combination thereof, etc.

For example, the fusion may include one or more NLSs, which can be located at or near N-terminal, C-terminal, internally, or combination thereof. In certain embodiments, conjugations do not affect the function of the original protein, such as the ability to bind a guide RNA/crRNA of the invention (described herein below) to form a complex, the RNase activity, and the ability to bind to and cleave a target RNA at a specific site, under the guidance of the crRNA that is at least partially complementary to the target RNA.

In a fourth aspect, the invention provides an isolated polynucleotide, e.g., an isolated polynucleotide that can serve as a direct repeat (DR) sequence for any one of the Cas13 proteins of SEQ ID NOs: 2-7 and 9-17, said isolated polynucleotide comprising: (i) any one of SEQ ID NOs: 19-24 and 26-34; (ii) a polynucleotide having 1, 2, 3, 4, or 5 nucleotides of deletion, addition, and/or substitution compared to any one of SEQ ID NOs: 19-24 and 26-34; (iii) a polynucleotide sharing at least 80%, 85%, 90%, 95% sequence identity with any one of SEQ ID NOs: 19-24 and 26-34; (iv) a polynucleotide that hybridize under stringent condition with any one of the polynucleotide of (i)-(iii) or a complement thereof; (v) a complement sequence of any polynucleotide of (i)-(iii).

Any polynucleotide of (ii)-(iv) has maintained the function of the original SEQ ID NOs: 19-24 and 26-34, which is to encode a direct repeat (DR) sequence of a crRNA in the subject Cas13c, Cas13d, Cas13e, and Cas13f system.

As used herein, “direct repeat sequence” may refer to the DNA coding sequence in the CRISPR locus, or to the RNA encoded by the same in crRNA. Thus when any of SEQ ID NOs: 19-24 and 26-34 is referred to in the context of an RNA molecule, such as crRNA, each T is understood to represent a U.

Thus in certain embodiments, the isolated polynucleotide is a DNA, which encodes a DR sequence for a crRNA of the subject Cas13c, Cas13d, Cas13e, and Cas13f system.

In certain other embodiments, the isolated polynucleotide is an RNA, which is a DR sequence for a crRNA of the subject Cas13c, Cas13d, Cas13e, and Cas13f system.

In a fifth aspect, the invention provides a complex comprising: (i) a protein composition that can be any one of the subject Cas13c, Cas13d, Cas13e, and Cas13f effector protein, or orthologs, homologs, derivatives, conjugates, functional fragments thereof, conjugates thereof, or fusions thereof; and (ii) a polynucleotide composition, comprising an isolated polynucleotide described in the 4th aspect of the invention (e.g., a DR sequence), and a spacer sequence complementary to at least a portion of a target RNA. In certain embodiments, the DR sequence is at the 3′ end of the spacer sequence.

In some embodiments, the polynucleotide composition is the guide RNA/crRNA of the subject Cas13e or Cas13f system, which does not include a tracrRNA.

In certain embodiments, for use with Cas13c, Cas13d, Cas13e, and Cas13f effector proteins, homologs, orthologs, derivatives, fusions, conjugates, or functional fragments thereof having RNase activity, the spacer sequence is at least about 10 nucleotides, or between 10-60, 15-50, 20-50, 25-40, 25-50, or 19-50 nucleotides. In certain embodiments, for use with Cas13c, Cas13d, Cas13e, and Cas13f effector proteins, homologs, orthologs, derivatives, fusion, conjugates, or functional fragments thereof having no RNase activity but ability to bind guide RNA and a target RNA complementary to the guide RNA, the spacer sequence is at least about 10 nucleotides, or between about 10-200, 15-180, 20-150, 25-125, 30-110, 35-100, 40-80, 45-60, 50-55, or about 50 nucleotides.

In certain embodiments, the DR sequence is between 15-36, 20-36, 22-36, or about 36 nucleotides. In certain embodiments, the DR sequence in the guide RNA has substantially the same secondary structure (including stems, bulges, and loop) as the RNA version of any one of SEQ ID NOs: 19-24 and 26-34.

In certain embodiments, the guide RNA is about 36 nucleotides longer than any of the spacer sequence lengths above, such as between 45-96, 55-86, 60-86, 62-86, or 63-86 nucleotides.

In a sixth aspect, the invention provides an isolated polynucleotide comprising: (i) a polynucleotide encoding any one of the Cas13c, Cas13d, Cas13e, and Cas13f effector proteins of SEQ ID NOs: 2-7 and 9-17, or orthologs, homologs, derivatives, functional fragments, fusions thereof; (ii) a polynucleotide of any one of SEQ ID NOs: 19-24 and 26-34; or (iii) a polynucleotide comprising (i) and (ii).

In some embodiments, the polynucleotide is not naturally occurring/naturally existing, such as excluding SEQ ID NOs: 75-89.

In some embodiments, the polynucleotide is codon-optimized for expression in a prokaryote. In some embodiments, the polynucleotide is codon-optimized for expression in a eukaryote, such as in human or human cell.

In a seventh aspect, the invention provides a vector comprising or encompassing any of the polynucleotide of the sixth aspect. The vector can be a cloning vector, or an expression vector. The vector can be a plasmid, phagemid, or cosmid, just to name a few. In certain embodiments, the vector can be used to express the polynucleotide in a mammalian cell, such as a human cell, any one of the Cas13c, Cas13d, Cas13e, and Cas13f effector proteins of SEQ ID NOs: 2-7 and 9-17, or orthologs, homologs, derivatives, functional fragments, fusions thereof; or any of the polynucleotide of the 4th aspect; or any of the complex of the 5th aspect.

In an eighth aspect, the invention provides a host cell comprising any of the polynucleotide of the 4th or 6th aspect, and/or the vector of the 7th aspect of the invention. The host cell can be a prokaryote such as E. coli, or a cell from a eukaryote such as yeast, insect, plant, animal (e.g., mammal including human and mouse). The host cell can be isolated primary cell (such as bone marrow cells for ex vivo therapy), or established cell lines such as tumor cell lines, 293T cells, or stem cells, iPCs, etc.

In a related aspect, the invention provides a eukaryotic cell comprising a Clustered Regularly Interspaced Short Palindromic Repeat (CRISPR)-Cas complex, said CRISPR-Cas complex comprising: (1) an RNA guide sequence comprising a spacer sequence capable of hybridizing to a target RNA, and a direct repeat (DR) sequence 3′ to the spacer sequence; and, (2) a CRISPR-associated protein (Cas) having an amino acid sequence of any one of SEQ ID NOs: 2-7 and 9-17, or a derivative or functional fragment of said Cas; wherein the Cas, the derivative, and the functional fragment of said Cas, are capable of (i) binding to the RNA guide sequence and (ii) targeting the target RNA.

In a ninth aspect, the invention provides a composition comprising: (i) a first (protein) composition selected from any one of the Cas13c, Cas13d, Cas13e, and Cas13f effector proteins of SEQ ID NOs: 2-7 and 9-17, or orthologs, homologs, derivatives, conjugates, functional fragments, fusions thereof; and (ii) a second (nucleotide) composition comprising an RNA encompassing a guide RNA/crRNA, particularly a spacer sequence, or a coding sequence for the same. The guide RNA may comprise a DR sequence, and a spacer sequence which can complement or hybridize with a target RNA. The guide RNA can form a complex with the first (protein) composition of (i). In some embodiment, the DR sequence can be the polynucleotide of the 4th aspect of the invention. In some embodiment, the DR sequence can be at the 3′-end of the guide RNA. In some embodiments, the composition (such as (i) and/or (ii)) is non-naturally occurring or modified from a naturally occurring composition. In some embodiments, at least a component of the composition is non-naturally occurring or modified from a naturally occurring component of the composition. In some embodiments, the target sequence is an RNA from a prokaryote or a eukaryote, such as a non-naturally existing RNA. The target RNA may be present inside a cell, such as in the cytosol or inside an organelle. In some embodiments, the protein composition may have an NLS that can be located at its N- or C-terminal, or internally.

In a tenth aspect, the invention provides a composition comprising one or more vectors of the 7th aspect of the invention, said one or more vectors comprise: (i) a first polynucleotide that encodes any one of the Cas13c, Cas13d, Cas13e, and Cas13f effector proteins of SEQ ID NOs: 2-7 and 9-17, or orthologs, homologs, derivatives, functional fragments, fusions thereof; optionally operably linked to a first regulatory element; and (ii) a second polynucleotide that encodes a guide RNA of the invention; optionally operably linked to a second regulatory element. The first and the second polynucleotides can be on different vectors, or on the same vector. The guide RNA can form a complex with the protein product encoded by the first polynucleotide, and comprises a DR sequence (such as any one of the 4th aspect) and a spacer sequence that can bind to/complement with a target RNA. In some embodiments, the first regulatory element is a promoter, such as an inducible promoter. In some embodiments, the second regulatory element is a promoter, such as an inducible promoter. In some embodiments, the composition (such as (i) and/or (ii)) is non-naturally occurring or modified from a naturally occurring composition. In some embodiments, at least a component of the composition is non-naturally occurring or modified from a naturally occurring component of the composition. In some embodiments, the target sequence is an RNA from a prokaryote or a eukaryote, such as a non-naturally existing RNA. The target RNA may be present inside a cell, such as in the cytosol or inside an organelle. In some embodiments, the protein composition may have an NLS that can be located at its N- or C-terminal, or internally.

In some embodiments, the vector is a plasmid. In some embodiment, the vector is a viral vector based on a retrovirus, a replication incompetent retrovirus, adenovirus, replication incompetent adenovirus, or AAV. In some embodiments, the vector can self-replicate in a host cell (e.g., having a bacterial replication origin sequence). In some embodiments, the vector can integrate into a host genome and be replicated therewith. In some embodiment, the vector is a cloning vector. In some embodiment, the vector is an expression vector.

The invention further provides a delivery composition for delivering any of the Cas13c, Cas13d, Cas13e, and Cas13f effector proteins of SEQ ID NOs: 2-7 and 9-17, or orthologs, homologs, derivatives, conjugates, functional fragments, fusions thereof of the 1st-3rd aspects of the invention; the polynucleotide of the 4th and/or 6th aspect of the invention; the complex of the 5th aspect of the invention; the vector of the 7th aspect of the invention; the cell of the 8th aspect of the invention, and the composition of the 9th and/or 10th aspects of the invention. The delivery can be through any one known in the art, such as transfection, lipofection, electroporation, gene gun, microinjection, sonication, calcium phosphate transfection, cation transfection, viral vector delivery, etc., using vehicles such as liposome(s), nanoparticle(s), exosome(s), microvesicle(s), a gene-gun or one or more viral vector(s).

The invention further provides a kit comprising any one or more of the following: any of the Cas13c, Cas13d, Cas13e, and Cas13f effector proteins of SEQ ID NOs: 2-7 and 9-17, or orthologs, homologs, derivatives, conjugates, functional fragments, fusions thereof of the 1st-3rd aspects of the invention; the polynucleotide of the 4th and/or 6th aspect of the invention; the complex of the 5th aspect of the invention; the vector of the 7th aspect of the invention; the cell of the 8th aspect of the invention, and the composition of the 9th and/or 10th aspects of the invention. In some embodiments, the kit may further comprise an instruction for how to use the kit components, and/or how to obtain additional components from 3rd party for use with the kit components. Any component of the kit can be stored in any suitable container.

With the inventions generally described herein above, more detailed descriptions for the various aspects of the invention are provided in separate sections below. However, it should be understood that, for simplicity and to reduce redundancy, certain embodiments of the invention are only described under one section or only described in the claims or examples. Thus it should also be understood that any one embodiment of the invention, including those described only under one aspect, section, or only in the claims or examples, can be combined with any other embodiment of the invention, unless specifically disclaimed or the combination is improper.

### 2. Novel Class 2, Type VI CRISPR RNA-Guided RNases, and Derivatives Thereof

In one aspect, the invention described herein provides two novel families of CRISPR Class 2, type VI effectors having two strictly conserved RX4-6H (RXXXXH) motifs, characteristic of Higher Eukaryotes and Prokaryotes Nucleotide-binding (HEPN) domains. Similar CRISPR Class 2, type VI effectors that contain two HEPN domains have been previously characterized and include, for example, CRISPR Cas13a (C2c2), Cas13b, Cas13c (Type VI-C), and Cas13d (Type VI-D).

HEPN domains have been shown to be RNase domains and confer the ability to bind to and cleave target RNA molecule. The target RNA may be any suitable form of RNA, including but not limited to mRNA, tRNA, ribosomal RNA, non-coding RNA, lncRNA (long non-coding RNA), and nuclear RNA. For example, in some embodiments, the Cas proteins recognize and cleave RNA targets located on the coding strand of open reading frames (ORFs).

In one embodiment, the disclosure provides additional members of CRISPR Class 2, type VI effectors, referred to herein generally as Type VI-C, Type VI-D, Type VI-E and VI-F CRISPR-Cas effector proteins, Cas13c, Cas13d, Cas13e or Cas13f. Direct comparison of the these newly identified CRISPR-Cas effector proteins with the previously identified effectors of these systems shows that the subject CRISPR-Cas effector proteins are significantly smaller (e.g., about 20% fewer amino acids) than even the previously identified Type VI-D/Cas13d effectors, and have less than 30% sequence similarity in one to one sequence alignments to other previously described effector proteins, including the phylogenetically closest relatives Cas13b.

These newly-identified CRISPR Class 2, type VI effectors can be used in a variety of applications, and are particularly suitable for therapeutic applications since they are significantly smaller than other effectors (e.g., existing CRISPR Cas13a, Cas13b, Cas13c, and Cas13d effectors) which allows for the packaging of the nucleic acids encoding the effectors and their guide RNA coding sequences into delivery systems having size limitations, such as the AAV vectors. Further, the lack of detectable collateral/non-specific RNase activity at selected range of spacer sequence lengths (such as about 30 nucleotides), upon activation of the specific RNase activity, makes these Cas effectors less prong to (if not immune from) potentially dangerous generalized off-target RNA digestion in target cells that are desirably not destroyed. On the other hand, at other selected spacer lengths such as about 30 nucleotides, significant collateral RNase activity exists for these Cas effectors, thus the subject Cas effectors can also be used in utilities depending on such collateral RNase activity.

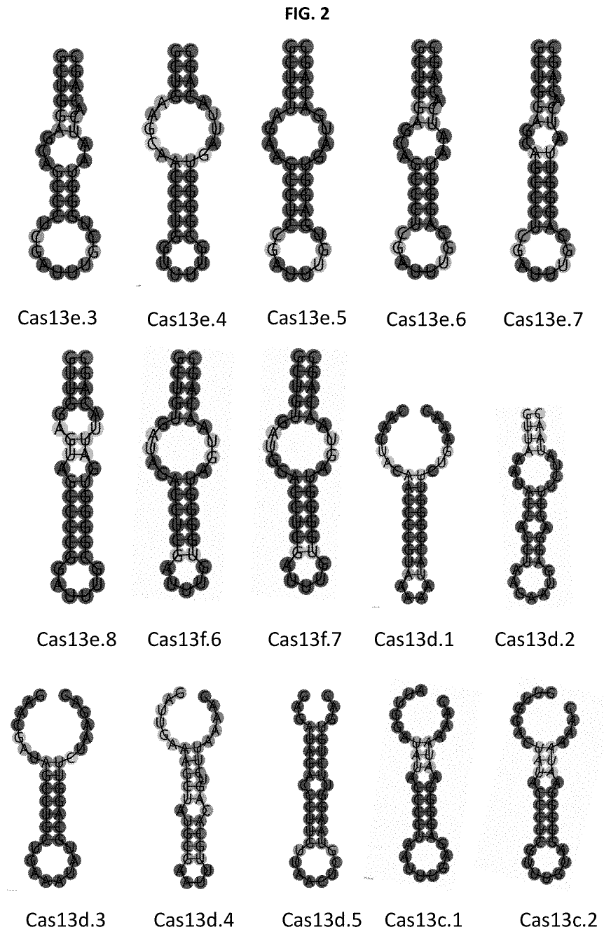

In bacteria, these CRISPR-Cas systems include a single effector (approximately 775 residues—under 900 residues) within close proximity to a CRISPR array. The CRISPR array includes direct repeat (DR) sequences typically 36 nucleotides in length, which are generally well conserved, both in sequences and secondary structures. Exemplary DR sequences for the new Cas13 proteins were provided in FIG. 2.

Data provided herein demonstrated that the crRNA is processed from the 5′-end, such that the DR sequences end up at the 3′-end of the mature crRNA.

The spacers contained in the Cas13c, Cas13d, Cas13e, and Cas13f CRISPR arrays are most commonly 30 nucleotides in length, with the majority of variation in length contained in the range of 29 to 30 nucleotides. However, a wide range of spacer length may be tolerated. For example, for use in a functional Cas13c, Cas13d, Cas13e, and Cas13f effector protein, or homologs, orthologs, derivatives, fusions, conjugates, or functional fragment thereof, the spacer can be between 10-60 nucleotides, 20-50 nucleotides, 25-45 nucleotides, 25-35 nucleotides, or about 27, 28, 29, 30, 31, 32, or 33 nucleotides. For use in dCas version of any of the above, however, the spacer can be between 10-200 nucleotides, 20-150 nucleotides, 25-100 nucleotides, 25-85 nucleotides, 35-75 nucleotides, 45-60 nucleotides, or about 46, 47, 48, 49, 50, 51, 52, 53, 54, or 55 nucleotides.

Exemplary Type VI-C, VI-D, VI-E, and VI-F CRISPR-Cas effector proteins are provided in the table below.

| Cas13e.1 | MAQVSKQTSKKRELSIDEYQGARKWCFTIAFNKALVNRDKNDGLFVESLLRHEKYSKHDWY |
|---|---|
| | DEDTRALIKCSTQAANAKAEALRNYFSHYRHSPGCLTFTAEDELRTIMERAYERAIFECRR |
| | RETEVIIEFPSLFEGDRITTAGVVFFVSFFVERRVLDRLYGAVSGLKKNEGQYKLTRKALS |
| | MYCLKDSRFTKAWDKRVLLFRDILAQLGRIPAEAYEYYHGEQGDKKRANDNEGTNPKRHKD |
| | KFIEFALHYLEAQHSEICFGRRHIVREEAGAGDEHKKHRTKGKVVVDFSKKDEDQSYYISK |
| | NNVIVRIDKNAGPRSYRMGLNELKYLVLLSLQGKGDDAIAKLYRYRQHVENILDVVKVTDK |
| | DNHVFLPRFVLEQHGIGRKAFKQRIDGRVKHVRGVWEKKKAATNEMTLHEKARDILQYVNE |
| | NCTRSFNPGEYNRLLVCLVGKDVENFQAGLKRLQLAERIDGRVYSIFAQTSTINEMHQVVC |
| | DQILNRLCRIGDQKLYDYVGLGKKDEIDYKQKVAWFKEHISIRRGFLRKKFWYDSKKGFAK |
| | LVEEHLESGGGQRDVGLDKKYYHIDAIGRFEGANPALYETLARDRLCLMMAQYFLGSVRKE |
| | LGNKIVWSNDSIELPVEGSVGNEKSIVFSVSDYGKLYVLDDAEFLGRICEYFMPHEKGKIR |
| | YHTVYEKGFRAYNDLQKKCVEAVLAFEEKVVKAKKMSEKEGAHYIDFREILAQTMCKEAEK |
| | TAVNKVRRAFFHHHLKFVIDEFGLFSDVMKKYGIEKEWKFPVK* (SEQ ID NO: 1) |
| Cas13e.3 | MAVDYSLKNEWYREINKSCFTVALNVAYDNCKAKGHENLLREAQRSKGGITNEQIKNVQTE |
| | IKTRLEDIRSHFSHFYHDEKSLIFEKDNIVKDFLESAYEKAQSSVIGSTRQSDYKGVVPPL |
| | FEPHDGMITAAGVVFLASFFCHRSNVYRMLGAVKGFKHTGKEELSDGAKRDYGFTRRLMAH |
| | YSLRDSYVIKAEETKSFRDLLGYLSRVPQQAVDWLNEHNQLSEDEKKEFLNQKPSDEESQE |
| | QSKTENTDRQADRMPRRSLRKTDKFILFAAKFIEDWAQKEKMDVTFARYQKTVTEDENKNQ |
| | DGKQVRDVQLKYEKDTKKLNPDFDYKWTYYIRNNHAIIQIKPDEYKQAVSARISENELKYL |
| | VLLIFQGKGWEAIKKIGDYIFHIGNKIKIGRFDHNEERRMPSFLKNPPADIIGEMVENRLK |
| | YIRDELNKVIETIKKEEPQNNKWLLYKGKKISIILKFISDSISDIKKRPDVNEYNTLRDML |
| | QKLDFDNFYERLKSYVSEGRIEQTLYDEIKGIKDISTLCIKICELRLAALEELEKEGGDDL |
| | NKYIGLAVQEKHKNYDDSNTPQKKAERFLESQFSVGKNFLRETFYDEYIKNRKSLYEIIKE |
| | KITGITPLNENRWYLMDKNPKEFESKDSKIIRGLCNIYIQDILCMKIALWYYENLSPSYKN |
| | KLKWDFIGQGFGYDRYKLSYKTDCGITIEFKLADLNRLDIIEKPKMIENICHSFILEKDVK |
| | KQTISWHEFRQDGIAKYRKLQKEVVEAVFEFENSLKIPDKNWLTQGYVPFNKNKRFEDKGF |
| | STFILEEAVRKGKIKSDDKEPLRKVRTDFFHEQFDSTDAERRIFDKYMPAKHDGKNKGGKM |
| | QEKQEKSYTRRI* (SEQ ID NO: 2) |
| Cas13e.4 | MEKEQGLYSIDRYQGAGKWCFAIGANRAWDNYNERPKLFSESLLRYEKATRRDWFDEETRG |
| | LIKKSDVRQRLRKIRCYFSHYCHDNTCLGFDPDDDLRKIMEKAYERAIFEQRKHLSTETDI |
| | ETPALFEPHGRITAAGVVFFCSFFVERRILNRLMGRIPGFKKTEGEYGATRQMFSKYCLRD |
| | SYSIRASDSNAVLFRDILGYLSRAPSQYYRHNKDQCDKDGHPERKKDKFINLALRYLESFV |
| | PARLRNHTLSVGRKEVVRMETNAVAEGEGEYRPYPPKAKVKVVFTEDDPERPYYITHNTVI |
| | LQTAKKEEDIHHCKVGVNELKYLVLLCLQGKAEKAVAGIEGYVRRIQGRFADHTNKVARDD |
| | DERLVRGLPEFVRVASGIETPDEVRELKSRLDHIRKKWQTKKAESAEAQLHRKARDVLRHI |
| | NWESQRPLGIEQYNRLLELLVNRDLESFAAEMKELKRRGLISEELLKSVEGIRNLNTLHVK |
| | VCNLVLTRLEHLVENDPEELKRHIGIVPREEKEGPSYEEKVRAFVQQPMMYRGFLRNSFFK |
| | GSGKSFAKLVEEELHKKGCPDVPLGTDYYLVRDLERDERKNRFHNDNAALYETLALDRLCV |
| | LMARDCLVRLNRNLEKHATRISWEATDAGDTICLELPRRDRDHESFRLSFGVRDYPKLYVM |
| | DDPVFLCGLMKHFFPDNQAIQYHELYSEGINKYTAMQAEGIAATLKLEEKTIKEKNMQIPA |
| | TGYIRFCEIVSQSDFAPGEKRVLKNVRNGLLHYHLEFEPTEWAEFREIMKREGFDTAKKRK |
| | STRKK* (SEQ ID NO: 3) |
| Cas13e.5 | MKIKNENTDKKTELYSIDKYKGRDKWCFAIVLNKAQTNLTENPDLFEQTITKYDRIRKEGW |
| | FDEETKKLIYIQENEHKIKGEIKTLAREVLKNLRNYFSHHFYKQDCLIFPKDNIVRIIMGR |
| | AYERSEYEIKKNLKEDISIELPALFEPEGKITTAGIVFFTSFFVERRFLHRLMGSVQGFTK |
| | TEGEYKITRDVFAKYCLRDSYSVKAQDNDAVMFRDIIGYLSRVPTESFQHIKNPKKQNESQ |
| | LSERKTDKFISFALKYLKDYGFEDLKGHYTAFFARSEIKKEKEDIEIKDDKKHKPHRMKSK |
| | IEIHFDKTKEDRFYIERNNVILKIQRKGGRANILRMGIYELKYLVLLCLSGKAREAINRID |
| | DYLNDLRNKIPHIENMNKEGIGEQIRSLPGFVRSQLGFVQIDDEKKKENRLDYVEKKWEKK |
| | RAESKELKLNRKGRDILRYINERCKKPLTIDRYNRILELLVEKNIEGFYHELEELRKTGRI |
| | EKNITQALVGEKNINALHIKICKLVQDELKSLEKEDLKKYIGLTPKEEKVVSFEEKLGRIL |
| | DKPVIYKGFLRYQFFKNDKKSFARLVEEIIKEKTGGLDVPIETEYYSISTLGRFDKANKTL |
| | YETLAMDRLCMMMARRYFLSLNKILAKRAQNIEWKKESGKEFIVFKFNMPQDTGKSISIRF |
| | SPKDYTKLYVKNDSEFLARLCQYFFPNEKAIDYHKLYSHGINKYTNFQKEGIEAILELEEK |
| | IIKKRKINSPENYLSFEEILNQSIYNDEEKNTLIQIRHSLLHYQILFSKNDLTKFYNVMKR |
| | EGIEKIWSLVI* (SEQ ID NO: 4) |
| Cas13e.6 | MAVNYSLREKWYRGVNKCCFTVALNIAVDNCKSKGCETLLKEAEHSKGGITDEQIQQSYTE |
| | VEKRLNDIRNYFSHFYHGDECLIFKKDDIVKRFMESVFATAVSNVVGGTKESDYKGVVPPL |
| | FEQSNEDYMITAAGVIFLASFFCHRSNVYRMLGAVKGFKHTGKEELSDGQKRDHGFTHRLL |
| | AHYSLRDSYSVKIEETKSFRDLLGYLSRVPQQAVDWLNERNELSEDEKKEFLNQKSSEEES |
| | PEQPEPENAEWRTEKTSRRSLRKTEKFILFAAKFIEDRAEKEKQDVTFARYQKTVTKEENK |
| | NQDGKQARVVRLKYEEDKKDDEKPREHFNLEWMYYIRNEHAIIQIKPKDKEAVAARISENE |
| | LKYLVLLIFEGKGGDAFNKLSDYIFRMTQKIKSGQINPNEARLPSFLKNPVKNITDKMVRN |
| | RLDYIRGQIKDVLEKINMEEPQNNKWLIYKGKKISLVLKFISDGISDIKKRPNVKEYDTLR |
| | DTLQKLDFNRFYERLKSYVSDGRLAAALYDKIKGIDDISELCKKVCELMFARLAELEKKGG |
| | FELYRYIGMEVQEKDEKYDEWNSPQKKAERFLESQFSIGENFLRESFYSEYCQKQECIDKE |
| | ISLNTSVKNRKSLVYIVKEKLKDIMPLHNDRWYLIDRNPKDFERKDSKVIKGLCNTYVQDV |
| | LCMKMARWYYGQLNPALKNNIKWDETGQGHGYDRYKLSYRTNFGITIEFKLADFTRLDIIE |
| | KSDMIENICRSFIKPNRTISWYDFKQDGVEEYRKRQYKAVRAVFAFEESLIIPGRDWLSQG |
| | FVPFIKNEEYVKKGFSLFVLDEAVRQLKIKGSDKDAMRQVRNDFFHEQFQAKDEQWKVFEG |
| | YLSCFMIDRPKGEKNKKRYNGNKK* (SEQ ID NO: 5) |
| Cas13e.7 | MEKYLIKNFEGINKSKFTVALNIANDNCKNKGIQELLKEAQRSKGGITDTQITEVQEHIKE |
| | RLNSVRNYFSHCYHEKKPLYFEANDPVKIFLEETFAKAVENLQGRFLSDKYKLTVPPLFEP |
| | NQNNTITAAGVIFLASFFCHRSYVYRMLGGIPGFKRSDKKKWGDGQKIDYGFTRKLMSFYS |
| | LRDSYSVNVQENKELTAFRDILGYLARVPGQAIDWLIEKGKLTKEEGKQFYLGEQSEEREE |
| | KAKKEEIKYALRKTDKFMLFAVRFIEDWAEQERIKVEFARYEKMTIVNENKKQDEKEERKV |
| | KFVSDEPTAAGWTYYIRNNHAIIKIIPDDKKKKAVSARISENELKYLVLTIIDGNGKNAIA |
| | YIGDYIFRTARQIENKSYNAESEKYAPAFVRGGQKKSVDKRIKYIRDEIQQVINDIEAEQE |
| | KQKNEQDAPAENRTWLIYKGKKISIILRYVNDNIAEYKKRLSVTEYNELRGYLQQLDFINF |
| | HRKLAEYQHHGRLPNGFAESINKFQDLSKLCIEVCERQKKKLQEMAAKGGIELEQYIGLAP |
| | KEENQEQNKYATKANNFIKVWLSIPENFLRQKFYDKFCKQQECKNKGSDKPDNTSVPQRKY |
| | FIAIIREKNIRPIHADKYYLLGQNPKDYERPDGKIIRQLCDVYCKDGLCMAMAKWYYENRL |
| | GKFKDLIEWQTGDDKQQHGYAGHTLEYQATEKIKIRFKLADFTRLDIIEPPERVKNICRQW |
| | ETELLKKTRDGTISWYDFKLNGLEPYRQWQGYAVADIFWFEESLKINETQWQGRTHMPFNF |
| | EKDKPLWCNILDEAVKQNKIEKQDTQALRRVRHDCFHEEFLANYEQLKIFKNLISDKAKDA |
| | KPKDKKSRKNEQKYGKR* (SEQ ID NO: 6) |
| Cas13e.8 | METTEEKKEIYSIDKYQGAGKWCFAIILNRARDNYEGNPHLLSESLLRFEAVNRRDWFDEE |
| | TRELIIQMDAETKTDSNLKPSTPAYKILIGIRNYFSHFYHTEECLYFKEDDPIRIILENAY |
| | KKAKQHHIERLEKETDIEFPALFESNDRITSAGIIFFSSLFVERRILNRLMGYVGGFKKTK |
| | GEYNITREIFSTYCLRDSYSIMAADSNAVIFRDILGYLSRVPSEYYQHNKEKCEKENKPKR |
| | KTKKFIYFALRYLEKFVLKEIKNQKVSIARMEVVREKSKEAEGEDEQHKPYPDKAKVKIVF |
| | DSIGKELPYYINHKTVIMKIQRNGEAVNFCKIGINELKYLVLLCLQGKSVDAIAKINGYID |
| | RIKKRLENPKTRIDIDSERDNEFIKGLPEFVKIQSGRTPDEEREKKSRIDYIRKKWEKKKE |
| | ESPKTELHRKGRDILRYINWHCEPPLGSEEYNHLLSLLVNKDLDGFENELKELKRTEQISK |
| | KLLEMLKGFSNLNELHLKVCSIVPVELVYLEKNDPEKLAEYIGLIPQKTDKEPPSYEEKVK |
| | NFIKQPMIYKGFLRDTFFSSGKTFAKLVEETFLHKYPHSDVPLGRDYYHVTTLDRFHKDNS |
| | ILYETLALDRLCVVMARKFHETLNQELAKESKQIVWENNTIILELPRSKTSSSDTFQICFD |
| | IKHYMKLYVMDDVEFLGGLMRHFFQKEKTIEYYNLYSFGINKYTEMQRNGIEAILRLEEKI |
| | IREKNIKRKPDKNYISFYEIMEASNYPDNDKKTLNKVRRALLHYHLKFEPADYNKFVAIMK |
| | REGLERKKKTGKATRRWVKK* (SEQ ID NO: 7) |
| Cas13f.1 | MNGIELKKEEAAFYFNQAELNLKAIEDNIFDKERRKTLLNNPQILAKMENFIFNFRDVTKN |
| | AKGEIDCLLLKLRELRNFYSHYVHKRDVRELSKGEKPILEKYYQFAIESTGSENVKLEIIE |
| | NDAWLADAGVLFFLCIFLKKSQANKLISGISGFKRNDDTGQPRRNLFTYFSIREGYKVVPE |
| | MQKHFLLFSLVNHLSNQDDYIEKAHQPYDIGEGLFFHRIASTFLNISGILRNMKFYTYQSK |
| | RLVEQRGELKREKDIFAWEEPFQGNSYFEINGHKGVIGEDELKELCYAFLIGNQDANKVEG |
| | RITQFLEKFRNANSVQQVKDDEMLKPEYFPANYFAESGVGRIKDRVLNRLNKAIKSNKAKK |
| | GEIIAYDKMREVMAFINNSLPVDEKLKPKDYKRYLGMVRFWDREKDNIKREFETKEWSKYL |
| | PSNFWTAKNLERVYGLAREKNAELFNKLKADVEKMDERELEKYQKINDAKDLANLRRLASD |
| | FGVKWEEKDWDEYSGQIKKQITDSQKLTIMKQRITAGLKKKHGIENLNLRITIDINKSRKA |
| | VLNRIAIPRGFVKRHILGWQESEKVSKKIREAECEILLSKEYEELSKQFFQSKDYDKMTRI |
| | NGLYEKNKLIALMAVYLMGQLRILFKEHTKLDDITKTTVDFKISDKVTVKIPFSNYPSLVY |
| | TMSSKYVDNIGNYGFSNKDKDKPILGKIDVIEKQRMEFIKEVLGFEKYLFDDKIIDKSKFA |
| | DTATHISFAEIVEELVEKGWDKDRLTKLKDARNKALHGEILTGTSFDETKSLINELKK* |
| | * (SEQ ID NO: 8) |
| Cas13f.6 | MENIKLEKQKAAFYFNQAELNLKAIEGNIFDKGRRKTLFDNPKILSKVENFIFNFKDVTKN |
| | AKGEIDCLLSKLMELRNFYSHYVHKPDVKELSKGEKPLLERYYQIAIEATGSENVKLEIIE |
| | NDKWLTDAGVLLFLCMFLKKSQANKLISGISGFKRNDTFGQPRRNLFNYFSVRERYKVVPD |
| | MQKHFLLFVLVNHLSEQDDYIEKAQQPYNIGEGLFFHRIASTFLNVSGILRNMEFYTYQSK |
| | RLKEQRGELKREKDIFTWEEPFQGNSYFEINGHKGVIGEDELKELCYALLSYNKSKYAVEQ |
| | IEKFLKGFGEVKSEQEIRDSDILNESYFPTNYFAESNIGSIKEKILNRLGKTDDSYKKTGT |
| | KIKPYDMMKEVMEFINNSLPADEKLKRKDYRRYLKMVRIWDSEKDNIKREFESKEWSKYFS |
| | SNFWMAKNLERVYGLAREKNAELFNKLKAVVEKMDEREFEKYRQINSAEDLASLRRLANDY |
| | GVKWEEKDWQEYSGQIKKQISDRQKLTIMKQRITAELKKKHGIENLNLRITIDSNKSRKAV |
| | LNRIAVPRGFVKEHILGWQGSEKVSKKTREAKCKILLSKEYEELSKQFFQTRNYDKMTQVN |
| | SLYEKNKLIAFMAVYLMGQLNIRFDKPTRLNELEKAEVDFKISDKVTAKIPFSQYPSLVYA |
| | MSSKYADSVGSYKFENDEKNKPFLGKIDIIEKQRMEFIKEVLGFEEYLFEKKIIDKSKFAD |
| | TATHISFREICDELIQKGWDENKLTNLKDARNAALHGEIPAETSFREAKPLINGLKK* |
| | (SEQ ID NO: 9) |
| Cas13f.7 | MINIELKKEEAAFYFNQANLNISGLDEVIEKQLPHIGSKKENAKKAIDKIFDNITVLKKVE |
| | NFVFYFKDVAKNERVELDALLLKLIDLRNFYSHYVHNDNVKILSDGEETLLEKYYQIAIEA |
| | TGSKDVKLEIIDNEKKLTDAGILFLLCMFLKKSQANKLISSISGFKRNDKEWQPRRNLFTY |
| | YSLREGYKVVPDMEKHFLLFTLVNHLSTQDENIENTQPSDDIGRGLFFHRIASTFLNISGI |
| | FNNMEFYPYQSNRLKERRGDIAPDKDSFAWIEPFQGNSYFKINGYKGVIGENELKELCFAV |
| | LLHNKSKYAVEQIEKFLKCFKEVQSKQEIIECDILDECYFPANYLNQPETKSLKEKLLSRI |
| | TGKINYSFDTAEKAFDKMKDVMEFINGCLPSDEKLKRKDYSRYLKMVRFWGGEKDNIKREF |
| | EGKKWTRFFPSELWHKRTLEDVYKFALKKNKKRLEELKVKIEGLNEDDLLKYQKVNNIKNL |
| | ENLRLLAHDLDLSWREKDWGEYSGQIKKQISDNQKLTIMKQRVIAELKKKHGIENINLRIS |
| | LDSNKSIQAVLNRIAIPKGFIKRHVLHLQENEKTSRKIREAKCKILLSKKYEYLSRKFLDE |
| | KNFDKLTQINGLYEKNRLIAFMVIYLLKQLGLELKNETKLIELKKTRVKYKISDKVAEDIP |
| | LSHYPSLVYAMSRKYVDNIDNYEFPDEYAKKAILDKVDIIENQRMEFIKQVLGFEKYLFDN |
| | NIIDKSKFTDVETHISFVKIHDELIEKGWDTEKLSKLKHARNKALHGEIPGGTSFEKAKLL |
| | INELKK* (SEQ ID NO: 10) |
| Cas13d.1 | MKKQKSKKTVSKTSGLKEALSVQGTVIMTSFGKGNMANLSYKIPSSQKPQNLNSSAGLKNV |
| | EVSGKKIKFQGRHPKIATTDNPLFKPQPGMDLLCLKDKLEMHYFGKTFDDNIHIQLIYQIL |
| | DIEKILAVHVNNIVFTLDNVLHPQKEELTEDFIGAGGWRINLDYQTLRGQTNKYDRFKNYI |
| | KRKELLYFGEAFYHENERRYEEDIFAILTLLSALRQFCFHSDLSSDESDHVNSFWLYQLED |
| | QLSDEFKETLSILWEEVTERIDSEFLKTNTVNLHILCHVFPKESKETIVRAYYEFLIKKSF |
| | KNMGFSIKKLREIMLEQSDLKSFKEDKYNSVRAKLYKLFDFIITYYYDHHAFEKEALVSSL |
| | RSSLTEENKEEIYIKTARTLASALGADFKKAAADVNAKNIRDYQKKANDYRISFEDIKIGN |
| | TGIGYFSELIYMLTLLLDGKEINDLLTTLINKFDNIISFIDILKKLNLEFKFKPEYADFFN |
| | MTNCRYTLEELRVINSIARMQKPSADARKIMYRDALRILGMDNRPDEEIDRELERTMPVGA |
| | DGKFIKGKQGFRNFIASNVIESSRFHYLVRYNNPHKTRTLVKNPNVVKFVLEGIPETQIKR |
| | YFDVCKGQEIPPTSDKSAQIDVLARIISSVDYKIFEDVPQSAKINKDDPSRNFSDALKKQR |
| | YQAIVSLYLTVMYLITKNLVYVNSRYVIAFHCLERDAFLHGVTLPKMNKKIVYSQLTTHLL |
| | TDKNYTTYGHLKNQKGHRKWYVLVKNNLQNSDITAVSSFRNIVAHISVVRNSNEYISGIGE |
| | LHSYFELYHYLVQSMIAKNNWYDTSHQPKTAEYLNNLKKHHTYCKDFVKAYCIPFGYVVPR |
| | YKNLTINELFDRNNPNPEPKEEV* (SEQ ID NO: 11) |
| Cas13d.2 | MKKSKVKLNGVKAVYHISPDVRVIAAFGRGNNSVLDKHIENGSVEELQNHSDIEVNISRKT |
| | YSFRKKSLKKAAGQFSVPDNTNDQLGIREELEKEIFGRKFDDNIHIQAAYAVNDIIKMLAV |
| | AANLAETAINGLDRENTENDMIGFCIIPHITYQTYADDKRSKFEGFIEKVKTQGTFSYFPD |
| | ILPKFKKEESEEESDNEKLYYIMCIISLIRNSATHSKSSNSDTTDYIFGEFNSVNKEALTT |
| | TADNLIKSKIDFINKEGFSKNQKNNIYRLLKAKADTPENTARLIRRLYAFTIRKQDKNLGF |
| | SLKKLRECAIRSIDRSIEYMKYLPSKKYDTVRSKLYTLMDFVVYSYLKYHKDGKKFSKEMV |
| | EQLRAAESDKVKDDIYRDEAEKLYNIEIISRTINALISDIKSDFDQPKHGNECYQPINDGM |
| | KEAEKDFITTDQLSLFTKFIYVLCQFLDGKNINILLSSLISKFQQIEAFNGDIRKLNLNIR |
| | DDGKIGYDSKKYSIFEKSGQIADDLDKLRGVIKMDINDLNAYETMIKDALRVIGVDESDIE |
| | SIYQTHFKTQDKKDSVSGFFRNNIINSRRFRYIIKYINPSDAYRIIQNENVRNYVLGRMND |
| | AIIDRYAHSVGIEDKVHDKRKVLSDILSKVKFDNFTKLTYINPKDKNKGEKAKEREKPKAI |
| | LGLYLTIVYLVVKSLVRINSQYVMAVYHLERDSRLCPGVSSNNLLSMTNHYCDRDNHLLKE |
| | KHIVKLERYKKTQKTQKTICTAYRNAIAHLSAVRKGVKYIGDIQKADSYFGIYHYCMQKLI |
| | YSANGQPFAEFVRSIFGDEKELDKLRNGSYSQAILRALNYPFGYNPARYKNLSYEKIFIRA |
| | WQDEDTNKKT* (SEQ ID NO: 12) |
| Cas13d.3 | MNKIHKKQGKTTAKSLGLKSVLKIENDLVVTTFGKKDNPMVVEQSINKASGEKELYVDEDQ |
| | VKFDSSLIKEKNILSLDSIQHSNHQIIVNIDQKDASEIGMDYLRLKPELEKEFFGKTFYDN |
| | VHIQIAYNLLDLKKIIGLHIGNAIQALENLGRDGSDLVGICDATKPLNYLDDVKQKADIGF |
| | MNRLKPYFMYFDGVLKLDNSKNKNGELNQLDIENWDVIRILSLIRQGCAHAGAYSSLLYTA |
| | QNNKVYADLINKALSIFSDDLDKFNKSFLKQSKMNLFILFDLYNCRFDRSLQEKIIKEYYR |
| | YVLYKDNKNLGFSLKNVRNLIIEGKYDEQERSGKLQTIRSKLNTLLDFYLYGYYQKNPTFV |
| | ENIVAKLRESKNDEDKEKVYEEEYHRLLSENNYLVDKKCSDIVYRINEAVKNRKIFVNANI |
| | NAVVEKVSCSCFPSLIYVLCKFLDGKEVNELTTAIINKLENIASLINALVTLKSYGGFSEQ |
| | YKIFDYPNINGLIDDFRMVKNLTSTKRKLKKASGGEDRIGRQLYADAINIFKEDSFVSAND |
| | EKGTGLDQYVNKFFSKDDLGARKVRNLLLNNIIKNRRFVYLIKYIDPKDCYKLVHNEKIVR |
| | FALGQYDESQMPLNQLQKYYDAVIENREGFRKCNDRKKIIDTLVSEINRVSIDGILDIGNR |
| | LVNRGNNDYINHQKQIISLYLTIAYLIVKGVVHTNSLYFIAWHAYERDNNFKFGNDGKDYL |
| | ALTKEYLTNKKKRVKQLLDHNIEEANNSLDSKYFSAYRNKVVHLNFCNIFVNYLDGIGDIH |
| | SYYDIYQYVIQKWSIAERSKDFIDPQYLTKLSNDLKQYRTYQRNFLKIINLPFAYNLARYK |
| | NLTIGDLFNDKYPLPKETVKEFYNEE* (SEQ ID NO: 13) |
| Cas13d.4 | MAKKKRMSAKERKQQQINLRIKKATEDSTKKVNTTVAVNNKPISKEIKKSKAKLAGVKWVI |
| | KANDDVAYISSFGKGNNSVLEKRIIGDVSSDVNKDSHMYVNPKYTKKNYEIKNGFSSGSSL |
| | TTHPNKPDKNSGMDALCLKTYFEKEIFKDKFNDNMHIQAIYNIFDIEKTLAKHITNIIYAV |
| | NSLDRSYIQSGNDTIGFGLNFNIPYAEYGGGKDSNGKPENKSAWEKRESFIKFYNNAKDRF |
| | GYFESVFYQNGKQISEEKFYIYLNILNFVRNSTFHYNNTSSHLYKERYCKINPKNNLKTDF |
| | EFVSYLNEFVKNKFKNVNKNFISNEKNNLYIILNAYGEDIEDVEVVKKYSKELYKLSVLKT |
| | NKNLGVNVKKLRESAIEYGYCPLPYDKEKEVAKLSSIKHKLYKTYDFVITHYLNSNDKLLL |
| | EIVEALRLSKNDDKKENVYKIYAEKIFKAEYVINPIKTISNLFAEKGDKLFNEKVSISEEY |
| | VEDIRIDKNIHNFTKVIFFLTCFLDGKEINDLLTNIISKLQVIEDHNNVIKAIANNNDAVY |
| | KDYSDKYAVFKNSGKIATELEAIKSIARMENKINKAFKEPLLKDAMLALGVSPNDLDEKYE |
| | KYFKTDVDADKDHQKVSTFLMNNVINNSRFKYVVKYINPADINRLAKNKHLVKFVLDQIPH |
| | KQIDSYYNSVSTVEEPSYKGKIQLLTKKITGLNFYSLFENCKIPNVEKEKKKAVITLYFTI |
| | IYILVKNLVNINGLYTLALYFVERDGFFYKKICEKKDKKKTNKDVDYLLLPEIFSGSKYRE |
| | ETKNLKLPKEKDREIMKKYLPNDEDRKEYNKFFKQYRNNIVHLNIIANLSKLTSTIDKEIN |
| | SYFEIFHYCAQRVMFDYCKNNNKVVL* (SEQ ID NO: 14) |
| Cas13d.5 | MKKNSNDKTNAKRMGIKSFIKNGDERFITTSIKNEFPVELKLDVIKKTCEPAHEPVSFDYD |
| | PKKIDFEKPVLKEKLTSGQSGQKLSTRLFIQKDRDICGIRRKYLEKIFNSNFIEEKKDSNL |
| | PMQIVAKVLSTEKVFSNALNKIISQFLSMPRGGVTDNHGEYEIIGNIINHKSLQELNKEKK |
| | TKRIKKYLQSVIKNQSYLYNKQFLLSLDESKGSRNDIDENELYDYIRFLAILRNGIAHVFY |
| | EKNEPETAKESLFRLVDFIKNDKKLEGAFAKIKIQVNTLYKCRKEEYIKKSGKNFEIIRKI |
| | YQNDKPDEKVKDWIRYDFDKSYKYIGLSVAKLGNYTSWAKDIDNLRDKSNPDSGYAGIMHR |
| | LNEFSVYLKVKALSTEEKDKYLKNLISKENCEEKDKYYKNIAQFFCSSDLKFANVLQMVKE |
| | IKKNKGCTSEDKNCKLCVDERKFNDLSVIVYFISCFLDNKDQNIFLSDLINRFGALSDLLR |
| | IQNKILGAGNKYNENYSFLKNERYVTEIKMELETIFALVKVSYKKEDKAFNRLLEDGLVMF |
| | GFSKDEAGMKVAGLKEIKEKKEGHYKNKSRSFLINSIVNSRKFAYLAKSIDPQKVPAIIKN |
| | EHIVRYILGRINKTNPGQIGRYWRYIMSQNHAGTDKVDDLTNEIIKINIKNILNDAGGWQK |
| | SKLNDNNNKKKLKYQQLIGLYLTVAYIFVKNMLECNARYFSAFAQIEKDYLIYTNSDEFYY |
| | IDKNKKNLVTERYLKLVKDIIEKNKNTVRKDKIFRKKRQRKHLADISKSIIEFEKLPCCIF |
| | TLLRNITEHLNVASNIDIIEGYGKRAGKYHKNAPASYFIFYHYIIQKILADKICTRNLLNI |
| | INTYGEPSISFIKIIYVPFAYNLPRYLNLTDARIFCNMDDK* (SEQ ID NO: 15) |
| Cas13c.1 | MKKLKNPSNRNSLPSIIISKFDSSKIYEIKVKYEKLARLDRLEIGDMSLDENLNILFKKVN |
| | FNGIDLEILNPLLLDFDSYTISGKLQKNSTNKTILTLKKDGKIIKYNVLEKDNKYFKNGKE |
| | FVIPKDVKEEGKRLVNDKFLLTIEDKKREENSLPKKRKKETQRDILKDETIEIYKRISSNS |
| | NIKSEDIYRIKRYMLFRSDMMFFYTFIDNFFYCLYKNKNEQLWNTNFKEKENLGKFIEFTL |
| | NDTLKNPRNGILKSYSKDLKVVQEDFVKIKDIFEKIRHALAHFDFTFIDNLLSNNIEFDFN |
| | IKLLNIVIEDSQDLYYEAKKEFIEDEKMDILDEKDISIKKLYTFYSKIDIKKPAFNKLINS |
| | FLIKDGVENSKLKEYIKEKYNCHYFIDIHDNKEYKKIYNEHKKLISENQNLQLNSKENGQK |
| | IKINNDRLEELKGKMNELTKANSLKRLEFKLRLAFGFIKVEYNIFKDFKNNFSEDIKKDMN |
| | IDLEKIKSYLDTSYSNNQFFNYKVYNKKTKQKDIDKDIFDDIEKETLKELVENDSLLKIIL |
| | IFYIFTPKELKGEFLGFIKKFYHDTKNIDKNTKDKEEPLEQIKQEVPLKLKILEKNLTILT |
| | IFNYSISLNIEYDKNNNSFYERGNKFKKIYKDLKISHNQEEFDKSLLAPLLKYYMNLYKLL |
| | NDFEIYLLLKYKNKDNLNKESLNKLINDEQLKHNDHYNFTTLLSEYFNFDPKKNKKYETLT |
| | ILRNSISHQKIDNLIYNLDKNKILEQRVKIVELIKEQRDIKETLKFDPINDFTMKTVQLLK |
| | SLENQSEKRDKIEEILKQQDLSANDFYNIYKLKGVESIKKELFIRLGKTKIEEKIQEDIAK |
| | GSI* (SEQ ID NO: 16) |
| Cas13c.2 | MNSIEKIKKPSNRNSIPSIIISDYDENKIKEIKVKYLKLARLDKITIQDMEIRDNIVEFKK |
| | ILLNGIEHTIKDNQKIEFDNYEITAYVRASKQRRDGKITQAKYVVTITDKYLRDNEKEKRF |
| | KSTERELPNDTLLMRYKQISGFDTLTSKDIYKIKRYIDFKNEMLFYFQFIEEFFSPLLPKG |
| | TNFYSLNIEQNKDKVVKYIVYRLNDDFKNQSLNQFIKKTDTIKYDFLKIQKILSDFRHALA |
| | HFDFDFIQKFFDDELDKNRFDISTISLIKTMLQEKEEKYYQEKNNYIEDSDTLTLFDEKES |
| | NFSKIHNFYIKISQKKPAFNKLINSFLSKDGVPNEELKSYLATKKIDFFEDIHSNKEYKKI |
| | YIKHKNLVVEKQKEESQEKPNGQKLKNYNDELQKLKDEMNKITKQNSLNRLEVKLRLAFGF |
| | IANEYNYNFKNFNDKFTLDVKKEQKIKVFKNSSNEKLKEYFESTFIEKRFFHFCVKFFNKK |
| | TKKEETKQKNIFNLIENETLEELVKESPLLQIITLLYLFIPKELQGEFVGFILKIYHHTKN |
| | ITNDTKEDEKSIEDTQNSFSLKLKILAKNLRGLQLFNYSLSHNTLYNTKEHFFYEKGNRWQ |
| | SVYKSLEISHNQDEFDIHLVIPVIKYYINLNKLIGDFEIYALLTYADKNSITEKLSDITKR |
| | DDLKFRGYYNFSTLLFKTFMINTNYEQNQKSTQYIKQTRNDIAHQNIENMLKAFENNEIFA |
| | QREEIVNYLQKEHKMQEILHYNPINDFTMKTVQYLKSLNIHSQKESKIADIHKKESLVPND |
| | YYLIYKLKVIELLKQKVIEAIGETKDEEKIKNAIAKEEQIKKGYNK* (SEQ ID NO: |
| | 17) |

In the sequences above, the two RX₄₋₆H (RXXXXH) motifs in each effector are double-underlined. Mutations at one or both such domains may create an RNase dead version (or “dCas”) of the Cas13c, Cas13d, Cas13e, and Cas13f effector proteins, homologs, orthologs, fusions, conjugates, derivatives, or functional fragments thereof, while substantially maintaining their ability to bind the guide RNA and the target RNA complementary to the guide RNA.

The corresponding DR coding sequences for the Cas effectors are listed below:

(SEQ ID NO: 18)

| | |
|---|---|
| Cas13e.1 | GCTGGAGCAGCCCCCGATTTGTGGGGTGATTACAGC |

(SEQ ID NO: 19)

| | |
|---|---|
| Cas13e.3 | GCTGGAGCAGCCCTCGATTTGCTGGGTAATCACAGC |

(SEQ ID NO: 20)

| | |
|---|---|
| Cas13e.4 | GCTGAAGCAACCCTGGTTTTGCGGGGTGATTACAGC |

(SEQ ID NO: 21)

| | |
|---|---|
| Cas13e.5 | GCTGTAGAAGCCTCCGATTTGTGAGGTGATGACAGC |

(SEQ ID NO: 22)

| | |
|---|---|
| Cas13e.6 | GCTGGAGCAGCCCTCGATTTGCAGGGTAATCACAGC |

(SEQ ID NO: 23)

| | |
|---|---|
| Cas13e.7 | GCTGGAGCAGCCCTCGATTTGCAGGGTTATCACAGC |

(SEQ ID NO: 24)

| | |
|---|---|
| Cas13e.8 | GTTGGAGTAGCCCCGGATTTGCGGGGTGATTACAGC |

(SEQ ID NO: 25)

| | |
|---|---|
| Cas13f.1 | GCTGTGATAGACCTCGATTTGTGGGGTAGTAACAGC |

(SEQ ID NO: 26)

| | |
|---|---|
| Cas13f.6 | GCTGTGATAGACCTCGATTTGTGGGGTAGTAACAGC |

(SEQ ID NO: 27)

| | |
|---|---|
| Cas13f.7 | GCTGTGATGGACCTCGATTTGTGGGGTAGTAACAGC |

(SEQ ID NO: 28)

| | |
|---|---|
| Cas13d.1 | CAACTACAACCCCGTAAAAATACGGGGTTCTGAAAC |

(SEQ ID NO: 29)

| | |
|---|---|
| Cas13d.2 | GTTAAATACCACCTAAGAATGAGGAGGTTCTATAAC |

(SEQ ID NO: 30)

| | |
|---|---|
| Cas13d.3 | GAACGATAGCCTGCTGAAATATGCAGGTTCTAAGAC |

(SEQ ID NO: 31)

| | |
|---|---|
| Cas13d.4 | GATTGAAAGCTATGCGAATTTGCACAGTCTTAAAAC |

(SEQ ID NO: 32)

| | |
|---|---|
| Cas13d.5 | GAGATAGACCCTTGTTAACTCGTAAGGTTCTGTGAC |

(SEQ ID NO: 33)

| | |
|---|---|
| Cas13c.1 | ATTGGATATACCCCTAATTTGAGAGGGGAATAAAAC |

(SEQ ID NO: 34)

| | |
|---|---|
| Cas13c.2 | GTTGGACTATACCCTCGTTTGTAGGGGGAATAAAAC |

Since the secondary structures of the DR sequences, including the location and size of the step, bulge, and loop structures, are likely more important than the specific nucleotide sequences that form such secondary structures, alternative or derivative DR sequences can also be used in the systems and methods of the invention, so long as these derivative or alternative DR sequences have a secondary structure that substantially resembles the secondary structure of an RNA encoded by any one of SEQ ID NOs: 19-24 and 26-34. For example, the derivative DR sequence may have ±1 or 2 base pair(s) in one or both stems, have ±1, 2, or 3 bases in either or both of the single strands in the bulge, and/or have ±1, 2, 3, or 4 bases in the loop region.

In some embodiments, a Type VI-C, VI-D, VI-E and VI-F CRISPR-Cas effector proteins include a “derivative” having an amino acid sequence with at least about 80% sequence identity to the amino acid sequence of any one of SEQ ID NOs: 2-7 and 9-17 above (e.g., 81%, 82%, 83%, 84%, 85%, 86%, 87% 88%, 89%, 90%, 91%, 92%, 93%, 94%, 95%, 96%, 97%, 98%, 99%, or 100%). Such derivative Cas effectors sharing significant protein sequence identity to any one of SEQ ID NOs: 2-7 and 9-17 have retained at least one of the functions of the Cas of SEQ ID NOs: 2-7 and 9-17 (see below), such as the ability to bind to and form a complex with a crRNA comprising at least one of the DR sequences of SEQ ID NOs: 19-24 and 26-34 (e.g., a DR sequence of the corresponding wild-type Cas protein from which the derivative is derived). For example, a Cas13e.3-e.8, f.6-f.7, d.1-d.5, and c.1-c.2 derivative may share 85% amino acid sequence identity to SEQ ID NO: 2, 3, 4, 5, 6, 7, 9, 10, 11, 12, 13, 14, 15, 16, or 17, respectively, and retains the ability to bind to and form a complex with a crRNA having a DR sequence of SEQ ID NO: 19, 20, 21, 22, 23, 24, 26, 27, 28, 29, 30, 31, 32, 33, and 34, respectively.

In some embodiments, the derivative comprises conserved amino acid residue substitutions. In some embodiments, the derivative comprises only conserved amino acid residue substitutions (i.e., all amino acid substitutions in the derivative are conserved substitutions, and there is no substitution that is not conserved).

In some embodiments, the derivative comprises no more than 1, 2, 3, 4, 5, 6, 7, 8, 9, or 10 amino acid insertions or deletions into any one of the wild-type sequences of SEQ ID NOs: 2-7 and 9-17. The insertion and/or deletion maybe clustered together, or separated throughout the entire length of the sequences, so long as at least one of the functions of the wild-type sequence is preserved. Such functions may include the ability to bind the guide/crRNA, the RNase activity, the ability to bind to and/or cleave the target RNA complementary to the guide/crRNA. In some embodiments, the insertions and/or deletions are not present in the RXXXXH motifs, or within 5, 10, 15, or 20 residues from the RXXXXH motifs.

In some embodiments, the derivative has retained the ability to bind guide RNA/crRNA.

In some embodiments, the derivative has retained the guide/crRNA-activated RNase activity.

In some embodiments, the derivative has retained the ability to bind target RNA and/or cleave the target RNA in the presence of the bound guide/crRNA that is complementary in sequence to at least a portion of the target RNA.

In other embodiments, the derivative has completely or partially lost the guide/crRNA-activated RNase activity, due to, for example, mutations in one or more catalytic residues of the RNA-guided RNase. Such derivatives are sometimes referred to as dCas, such as dCas13e.3, etc.

Thus in certain embodiments, the derivative may be modified to have diminished nuclease/RNase activity, e.g., nuclease inactivation of at least 50%, at least 60%, at least 70%, at least 80%, at least 90%, at least 95%, at least 97%, or 100% as compared with the counterpart wild type proteins. The nuclease activity can be diminished by several methods known in the art, e.g., introducing mutations into the nuclease (catalytic) domains of the proteins. In some embodiments, catalytic residues for the nuclease activities are identified, and these amino acid residues can be substituted by different amino acid residues (e.g., glycine or alanine) to diminish the nuclease activity. In some embodiments, the amino acid substitution is a conservative amino acid substitution. In some embodiments, the amino acid substitution is a non-conservative amino acid substitution.

In some embodiments, the modification comprises one or more mutations (e g, amino acid deletions, insertions, or substitutions) in at least one HEPN domain. In some embodiments, there is one, two, three, four, five, six, seven, eight, nine, or more amino acid substitutions in at least one HEPN domain. For example, in some embodiments, the one or more mutations comprise a substitution (e.g., an alanine substitution) at an amino acid residue corresponding to R84, H89, R739, H744, R740, H745 of SEQ ID NO: 1, or R97, H102, R770, H775 of SEQ ID NO: 2, or R77, H82, R764, H769 of SEQ ID NO: 3, or R79, H84, R766A, H771 of SEQ ID NO: 4, or R79, H84, R766, H771 of SEQ ID NO: 5, or R89, H94, R773, H778 of SEQ ID NO: 6, or R89, H94, R777, H782 of SEQ ID NO: 7.

In certain embodiments, the one or more mutations or the two or more mutations may be in a catalytically active domain of the effector protein comprising a HEPN domain, or a catalytically active domain which is homologous to a HEPN domain. In certain embodiments, the effector protein comprises one or more of the following mutations: R84A, H89A, R739A, H744A, R740A, H745A (wherein amino acid positions correspond to amino acid positions of Cas13e.3). The skilled person will understand that corresponding amino acid positions in different Cas13c, Cas13d, Cas13e, and Cas13f proteins may be mutated to the same effect. In certain embodiments, one or more mutations abolish catalytic activity of the protein completely or partially (e.g. altered cleavage rate, altered specificity, etc.).

Other exemplary (catalytic) residue mutations include: R97A, H102A, R770A, H775A of Cas13e.2, or R77A, H82A, R764A, H769A of Cas13f.1, or R79A, H84A, R766A, H771A of Cas13f.2, or R79A, H84A, R766A, H771A of Cas13f.3, or R89A, H94A, R773A, H778A of Cas13f.4, or R89A, H94A, R777A, H782A of Cas13f.5. In certain embodiments, any of the R and/or H residues herein may be replaced not be A but by G, V, or I.

The presence of at least one of these mutations results in a derivative having reduced or diminished RNase activity as compared to the corresponding wild-type protein lacking the mutations.

In certain embodiments, the effector protein as described herein is a “dead” effector protein, such as a dead Cas13c, Cas13d, Cas13e, or Cas13f effector protein (i.e. dCas13c, dCas13d, dCas13e, and dCas13f). In certain embodiments, the effector protein has one or more mutations in HEPN domain 1 (N-terminal). In certain embodiments, the effector protein has one or more mutations in HEPN domain 2 (C-terminal). In certain embodiments, the effector protein has one or more mutations in HEPN domain 1 and HEPN domain 2.

The inactivated Cas or derivative or functional fragment thereof can be fused or associated with one or more heterologous/functional domains (e.g., via fusion protein, linker peptides, “GS” linkers, etc.). These functional domains can have various activities, e.g., methylase activity, demethylase activity, transcription activation activity, transcription repression activity, transcription release factor activity, histone modification activity, RNA cleavage activity, DNA cleavage activity, nucleic acid binding activity, base-editing activity, and switch activity (e.g., light inducible). In some embodiments, the functional domains are Krüppel associated box (KRAB), SID (e.g. SID4X), VP64, VPR, VP16, FokI, P65, HSF1, MyoD1, Adenosine Deaminase Acting on RNA such as ADAR1, ADAR2, APOBEC, cytidine deaminase (AID), TAD, mini-SOG, APEX, and biotin-APEX.

In some embodiments, the functional domain is a base editing domain, e.g., ADAR1 (including wild-type or ADAR2DD version thereof, with or without the E1008Q and/or the E488Q mutation(s)), ADAR2 (including wild-type or ADAR2DD version thereof, with or without the E1008Q and/or the E488Q mutation(s)), APOBEC, or AID.

In some embodiments, the functional domain may comprise one or more nuclear localization signal (NLS) domains. The one or more heterologous functional domains may comprise at least two or more NLS domains. The one or more NLS domain(s) may be positioned at or near or in proximity to a terminus of the effector protein (e.g., Cas13c, Cas13d, Cas13e, or Cas13f effector proteins) and if two or more NLSs, each of the two may be positioned at or near or in proximity to a terminus of the effector protein (e.g., Cas13c, Cas13d, Cas13e, or Cas13f effector proteins).

In some embodiments, at least one or more heterologous functional domains may be at or near the amino-terminus of the effector protein and/or wherein at least one or more heterologous functional domains is at or near the carboxy-terminus of the effector protein. The one or more heterologous functional domains may be fused to the effector protein. The one or more heterologous functional domains may be tethered to the effector protein. The one or more heterologous functional domains may be linked to the effector protein by a linker moiety.

In some embodiments, multiple (e.g., two, three, four, five, six, seven, eight, or more) identical or different functional domains are present.

In some embodiments, the functional domain (e.g., a base editing domain) is further fused to an RNA-binding domain (e.g., MS2).

In some embodiments, the functional domain is associated to or fused via a linker sequence (e.g., a flexible linker sequence or a rigid linker sequence). Exemplary linker sequences and functional domain sequences are provided in table below.

### Amino Acid Sequences of Motifs and Functional Domains in Engineered Variants of Type VI-C, VI-D, VI-E and VI-F CRISPR Cas Effectors

| | |
|---|---|
| Linker 1 | GS |
| Linker 2 | GSGGGGS |
| | (SEQ ID NO: 35) |
| Linker 3 | GGGGSGGGGSGGGGS |
| | (SEQ ID NO: 36) |
| ADAR1DD- | SLGTGNRCVKGDSLSLKGETVNDCHAEIISRRG |
| WT | FIRFLYSELMKYNSQTAKDSIFEPAKGGEKLQI |
| | KKTVSFHLYISTAPCGDGALFDKSCSDRAMEST |
| | ESRHYPVFENPKQGKLRTKVENGEGTIPVESSD |
| | IVPTWDGIRLGERLRTMSCSDKILRWNVLGLQG |
| | ALLTHFLQPIYLKSVTLGYLFSQGHLTRAICCR |
| | VTRDGSAFEDGLRHPFIVNHPKVGRVSIYDSKR |
| | QSGKTKETSVNWCLADGYDLEILDGTRGTVDGP |
| | RNELSRVSKKNIFLLFKKLCSFRYRRDLLRLSY |
| | GEAKKAARDYETAKNYFKKGLKDMGYGNWISKP |
| | QEEKNF |
| | (SEQ ID NO: 37) |
| ADAR1DD- | SLGTGNRCVKGDSLSLKGETVNDCHAEIISRRG |
| E1008Q | FIRFLYSELMKYNSQTAKDSIFEPAKGGEKLQI |
| | KKTVSFHLYISTAPCGDGALFDKSCSDRAMEST |
| | ESRHYPVFENPKQGKLRTKVENGQGTIPVESSD |
| | IVPTWDGIRLGERLRTMSCSDKILRWNVLGLQG |
| | ALLTHFLQPIYLKSVTLGYLFSQGHLTRAICCR |
| | VTRDGSAFEDGLRHPFIVNHPKVGRVSIYDSKR |
| | QSGKTKETSVNWCLADGYDLEILDGTRGTVDGP |
| | RNELSRVSKKNIFLLFKKLCSFRYRRDLLRLSY |
| | GEAKKAARDYETAKNYFKKGLKDMGYGNWISKP |
| | QEEKNF |
| | (SEQ ID NO: 38) |
| ADAR2DD- | QLHLPQVLADAVSRLVLGKFGDLTDNFSSPHAR |
| WT | RKVLAGVVMTTGTDVKDAKVISVSTGTKCINGE |
| | YMSDRGLALNDCHAEIISRRSLLRFLYTQLELY |
| | LNNKDDQKRSIFQKSERGGFRLKENVQFHLYIS |
| | TSPCGDARIFSPHEPILEEPADRHPNRKARGQL |
| | RTKIESGEGTIPVRSNASIQTWDGVLQGERLLT |
| | MSCSDKIARWNVVGIQGSLLSIFVEPIYFSSII |
| | LGSLYHGDHLSRAMYQRISNIEDLPPLYTLNKP |
| | LLSGISNAEARQPGKAPNFSVNWTVGDSAIEVI |
| | NATTGKDELGRASRLCKHALYCRWMRVHGKVPS |
| | HLLRSKITKPNVYHESKLAAKEYQAAKARLFTA |
| | FIKAGLGAWVEKPTEQDQFSLT |
| | (SEQ ID NO: 39) |
| ADAR2DD- | QLHLPQVLADAVSRLVLGKFGDLTDNFSSPHAR |
| E488Q | KRKVLAGVVMTTGTDVKDAKVISVSTGTCINGE |
| | YMSDRGLALNDCHAEIISRRSLLRFLYTQLELY |
| | LNNKDDQKRSIFQKSERGGFRLKENVQFHLYIS |
| | TSPCGDARIFSPHEPILEEPADRHPNRKARGQL |
| | RTKIESGQGTIPVRSNASIQTWDGVLQGERLLT |
| | MSCSDKIARWNVVGIQGSLLSIFVEPIYFSSII |
| | LGSLYHGDHLSRAMYQRISNIEDLPPLYTLNKP |
| | LLSGISNAEARQPGKAPNFSVNWTVGDSAIEVI |
| | NATTGKDELGRASRLCKHALYCRWMRVHGKVPS |
| | HLLRSKITKPNVYHESKLAAKEYQAAKARLFTA |
| | FIKAGLGAWVEKPTEQDQFSLT |
| | (SEQ ID NO: 40) |
| AID- | MDSLLMNRRKFLYQFKNVRWAKGRRETYLCYVV |
| AP0BEC1 | KRRDSATSFSLDFGYLRNKNGCHVELLFLRYIS |
| | DWDLDPGRCYRVTWFTSWSPCYDCARHVADFLR |
| | GNPNLSLRIFTARLYFCEDRKAEPEGLRRLHRA |
| | GVQIAIMTFKDYFYCWNTFVENHERTFKAWEGL |
| | HENSVRLSRQLRRILLPLYEVDDLRDAFRTLGL |
| | (SEQ ID NO: 41) |
| Lamprey_ | MTDAEYVRIHEKLDIYTFKKQFFNNKKSVSHRC |
| AID- | YVLFELKRRGERRACFWGYAVNKPQSGTERGIH |
| APOBEC1 | AEIFSIRKVEEYLRDNPGQFTINWYSSWSPCAD |
| | CAEKILEWYNQELRGNGHTLKIWACKLYYEKNA |
| | RNQIGLWNLRDNGVGLNVMVSEHYQCCRKIFIQ |
| | SSHNQLNENRWLEKTLKRAEKRRSELSIMIQVK |
| | ILHTTKSPAV |
| | (SEQ ID NO: 42) |
| APOBEC1_ | MSSETGPVAVDPTLRRRIEPHEFEVFFDPRELR |
| BE1 | KETCLLYEINWGGRHSIWRHTSQNTNKHVEVNF |
| | IEKFTTERYFCPNTRCSITWFLSWSPCGECSRA |
| | ITEFLSRYPHVTLFIYIARLYHHADPRNRQGLR |
| | DLISSGVTIQIMTEQESGYCWRNFVNYSPSNEA |
| | HWPRYPHLWVRLYVLELYCIILGLPPCLNILRR |
| | KQPQLTFFTIALQSCHYQRLPPHILWATGLK |
| | (SEQ ID NO: 43) |

The positioning of the one or more functional domains on the inactivated Cas proteins is one that allows for correct spatial orientation for the functional domain to affect the target with the attributed functional effect. For example, if the functional domain is a transcription activator (e.g., VP16, VP64, or p65), the transcription activator is placed in a spatial orientation that allows it to affect the transcription of the target. Likewise, a transcription repressor is positioned to affect the transcription of the target, and a nuclease (e.g., FokI) is positioned to cleave or partially cleave the target. In some embodiments, the functional domain is positioned at the N-terminus of the Cas/dCas. In some embodiments, the functional domain is positioned at the C-terminus of the Cas/dCas. In some embodiments, the inactivated CRISPR-associated protein (dCas) is modified to comprise a first functional domain at the N-terminus and a second functional domain at the C-terminus.

Various examples of inactivated CRISPR-associated proteins fused with one or more functional domains and methods of using the same are described, e.g., in International Publication No. WO 2017/219027, which is incorporated herein by reference in its entirety, and in particular with respect to the features described herein.

In some embodiments, a Type VI-C, VI-D, VI-E and VI-F CRISPR-Cas effector proteins includes the amino acid sequence of any one of SEQ ID NOs: 2-7 and 9-17 above. In some embodiments, a Type VI-C, VI-D, VI-E and VI-F CRISPR-Cas effector proteins excludes the naturally occurring amino acid sequence of any one of SEQ ID NOs: 2-7 and 9-17 above.

In some embodiments, instead of using full-length wild-type (SEQ ID NOs: 2-7 and 9-17) or derivative Type VI-C, VI-D, VI-E and VI-F Cas effectors, “functional fragments” thereof can be used.

A “functional fragment,” as used herein, refers to a fragment of a wild-type protein of any one of SEQ ID NOs: 2-7 and 9-17, or a derivative thereof, that has less-than full-length sequence. The deleted residues in the functional fragment can be at the N-terminus, the C-terminus, and/or internally. The functional fragment retains at least one function of the wild-type Type VI-C, VI-D, VI-E and VI-F Cas, or at least one function of its derivative. Thus a functional fragment is defined specifically with respect to the function at issue. For example, a functional fragment, wherein the function is the ability to bind crRNA and target RNA, may not be a functional fragment with respect to the RNase function, because losing the RXXXXH motifs at both ends of the Cas may not affect its ability to bind a crRNA and target RNA, but may eliminate destroy the RNase activity.

In some embodiments, compared to full-length sequences SEQ ID NOs: 2-7 and 9-17, the Type VI-C, VI-D, VI-E and VI-F CRISPR-Cas effector proteins or derivatives thereof or functional fragments thereof lacks about 30, 60, 90, 120, 150, or about 180 residues from the N-terminus.

In some embodiments, compared to full-length sequences SEQ ID NOs: 2-7 and 9-17, the Type VI-C, VI-D, VI-E and VI-F CRISPR-Cas effector proteins or derivatives thereof or functional fragments thereof lacks about 30, 60, 90, 120, or about 150 residues from the C-terminus.

In some embodiments, compared to full-length sequences SEQ ID NOs: 2-7 and 9-17, the Type VI-C, VI-D, VI-E and VI-F CRISPR-Cas effector proteins or derivatives thereof or functional fragments thereof lacks about 30, 60, 90, 120, 150, or about 180 residues from the N-terminus, and lacks about 30, 60, 90, 120, or about 150 residues from the C-terminus.

In some embodiments, the Type VI-C, VI-D, VI-E and VI-F CRISPR-Cas effector proteins or derivatives thereof or functional fragments thereof have RNase activity, e.g., guide/crRNA-activated specific RNase activity.

In some embodiments, the Type VI-C, VI-D, VI-E and VI-F CRISPR-Cas effector proteins or derivatives thereof or functional fragments thereof have no substantial/detectable collateral RNase activity.

Here, “collateral RNase activity” refers to the non-specific RNase activity observed in certain other Class 2, type VI RNA-guided RNases, such as Cas13a. A complex comprising Cas13a, for example, upon activation by binding to a target nucleic acid (e.g., a target RNA), a conformational change results, which in turn causes the complex to act as a non-specific RNase, cleaving and/or degrading nearby RNA molecules (e.g., ssRNA or dsRNA molecules) (i.e., “collateral” effects).

In certain embodiments, a complex comprised of (but not limited to) the Type VI-C, VI-D, VI-E and VI-F CRISPR-Cas effector proteins or derivatives thereof or functional fragments thereof and a crRNA does not exhibit significant collateral RNase activity subsequent to target recognition. This “collateral-free” embodiment may comprise wild-type, engineered/derivative effector proteins, or functional fragments thereof.

In some embodiments, the Type VI-C, VI-D, VI-E and VI-F CRISPR-Cas effector proteins or derivatives thereof or functional fragments thereof recognizes and cleaves the target RNA without any additional requirements adjacent to or flanking the protospacer (i.e., protospacer adjacent motif “PAM” or protospacer flanking sequence “PFS” requirements).

The present disclosure also provides a split version of the CRISPR-associated proteins described herein (e.g., a Type VI-C, VI-D, VI-E and VI-F CRISPR-Cas effector protein). The split version of the CRISPR-associated protein may be advantageous for delivery. In some embodiments, the CRISPR-associated proteins are split into two parts of the enzyme, which together substantially comprise a functioning CRISPR-associated protein.

The split can be done in a way that the catalytic domain(s) are unaffected. The CRISPR-associated protein may function as a nuclease or may be an inactivated enzyme, which is essentially a RNA-binding protein with very little or no catalytic activity (e.g., due to mutation(s) in its catalytic domains). Split enzymes are described, e.g., in Wright et al., “Rational design of a split-Cas9 enzyme complex,” Proc. Nat'l. Acad. Sci. 112(10): 2984-2989, 2015, which is incorporated herein by reference in its entirety.

For example, in some embodiments, the nuclease lobe and α-helical lobe are expressed as separate polypeptides. Although the lobes do not interact on their own, the crRNA recruits them into a ternary complex that recapitulates the activity of full-length CRISPR-associated proteins and catalyzes site-specific DNA cleavage. The use of a modified crRNA abrogates split-enzyme activity by preventing dimerization, allowing for the development of an inducible dimerization system.

In some embodiments, the split CRISPR-associated protein can be fused to a dimerization partner, e.g., by employing rapamycin sensitive dimerization domains. This allows the generation of a chemically inducible CRISPR-associated protein for temporal control of the activity of the protein. The CRISPR-associated protein can thus be rendered chemically inducible by being split into two fragments and rapamycin-sensitive dimerization domains can be used for controlled re-assembly of the protein.

The split point is typically designed in silico and cloned into the constructs. During this process, mutations can be introduced to the split CRISPR-associated protein and non-functional domains can be removed.

In some embodiments, the two parts or fragments of the split CRISPR-associated protein (i.e., the N-terminal and C-terminal fragments), can form a full CRISPR-associated protein, comprising, e.g., at least 70%, at least 80%, at least 90%, at least 95%, or at least 99% of the sequence of the wild-type CRISPR-associated protein.

The CRISPR-associated proteins described herein (e.g., a Type VI-C, VI-D, VI-E and VI-F CRISPR-Cas effector protein) can be designed to be self-activating or self-inactivating. For example, the target sequence can be introduced into the coding construct of the CRISPR-associated protein. Thus, the CRISPR-associated protein can cleave the target sequence, as well as the construct encoding the protein thereby self-inactivating their expression. Methods of constructing a self-inactivating CRISPR system are described, e.g., in Epstein and Schaffer, Mol. Ther. 24: S50, 2016, which is incorporated herein by reference in its entirety.

In some other embodiments, an additional crRNA, expressed under the control of a weak promoter (e.g., 7SK promoter), can target the nucleic acid sequence encoding the CRISPR-associated protein to prevent and/or block its expression (e.g., by preventing the transcription and/or translation of the nucleic acid). The transfection of cells with vectors expressing the CRISPR-associated protein, the crRNAs, and crRNAs that target the nucleic acid encoding the CRISPR-associated protein can lead to efficient disruption of the nucleic acid encoding the CRISPR-associated protein and decrease the levels of CRISPR-associated protein, thereby limiting the genome editing activity.

In some embodiments, the genome editing activity of the CRISPR-associated protein can be modulated through endogenous RNA signatures (e.g., miRNA) in mammalian cells. A CRISPR-associated protein switch can be made by using a miRNA-complementary sequence in the 5′-UTR of mRNA encoding the CRISPR-associated protein. The switches selectively and efficiently respond to miRNA in the target cells. Thus, the switches can differentially control the genome editing by sensing endogenous miRNA activities within a heterogeneous cell population. Therefore, the switch systems can provide a framework for cell-type selective genome editing and cell engineering based on intracellular miRNA information (see, e.g., Hirosawa et al., Nucl. Acids Res. 45(13): e118, 2017).

The CRISPR-associated proteins (e.g., Type VI-C, VI-D, VI-E and VI-F CRISPR-Cas effector proteins) can be inducibly expressed, e.g., their expression can be light-induced or chemically-induced. This mechanism allows for activation of the functional domain in the CRISPR-associated proteins. Light inducibility can be achieved by various methods known in the art, e.g., by designing a fusion complex wherein CRY2 PHR/CIBN pairing is used in split CRISPR-associated proteins (see, e.g., Konermann et al., “Optical control of mammalian endogenous transcription and epigenetic states,” Nature 500:7463, 2013).

Chemical inducibility can be achieved, e.g., by designing a fusion complex wherein FKBP/FRB (FK506 binding protein/FKBP rapamycin binding domain) pairing is used in split CRISPR-associated proteins. Rapamycin is required for forming the fusion complex, thereby activating the CRISPR-associated proteins (see, e.g., Zetsche et al., “A split-Cas9 architecture for inducible genome editing and transcription modulation,” Nature Biotech. 33:2:139-42, 2015).

Furthermore, expression of the CRISPR-associated proteins can be modulated by inducible promoters, e.g., tetracycline or doxycycline controlled transcriptional activation (Tet-On and Tet-Off expression system), hormone inducible gene expression system (e.g., an ecdysone inducible gene expression system), and an arabinose-inducible gene expression system. When delivered as RNA, expression of the RNA targeting effector protein can be modulated via a riboswitch, which can sense a small molecule like tetracycline (see, e.g., Goldfless et al., “Direct and specific chemical control of eukaryotic translation with a synthetic RNA-protein interaction,” Nucl. Acids Res. 40:9: e64-e64, 2012).

Various embodiments of inducible CRISPR-associated proteins and inducible CRISPR systems are described, e.g., in U.S. Pat. No. 8,871,445, US Publication No. 2016/0208243, and International Publication No. WO 2016/205764, each of which is incorporated herein by reference in its entirety.

In some embodiments, the CRISPR-associated proteins include at least one (e.g., 1, 2, 3, 4, 5, 6, 7, 8, 9, or 10) Nuclear Localization Signal (NLS) attached to the N-terminal or C-terminal of the protein. Non-limiting examples of NLSs include an NLS sequence derived from: the NLS of the SV40 virus large T-antigen, having the amino acid sequence PKKKRKV (SEQ ID NO: 44); the NLS from nucleoplasmin (e.g., the nucleoplasmin bipartite NLS with the sequence KRPAATKKAGQAKKKK (SEQ ID NO: 45)); the c-myc NLS having the amino acid sequence PAAKRVKLD (SEQ ID NO: 46) or RQRRNELKRSP (SEQ ID NO: 47); the hRNPA1 M9 NLS having the sequence NQSSNFGPMKGGNFGGRSSGPYGGGGQYFAKPRNQGGY (SEQ ID NO: 48); the sequence RMRIZFKNKGKDTAELRRRRVEVSVELRKAKKDEQIL KRRNV (SEQ ID NO: 49) of the IBB domain from importin-alpha; the sequences VSRKRPRP (SEQ ID NO: 50) and PPKKARED (SEQ ID NO: 51) of the myoma T protein; the sequence PQPKKKPL (SEQ ID NO: 59) of human p53; the sequence SALIKKKKKMAP (SEQ ID NO: 52) of mouse c-abl IV; the sequences DRLRR (SEQ ID NO: 53) and PKQKKRK (SEQ ID NO: 54) of the influenza virus NS1; the sequence RKLKKKIKKL (SEQ ID NO: 55) of the Hepatitis virus delta antigen; the sequence REKKKFLKRR (SEQ ID NO: 56) of the mouse Mx1 protein; the sequence KRKGDEVDGVDEVAKKKSKK (SEQ ID NO: 57) of the human poly(ADP-ribose) polymerase; and the sequence RKCLQAGMNLEARKTKK (SEQ ID NO: 58) of the human glucocorticoid receptor. In some embodiments, the CRISPR-associated protein comprises at least one (e.g., 1, 2, 3, 4, 5, 6, 7, 8, 9, or 10) Nuclear Export Signal (NES) attached the N-terminal or C-terminal of the protein. In a preferred embodiment a C-terminal and/or N-terminal NLS or NES is attached for optimal expression and nuclear targeting in eukaryotic cells, e.g., human cells.

In some embodiments, the CRISPR-associated proteins described herein are mutated at one or more amino acid residues to alter one or more functional activities.

For example, in some embodiments, the CRISPR-associated protein is mutated at one or more amino acid residues to alter its helicase activity.

In some embodiments, the CRISPR-associated protein is mutated at one or more amino acid residues to alter its nuclease activity (e.g., endonuclease activity or exonuclease activity).

In some embodiments, the CRISPR-associated protein is mutated at one or more amino acid residues to alter its ability to functionally associate with a guide RNA.

In some embodiments, the CRISPR-associated protein is mutated at one or more amino acid residues to alter its ability to functionally associate with a target nucleic acid.

In some embodiments, the CRISPR-associated proteins described herein are capable of cleaving a target RNA molecule.

In some embodiments, the CRISPR-associated protein is mutated at one or more amino acid residues to alter its cleaving activity. For example, in some embodiments, the CRISPR-associated protein may comprise one or more mutations that render the enzyme incapable of cleaving a target nucleic acid.

In some embodiments, the CRISPR-associated protein is capable of cleaving the strand of the target nucleic acid that is complementary to the strand to which the guide RNA hybridizes.

In some embodiments, a CRISPR-associated protein described herein can be engineered to have a deletion in one or more amino acid residues to reduce the size of the enzyme while retaining one or more desired functional activities (e.g., nuclease activity and the ability to interact functionally with a guide RNA). The truncated CRISPR-associated protein can be advantageously used in combination with delivery systems having load limitations.

In some embodiments, the CRISPR-associated proteins described herein can be fused to one or more peptide tags, including a His-tag, GST-tag, a V5-tag, FLAG-tag, HA-tag, VSV-G-tag, Trx-tag, or myc-tag.

In some embodiments, the CRISPR-associated proteins described herein can be fused to a detectable moiety such as GST, a fluorescent protein (e.g., GFP, HcRed, DsRed, CFP, YFP, or BFP), or an enzyme (such as HRP or CAT).

In some embodiments, the CRISPR-associated proteins described herein can be fused to MBP, LexA DNA binding domain, or Gal4 DNA-binding domain.

In some embodiments, the CRISPR-associated proteins described herein can be linked to or conjugated with a detectable label such as a fluorescent dye, including FITC and DAPI.

In any of the embodiments herein, the linkage between the CRISPR-associated proteins described herein and the other moiety can be at the N- or C-terminal of the CRISPR-associated proteins, and sometimes even internally via covalent chemical bonds. The linkage can be effected by any chemical linkage known in the art, such as peptide linkage, linkage through the side chain of amino acids such as D, E, S, T, or amino acid derivatives (Ahx, β-Ala, GABA or Ava), or PEG linkage.

### 3. Polynucleotides

The invention also provides nucleic acids encoding the proteins and guide RNAs (e.g., a crRNA) described herein (e.g., a CRISPR-associated protein or an accessory protein).

In some embodiments, the nucleic acid is a synthetic nucleic acid. In some embodiments, the nucleic acid is a DNA molecule. In some embodiments, the nucleic acid is an RNA molecule (e.g., an mRNA molecule encoding the Cas, derivative or functional fragment thereof). In some embodiments, the mRNA is capped, polyadenylated, substituted with 5-methyl cytidine, substituted with pseudouridine, or a combination thereof.

In some embodiments, the nucleic acid (e.g., DNA) is operably linked to a regulatory element (e.g., a promoter) in order to control the expression of the nucleic acid. In some embodiments, the promoter is a constitutive promoter. In some embodiments, the promoter is an inducible promoter. In some embodiments, the promoter is a cell-specific promoter. In some embodiments, the promoter is an organism-specific promoter.

Suitable promoters are known in the art and include, for example, a pol I promoter, a pol II promoter, a pol III promoter, a T7 promoter, a U6 promoter, a H1 promoter, retroviral Rous sarcoma virus LTR promoter, a cytomegalovirus (CMV) promoter, an EF-1α promoter, a CAG promoter, a CBA promoter, a SV40 promoter, a dihydrofolate reductase promoter, and a β-actin promoter. For example, a U6 promoter can be used to regulate the expression of a guide RNA molecule described herein.

In some embodiments, the nucleic acid(s) are present in a vector (e.g., a viral vector or a phage). The vector can be a cloning vector, or an expression vector. The vectors can be plasmids, phagemids, Cosmids, etc. The vectors may include one or more regulatory elements that allow for the propagation of the vector in a cell of interest (e.g., a bacterial cell or a mammalian cell). In some embodiments, the vector includes a nucleic acid encoding a single component of a CRISPR-associated (Cas) system described herein. In some embodiments, the vector includes multiple nucleic acids, each encoding a component of a CRISPR-associated (Cas) system described herein.

In one aspect, the present disclosure provides nucleic acid sequences that are at least 50%, 55%, 60%, 65%, 70%, 75%, 80%, 85%, 90%, 91%, 92%, 93%, 94%, 95%, 96%, 97%, 98%, 99%, or 100% identical to the nucleic acid sequences described herein, i.e., nucleic acid sequences encoding the Cas proteins, derivatives, functional fragments, or guide/crRNA, including the DR sequences of SEQ ID NOs: 19-24 and 26-34.

In certain embodiments, the subject nucleic acid sequences are at least 50%, 55%, 60%, 65%, 70%, 75%, 80%, 85%, 90%, 91%, 92%, 93%, 94%, 95%, 96%, 97%, 98%, 99%, or 100% identical to any one of SEQ ID NOs: 90-102.

In another aspect, the present disclosure also provides nucleic acid sequences encoding amino acid sequences that are at least 50%, 55%, 60%, 65%, 70%, 75%, 80%, 85%, 90%, 91%, 92%, 93%, 94%, 95%, 96%, 97%, 98%, 99%, or 100% identical to the amino acid sequences described herein, such as SEQ ID NOs: 2-7 and 9-17.

In some embodiments, the nucleic acid sequences have at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is the same as the sequences described herein. In some embodiments, the nucleic acid sequences have at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is different from the sequences described herein.

In related embodiments, the invention provides amino acid sequences having at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is the same as the sequences described herein. In some embodiments, the amino acid sequences have at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is different from the sequences described herein.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In general, the length of a reference sequence aligned for comparison purposes should be at least 80% of the length of the reference sequence, and in some embodiments is at least 90%, 95%, or 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For purposes of the present disclosure, the comparison of sequences and determination of percent identity between two sequences can be accomplished using a Blossum 62 scoring matrix with a gap penalty of 12, a gap extend penalty of 4, and a frameshift gap penalty of 5.

The proteins described herein (e.g., CRISPR-associated proteins or accessory proteins) can be delivered or used as either nucleic acid molecules or polypeptides.

In certain embodiments, the nucleic acid molecule encoding the CRISPR-associated proteins, derivatives or functional fragments thereof are codon-optimized for expression in a host cell or organism. The host cell may include established cell lines (such as 293T cells) or isolated primary cells. The nucleic acid can be codon optimized for use in any organism of interest, in particular human cells or bacteria. For example, the nucleic acid can be codon-optimized for any prokaryotes (such as E. coli), or any eukaryotes such as human and other non-human eukaryotes including yeast, worm, insect, plants and algae (including food crop, rice, corn, vegetables, fruits, trees, grasses), vertebrate, fish, non-human mammal (e.g., mice, rats, rabbits, dogs, birds (such as chicken), livestock (cow or cattle, pig, horse, sheep, goat etc.), or non-human primates). Codon usage tables are readily available, for example, at the “Codon Usage Database” available at www.kazusa.orjp/codon/, and these tables can be adapted in a number of ways. See Nakamura et al., Nucl. Acids Res. 28:292, 2000 (incorporated herein by reference in its entirety). Computer algorithms for codon optimizing a particular sequence for expression in a particular host cell are also available, such as Gene Forge (Aptagen; Jacobus, Pa.).

An example of a codon optimized sequence, is in this instance a sequence optimized for expression in a eukaryote, e.g., humans (i.e. being optimized for expression in humans), or for another eukaryote, animal or mammal as herein discussed; see, e.g., SaCas9 human codon optimized sequence in WO 2014/093622 (PCT/US2013/074667). Whilst this is preferred, it will be appreciated that other examples are possible and codon optimization for a host species other than human, or for codon optimization for specific organs is known. In general, codon optimization refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at http://www.kazusa.orjp/codon/ and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, Pa.), are also available. In some embodiments, one or more codons (e.g., 1, 2, 3, 4, 5, 10, 15, 20, 25, 50, or more, or all codons) in a sequence encoding a Cas correspond to the most frequently used codon for a particular amino acid.

In certain embodiments, the subject nucleic acid sequences are codon optimized for mammalian (e.g., human) expression. Exemplary codon optimized sequences include any one of SEQ ID NOs: 90-102. In certain embodiments, the subject nucleic acid sequences are at least 50%, 55%, 60%, 65%, 70%, 75%, 80%, 85%, 90%, 91%, 92%, 93%, 94%, 95%, 96%, 97%, 98%, 99%, or 100% identical to any one of SEQ ID NOs: 90-102.

### 4. RNA Guides or crRNA

In some embodiments, the CRISPR systems described herein include at least RNA guide (e.g., a gRNA or a crRNA).

The architecture of multiple RNA guides is known in the art (see, e.g., International Publication Nos. WO 2014/093622 and WO 2015/070083, the entire contents of each of which are incorporated herein by reference).

In some embodiments, the CRISPR systems described herein include multiple RNA guides (e.g., one, two, three, four, five, six, seven, eight, or more RNA guides).

In some embodiments, the RNA guide includes a crRNA. In some embodiments, the RNA guide includes a crRNA but not a tracrRNA.

Sequences for guide RNAs from multiple CRISPR systems are generally known in the art, see, for example, Grissa et al. (Nucleic Acids Res. 35 (web server issue): W52-7, 2007; Grissa et al., BMC Bioinformatics 8:172, 2007; Grissa et al., Nucleic Acids Res. 36 (web server issue): W145-8, 2008; and Moller and Liang, PeerJ 5: e3788, 2017; the CRISPR database at: crispr.i2bc.paris-saclayfr/crispr/BLAST/CRISPRsBlast.php; and MetaCRAST available at: github.com/molleraj/MetaCRAST). All incorporated herein by reference.

In some embodiments, the crRNA includes a direct repeat (DR) sequence and a spacer sequence. In certain embodiments, the crRNA comprises, consists essentially of, or consists of a direct repeat sequence linked to a guide sequence or spacer sequence, at the 5′- or 3′-end of the spacer sequence.

In general, the Cas protein forms a complex with the mature crRNA, which spacer sequence directs the complex to a sequence-specific binding with the target RNA that is complementary to the spacer sequence, and/or hybridizes to the spacer sequence. The resulting complex comprises the Cas protein and the mature crRNA bound to the target RNA.

The direct repeat sequences for the Cas13e and Cas13f systems are generally well conserved, especially at the ends, with a GCTG for Cas13e and GCTGT for Cas13f at the 5′-end, reverse complementary to a CAGC for Cas13e and ACAGC for Cas13f at the 3′ end. The DR sequence for Cas13e.8 comprises GTTG at the 5′ end and the complement CAGC at the 3′ end. This conservation suggests strong base pairing for an RNA stem-loop structure that potentially interacts with the protein(s) in the locus.

In some embodiments, the direct repeat sequence, when in RNA, comprises the general secondary structure of 5′-S1a-Ba-S2a-L-S2b-Bb-S1b-3′, wherein segments S1a and S1b are reverse complement sequences and form a first stem (S1) having 4-5 nucleotides in Cas13e (Cas13e.3-Cas13.7) and 5 nucleotides in Cas13f.6 and Cas13f.7; segments Ba and Bb do not base pair with each other and form a symmetrical or nearly symmetrical bulge (B), and have 2-5 nucleotides or 2 (Ba) and 1 (Bb) or 3 (Ba) and 2 (Bb) nucleotides in Cas13e (Cas13e.3-Cas13.7), and 5 (Ba) and 4 (Bb) nucleotides respectively in Cas13f.6 and Cas13f.7; segments S2a and S2b are reverse complement sequences and form a second stem (S2) having 4-6 base pairs in Cas13e (Cas13e.3-Cas13.7) and either 6 base pairs in Cas13f.6 and Cas13f.7; and L is an 6 to 10-nucleotide loop in Cas13e (Cas13e.3-Cas13.7) and a 5-nucleotide loop in Cas13f. See and table below.

| Cas DR | | | | | | | |
|---|---|---|---|---|---|---|---|
| se- | | | | | | | |
| quences | S1a | Ba | S2a | L | S2b | Bb | S1b |
| Cas13e.3 | GCTGGA | GCA | GCCC | TCGATT | GGGT | AA | TCAC |
| | | | | TGCT | | | AGC |
| | | | | (SEQ | | | |
| | | | | ID | | | |
| | | | | NO: | | | |
| | | | | 63) | | | |
| Cas13e.4 | GCTG | AAGCA | ACCCTG | GTTTTG | CGGGGT | GATTA | CAGC |
| Cas13e.5 | GCTGT | AGAA | GCCTC | CGATTT | GAGGT | GATG | ACAGC |
| | | | | GT | | | |
| Cas13e.6 | GCTGGA | GCA | GCCCT | CGATTT | AGGGT | AA | TCAC |
| | | | | GC | | | AGC |
| Cas13e.7 | GCTGGA | GC | AGCCCT | CGATTT | AGGGTT | A | TCAC |
| | | | | GC | | | AGC |
| Cas13f.6 | GCTGT | GATAG | ACCTCG | ATTTG | TGGGGT | AG TA | ACAGC |
| Cas13f.7 | GCTGT | GATGG | ACCTCG | ATTTG | TGGGGT | AG TA | ACAGC |

In certain embodiments, S1a has a sequence of GCUG in Cas13e and GCUGU in Cas13f.

In certain embodiments, S2a has a sequence of GCCCC in Cas13e and A/G CCUC G/A in Cas13f (wherein the first A or G may be absent).

In some embodiments, the direct repeat sequence, when in RNA, comprises the general secondary structure of 5′-S1a-B1a-S2a-B2a-S3a-L-S3b-B2b-S2b-Bb-S1b-3′, wherein segments S1a and S1b are reverse complement sequences and form a first stem (S1) having 4 nucleotides in Cas13e (e.g., Cas13e.8) and Cas13d (e.g., Cas13d.2); segments B1a and B1b do not base pair with each other and form a symmetrical or nearly symmetrical bulge (B1), and have 2 nucleotides in Cas13e.8, and 3 (B1a) and 4 (Bib) nucleotide respectively in Cas13d.2; segments S2a and S2b are reverse complement sequences and form a second stem (S2) having 2 base pairs in Cas13e.8 and 3 base pairs in Cas13d.2; segments B2a and B2b do not base pair with each other and form a symmetrical bulge (B2), and have 1 nucleotide in Cas13e.8 and Cas13d.2; segment S3a and S3b are reverse complement sequences and form a third stem (S3) having 6-base pairs in Cas13e.8 and 3 nucleotides in Cas13d.2; and L is a 6 or 7 nucleotide loop in Cas13e.8 and Cas13d.2, respectively. See FIG. 2 and table below.

| Cas DR | | | | | | | | | | | |
|---|---|---|---|---|---|---|---|---|---|---|---|
| se- | | | | | | | | | | | |
| quences | S1a | B1a | S2a | B2a | S3a | L | S3b | B2b | S2b | B1b | S1b |
| Cas13e.8 | GTTG | GA | GT | A | GCC | GAT | CGG | G | AT | TA | CAGC |
| | | | | | CCG | TTG | GGT | | | | |
| Cas13d.2 | GTTA | AAT | ACC | A | CCT | AAGA | AGG | A | GGT | TCTA | TAAC |
| | | | | | | ATG | | | | | |

In some embodiments, the direct repeat sequence, when in RNA, comprises the general secondary structure of 5′-Aa-Sa-L-Sb-Ab-3′, wherein segments Aa and Ab do not base pair with each other and form arms at the end of the DR sequence and having 7 nucleotides in Cas13d.1 and Cas13d.3; segments Sa and Sb are reverse complement sequences and for a stem (s) having 9 base pairs (Cas13d.1) and 7 base pairs (Cas13d.3); and L is a 4 nucleotide loop in Cas13d.1 and 8 nucleotide loop in Cas13e.3. See FIG. 2 and table below.

| Cas DR | | | | | |
|---|---|---|---|---|---|
| se- | | | | | |
| quences | Arm-a | S1a | L | S1b | Arm-b |
| Cas13d.1 | CAACTAC | AACC | AAAA | TACGG | CTGAAAC |
| | | CCGTA | | GGTT | |
| Cas13d.3 | GAACGAT | AGCCTGC | TGAAATAT | GCAGGTT | CTAAGAC |

In some embodiments, the direct repeat sequences, when in RNA, comprises the general secondary structure of 5′-Aa-S1a-Ba-S2a-L-S2b-Bb-S1b-Ab-3′, wherein segments Aa and Ab do not base pair with each other and form arms at the ends of the DR sequence and having 3-5 nucleotides in Cas13d and 3-7 nucleotides in Cas13c; segments S1a and S1b are reverse complement sequences and form a first stem (S1) having 5-6 base pair in Cas13d and 3 base pairs in Cas13c; segments B1a and B1b do not base pair with each other and form a symmetrical bulge (B1), and have 1 nucleotides in Cas13d and Cas13c; segments S2a and S2b are reverse complement sequences and form a second stem (S21) having 4-5 base pair in Cas13d and 5 base pairs in Cas13c; and L is a 4 or 8 nucleotide loop in Cas13d and 6 or 8 nucleotide loop in Cas13c. See FIG. 2 and table below.

| Cas DR | | | | | | | | | |
|---|---|---|---|---|---|---|---|---|---|
| se- | Arm- | | | | | | | | Arm- |
| quences | a | S1a | Ba | S2a | L | S2b | Bb | S1b | b |
| Cas13d.4 | GATT | AAGCT | A | TGCG | AATT | TGCA | C | AGT | AAAA |
| | GA | | | | | | | CTT | C |
| Cas13d.5 | GAG | ATAGA | C | CCTTG | TTAA | TAAGG | T | TCTGT | GAC |
| | | | | | CTCG | | | | |
| Cas13c.1 | ATTG | TAT | A | CCCCT | AATT | AGGGG | A | ATA | AAAC |
| | GA | | | | TGAG | | | | |
| Cas13c.2 | GTTG | TAT | A | CCCTC | GTTT | GGGGG | A | ATA | AAAC |
| | GAC | | | | GTA | | | | |

In some embodiments, the direct repeat sequence comprises or consists of a nucleic acid sequence of SEQ ID NOs: 19-24 and 26-34.

As used herein, “direct repeat sequence” or “DR sequence” may refer to the DNA coding sequence in the CRISPR locus, or to the RNA encoded by the same in crRNA. Thus when any of SEQ ID NOs: 19-24 and 26-34 is referred to in the context of an RNA molecule, such as crRNA, each T is understood to represent a U.

In some embodiments, the direct repeat sequence comprises or consists of a nucleic acid sequence having up to 1, 2, 3, 4, 5, 6, 7, or 8 nucleotides of deletion, insertion, or substitution of SEQ ID NOs: 19-24 and 26-34. In some embodiments, the direct repeat sequence comprises or consists of a nucleic acid sequence having at least 80%, 85%, 90%, 95%, or 97% of sequence identity with SEQ ID NOs: 19-24 and 26-34 (e.g., due to deletion, insertion, or substitution of nucleotides in SEQ ID NOs: 19-24 and 26-34). In some embodiments, the direct repeat sequence comprises or consists of a nucleic acid sequence that is not identical to any one of SEQ ID NOs: 19-24 and 26-34, but can hybridize with a complement of any one of SEQ ID NOs: 19-24 and 26-34 under stringent hybridization conditions, or can bind to a complement of any one of SEQ ID NOs: 19-24 and 26-34 under physiological conditions.

In certain embodiments, the deletion, insertion, or substitution does not change the overall secondary structure of that of SEQ ID NOs: 19-24 and 26-34 (e.g., the relative locations and/or sizes of the stems and bulges and loop do not significantly deviate from that of the original stems, bulges, and loop). For example, the deletion, insert, or substitution may be in the bulge or loop region so that the overall symmetry of the bulge remains largely the same. The deletion, insertion, or substitution may be in the stems so that the length of the stems do not significantly deviate from that of the original stems (e.g., adding or deleting one base pair in each of the two stems correspond to 4 total base changes).

In certain embodiments, the deletion, insertion, or substitution results in a derivative DR sequence that may have ±1 or 2 base pair(s) in one or both stems, have ±1, 2, or 3 bases in either or both of the single strands in the bulge, and/or have ±1, 2, 3, or 4 bases in the loop region.

In certain embodiments, any of the above direct repeat sequences that is different from any one of SEQ ID NOs: 19-24 and 26-34 retains the ability to function as a direct repeat sequence in the Cas13e or Cas13f proteins, as the DR sequence of SEQ ID NOs: 19-24 and 26-34.

In some embodiments, the direct repeat sequence comprises or consists of a nucleic acid having a nucleic acid sequence of any one of SEQ ID NOs: 19-24 and 26-34, with a truncation of the initial three, four, five, six, seven, or eight 3′ nucleotides.

In some embodiments, the Cas protein comprises the amino acid sequence of SEQ ID NO: 2 and the crRNA comprises a direct repeat sequence, wherein the direct repeat sequence comprises or consists of the nucleic acid sequence of SEQ ID NO: 19.

In some embodiments, the Cas protein comprises the amino acid sequence of SEQ ID NO: 3 and the crRNA comprises a direct repeat sequence, wherein the direct repeat sequence comprises or consists of the nucleic acid sequence of SEQ ID NO: 20.

In some embodiments, the Cas protein comprises the amino acid sequence of SEQ ID NO: 4 and the crRNA comprises a direct repeat sequence, wherein the direct repeat sequence comprises or consists of the nucleic acid sequence of SEQ ID NO: 21.

In some embodiments, the Cas protein comprises the amino acid sequence of SEQ ID NO: 5 and the crRNA comprises a direct repeat sequence, wherein the direct repeat sequence comprises or consists of the nucleic acid sequence of SEQ ID NO: 22.

In some embodiments, the Cas protein comprises the amino acid sequence of SEQ ID NO: 6 and the crRNA comprises a direct repeat sequence, wherein the direct repeat sequence comprises or consists of the nucleic acid sequence of SEQ ID NO: 23.

In some embodiments, the Cas protein comprises the amino acid sequence of SEQ ID NO: 7 and the crRNA comprises a direct repeat sequence, wherein the direct repeat sequence comprises or consists of the nucleic acid sequence of SEQ ID NO: 24.

In some embodiments, the Cas protein comprises the amino acid sequence of SEQ ID NO: 9 and the crRNA comprises a direct repeat sequence, wherein the direct repeat sequence comprises or consists of the nucleic acid sequence of SEQ ID NO: 26.

In some embodiments, the Cas protein comprises the amino acid sequence of SEQ ID NO: 10 and the crRNA comprises a direct repeat sequence, wherein the direct repeat sequence comprises or consists of the nucleic acid sequence of SEQ ID NO: 27.

In some embodiments, the Cas protein comprises the amino acid sequence of SEQ ID NO: 11 and the crRNA comprises a direct repeat sequence, wherein the direct repeat sequence comprises or consists of the nucleic acid sequence of SEQ ID NO: 28.

In some embodiments, the Cas protein comprises the amino acid sequence of SEQ ID NO: 12 and the crRNA comprises a direct repeat sequence, wherein the direct repeat sequence comprises or consists of the nucleic acid sequence of SEQ ID NO: 29.

In some embodiments, the Cas protein comprises the amino acid sequence of SEQ ID NO: 13 and the crRNA comprises a direct repeat sequence, wherein the direct repeat sequence comprises or consists of the nucleic acid sequence of SEQ ID NO: 30.

In some embodiments, the Cas protein comprises the amino acid sequence of SEQ ID NO: 14 and the crRNA comprises a direct repeat sequence, wherein the direct repeat sequence comprises or consists of the nucleic acid sequence of SEQ ID NO: 31.

In some embodiments, the Cas protein comprises the amino acid sequence of SEQ ID NO: 15 and the crRNA comprises a direct repeat sequence, wherein the direct repeat sequence comprises or consists of the nucleic acid sequence of SEQ ID NO: 32.

In some embodiments, the Cas protein comprises the amino acid sequence of SEQ ID NO: 16 and the crRNA comprises a direct repeat sequence, wherein the direct repeat sequence comprises or consists of the nucleic acid sequence of SEQ ID NO: 33.

In some embodiments, the Cas protein comprises the amino acid sequence of SEQ ID NO: 17 and the crRNA comprises a direct repeat sequence, wherein the direct repeat sequence comprises or consists of the nucleic acid sequence of SEQ ID NO: 34. In classic CRISPR systems, the degree of complementarity between a guide sequence (e.g., a crRNA) and its corresponding target sequence can be about 50%, 60%, 75%, 80%, 85%, 90%, 95%, 97.5%, 99%, or 100%. In some embodiments, the degree of complementarity is 90-100%.

The guide RNAs can be about 5, 10, 11, 12, 13, 14, 15, 16, 17, 18, 19, 20, 21, 22, 23, 24, 25, 26, 27, 28, 29, 30, 35, 40, 45, 50, 75, 100, 125, 150, 175, 200 or more nucleotides in length. For example, for use in a functional Cas13c, Cas13d, Cas13e, or Cas13f effector protein, or homologs, orthologs, derivatives, fusions, conjugates, or functional fragment thereof, the spacer can be between 10-60 nucleotides, 20-50 nucleotides, 25-45 nucleotides, 25-35 nucleotides, or about 27, 28, 29, 30, 31, 32, or 33 nucleotides. For use in dCas version of any of the above, however, the spacer can be between 10-200 nucleotides, 20-150 nucleotides, 25-100 nucleotides, 25-85 nucleotides, 35-75 nucleotides, 45-60 nucleotides, or about 46, 47, 48, 49, 50, 51, 52, 53, 54, or 55 nucleotides.

To reduce off-target interactions, e.g., to reduce the guide interacting with a target sequence having low complementarity, mutations can be introduced to the CRISPR systems so that the CRISPR systems can distinguish between target and off-target sequences that have greater than 80%, 85%, 90%, or 95% complementarity. In some embodiments, the degree of complementarity is from 80% to 95%, e.g., about 83%, 84%, 85%, 86%, 87%, 88%, 89%, 90%, 91%, 92%, 93%, 94%, or 95% (for example, distinguishing between a target having 18 nucleotides from an off-target of 18 nucleotides having 1, 2, or 3 mismatches). Accordingly, in some embodiments, the degree of complementarity between a guide sequence and its corresponding target sequence is greater than 94.5%, 95%, 95.5%, 96%, 96.5%, 97%, 97.5%, 98%, 98.5%, 99%, 99.5%, or 99.9%. In some embodiments, the degree of complementarity is 100%.

It is known in the field that complete complementarity is not required, provided there is sufficient complementarity to be functional. Modulations of cleavage efficiency can be exploited by introduction of mismatches, e.g., one or more mismatches, such as 1 or 2 mismatches between spacer sequence and target sequence, including the position of the mismatch along the spacer/target. The more central (i.e., not at the 3′ or 5′-ends) a mismatch, e.g., a double mismatch, is located; the more cleavage efficiency is affected. Accordingly, by choosing mismatch positions along the spacer sequence, cleavage efficiency can be modulated. For example, if less than 100% cleavage of targets is desired (e.g., in a cell population), 1 or 2 mismatches between spacer and target sequence can be introduced in the spacer sequences.

Type VI CRISPR-Cas effectors have been demonstrated to employ more than one RNA guide, thus enabling the ability of these effectors, and systems and complexes that include them, to target multiple nucleic acids. In some embodiments, the CRISPR systems described herein include multiple RNA guides (e.g., two, three, four, five, six, seven, eight, nine, ten, fifteen, twenty, thirty, forty, or more) RNA guides. In some embodiments, the CRISPR systems described herein include a single RNA strand or a nucleic acid encoding a single RNA strand, wherein the RNA guides are arranged in tandem. The single RNA strand can include multiple copies of the same RNA guide, multiple copies of distinct RNA guides, or combinations thereof. The processing capability of the Type VI-C, VI-D, VI-E and VI-F CRISPR-Cas effector proteins described herein enables these effectors to be able to target multiple target nucleic acids (e.g., target RNAs) without a loss of activity. In some embodiments, the Type VI-C, VI-D, VI-E and VI-F CRISPR-Cas effector proteins may be delivered in complex with multiple RNA guides directed to different target RNA. In some embodiments, the Type VI-C, VI-D, VI-E and VI-F CRISPR-Cas effector proteins may be co-delivered with multiple RNA guides, each specific for a different target nucleic acid. Methods of multiplexing using CRISPR-associated proteins are described, for example, in U.S. Pat. No. 9,790,490 B2, and EP 3009511 B1, the entire contents of each of which are expressly incorporated herein by reference.

The spacer length of crRNAs can range from about 10-50 nucleotides, such as 15-50 nucleotides, 20-50 nucleotides, 25-50 nucleotide, or 19-50 nucleotides. In some embodiments, the spacer length of a guide RNA is at least 16 nucleotides, at least 17 nucleotides, at least 18 nucleotides, at least 19 nucleotides, at least 20 nucleotides, at least 21 nucleotides, or at least 22 nucleotides. In some embodiments, the spacer length is from 15 to 17 nucleotides (e.g., 15, 16, or 17 nucleotides), from 17 to 20 nucleotides (e.g., 17, 18, 19, or 20 nucleotides), from 20 to 24 nucleotides (e.g., 20, 21, 22, 23, or 24 nucleotides), from 23 to 25 nucleotides (e.g., 23, 24, or 25 nucleotides), from 24 to 27 nucleotides, from 27 to 30 nucleotides, from 30 to 45 nucleotides (e.g., 30, 31, 32, 33, 34, 35, 36, 37, 38, 39, 40, 41, 42, 43, 44, or 45 nucleotides), from 30 or 35 to 40 nucleotides, from 41 to 45 nucleotides, from 45 to 50 nucleotides (e.g., 45, 46, 47, 48, 49, or 50 nucleotides), or longer. In some embodiments, the spacer length is from about 15 to about 42 nucleotides.

In some embodiments, the direct repeat length of the guide RNA is 15-36 nucleotides, is at least 16 nucleotides, is from 16 to 20 nucleotides (e.g., 16, 17, 18, 19, or 20 nucleotides), is from 20-30 nucleotides (e.g., 20, 21, 22, 23, 24, 25, 26, 27, 28, 29, or 30 nucleotides), is from 30-40 nucleotides (e.g., 30, 31, 32, 33, 34, 35, 36, 37, 38, 39, or 40 nucleotides), or is about 36 nucleotides (e.g., 33, 34, 35, 36, 37, 38, or 39 nucleotides). In some embodiments, the direct repeat length of the guide RNA is 36 nucleotides.

In some embodiments, the overall length of the crRNA/guide RNA is about 36 nucleotides longer than any one of the spacer sequence length described herein above. For example, the overall length of the crRNA/guide RNA may be between 45-86 nucleotides, or 60-86 nucleotides, 62-86 nucleotides, or 63-86 nucleotides.

The crRNA sequences can be modified in a manner that allows for formation of a complex between the crRNA and CRISPR-associated protein and successful binding to the target, while at the same time not allowing for successful nuclease activity (i.e., without nuclease activity/without causing indels). These modified guide sequences are referred to as “dead crRNAs,” “dead guides,” or “dead guide sequences.” These dead guides or dead guide sequences may be catalytically inactive or conformationally inactive with regard to nuclease activity. Dead guide sequences are typically shorter than respective guide sequences that result in active RNA cleavage. In some embodiments, dead guides are 5%, 10%, 20%, 30%, 40%, or 50%, shorter than respective guide RNAs that have nuclease activity. Dead guide sequences of guide RNAs can be from 13 to 15 nucleotides in length (e.g., 13, 14, or 15 nucleotides in length), from 15 to 19 nucleotides in length, or from 17 to 18 nucleotides in length (e.g., 17 nucleotides in length).

Thus, in one aspect, the disclosure provides non-naturally occurring or engineered CRISPR systems including a functional CRISPR-associated protein as described herein, and a crRNA, wherein the crRNA comprises a dead crRNA sequence whereby the crRNA is capable of hybridizing to a target sequence such that the CRISPR system is directed to a genomic locus of interest in a cell without detectable nuclease activity (e.g., RNase activity).

A detailed description of dead guides is described, e.g., in International Publication No. WO 2016/094872, which is incorporated herein by reference in its entirety.

Guide RNAs (e.g., crRNAs) can be generated as components of inducible systems. The inducible nature of the systems allows for spatio-temporal control of gene editing or gene expression. In some embodiments, the stimuli for the inducible systems include, e.g., electromagnetic radiation, sound energy, chemical energy, and/or thermal energy.

In some embodiments, the transcription of guide RNA (e.g., crRNA) can be modulated by inducible promoters, e.g., tetracycline or doxycycline controlled transcriptional activation (Tet-On and Tet-Off expression systems), hormone inducible gene expression systems (e.g., ecdysone inducible gene expression systems), and arabinose-inducible gene expression systems. Other examples of inducible systems include, e.g., small molecule two-hybrid transcription activations systems (FKBP, ABA, etc.), light inducible systems (Phytochrome, LOV domains, or cryptochrome), or Light Inducible Transcriptional Effector (LITE). These inducible systems are described, e.g., in WO 2016205764 and U.S. Pat. No. 8,795,965, both of which are incorporated herein by reference in the entirety.

Chemical modifications can be applied to the crRNA's phosphate backbone, sugar, and/or base. Backbone modifications such as phosphorothioates modify the charge on the phosphate backbone and aid in the delivery and nuclease resistance of the oligonucleotide (see, e.g., Eckstein, “Phosphorothioates, essential components of therapeutic oligonucleotides,” Nucl. Acid Ther., 24, pp. 374-387, 2014); modifications of sugars, such as 2′-O-methyl (2′-OMe), 2′-F, and locked nucleic acid (LNA), enhance both base pairing and nuclease resistance (see, e.g., Allerson et al. “Fully 2′-modified oligonucleotide duplexes with improved in vitro potency and stability compared to unmodified small interfering RNA,” J. Med. Chem. 48.4: 901-904, 2005). Chemically modified bases such as 2-thiouridine or N6-methyladenosine, among others, can allow for either stronger or weaker base pairing (see, e.g., Bramsen et al., “Development of therapeutic-grade small interfering RNAs by chemical engineering,” Front. Genet., 2012 Aug. 20; 3:154). Additionally, RNA is amenable to both 5′ and 3′ end conjugations with a variety of functional moieties including fluorescent dyes, polyethylene glycol, or proteins.

A wide variety of modifications can be applied to chemically synthesized crRNA molecules. For example, modifying an oligonucleotide with a 2′-OMe to improve nuclease resistance can change the binding energy of Watson-Crick base pairing. Furthermore, a 2′-OMe modification can affect how the oligonucleotide interacts with transfection reagents, proteins or any other molecules in the cell. The effects of these modifications can be determined by empirical testing.

In some embodiments, the crRNA includes one or more phosphorothioate modifications. In some embodiments, the crRNA includes one or more locked nucleic acids for the purpose of enhancing base pairing and/or increasing nuclease resistance.

A summary of these chemical modifications can be found, e.g., in Kelley et al., “Versatility of chemically synthesized guide RNAs for CRISPR-Cas9 genome editing,” J. Biotechnol. 233:74-83, 2016; WO 2016205764; and U.S. Pat. No. 8,795,965 B2; each which is incorporated by reference in its entirety.

The sequences and the lengths of the RNA guides (e.g., crRNAs) described herein can be optimized. In some embodiments, the optimized length of an RNA guide can be determined by identifying the processed form of crRNA (i.e., a mature crRNA), or by empirical length studies for crRNA tetraloops.

The crRNAs can also include one or more aptamer sequences. Aptamers are oligonucleotide or peptide molecules have a specific three-dimensional structure and can bind to a specific target molecule. The aptamers can be specific to gene effectors, gene activators, or gene repressors. In some embodiments, the aptamers can be specific to a protein, which in turn is specific to and recruits and/or binds to specific gene effectors, gene activators, or gene repressors. The effectors, activators, or repressors can be present in the form of fusion proteins. In some embodiments, the guide RNA has two or more aptamer sequences that are specific to the same adaptor proteins. In some embodiments, the two or more aptamer sequences are specific to different adaptor proteins. The adaptor proteins can include, e.g., MS2, PP7, Qβ, F2, GA, fr, JP501, M12, R17, BZ13, JP34, JP500, KU1, M11, MX1, TW18, VK, SP, FI, ID2, NL95, TW19, AP205, ϕkCb5, ϕkCb8r, ϕkCb12r, ϕkCb23r, 7s, and PRR1. Accordingly, in some embodiments, the aptamer is selected from binding proteins specifically binding any one of the adaptor proteins as described herein. In some embodiments, the aptamer sequence is a MS2 binding loop (5′-ggcccAACAUGAGGAUCACCCAUGUCUGCAGgggcc-3′ (SEQ ID NO: 67)). In some embodiments, the aptamer sequence is a QBeta binding loop (5′-ggcccAUGCUGUCUAAGACA GCAUgggcc-3′ (SEQ ID NO: 68)). In some embodiments, the aptamer sequence is a PP7 binding loop (5′-ggcccUAAGGGUUUAUAUGGAAACCCUUAgggcc-3′ (SEQ ID NO: 69)). A detailed description of aptamers can be found, e.g., in Nowak et al., “Guide RNA engineering for versatile Cas9 functionality,” Nucl. Acid. Res., 44(20):9555-9564, 2016; and WO 2016205764, which are incorporated herein by reference in their entirety.

In certain embodiments, the methods make use of chemically modified guide RNAs. Examples of guide RNA chemical modifications include, without limitation, incorporation of 2′-O-methyl (M), 2′-O-methyl 3′-phosphorothioate (MS), or 2′-O-methyl 3′-thioPACE (MSP) at one or more terminal nucleotides. Such chemically modified guide RNAs can comprise increased stability and increased activity as compared to unmodified guide RNAs, though on-target vs. off-target specificity is not predictable. See, Hendel, Nat Biotechnol. 33(9):985-9, 2015, incorporated by reference). Chemically modified guide RNAs may further include, without limitation, RNAs with phosphorothioate linkages and locked nucleic acid (LNA) nucleotides comprising a methylene bridge between the 2′ and 4′ carbons of the ribose ring.

The invention also encompasses methods for delivering multiple nucleic acid components, wherein each nucleic acid component is specific for a different target locus of interest thereby modifying multiple target loci of interest. The nucleic acid component of the complex may comprise one or more protein-binding RNA aptamers. The one or more aptamers may be capable of binding a bacteriophage coat protein. The bacteriophage coat protein may be selected from the group comprising Qβ, F2, GA, fr, JP501, MS2, M12, R17, BZ13, JP34, JP500, KU1, M11, MX1, TW18, VK, SP, FI, ID2, NL95, TW19, AP205, ϕCb5, ϕCb8r, ϕCb12r, ϕCb23r, 7s and PRR1. In certain embodiments, the bacteriophage coat protein is MS2.

### 5. Target RNA

The target RNA can be any RNA molecule of interest, including naturally-occurring and engineered RNA molecules. The target RNA can be an mRNA, a tRNA, a ribosomal RNA (rRNA), a microRNA (miRNA), an interfering RNA (siRNA), a ribozyme, a riboswitch, a satellite RNA, a microswitch, a microzyme, or a viral RNA.

In some embodiments, the target nucleic acid is associated with a condition or disease (e.g., an infectious disease or a cancer).

Thus, in some embodiments, the systems described herein can be used to treat a condition or disease by targeting these nucleic acids. For instance, the target nucleic acid associated with a condition or disease may be an RNA molecule that is overexpressed in a diseased cell (e.g., a cancer or tumor cell). The target nucleic acid may also be a toxic RNA and/or a mutated RNA (e.g., an mRNA molecule having a splicing defect or a mutation). The target nucleic acid may also be an RNA that is specific for a particular microorganism (e.g., a pathogenic bacteria).

### 6. Complex and Cell

One aspect of the invention provides a CRISPR/Cas13c, CRISPR/Cas13d, CRISPR/Cas13e, or CRISPR/Cas13f complex comprising (1) any of the Cas13c/Cas13d/Cas13e/Cas13f effector proteins, homologs, orthologs, fusions, derivative, conjugates, or functional fragments thereof as described herein, and (2) any of the guide RNA described herein, each including a spacer sequence designed to be at least partially complementary to a target RNA, and a DR sequence compatible with the Cas13c/Cas13d/Cas13e/Cas13f effector proteins, homologs, orthologs, fusions, derivatives, conjugates, or functional fragments thereof.

In certain embodiments, the complex further comprises the target RNA bound by the guide RNA.

In certain embodiments, the complex is not naturally existing/occurring. For example, at least one of the components of the complex is not naturally existing/occurring. In certain embodiments, the Cas13c/Cas13d/Cas13e/Cas13f effector protein, homolog, ortholog, fusion, derivative, conjugate, or functional fragment thereof is not naturally occurring/existing due to, for example, the existence of at least one amino acid mutation (deletion, insertion, and/or substitution) as compared to a wild-type protein. In certain embodiments, the DR sequence is not naturally occurring/existing, i.e., not any one of SEQ ID NOs: 19-24 and 26-34, due to, for example, addition, deletion, and/or substitution of at least one nucleotide base in the wild-type sequence. In certain embodiments, the spacer sequence is not naturally occurring, in that it is not present or encoded by any spacer sequences present in the wild-type CRISPR locus of a prokaryote in which the subject Cas13c, Cas13d, Cas13e, or Cas13f exists. The spacer sequence may be not naturally existing when it is not 100% complementary to a naturally-occurring bacterialphage nucleic acid.

In a related aspect, the invention also provides a cell comprising any of the complex of the invention.

In certain embodiments, the cell is a prokaryote.

In certain embodiments, the cell is a eukaryote. When the cell is a eukaryote, the complex in the eukaryotic cell can be a naturally existing Cas13c/Cas13d/Cas13e/Cas13f complex in a prokaryote from which the Cas13c/Cas13d/Cas13e/Cas13f is isolated.

### 7. Methods of Using CRISPR Systems

The CRISPR systems described herein have a wide variety of utilities including modifying (e.g., deleting, inserting, translocating, inactivating, or activating) a target polynucleotide or nucleic acid in a multiplicity of cell types. The CRISPR systems have a broad spectrum of applications in, e.g., DNA/RNA detection (e.g., specific high sensitivity enzymatic reporter unlocking (SHERLOCK)), tracking and labeling of nucleic acids, enrichment assays (extracting desired sequence from background), controlling interfering RNA or miRNA, detecting circulating tumor DNA, preparing next generation library, drug screening, disease diagnosis and prognosis, and treating various genetic disorders.

### DNA/RNA Detection

In one aspect, the CRISPR systems described herein can be used in DNA or RNA detection. As shown in the examples, the Cas13c, Cas13d, Cas13e, and Cas13f proteins of the invention exhibit non-specific/collateral RNase activity upon activation of its guide RNA-dependent specific RNase activity when the spacer sequence is about 30 nucleotides. Thus the CRISPR-associated proteins of the invention can be reprogrammed with CRISPR RNAs (crRNAs) to provide a platform for specific RNA sensing. By choosing specific spacer sequence length, and upon recognition of its RNA target, activated CRISPR-associated proteins engage in “collateral” cleavage of nearby non-targeted RNAs. This crRNA-programmed collateral cleavage activity allows the CRISPR systems to detect the presence of a specific RNA by triggering programmed cell death or by nonspecific degradation of labeled RNA.

The SHERLOCK method (Specific High Sensitivity Enzymatic Reporter UnLOCKing) provides an in vitro nucleic acid detection platform with attomolar sensitivity based on nucleic acid amplification and collateral cleavage of a reporter RNA, allowing for real-time detection of the target. To achieve signal detection, the detection can be combined with different isothermal amplification steps. For example, recombinase polymerase amplification (RPA) can be coupled with T7 transcription to convert amplified DNA to RNA for subsequent detection. The combination of amplification by RPA, T7 RNA polymerase transcription of amplified DNA to RNA, and detection of target RNA by collateral RNA cleavage-mediated release of reporter signal is referred as SHERLOCK. Methods of using CRISPR in SHERLOCK are described in detail, e.g., in Gootenberg, et al. “Nucleic acid detection with CRISPR-Cas13a/C2c2,” Science, 2017 Apr. 28; 356(6336):438-442, which is incorporated herein by reference in its entirety.

The CRISPR-associated proteins can be used in Northern blot assays, which use electrophoresis to separate RNA samples by size. The CRISPR-associated proteins can be used to specifically bind and detect the target RNA sequence. The CRISPR-associated proteins can also be fused to a fluorescent protein (e.g., GFP) and used to track RNA localization in living cells. More particularly, the CRISPR-associated proteins can be inactivated in that they no longer cleave RNAs as described above. Thus, CRISPR-associated proteins can be used to determine the localization of the RNA or specific splice variants, the level of mRNA transcripts, up- or down-regulation of transcripts and disease-specific diagnosis. The CRISPR-associated proteins can be used for visualization of RNA in (living) cells using, for example, fluorescent microscopy or flow cytometry, such as fluorescence-activated cell sorting (FACS), which allows for high-throughput screening of cells and recovery of living cells following cell sorting. A detailed description regarding how to detect DNA and RNA can be found, e.g., in International Publication No. WO 2017/070605, which is incorporated herein by reference in its entirety.

In some embodiments, the CRISPR systems described herein can be used in multiplexed error-robust fluorescence in situ hybridization (MERFISH). These methods are described in, e.g., Chen et al., “Spatially resolved, highly multiplexed RNA profiling in single cells,” Science, 2015 Apr. 24; 348(6233):aaa6090, which is incorporated herein by reference herein in its entirety.

In some embodiments, the CRISPR systems described herein can be used to detect a target RNA in a sample (e.g., a clinical sample, a cell, or a cell lysate). The collateral RNase activity of the Type VI-C, VI-D, VI-E and VI-F CRISPR-Cas effector proteins described herein is activated when the effector proteins bind to a target nucleic acid when the spacer sequence is of a specific chosen length (such as about 30 nucleotides). Upon binding to the target RNA of interest, the effector protein cleaves a labeled detector RNA to generate a signal (e.g., an increased signal or a decreased signal) thereby allowing for the qualitative and quantitative detection of the target RNA in the sample. The specific detection and quantification of RNA in the sample allows for a multitude of applications including diagnostics. In some embodiments, the methods include contacting a sample with: i) an RNA guide (e.g., crRNA) and/or a nucleic acid encoding the RNA guide, wherein the RNA guide consists of a direct repeat sequence and a spacer sequence capable of hybridizing to the target RNA; (ii) a Type VI-C, VI-D, VI-E and VI-F CRISPR-Cas effector protein (Cas13c, Cas13d, Cas13e, or Cas13f) and/or a nucleic acid encoding the effector protein; and (iii) a labeled detector RNA; wherein the effector protein associates with the RNA guide to form a complex; wherein the RNA guide hybridizes to the target RNA; and wherein upon binding of the complex to the target RNA, the effector protein exhibits collateral RNase activity and cleaves the labeled detector RNA; and b) measuring a detectable signal produced by cleavage of the labeled detector RNA, wherein said measuring provides for detection of the single-stranded target RNA in the sample. In some embodiments, the methods further comprise comparing the detectable signal with a reference signal and determining the amount of target RNA in the sample. In some embodiments, the measuring is performed using gold nanoparticle detection, fluorescence polarization, colloid phase transition/dispersion, electrochemical detection, and semiconductor based-sensing. In some embodiments, the labeled detector RNA includes a fluorescence-emitting dye pair, a fluorescence resonance energy transfer (FRET) pair, or a quencher/fluor pair. In some embodiments, upon cleavage of the labeled detector RNA by the effector protein, an amount of detectable signal produced by the labeled detector RNA is decreased or increased. In some embodiments, the labeled detector RNA produces a first detectable signal prior to cleavage by the effector protein and a second detectable signal after cleavage by the effector protein. In some embodiments, a detectable signal is produced when the labeled detector RNA is cleaved by the effector protein. In some embodiments, the labeled detector RNA comprises a modified nucleobase, a modified sugar moiety, a modified nucleic acid linkage, or a combination thereof. In some embodiments, the methods include the multi-channel detection of multiple independent target RNAs in a sample (e.g., two, three, four, five, six, seven, eight, nine, ten, fifteen, twenty, thirty, forty, or more target RNAs) by using multiple Type VI-C, VI-D, VI-E and VI-F CRISPR-Cas (Cas13c, Cas13d, Cas13e, and/or Cas13f) systems, each including a distinct orthologous effector protein and corresponding RNA guides, allowing for the differentiation of multiple target RNAs in the sample. In some embodiments, the methods include the multi-channel detection of multiple independent target RNAs in a sample, with the use of multiple instances of Type VI-C, VI-D, VI-E and VI-F CRISPR-Cas systems, each containing an orthologous effector protein with differentiable collateral RNase substrates. Methods of detecting an RNA in a sample using CRISPR-associated proteins are described, for example, in U.S. Patent Publication No. 2017/0362644, the entire contents of which are incorporated herein by reference.

### Tracking and Labeling of Nucleic Acids

Cellular processes depend on a network of molecular interactions among proteins, RNAs, and DNAs. Accurate detection of protein-DNA and protein-RNA interactions is key to understanding such processes. In vitro proximity labeling techniques employ an affinity tag combined with, a reporter group, e.g., a photoactivatable group, to label polypeptides and RNAs in the vicinity of a protein or RNA of interest in vitro. After UV irradiation, the photoactivatable groups react with proteins and other molecules that are in close proximity to the tagged molecules, thereby labelling them. Labelled interacting molecules can subsequently be recovered and identified. The CRISPR-associated proteins can for instance be used to target probes to selected RNA sequences. These applications can also be applied in animal models for in vivo imaging of diseases or difficult-to culture cell types. The methods of tracking and labeling of nucleic acids are described, e.g., in U.S. Pat. No. 8,795,965, WO 2016205764, and WO 2017070605; each of which is incorporated herein by reference herein in its entirety.

RNA Isolation, Purification, Enrichment, and/or Depletion

The CRISPR systems (e.g., CRISPR-associated proteins) described herein can be used to isolate and/or purify the RNA. The CRISPR-associated proteins can be fused to an affinity tag that can be used to isolate and/or purify the RNA-CRISPR-associated protein complex. These applications are useful, e.g., for the analysis of gene expression profiles in cells.

In some embodiments, the CRISPR-associated proteins can be used to target a specific noncoding RNA (ncRNA) thereby blocking its activity. In some embodiments, the CRISPR-associated proteins can be used to specifically enrich a particular RNA (including but not limited to increasing stability, etc.), or alternatively, to specifically deplete a particular RNA (e.g., particular splice variants, isoforms, etc.).

These methods are described, e.g., in U.S. Pat. No. 8,795,965, WO 2016205764, and WO 2017070605; each of which is incorporated herein by reference herein in its entirety.

### High-Throughput Screening

The CRISPR systems described herein can be used for preparing next generation sequencing (NGS) libraries. For example, to create a cost-effective NGS library, the CRISPR systems can be used to disrupt the coding sequence of a target gene, and the CRISPR-associated protein transfected clones can be screened simultaneously by next-generation sequencing (e.g., on the Ion Torrent PGM system). A detailed description regarding how to prepare NGS libraries can be found, e.g., in Bell et al., “A high-throughput screening strategy for detecting CRISPR-Cas9 induced mutations using next-generation sequencing,” BMC Genomics, 15.1 (2014): 1002, which is incorporated herein by reference in its entirety.

### Engineered Microorganisms

Microorganisms (e.g., E. coli, yeast, and microalgae) are widely used for synthetic biology. The development of synthetic biology has a wide utility, including various clinical applications. For example, the programmable CRISPR systems can be used to split proteins of toxic domains for targeted cell death, e.g., using cancer-linked RNA as target transcript. Further, pathways involving protein-protein interactions can be influenced in synthetic biological systems with, e.g., fusion complexes with the appropriate effectors such as kinases or enzymes.

In some embodiments, crRNAs that target phage sequences can be introduced into the microorganism. Thus, the disclosure also provides methods of vaccinating a microorganism (e.g., a production strain) against phage infection.

In some embodiments, the CRISPR systems provided herein can be used to engineer microorganisms, e.g., to improve yield or improve fermentation efficiency. For example, the CRISPR systems described herein can be used to engineer microorganisms, such as yeast, to generate biofuel or biopolymers from fermentable sugars, or to degrade plant-derived lignocellulose derived from agricultural waste as a source of fermentable sugars. More particularly, the methods described herein can be used to modify the expression of endogenous genes required for biofuel production and/or to modify endogenous genes, which may interfere with the biofuel synthesis. These methods of engineering microorganisms are described e.g., in Verwaal et al., “CRISPR/Cpf1 enables fast and simple genome editing of Saccharomyces cerevisiae,” Yeast doi: 10.1002/yea.3278, 2017; and Hlavova et al., “Improving microalgae for biotechnology—from genetics to synthetic biology,” Biotechnol. Adv., 33:1194-203, 2015, both of which are incorporated herein by reference in the entirety.

In some embodiments, the CRISPR systems provided herein can be used to induce death or dormancy of a cell (e.g., a microorganism such as an engineered microorganism). These methods can be used to induce dormancy or death of a multitude of cell types including prokaryotic and eukaryotic cells, including, but not limited to mammalian cells (e.g., cancer cells, or tissue culture cells), protozoans, fungal cells, cells infected with a virus, cells infected with an intracellular bacteria, cells infected with an intracellular protozoan, cells infected with a prion, bacteria (e.g., pathogenic and non-pathogenic bacteria), protozoans, and unicellular and multicellular parasites. For instance, in the field of synthetic biology it is highly desirable to have mechanisms of controlling engineered microorganisms (e.g., bacteria) in order to prevent their propagation or dissemination. The systems described herein can be used as “kill-switches” to regulate and/or prevent the propagation or dissemination of an engineered microorganism. Further, there is a need in the art for alternatives to current antibiotic treatments. The systems described herein can also be used in applications where it is desirable to kill or control a specific microbial population (e.g., a bacterial population). For example, the systems described herein may include an RNA guide (e.g., a crRNA) that targets a nucleic acid (e.g., an RNA) that is genus-, species-, or strain-specific, and can be delivered to the cell. Upon complexing and binding to the target nucleic acid, the collateral RNase activity of the Type VI-C, VI-D, VI-E and VI-F CRISPR-Cas effector proteins is activated leading to the cleavage of non-target RNA within the microorganisms, ultimately resulting in dormancy or death. In some embodiments, the methods comprise contacting the cell with a system described herein including a Type VI-C, VI-D, VI-E and VI-F CRISPR-Cas effector proteins or a nucleic acid encoding the effector protein, and a RNA guide (e.g., a crRNA) or a nucleic acid encoding the RNA guide, wherein the spacer sequence is complementary to at least 15 nucleotides (e.g., 16, 17, 18, 19, 20, 21, 22, 23, 24, 25, 26, 27, 28, 29, 30, 35, 40, 45, 50 or more nucleotides) of a target nucleic acid (e.g., a genus-, strain-, or species-specific RNA guide). Without wishing to be bound by any particular theory, the cleavage of non-target RNA by the Type VI-C, VI-D, VI-E and VI-F CRISPR-Cas effector proteins may induce programmed cell death, cell toxicity, apoptosis, necrosis, necroptosis, cell death, cell cycle arrest, cell anergy, a reduction of cell growth, or a reduction in cell proliferation. For example, in bacteria, the cleavage of non-target RNA by the Type VI-C, VI-D, VI-E and VI-F CRISPR-Cas effector proteins may be bacteriostatic or bactericidal.

### Application in Plants

The CRISPR systems described herein have a wide variety of utility in plants. In some embodiments, the CRISPR systems can be used to engineer genomes of plants (e.g., improving production, making products with desired post-translational modifications, or introducing genes for producing industrial products). In some embodiments, the CRISPR systems can be used to introduce a desired trait to a plant (e.g., with or without heritable modifications to the genome), or regulate expression of endogenous genes in plant cells or whole plants.

In some embodiments, the CRISPR systems can be used to identify, edit, and/or silence genes encoding specific proteins, e.g., allergenic proteins (e.g., allergenic proteins in peanuts, soybeans, lentils, peas, green beans, and mung beans). A detailed description regarding how to identify, edit, and/or silence genes encoding proteins is described, e.g., in Nicolaou et al., “Molecular diagnosis of peanut and legume allergy,” Curr. Opin. Allergy Clin. Immunol. 11(3):222-8, 2011, and WO 2016205764 A1; both of which are incorporated herein by reference in the entirety.

### Gene Drives

Gene drive is the phenomenon in which the inheritance of a particular gene or set of genes is favorably biased. The CRISPR systems described herein can be used to build gene drives. For example, the CRISPR systems can be designed to target and disrupt a particular allele of a gene, causing the cell to copy the second allele to fix the sequence. Because of the copying, the first allele will be converted to the second allele, increasing the chance of the second allele being transmitted to the offspring. A detailed method regarding how to use the CRISPR systems described herein to build gene drives is described, e.g., in Hammond et al., “A CRISPR-Cas9 gene drive system targeting female reproduction in the malaria mosquito vector Anopheles gambiae,” Nat. Biotechnol. 34(1):78-83, 2016, which is incorporated herein by reference in its entirety.

### Pooled-Screening

As described herein, pooled CRISPR screening is a powerful tool for identifying genes involved in biological mechanisms such as cell proliferation, drug resistance, and viral infection. Cells are transduced in bulk with a library of guide RNA (gRNA)-encoding vectors described herein, and the distribution of gRNAs is measured before and after applying a selective challenge. Pooled CRISPR screens work well for mechanisms that affect cell survival and proliferation, and they can be extended to measure the activity of individual genes (e.g., by using engineered reporter cell lines). Arrayed CRISPR screens, in which only one gene is targeted at a time, make it possible to use RNA-seq as the readout. In some embodiments, the CRISPR systems as described herein can be used in single-cell CRISPR screens. A detailed description regarding pooled CRISPR screenings can be found, e.g., in Datlinger et al., “Pooled CRISPR screening with single-cell transcriptome read-out,” Nat. Methods. 14(3):297-301, 2017, which is incorporated herein by reference in its entirety.

### Saturation Mutagenesis (Bashing)

The CRISPR systems described herein can be used for in situ saturating mutagenesis. In some embodiments, a pooled guide RNA library can be used to perform in situ saturating mutagenesis for particular genes or regulatory elements. Such methods can reveal critical minimal features and discrete vulnerabilities of these genes or regulatory elements (e.g., enhancers). These methods are described, e.g., in Canver et al., “BCL11A enhancer dissection by Cas9-mediated in situ saturating mutagenesis,” Nature 527(7577):192-7, 2015, which is incorporated herein by reference in its entirety.

### RNA-Related Applications

The CRISPR systems described herein can have various RNA-related applications, e.g., modulating gene expression, degrading a RNA molecule, inhibiting RNA expression, screening RNA or RNA products, determining functions of lincRNA or non-coding RNA, inducing cell dormancy, inducing cell cycle arrest, reducing cell growth and/or cell proliferation, inducing cell anergy, inducing cell apoptosis, inducing cell necrosis, inducing cell death, and/or inducing programmed cell death. A detailed description of these applications can be found, e.g., in WO 2016/205764 A1, which is incorporated herein by reference in its entirety. In different embodiments, the methods described herein can be performed in vitro, in vivo, or ex vivo.

For example, the CRISPR systems described herein can be administered to a subject having a disease or disorder to target and induce cell death in a cell in a diseased state (e.g., cancer cells or cells infected with an infectious agent). For instance, in some embodiments, the CRISPR systems described herein can be used to target and induce cell death in a cancer cell, wherein the cancer cell is from a subject having a Wilms' tumor, Ewing sarcoma, a neuroendocrine tumor, a glioblastoma, a neuroblastoma, a melanoma, skin cancer, breast cancer, colon cancer, rectal cancer, prostate cancer, liver cancer, renal cancer, pancreatic cancer, lung cancer, biliary cancer, cervical cancer, endometrial cancer, esophageal cancer, gastric cancer, head and neck cancer, medullary thyroid carcinoma, ovarian cancer, glioma, lymphoma, leukemia, myeloma, acute lymphoblastic leukemia, acute myelogenous leukemia, chronic lymphocytic leukemia, chronic myelogenous leukemia, Hodgkin's lymphoma, non-Hodgkin's lymphoma, or urinary bladder cancer.

### Modulating Gene Expression

The CRISPR systems described herein can be used to modulate gene expression. The CRISPR systems can be used, together with suitable guide RNAs, to target gene expression, via control of RNA processing. The control of RNA processing can include, e.g., RNA processing reactions such as RNA splicing (e.g., alternative splicing), viral replication, and tRNA biosynthesis. The RNA targeting proteins in combination with suitable guide RNAs can also be used to control RNA activation (RNAa). RNA activation is a small RNA-guided and Argonaute (Ago)-dependent gene regulation phenomenon in which promoter-targeted short double-stranded RNAs (dsRNAs) induce target gene expression at the transcriptional/epigenetic level. RNAa leads to the promotion of gene expression, so control of gene expression may be achieved that way through disruption or reduction of RNAa. In some embodiments, the methods include the use of the RNA targeting CRISPR as substitutes for e.g., interfering ribonucleic acids (such as siRNAs, shRNAs, or dsRNAs). The methods of modulating gene expression are described, e.g., in WO 2016205764, which is incorporated herein by reference in its entirety.

### Controlling RNA Interference

Control over interfering RNAs or microRNAs (miRNA) can help reduce off-target effects by reducing the longevity of the interfering RNAs or miRNAs in vivo or in vitro. In some embodiments, the target RNAs can include interfering RNAs, i.e., RNAs involved in the RNA interference pathway, such as small hairpin RNAs (shRNAs), small interfering (siRNAs), etc. In some embodiments, the target RNAs include, e.g., miRNAs or double stranded RNAs (dsRNA).

In some embodiments, if the RNA targeting protein and suitable guide RNAs are selectively expressed (for example spatially or temporally under the control of a regulated promoter, for example a tissue- or cell cycle-specific promoter and/or enhancer), this can be used to protect the cells or systems (in vivo or in vitro) from RNA interference (RNAi) in those cells. This may be useful in neighboring tissues or cells where RNAi is not required or for the purposes of comparison of the cells or tissues where the CRISPR-associated proteins and suitable crRNAs are and are not expressed (i.e., where the RNAi is not controlled and where it is, respectively). The RNA targeting proteins can be used to control or bind to molecules comprising or consisting of RNAs, such as ribozymes, ribosomes, or riboswitches. In some embodiments, the guide RNAs can recruit the RNA targeting proteins to these molecules so that the RNA targeting proteins are able to bind to them. These methods are described, e.g., in WO 2016205764 and WO 2017070605, both of which are incorporated herein by reference in the entirety.

### Modifying Riboswitches and Controlling Metabolic Regulations

Riboswitches are regulatory segments of messenger RNAs that bind small molecules and in turn regulate gene expression. This mechanism allows the cell to sense the intracellular concentration of these small molecules. A specific riboswitch typically regulates its adjacent gene by altering the transcription, the translation or the splicing of this gene. Thus, in some embodiments, the riboswitch activity can be controlled by the use of the RNA targeting proteins in combination with suitable guide RNAs to target the riboswitches. This may be achieved through cleavage of, or binding to, the riboswitch. Methods of using CRISPR systems to control riboswitches are described, e.g., in WO 2016205764 and WO 2017070605, both of which are incorporated herein by reference in their entireties.

### RNA Modification

In some embodiments, the CRISPR-associated proteins described herein can be fused to a base-editing domain, such as ADAR1, ADAR2, APOBEC, or activation-induced cytidine deaminase (AID), and can be used to modify an RNA sequence (e.g., an mRNA). In some embodiments, the CRISPR-associated protein includes one or more mutations (e.g., in a catalytic domain), which renders the CRISPR-associated protein incapable of cleaving RNA.

In some embodiments, the CRISPR-associated proteins can be used with an RNA-binding fusion polypeptide comprising a base-editing domain (e.g., ADAR1, ADAR2, APOBEC, or AID) fused to an RNA-binding domain, such as MS2 (also known as MS2 coat protein), Qbeta (also known as Qbeta coat protein), or PP7 (also known as PP7 coat protein). The amino acid sequences of the RNA-binding domains MS2, Qbeta, and PP7 are provided below:

MS2 (MS2 coat protein)
(SEQ ID NO: 60)
MASNFTQFVLVDNGGTGDVTVAPSNFANGVAEWISSNSRSQAYKVTC
SVRQSSAQKRKYTIKVEVPKVATQTVGGVELPVAAWRSYLNMELTIP
IFATNSDCELIVKAMQGLLKDGNPIPSAIAANSGIY
Qbeta (Qbeta coat protein)
(SEQ ID NO: 61)
MAKLETVTLGNIGKDGKQTLVLNPRGVNPTNGVASLSQAGAVPALEK
RVTVSVSQPSRNRKNYKVQVKIQNPTACTANGSCDPSVTRQAYADVT
FSFTQYSTDEERAFVRTELAALLASPLLIDAIDQLNPAY
PP7 (PP7 coat protein)
(SEQ ID NO: 62)
MSKTIVLSVGEATRTLTEIQSTADRQIFEEKVGPLVGRLRLTASLRQ
NGAKTAYRVNLKLDQADVVDCSTSVCGELPKVRYTQVWSHDVTIVAN
STEASRKSLYDLTKSLVVQATSEDLVVNLVPLGR

In some embodiments, the RNA binding domain can bind to a specific sequence (e.g., an aptamer sequence) or secondary structure motifs on a crRNA of the system described herein (e.g., when the crRNA is in an effector-crRNA complex), thereby recruiting the RNA binding fusion polypeptide (which has a base-editing domain) to the effector complex. For example, in some embodiments, the CRISPR system includes a CRISPR associated protein, a crRNA having an aptamer sequence (e.g., an MS2 binding loop, a QBeta binding loop, or a PP7 binding loop), and a RNA-binding fusion polypeptide having a base-editing domain fused to an RNA-binding domain that specifically binds to the aptamer sequence. In this system, the CRISPR-associated protein forms a complex with the crRNA having the aptamer sequence. Further the RNA-binding fusion polypeptide binds to the crRNA (via the aptamer sequence) thereby forming a tripartite complex that can modify a target RNA.

Methods of using CRISPR systems for base editing are described, e.g., in International Publication No. WO 2017/219027, which is incorporated herein by reference in its entirety, and in particular with respect to its discussion of RNA modification.

### RNA Splicing

In some embodiments, an inactivated CRISPR-associated protein described herein (e.g., a CRISPR associated protein having one or more mutations in a catalytic domain) can be used to target and bind to specific splicing sites on RNA transcripts. Binding of the inactivated CRISPR-associated protein to the RNA may sterically inhibit interaction of the spliceosome with the transcript, enabling alteration in the frequency of generation of specific transcript isoforms. Such method can be used to treat disease through exon skipping such that an exon having a mutation may be skipped in a mature protein. Methods of using CRISPR systems to alter splicing are described, e.g., in International Publication No. WO 2017/219027, which is incorporated herein by reference in its entirety, and in particular with respect to its discussion of RNA splicing.

### Therapeutic Applications

The CRISPR systems described herein can have various therapeutic applications. Such applications may be based on one or more of the abilities below, both in vitro and in vivo, of the subject CRISPR/Cas13c, Cas13d, Cas13e or Cas13f systems: induce cellular senescence, induce cell cycle arrest, inhibit cell growth and/or proliferation, induce apoptosis, induce necrosis, etc.

In some embodiments, the new CRISPR systems can be used to treat various diseases and disorders, e.g., genetic disorders (e.g., monogenetic diseases), diseases that can be treated by nuclease activity (e.g., Pcsk9 targeting, Duchenne Muscular Dystrophy (DMD), BCL11a targeting), and various cancers, etc.

In some embodiments, the CRISPR systems described herein can be used to edit a target nucleic acid to modify the target nucleic acid (e.g., by inserting, deleting, or mutating one or more nucleic acid residues). For example, in some embodiments the CRISPR systems described herein comprise an exogenous donor template nucleic acid (e.g., a DNA molecule or a RNA molecule), which comprises a desirable nucleic acid sequence. Upon resolution of a cleavage event induced with the CRISPR system described herein, the molecular machinery of the cell will utilize the exogenous donor template nucleic acid in repairing and/or resolving the cleavage event. Alternatively, the molecular machinery of the cell can utilize an endogenous template in repairing and/or resolving the cleavage event. In some embodiments, the CRISPR systems described herein may be used to alter a target nucleic acid resulting in an insertion, a deletion, and/or a point mutation). In some embodiments, the insertion is a scarless insertion (i.e., the insertion of an intended nucleic acid sequence into a target nucleic acid resulting in no additional unintended nucleic acid sequence upon resolution of the cleavage event). Donor template nucleic acids may be double stranded or single stranded nucleic acid molecules (e.g., DNA or RNA). Methods of designing exogenous donor template nucleic acids are described, for example, in International Publication No. WO 2016/094874 A1, the entire contents of which are expressly incorporated herein by reference.

In one aspect, the CRISPR systems described herein can be used for treating a disease caused by overexpression of RNAs, toxic RNAs, and/or mutated RNAs (e.g., splicing defects or truncations). For example, expression of toxic RNAs may be associated with the formation of nuclear inclusions and late-onset degenerative changes in brain, heart, or skeletal muscle. In some embodiments, the disorder is myotonic dystrophy. In myotonic dystrophy, the main pathogenic effect of the toxic RNAs is to sequester binding proteins and compromise the regulation of alternative splicing (see, e.g., Osborne et al., “RNA-dominant diseases,” Hum. Mol. Genet., 2009 Apr. 15; 18(8):1471-81). Myotonic dystrophy (dystrophia myotonica (DM)) is of particular interest to geneticists because it produces an extremely wide range of clinical features. The classical form of DM, which is now called DM type 1 (DM1), is caused by an expansion of CTG repeats in the 3′-untranslated region (UTR) of DMPK, a gene encoding a cytosolic protein kinase. The CRISPR systems as described herein can target overexpressed RNA or toxic RNA, e.g., the DMPK gene or any of the mis-regulated alternative splicing in DM1 skeletal muscle, heart, or brain.

The CRISPR systems described herein can also target trans-acting mutations affecting RNA-dependent functions that cause various diseases such as, e.g., Prader Willi syndrome, Spinal muscular atrophy (SMA), and Dyskeratosis congenita. A list of diseases that can be treated using the CRISPR systems described herein is summarized in Cooper et al., “RNA and disease,” Cell, 136.4 (2009): 777-793, and WO 2016/205764 A1, both of which are incorporated herein by reference in the entirety. Those of skill in this field will understand how to use the new CRISPR systems to treat these diseases.

The CRISPR systems described herein can also be used in the treatment of various tauopathies, including, e.g., primary and secondary tauopathies, such as primary age-related tauopathy (PART)/Neurofibrillary tangle (NFT)-predominant senile dementia (with NFTs similar to those seen in Alzheimer Disease (AD), but without plaques), dementia pugilistica (chronic traumatic encephalopathy), and progressive supranuclear palsy. A useful list of tauopathies and methods of treating these diseases are described, e.g., in WO 2016205764, which is incorporated herein by reference in its entirety.

The CRISPR systems described herein can also be used to target mutations disrupting the cis-acting splicing codes that can cause splicing defects and diseases. These diseases include, e.g., motor neuron degenerative disease that results from deletion of the SMN1 gene (e.g., spinal muscular atrophy), Duchenne Muscular Dystrophy (DMD), frontotemporal dementia, and Parkinsonism linked to chromosome 17 (FTDP-17), and cystic fibrosis.

The CRISPR systems described herein can further be used for antiviral activity, in particular against RNA viruses. The CRISPR-associated proteins can target the viral RNAs using suitable guide RNAs selected to target viral RNA sequences.

The CRISPR systems described herein can also be used to treat a cancer in a subject (e.g., a human subject). For example, the CRISPR-associated proteins described herein can be programmed with crRNA targeting a RNA molecule that is aberrant (e.g., comprises a point mutation or are alternatively-spliced) and found in cancer cells to induce cell death in the cancer cells (e.g., via apoptosis).

The CRISPR systems described herein can also be used to treat an autoimmune disease or disorder in a subject (e.g., a human subject). For example, the CRISPR-associated proteins described herein can be programmed with crRNA targeting a RNA molecule that is aberrant (e.g., comprises a point mutation or are alternatively-spliced) and found in cells responsible for causing the autoimmune disease or disorder.

Further, the CRISPR systems described herein can also be used to treat an infectious disease in a subject. For example, the CRISPR-associated proteins described herein can be programmed with crRNA targeting a RNA molecule expressed by an infectious agent (e.g., a bacteria, a virus, a parasite or a protozoan) in order to target and induce cell death in the infectious agent cell. The CRISPR systems may also be used to treat diseases where an intracellular infectious agent infects the cells of a host subject. By programming the CRISPR-associated protein to target a RNA molecule encoded by an infectious agent gene, cells infected with the infectious agent can be targeted and cell death induced.

Furthermore, in vitro RNA sensing assays can be used to detect specific RNA substrates. The CRISPR-associated proteins can be used for RNA-based sensing in living cells. Examples of applications are diagnostics by sensing of, for examples, disease-specific RNAs.

A detailed description of therapeutic applications of the CRISPR systems described herein can be found, e.g., in U.S. Pat. No. 8,795,965, EP 3009511, WO 2016205764, and WO 2017070605; each of which is incorporated herein by reference in its entirety.

### Cells and Progenies Thereof

In certain embodiments, the methods of the invention can be used to introduce the CRISPR systems described herein into a cell, and cause the cell and/or its progeny to alter the production of one or more cellular produces, such as antibody, starch, ethanol, or any other desired products. Such cells and progenies thereof are within the scope of the invention.

In certain embodiments, the methods and/or the CRISPR systems described herein lead to modification of the translation and/or transcription of one or more RNA products of the cells. For example, the modification may lead to increased transcription/translation/expression of the RNA product. In other embodiments, the modification may lead to decreased transcription/translation/expression of the RNA product.

In certain embodiments, the cell is a prokaryotic cell.

In certain embodiments, the cell is a eukaryotic cell, such as a mammalian cell, including a human cell (a primary human cell or an established human cell line). In certain embodiments, the cell is a non-human mammalian cell, such as a cell from a non-human primate (e.g., monkey), a cow/bull/cattle, sheep, goat, pig, horse, dog, cat, rodent (such as rabbit, mouse, rat, hamster, etc). In certain embodiments, the cell is from fish (such as salmon), bird (such as poultry bird, including chick, duck, goose), reptile, shellfish (e.g., oyster, claim, lobster, shrimp), insect, worm, yeast, etc. In certain embodiments, the cell is from a plant, such as monocot or dicot. In certain embodiment, the plant is a food crop such as barley, cassava, cotton, groundnuts or peanuts, maize, millet, oil palm fruit, potatoes, pulses, rapeseed or canola, rice, rye, sorghum, soybeans, sugar cane, sugar beets, sunflower, and wheat. In certain embodiment, the plant is a cereal (barley, maize, millet, rice, rye, sorghum, and wheat). In certain embodiment, the plant is a tuber (cassava and potatoes). In certain embodiment, the plant is a sugar crop (sugar beets and sugar cane). In certain embodiment, the plant is an oil-bearing crop (soybeans, groundnuts or peanuts, rapeseed or canola, sunflower, and oil palm fruit). In certain embodiment, the plant is a fiber crop (cotton). In certain embodiment, the plant is a tree (such as a peach or a nectarine tree, an apple or pear tree, a nut tree such as almond or walnut or pistachio tree, or a citrus tree, e.g., orange, grapefruit or lemon tree), a grass, a vegetable, a fruit, or an algae. In certain embodiment, the plant is a nightshade plant; a plant of the genus Brassica; a plant of the genus Lactuca; a plant of the genus Spinacia; a plant of the genus Capsicum; cotton, tobacco, asparagus, carrot, cabbage, broccoli, cauliflower, tomato, eggplant, pepper, lettuce, spinach, strawberry, blueberry, raspberry, blackberry, grape, coffee, cocoa, etc.

A related aspect provides cells or progenies thereof modified by the methods of the invention using the CRISPR systems described herein.

In certain embodiments, the cell is modified in vitro, in vivo, or ex vivo.

In certain embodiments, the cell is a stem cell.

### 7. Delivery

Through this disclosure and the knowledge in the art, the CRISPR systems described herein, or any of the components thereof described herein (Cas proteins, derivatives, functional fragments or the various fusions or adducts thereof, and guide RNA/crRNA), nucleic acid molecules thereof, and/or nucleic acid molecules encoding or providing components thereof, can be delivered by various delivery systems such as vectors, e.g., plasmids and viral delivery vectors, using any suitable means in the art. Such methods include (and are not limited to) electroporation, lipofection, microinjection, transfection, sonication, gene gun, etc.

In certain embodiments, the CRISPR-associated proteins and/or any of the RNAs (e.g., guide RNAs or crRNAs) and/or accessory proteins can be delivered using suitable vectors, e.g., plasmids or viral vectors, such as adeno-associated viruses (AAV), lentiviruses, adenoviruses, retroviral vectors, and other viral vectors, or combinations thereof. The proteins and one or more crRNAs can be packaged into one or more vectors, e.g., plasmids or viral vectors. For bacterial applications, the nucleic acids encoding any of the components of the CRISPR systems described herein can be delivered to the bacteria using a phage. Exemplary phages, include, but are not limited to, T4 phage, Mu, λ phage, T5 phage, T7 phage, T3 phage, Φ29, M13, MS2, Qβ, and ΦX174.

In some embodiments, the vectors, e.g., plasmids or viral vectors, are delivered to the tissue of interest by, e.g., intramuscular injection, intravenous administration, transdermal administration, intranasal administration, oral administration, or mucosal administration. Such delivery may be either via a single dose, or multiple doses. One skilled in the art understands that the actual dosage to be delivered herein may vary greatly depending upon a variety of factors, such as the vector choices, the target cells, organisms, tissues, the general conditions of the subject to be treated, the degrees of transformation/modification sought, the administration routes, the administration modes, the types of transformation/modification sought, etc.

In certain embodiments, the delivery is via adenoviruses, which can be at a single dose containing at least 1×10⁵ particles (also referred to as particle units, pu) of adenoviruses. In some embodiments, the dose preferably is at least about 1×10⁶ particles, at least about 1×10⁷ particles, at least about 1×10⁸ particles, and at least about 1×10⁹ particles of the adenoviruses. The delivery methods and the doses are described, e.g., in WO 2016205764 A1 and U.S. Pat. No. 8,454,972 B2, both of which are incorporated herein by reference in the entirety.

In some embodiments, the delivery is via plasmids. The dosage can be a sufficient number of plasmids to elicit a response. In some cases, suitable quantities of plasmid DNA in plasmid compositions can be from about 0.1 to about 2 mg. Plasmids will generally include (i) a promoter; (ii) a sequence encoding a nucleic acid-targeting CRISPR-associated proteins and/or an accessory protein, each operably linked to a promoter (e.g., the same promoter or a different promoter); (iii) a selectable marker; (iv) an origin of replication; and (v) a transcription terminator downstream of and operably linked to (ii). The plasmids can also encode the RNA components of a CRISPR complex, but one or more of these may instead be encoded on different vectors. The frequency of administration is within the ambit of the medical or veterinary practitioner (e.g., physician, veterinarian), or a person skilled in the art.

In another embodiment, the delivery is via liposomes or lipofection formulations and the like, and can be prepared by methods known to those skilled in the art. Such methods are described, for example, in WO 2016205764 and U.S. Pat. Nos. 5,593,972; 5,589,466; and 5,580,859; each of which is incorporated herein by reference in its entirety.

In some embodiments, the delivery is via nanoparticles or exosomes. For example, exosomes have been shown to be particularly useful in delivery RNA.

Further means of introducing one or more components of the new CRISPR systems to the cell is by using cell penetrating peptides (CPP). In some embodiments, a cell penetrating peptide is linked to the CRISPR-associated proteins. In some embodiments, the CRISPR-associated proteins and/or guide RNAs are coupled to one or more CPPs to effectively transport them inside cells (e.g., plant protoplasts). In some embodiments, the CRISPR-associated proteins and/or guide RNA(s) are encoded by one or more circular or non-circular DNA molecules that are coupled to one or more CPPs for cell delivery.

CPPs are short peptides of fewer than 35 amino acids derived either from proteins or from chimeric sequences capable of transporting biomolecules across cell membrane in a receptor independent manner CPPs can be cationic peptides, peptides having hydrophobic sequences, amphipathic peptides, peptides having proline-rich and anti-microbial sequences, and chimeric or bipartite peptides. Examples of CPPs include, e.g., Tat (which is a nuclear transcriptional activator protein required for viral replication by HIV type 1), penetratin, Kaposi fibroblast growth factor (FGF) signal peptide sequence, integrin β3 signal peptide sequence, polyarginine peptide Args sequence, Guanine rich-molecular transporters, and sweet arrow peptide. CPPs and methods of using them are described, e.g., in Hällbrink et al., “Prediction of cell-penetrating peptides,” Methods Mol. Biol., 2015; 1324:39-58; Ramakrishna et al., “Gene disruption by cell-penetrating peptide-mediated delivery of Cas9 protein and guide RNA,” Genome Res., 2014 June; 24(6):1020-7; and WO 2016205764 A1; each of which is incorporated herein by reference in its entirety.

Various delivery methods for the CRISPR systems described herein are also described, e.g., in U.S. Pat. No. 8,795,965, EP 3009511, WO 2016205764, and WO 2017070605; each of which is incorporated herein by reference in its entirety.

### 8. Kits

Another aspect of the invention provides a kit, comprising any two or more components of the subject CRISPR/Cas system described herein, such as the Cas13c, Cas13d, Cas13e and Cas13f proteins, derivatives, functional fragments or the various fusions or adducts thereof, guide RNA/crRNA, complexes thereof, vectors encompassing the same, or host encompassing the same.

In certain embodiments, the kit further comprise an instruction to use the components encompassed therein, and/or instructions for combining with additional components that may be available elsewhere.

In certain embodiments, the kit further comprise one or more nucleotides, such as nucleotide(s) corresponding to those useful to insert the guide RNA coding sequence into a vector and operably linking the coding sequence to one or more control elements of the vector.

In certain embodiments, the kit further comprise one or more buffers that may be used to dissolve any of the components, and/or to provide suitable reaction conditions for one or more of the components. Such buffers may include one or more of PBS, HEPES, Tris, MOPS, Na₂CO₃, NaHCO₃, NaB, or combinations thereof. In certain embodiments, the reaction condition includes a proper pH, such as a basic pH. In certain embodiments, the pH is between 7-10.

In certain embodiments, any one or more of the kit components may be stored in a suitable container.

### EXAMPLES

### Example 1 Identification of Novel Cas13c, Cas13d, Cas13e and Cas13f Systems

A computational pipeline was used to produce an expanded database of class 2 CRISPR-Cas systems from genomic and metagenomic sources. Genome and metagenome sequences were downloaded from NCBI (Benson et al., 2013; Pruitt et al., 2012), NCBI whole genome sequencing (WGS), and DOE JGI Integrated Microbial Genomes (Markowitz et al., 2012). Proteins were predicted (Prodigal (Hyatt et al., 2010) in anon mode) on all contigs at least 5 kb in length, and de-duplicated (i.e., removing identical protein sequences) to construct a complete protein database. Proteins larger than 600 residues were considered as Large Proteins (LPs). Since the currently identified Cas13 proteins are mostly larger than 900 residues in size, in order to reduce the complexity of calculation, only Large Proteins were considered further.

CRISPR arrays were identified using Piler-CR (Edgar, PILER-CR: Fast and accurate identification of CRISPR repeats. BMC Bioinformatics 8:18, 2007), using all default parameters. Non-redundant Large Protein sequence-encoding ORFs located within ±10 kb from the CRISPR arrays were grouped into CRISPR-proximal Large Protein encoding clusters, and the encoded LPs were defined as Cas-LPs.

First, BLASTP was used to conduct pairwise alignment between the Cas-LPs, and BLASTP alignment results with Evalue <1E-10 were obtained. MCL was then used to further cluster the Cas-LPs based on the BLASTP results to create families of Cas proteins.

Next, BLASTP was used to align Cas-LPs to all LPs and BLASP alignment results with Evalue<1E-10 were obtained. Cas-LPs families were further expanded according to the BLASTP alignment results. The Cas-LP families were obtained for further analysis with no more than double increase after expansion.

For functional characterization of the candidate Cas proteins, protein family databases Pfam (Finn et al., 2014), NR database, and Cas proteins in NCBI were used to annotate the candidate Cas proteins. Multiple sequence alignment was then conducted for each candidate Cas effector proteins using MAFFT (Katoh and Standley, 2013). JPred and HHpred were then used to analyze conserved regions in these proteins, to identify candidate Cas proteins/families having two conserved RXXXXH motifs.

This analysis led to the identification of fifteen novel Cas13 effector proteins falling within four new Cas13 families different from previously identified Class 2 CRISPR-Cas systems. These include Cas13e.3 (SEQ ID NO: 2), Cas13e.4 (SEQ ID NO: 3), Cas13e.5 (SEQ ID NO: 4), Cas13e.6 (SEQ ID NO: 5), Cas13e.7 (SEQ ID NO: 6), and Cas13e.8 (SEQ ID NO: 7) of the Cas13e family; Cas13f.6 (SEQ ID NO: 9) and Cas13f.7 (SEQ ID NO: 10) of the Cas13f family; Cas13d.1 (SEQ ID NO: 11), Cas13d.2 (SEQ ID NO: 12), Cas13d.3 (SEQ ID NO: 13), Cas13d.4 (SEQ ID NO: 14), and Cas13d.5 (SEQ ID NO: 15) of the Cas13d family; and Cas13c.1 (SEQ ID NO: 16) and Cas13c.2 (SEQ ID NO: 17) of the Cas13c family See below.

The previously identified Cas13e.1 (SEQ ID NO: 1) and Cas13f.1 (SEQ ID NO: 8) are also listed below.

### Cas13e.1 (SEQ ID NO: 1)

### Cas13e.3 (SEQ ID NO: 2)

### Cas13e.4 (SEQ ID NO: 3)

### Cas13e.5 (SEQ ID NO: 4)

### Cas13e.6 (SEQ ID NO: 5)

### Cas13e.7 (SEQ ID NO: 6)

### Cas13e.8 (SEQ ID NO: 7)

### Cas13f.1 (SEQ ID NO: 8)

### Cas13f.6 (SEQ ID NO: 9)

### Cas13f.7 (SEQ ID NO: 10)

### Cas13d.1 (SEQ ID NO: 11)

### Cas13d.2 (SEQ ID NO: 12)

### Cas13d.3 (SEQ ID NO: 13)

### Cas13d.4 (SEQ ID NO: 14)

### Cas13d.5 (SEQ ID NO: 15)

### Cas13c.1 (SEQ ID NO: 16)

### Cas13c.2 (SEQ ID NO: 17)

DNA encoding the corresponding Direct Repeat (DR) sequences in the respective pre-crRNA sequences are SEQ ID NOs: 19-24 and 26-34, for the Cas13 effector enzymes of SEQ ID NOs. 2-7 and 9-17, respectively.

(SEQ ID NO: 19)
GCTGGAGCAGCCCTCGATTTGCTGGGTAATCACAGC
(SEQ ID NO: 20)
GCTGAAGCAACCCTGGTTTTGCGGGGTGATTACAGC
(SEQ ID NO: 21)
GCTGTAGAAGCCTCCGATTTGTGAGGTGATGACAGC
(SEQ ID NO: 22)
GCTGGAGCAGCCCTCGATTTGCAGGGTAATCACAGC
(SEQ ID NO: 23)
GCTGGAGCAGCCCTCGATTTGCAGGGTTATCACAGC
(SEQ ID NO: 24)
GTTGGAGTAGCCCCGGATTTGCGGGGTGATTACAGC
(SEQ ID NO: 26)
GCTGTGATAGACCTCGATTTGTGGGGTAGTAACAGC
(SEQ ID NO: 27)
GCTGTGATGGACCTCGATTTGTGGGGTAGTAACAGC
(SEQ ID NO: 28)
CAACTACAACCCCGTAAAAATACGGGGTTCTGAAAC
(SEQ ID NO: 29)
GTTAAATACCACCTAAGAATGAGGAGGTTCTATAAC
(SEQ ID NO: 30)
GAACGATAGCCTGCTGAAATATGCAGGTTCTAAGAC
(SEQ ID NO: 31)
GATTGAAAGCTATGCGAATTTGCACAGTCTTAAAAC
(SEQ ID NO: 32)
GAGATAGACCCTTGTTAACTCGTAAGGTTCTGTGAC
(SEQ ID NO: 33)
ATTGGATATACCCCTAATTTGAGAGGGGAATAAAAC
(SEQ ID NO: 34)
GTTGGACTATACCCTCGTTTGTAGGGGGAATAAAAC

Natural (wild-type) DNA coding sequences for Cas13e.3, Cas13e.4, Cas13e.5, Cas13e.6, Cas13e.7, Cas13e.8, Cas13f.6, Cas13f.7, Cas13d.1, Cas13d.2, Cas13d.3, Cas13d.4, Cas13d.5, Cas13c.1, and Cas13c.2 proteins are SEQ ID NOs: 75-89, respectively.

Cas13e.3 wild-type (SEQ ID NO: 75)
Cas13e.4 wild-type (SEQ ID NO: 76)
Cas13e.5 wild-type (SEQ ID NO: 77)
Cas13e.6 wild-type (SEQ ID NO: 78)
Cas13e.7 wild-type (SEQ ID NO: 79)
Cas13e.8 wild-type (SEQ ID NO: 80)
Cas13f.6 wild-type (SEQ ID NO: 81)
Cas13f.7 wild-type (SEQ ID NO: 82)
Cas13d.1 wild-type (SEQ ID NO: 83)
Cas13d.2 wild-type (SEQ ID NO: 84)
Cas13d.3 wild-type (SEQ ID NO: 85)
Cas13d.4 wild-type (SEQ ID NO: 86)
Cas13d.5 wild-type (SEQ ID NO: 87)
Cas13c.1 wild-type (SEQ ID NO: 88)
Cas13c.2 wild-type (SEQ ID NO: 89)

Human codon-optimized coding sequences for the fifteen Cas13c, Cas13d, Cas13e and Cas13f proteins (i.e., Cas13c.1, Cas13c.2, Cas13d.1, Cas13d.2, Cas13d.3, Cas13d.4, Cas13d.5, Cas13e.3, Cas13e.4, Cas13e.5, Cas13e.6, Cas13e.7, Cas13e.8, Cas13f.6, and Cas13f.7), generated for further functional experiments, are SEQ ID NOs: 103-104, 98-102, and 90-97, respectively.

Cas13e.3 (codon optimized) (SEQ ID NO: 90)
Cas13e.4 (codon optimized) (SEQ ID NO: 91)
Cas13e.5 (codon optimized) (SEQ ID NO: 92)
Cas13e.6 (codon optimized) (SEQ ID NO: 93)
Cas13e.7 (codon optimized) (SEQ ID NO: 94)
Cas13e.8 (codon optimized) (SEQ ID NO: 95)
Cas13f.6 (codon optimized) (SEQ ID NO: 96)
Cas13f.7 (codon optimized) (SEQ ID NO: 97)
Cas13d.1 (codon optimized) (SEQ ID NO: 98)
Cas13d.2 (codon optimized) (SEQ ID NO: 99)
Cas13d.3 (codon optimized) (SEQ ID NO: 100)
Cas13d.4 (codon optimized) (SEQ ID NO: 101)
Cas13d.5 (codon optimized) (SEQ ID NO: 102)
Cas13c.1 (Codon optimized) (SEQ ID NO: 103)
Cas13c.2 (codon optimized) (SEQ ID NO: 104)

Amino acid sequences for Cas13e.3, Cas13e.4, Cas13e.5, Cas13e.6, Cas13e.7, Cas13e.8, Cas13f.6, Cas13f.7, Cas13d.1, Cas13d.2, Cas13d.3, Cas13d.4, Cas13d.5, Cas13c.1, and Cas13c.2 proteins are SEQ ID NOs: 2-7 and 9-17, respectively.

Cas13e.3 protein (SEQ ID NO: 2)
Cas13e.4 protein (SEQ ID NO: 3)
Cas13e.5 protein (SEQ ID NO: 4)
Cas13e.6 protein (SEQ ID NO: 5)
Cas13e.7 protein (SEQ ID NO: 6)
Cas13e.8 protein (SEQ ID NO: 7)
Cas13f.6 protein (SEQ ID NO: 9)
Cas13f.7 protein (SEQ ID NO: 10)
Cas13d.1 protein (SEQ ID NO: 11)
Cas13d.2 protein (SEQ ID NO: 12)
Cas13d.3 protein (SEQ ID NO: 13)
Cas13d.4 protein (SEQ ID NO: 14)
Cas13d.5 protein (SEQ ID NO: 15)
Cas13c.1 protein (SEQ ID NO: 16)
Cas13c.2 protein (SEQ ID NO: 17)

Amino acid sequences for Cas13e.1 protein is SEQ ID NO: 1.

Cas13e.1 protein (SEQ ID NO: 1)

For example, in the Cas13e family, each DR sequence forms a secondary structure consisting of a 4-base pair stem (5′-GCUG-3′), a 5-base pair stem (5′-GCUGU-3′, or a 6-base pair stem including 1 nucleotide bulge (5′-GCUGGA-3′ followed by a symmetrical bulge of 5+5 nucleotides, 4+4 nucleotides, or 3+3 nucleotides, or two symmetrical bugles of 2+2 nucleotides and 1+1 nucleotides, or an unsymmetrical bulge of 2+1 nucleotides, (excluding the 4, 5 or 6 stem nucleotides), further followed by a 4-base pair stem (5′-GCCC-3′), 5-base pair stem (5′-GCC C/U C/U-3′), or 6-base pair stem (5′-A/G C/G CC U/C G/U-5′, and a terminal 6-base loop (5′-G A/U UUUG-3′), 8-base loop (5′-CGAUUUG U/C-3′, or 10-base loop (5′-UCGAUUUGCU-3′ (SEQ ID NO: 105)) (excluding the 2 stem nucleotides).

Likewise, in the Cas13f family, with one exception, each DR sequence forms a secondary structure consisting of a 5-base pair stem (5′-GCUGU-3′), followed by a nearly symmetrical bulge of 5+4 nucleotides (excluding the 4 stem nucleotides), further followed by a 6-base pair stem (5′-A/G CCUCG-3′), and a terminal 5-base loop (5′-AUUUG-3′, excluding the 2 stem nucleotides).

In the Cas13d family, with one exception, each DR sequence has single-stranded ends followed by a stem, a bulge in Cas13d.4 and Cas13d.5 DR sequences, ending in a loop. Cas13d.1 DR sequences has 7 single-stranded nucleotides on each 5′ and 3 end, a 9-base pair stem (5′-AACCCCGUA-3′), and a 4-base loop (5′-AAAA-3′). Cas13d.2 has a 4-base pair stem (5′-GUUA-3′), 1+1 nucleotide symmetrical bulge, a 3-base pair stem (5′-CCU-3′), and a 7-base stem (5′-AAGAAUG-3′). Cas13d.3 has a 7 single-stranded nucleotides on each 5′ and 3 end, 7-base pair stem (5′-AGCCUGC-3′), and a 8-base loop (5′-UGAAAUAU-3′). Cas13d.4 has 6 and 5 single stranded nucleotides on 5′ and 3′ ends, respectively, 5-base pair stem (5′-AAGCU-3′) with a single nucleotide bulge, 1+1 nucleotide symmetrical bulge, a 4-base pair stem (5′-UGCG-3′), A 4-base loop (5′-AAUU-3′). Cas13d.5 has 3 single-stranded nucleotides on each 5′ and 3′ end, 5′-base stem (5′-AUAGA-3′, 1+1 nucleotide symmetrical bulge, 5-base stem (5′-CCUUG-3′, and a 8-base loop (5′-UUAACUCG-3′).

In the Cas13c family, each DR sequence as 6 or 7 single-stranded nucleotides on the 5′ end and 4 single-stranded nucleotides on the 3′ end, 3′-base stem (5′-UAU-3′), 1+1 nucleotide symmetrical bulge, 5-base pair stem (5′-CCC C/U C/U-3′), and a 7 or 8-base loop (5′-AAUUUGAG-3′ or 5′-GUUUGUA-3′).

Further, in terms of the locations of the RXXXXH motifs with respect to the N- and C-termini of the Cas proteins, Cas13e and Cas13f proteins, and to a lesser extent Cas13b proteins, have their RXXXXH motifs closer to their N- and C-termini, as compared to the Cas13a, Cas13c, and Cas13d.

I-TASSER was then used to predict 3D structures for Cas13e proteins, followed by visualization of the predicted structures using PyMOL. Although the two RXXXXH motifs are located very close to the N- and C-termini of Cas13e.1, they are very close by in the 3D structure.

### Example 2 Knockdown of Fluorescent Reporter Protein mRNA Expression Using

Cas13 Protein in Mammalian Cells

The cleavage activity of the 15 new Cas13 protein subtypes identified in Example 1 was demonstrated in mammalian cells in this Example.

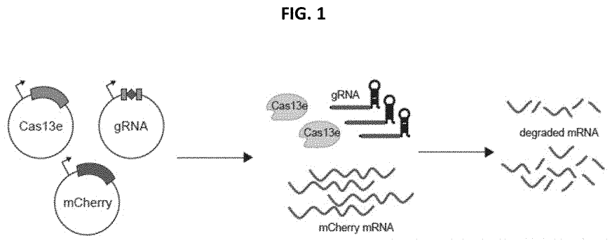

Briefly, HEK293T cells were cultured in 24-well tissue culture plates according to standard protocol, and were used for plasmid transfection using PEI reagents to introduce the three plasmids encoding one of the subject Cas13c, Cas13d, Cas13e, or Cas13f proteins, the mCherry-targeting gRNA (or the LacZ-targeting sgRNA as negative control), and the mCherry coding sequence, respectively. That is, in a negative control experiment, instead of using the plasmid encoding the mCherry-targeting sgRNA, a control plasmid encoding a non-Target-gRNA (i.e., LacZ-sgRNA) was used. A BFP coding sequence and an EGFP coding sequence were separately present in the Cas13-encoding plasmid and the sgRNA-encoding plasmid, thus expression of BFP and EGFP was used as an internal control for transfection success/efficiency. See schematic illustration in FIG. 1. Transfected HEK293T cells were then incubated at 37° C. under 5% CO₂ for about 24 hours, before the FACS sorted cells 48 hours post transfection were subject to examination under the fluorescent microscope.

Three different mCherry-targeting sgRNA's (i.e., mCherry-sg1, mCherry-sg2, and mCherry-sg3), were designed against different regions of the mCherry target mRNA. Cells successfully expressing both the BFP and EGFP reporters were selected for analysis. In these cells, the average fluorescent intensity of mCherry, as normalized against that of control cells transfected by the LacZ-targeting sgRNA but not any of the mCherry-targeting sgRNA's. That is, the average mCherry fluorescent intensity in the control cells was arbitrarily set at 1.

The sgRNA sequences were provided below:

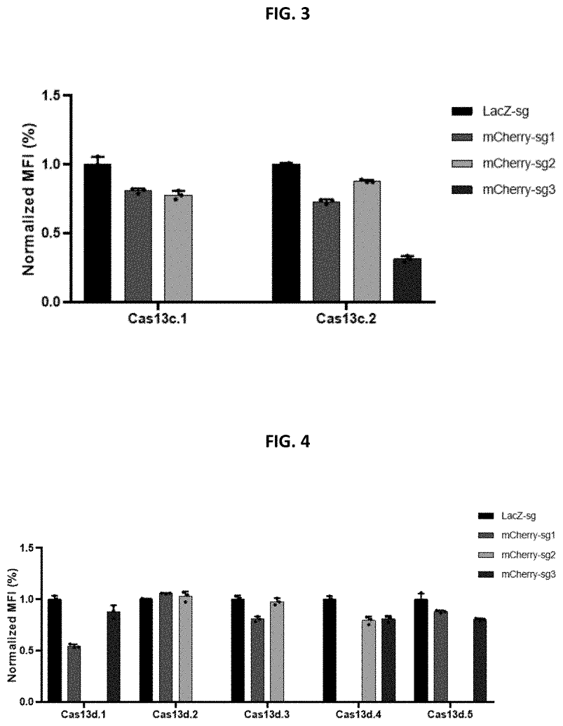

mCherry-sg1:
(SEQ ID NO: 71)
gcagcttcaccttgtagatgaactcgccgt
mCherry-sg2:
(SEQ ID NO: 72)
gttcatcacgcgctcccacttgaagccctc
mCherry-sg3:
(SEQ ID NO: 73)
tgcttcacgtaggccttggagccgtacatg
LacZ-sg:
(SEQ ID NO: 74)
cgtctggccttcctgtagccagctttcatc
The results of these experiments were shown in
FIGs. 3-7.

Specifically, FIG. 3 shows that Cas13c.1 and Cas13c.2 only had up to about 25% mCherry knock-down with mCherry-sg1 and mCherry-sg2, while Cas13c.1 and Cas13c.2 had over 70-100% knock-down of target mCherry mRNA when mCherry-sg3 was used, compared to the LacZ control.

FIG. 4 shows that Cas13d.1 had about 50% knock-down of mCherry mRNA with mCherry-sg1, about 100% knock-down of mCherry mRNA with mCherry-sg2, and up to about 15% of mCherry mRNA knock-down. Cas13d.2 had minimal mCherry knock-down with either mCherry-sg1 or mCherry-sg2, and had about 100% knock-down of mCherry mRNA with mCherry-sg3. Cas13d.3 had about 15% mCherry knock-down with mCherry-sg1, minimal knock-down with mCherry-sg2, and had about 100% knock-down of mCherry mRNA with mCherry-sg3. Cas13d.4 had about 100% knock-down of mCherry mRNA with mCherry-sg1, and about 20% knock-down with either mCherry-sg2 or mCherry-sg3. Cas13d.5 has about 10% knock-down of mCherry mRNA with mCherry-sg1, about 100% knock-down of mCherry mRNA with mCherry-sg2, and about 15% knock-down with mCherry-sg3. Among them, Cas13d.1 and Cas13d.5 had the most robust knock-down with mCherry-sg3, Cas13d.2 had the most robust knock-down with mCherry-sg-3, and Cas13d.3 and Cas13d.4 had the most robust knock-down efficiency when paired with mCherry-sg3.

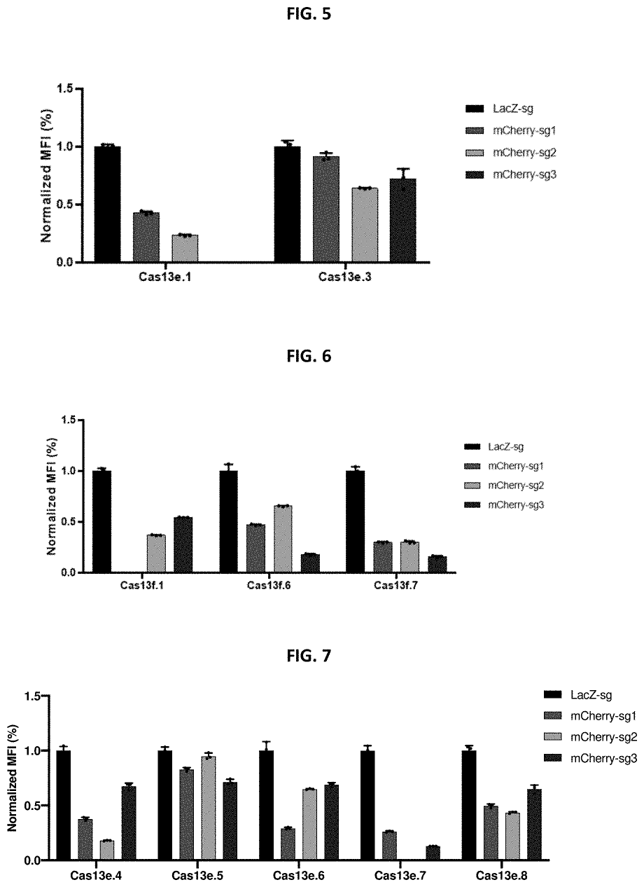

FIG. 5 shows that Cas13e.3 had marginal mCherry mRNA knock-down with mCherry-sg1, and about 30% knock-down with mCherry-sg2, and about 25% knock-down with mCherry-sg3. While the Cas13e.1, as a control, had about 55%, 75%, and 100% knock-down when paired with mCherry sg1, sg2, and sg3, respectively.

FIG. 6 shows that Cas13f.6 had about 50%, 30%, and 80% mCherry mRNA knock-down when paired with mCherry sg1, sg2, and sg3, respectively. Cas13f.7 had about 70%, 70%, and 80% mCherry mRNA knock-down when paired with mCherry sg1, sg2, and sg3, respectively. While the Cas13f.1, as a positive control, had about 100%, 60%, and 50% knock-down when paired with mCherry sg1, sg2, and sg3, respectively.

FIG. 7 shows that Cas13e.4 had about 60%, 75%, and 40% mCherry mRNA knock-down when paired with mCherry sg1, sg2, and sg3, respectively. Cas13e.5 had about 20%, 5%, and 40% mCherry mRNA knock-down when paired with mCherry sg1, sg2, and sg3, respectively. Cas13e.6 had about 75%, 40%, and 40% mCherry mRNA knock-down when paired with mCherry sg1, sg2, and sg3, respectively. Cas13e.7 had about 75%, 100%, and 90% mCherry mRNA knock-down when paired with mCherry sg1, sg2, and sg3, respectively. Cas13e.8 had about 50%, 55%, and 40% mCherry mRNA knock-down when paired with mCherry sg1, sg2, and sg3, respectively.

The above data demonstrates that the 15 newly identified Cas13c, 13d, 13e, and 13f proteins all have significant guide-RNA-specific knockdown activity against the tested target gene mCherry. It appears that different Cas13 effector enzymes prefer different sgRNA for most efficient knockdown.

What is claimed is:

1. A Clustered Regularly Interspaced Short Palindromic Repeat (CRISPR)-Cas complex, comprising:
(1) an RNA guide sequence comprising a spacer sequence capable of hybridizing to a target RNA, and a direct repeat (DR) sequence 5′ or 3′ to the spacer sequence; and,
(2) a CRISPR-associated protein (Cas) having an amino acid sequence of any one of SEQ ID NOs: 2-7 and 9-17, or a derivative or functional fragment of said Cas;
wherein the Cas, the derivative, and the functional fragment of said Cas, are capable of (i) binding to the RNA guide sequence and (ii) targeting the target RNA, with the proviso that the spacer sequence is not 100% complementary to a naturally-occurring bacterialphage nucleic acid when the complex comprises the Cas of any one of SEQ ID NOs: 2-7 and 9-17.

2. The CRISPR-Cas complex of claim 1, wherein the DR sequence has substantially the same secondary structure as the secondary structure of any one of SEQ ID NOs: 19-24 and 26-34.

3. The CRISPR-Cas complex of claim 1, wherein the DR sequence is encoded by any one of SEQ ID NOs: 19-24 and 26-34.

4. The CRISPR-Cas complex of claim 1, 2, or 3, wherein the target RNA is encoded by a eukaryotic DNA.

5. The CRISPR-Cas complex of claim 4, wherein the eukaryotic DNA is a non-human mammalian DNA, a non-human primate DNA, a human DNA, a plant DNA, an insect DNA, a bird DNA, a reptile DNA, a rodent DNA, a fish DNA, a worm/nematode DNA, a yeast DNA.

6. The CRISPR-Cas complex of any one of claims 1-5, wherein the target RNA is an mRNA.

7. The CRISPR-Cas complex of any one of claims 1-6, wherein the spacer sequence is between 15-55 nucleotides, between 25-35 nucleotides, or about 30 nucleotides.

8. The CRISPR-Cas complex of any one of claims 1-7, wherein the spacer sequence is 90-100% complementary to the target RNA.

9. The CRISPR-Cas complex of any one of claims 1-8, wherein the derivative is at least about 90%, 95%, 96%, 97%, 98%, 99% identical to any one of SEQ ID NOs: 2-7 and 9-17, or comprises conserved amino acid substitutions of one or more residues of any one of SEQ ID NOs: 2-7 and 9-17.

10. The CRISPR-Cas complex of claim 9, wherein the derivative comprises only conserved amino acid substitutions.

11. The CRISPR-Cas complex of any one of claims 1-10, wherein the derivative has identical sequence to wild-type Cas of any one of SEQ ID NOs: 2-7 and 9-17 in the HEPN domain or the RXXXXH motif.

12. The CRISPR-Cas complex of any one of claims 1-9, wherein the derivative is capable of binding to the RNA guide sequence hybridized to the target RNA, but has no RNase catalytic activity due to a mutation in the RNase catalytic site of the Cas.

13. The CRISPR-Cas complex of claim 12, wherein the derivative has an N-terminal deletion of no more than 210 residues, and/or a C-terminal deletion of no more than 180 residues.

14. The CRISPR-Cas complex of claim 13, wherein the derivative has an N-terminal deletion of about 180 residues, and/or a C-terminal deletion of about 150 residues.

15. The CRISPR-Cas complex of any one of claims 12-14, wherein the derivative further comprises an RNA base-editing domain.

16. The CRISPR-Cas complex of claim 15, wherein the RNA base-editing domain is an adenosine deaminase, such as a double-stranded RNA-specific adenosine deaminase (e.g., ADAR1 or ADAR2); apolipoprotein B mRNA editing enzyme; catalytic polypeptide-like (APOBEC); or activation-induced cytidine deaminase (AID).

17. The CRISPR-Cas complex of claim 16, wherein the ADAR has E488Q/T375G double mutation or is ADAR2DD.

18. The CRISPR-Cas complex of any one of claims 15-17, wherein the base-editing domain is further fused to an RNA-binding domain, such as MS2.

19. The CRISPR-Cas complex of any one of claims 12-14, wherein the derivative further comprises an RNA methyltransferase, a RNA demethylase, an RNA splicing modifier, a localization factor, or a translation modification factor.

20. The CRISPR-Cas complex of any one of claims 1-19, wherein the Cas, the derivative, or the functional fragment comprises a nuclear localization signal (NLS) sequence or a nuclear export signal (NES).

21. The CRISPR-Cas complex of any one of claims 1-20, wherein targeting of the target RNA results in a modification of the target RNA.

22. The CRISPR-Cas complex of claim 21, wherein the modification of the target RNA is a cleavage of the target RNA.

23. The CRISPR-Cas complex of claim 21, wherein the modification of the target RNA is deamination of an adenosine (A) to an inosine (I).

24. The CRISPR-Cas complex of any one of claims 1-23, further comprising a target RNA comprising a sequence capable of hybridizing to the spacer sequence.

25. A fusion protein, comprising (1) the Cas, the derivative thereof, or the functional fragment thereof, of any one of claims 1-24, and (2) a heterologous functional domain.

26. The fusion protein of claim 25, wherein the heterologous functional domain comprises: a nuclear localization signal (NLS), a reporter protein or a detection label (e.g., GST, HRP, CAT, GFP, HcRed, DsRed, CFP, YFP, BFP), a localization signal, a protein targeting moiety, a DNA binding domain (e.g., MBP, Lex A DBD, Gal4 DBD), an epitope tag (e.g., His, myc, V5, FLAG, HA, VSV-G, Trx, etc), a transcription activation domain (e.g., VP64 or VPR), a transcription inhibition domain (e.g., KRAB moiety or SID moiety), a nuclease (e.g., FokI), a deamination domain (e.g., ADAR1, ADAR2, APOBEC, AID, or TAD), a methylase, a demethylase, a transcription release factor, an HDAC, a polypeptide having ssRNA cleavage activity, a polypeptide having dsRNA cleavage activity, a polypeptide having ssDNA cleavage activity, a polypeptide having dsDNA cleavage activity, a DNA or RNA ligase, or any combination thereof.

27. The fusion protein of claim 25 or 26, wherein the heterologous functional domain is fused N-terminally, C-terminally, or internally in the fusion protein.

28. A conjugate, comprising (1) the Cas, the derivative thereof, or the functional fragment thereof, of any one of claims 1-24, conjugated to (2) a heterologous functional moiety.

29. The conjugate of claim 28, wherein the heterologous functional moiety comprises: a nuclear localization signal (NLS), a reporter protein or a detection label (e.g., GST, HRP, CAT, GFP, HcRed, DsRed, CFP, YFP, BFP), a localization signal, a protein targeting moiety, a DNA binding domain (e.g., MBP, Lex A DBD, Gal4 DBD), an epitope tag (e.g., His, myc, V5, FLAG, HA, VSV-G, Trx, etc), a transcription activation domain (e.g., VP64 or VPR), a transcription inhibition domain (e.g., KRAB moiety or SID moiety), a nuclease (e.g., FokI), a deamination domain (e.g., ADAR1, ADAR2, APOBEC, AID, or TAD), a methylase, a demethylase, a transcription release factor, an HDAC, a polypeptide having ssRNA cleavage activity, a polypeptide having dsRNA cleavage activity, a polypeptide having ssDNA cleavage activity, a polypeptide having dsDNA cleavage activity, a DNA or RNA ligase, or any combination thereof.

30. The conjugate of claim 28 or 29, wherein the heterologous functional moiety is conjugated N-terminally, C-terminally, or internally with respect to the Cas, the derivative thereof, or the functional fragment thereof.

31. A polynucleotide encoding any one of SEQ ID NOs: 2-7 and 9-17, or a derivative thereof, or a functional fragment thereof, or a fusion protein thereof, or a polynucleotide at least about 70%, 80%, 85%, 90%, 95%, 96%, 97%, 98%, 99% identical thereto, provided that the polynucleotide is not any one of SEQ ID NOs: 1 and 8.

32. The polynucleotide of claim 31, which is codon-optimized for expression in a cell.

33. The polynucleotide of claim 32, wherein the cell is a eukaryotic cell.

34. A non-naturally occurring polynucleotide comprising a derivative of any one of SEQ ID NOs: 19-24 and 26-34, wherein said derivative (i) has one or more (e.g., 1, 2, 3, 4, 5, 6, 7, 8, 9 or 10) nucleotides additions, deletions, or substitutions compared to any one of SEQ ID NOs: 19-24 and 26-34; (ii) has at least 20%, 30%, 40%, 50%, 60%, 70%, 80%, 90%, 95%, or 97% sequence identity to any one of SEQ ID NOs: 19-24 and 26-34; (iii) hybridize under stringent conditions with any one of SEQ ID NOs: 19-24 and 26-34 or any of (i) and (ii); or (iv) is a complement of any of (i)-(iii), provided that the derivative is not any one of SEQ ID NOs: 19-24 and 26-34, and that the derivative encodes an RNA (or is an RNA) that has maintained substantially the same secondary structure as any of the RNA encoded by SEQ ID NOs: 19-24 and 26-34.

35. The non-naturally occurring polynucleotide of claim 34, wherein the derivative functions as a DR sequence for any one of the Cas, the derivative thereof, or the functional fragment thereof, of any one of claims 1-24.

36. A vector comprising the polynucleotide of any one of claims 31-35.

37. The vector of claim 36, wherein the polynucleotide is operably linked to a promoter and optionally an enhancer.

38. The vector of claim 37, wherein the promoter is a constitutive promoter, an inducible promoter, a ubiquitous promoter, or a tissue specific promoter.

39. The vector of any one of claims 36-38, which is a plasmid.

40. The vector of any one of claims 36-38, which is a retroviral vector, a phage vector, an adenoviral vector, a herpes simplex viral (HSV) vector, an AAV vector, or a lentiviral vector.

41. The vector of claim 40, wherein the AAV vector is a recombinant AAV vector of the serotype AAV1, AAV2, AAV4, AAV5, AAV6, AAV7, AAVrh74, AAV8, AAV9, AAV10, AAV 11, AAV 12, or AAV 13.

42. A delivery system comprising (1) a delivery vehicle, and (2) the CRISPR-Cas complex of any one of claims 1-24, the fusion protein of any one of claims 25-27, the conjugate of any one of claims 28-30, the polynucleotide of any one of claims 31-33, or the vector of any one of claims 36-41.

43. The delivery system of claim 42, wherein the delivery vehicle is a nanoparticle, a liposome, an exosome, a microvesicle, or a gene-gun.

44. A cell or a progeny thereof, comprising the CRISPR-Cas complex of any one of claims 1-24, the fusion protein of any one of claims 25-27, the conjugate of any one of claims 28-30, the polynucleotide of any one of claims 31-33, or the vector of any one of claims 36-41.

45. The cell or progeny thereof of claim 44, which is a eukaryotic cell (e.g., a non-human mammalian cell, a human cell, or a plant cell) or a prokaryotic cell (e.g., a bacteria cell).

46. A non-human multicellular eukaryote comprising the cell of claim 44 or 45.

47. The non-human multicellular eukaryote of claim 46, which is an animal (e.g., rodent or primate) model for a human genetic disorder.

48. A method of modifying a target RNA, the method comprising contacting the target RNA with the CRISPR-Cas complex of any one of claims 1-24, wherein the spacer sequence is complementary to at least 15 nucleotides of the target RNA; wherein the Cas, the derivative, or the functional fragment associates with the RNA guide sequence to form the complex; wherein the complex binds to the target RNA; and wherein upon binding of the complex to the target RNA, the Cas, the derivative, or the functional fragment modifies the target RNA.

49. The method of claim 48, wherein the target RNA is modified by cleavage by the Cas.

50. The method of claim 48, wherein the target RNA is modified by deamination by a derivative comprising a double-stranded RNA-specific adenosine deaminase.

51. The method of any one of claim 48-50, wherein the target RNA is an mRNA, a tRNA, an rRNA, a non-coding RNA, an lncRNA, or a nuclear RNA.

52. The method of any one of claims 48-51, wherein upon binding of the complex to the target RNA, the Cas, the derivative, and the functional fragment does not exhibit substantial (or detectable) collateral RNase activity.

53. The method of any one of claims 48-52, wherein the target RNA is within a cell.

54. The method of claim 53, wherein the cell is a cancer cell.

55. The method of claim 53, wherein the cell is infected with an infectious agent.

56. The method of claim 55, wherein the infectious agent is a virus, a prion, a protozoan, a fungus, or a parasite.

57. The method of any one of claims 53-56, wherein the CRISPR-Cas complex is encoded by a first polynucleotide encoding any one of SEQ ID NOs: 2-7 and 9-17, or a derivative or functional fragment thereof, and a second polynucleotide comprising any one of SEQ ID NOs: 19-24 and 26-34 and a sequence encoding a spacer RNA capable of binding to the target RNA, wherein the first and the second polynucleotides are introduced into the cell.

58. The method of claim 57, wherein the first and the second polynucleotides are introduced into the cell by the same vector.

59. The method of any one of claims 53-58, which cases one or more of: (i) in vitro or in vivo induction of cellular senescence; (ii) in vitro or in vivo cell cycle arrest; (iii) in vitro or in vivo cell growth inhibition and/or cell growth inhibition; (iv) in vitro or in vitro induction of anergy; (v) in vitro or in vitro induction of apoptosis; and (vi) in vitro or in vitro induction of necrosis.

60. A method of treating a condition or disease in a subject in need thereof, the method comprising administering to the subject a composition comprising the CRISPR-Cas complex of any one of claims 1-24 or a polynucleotide encoding the same; wherein the spacer sequence is complementary to at least 15 nucleotides of a target RNA associated with the condition or disease; wherein the Cas, the derivative, or the functional fragment associates with the RNA guide sequence to form the complex; wherein the complex binds to the target RNA; and wherein upon binding of the complex to the target RNA, the Cas, the derivative or the functional fragment cleaves the target RNA, thereby treating the condition or disease in the subject.

61. The method of claim 60, wherein the condition or disease is a cancer or an infectious disease.

62. The method of claim 61, wherein the cancer is Wilms' tumor, Ewing sarcoma, a neuroendocrine tumor, a glioblastoma, a neuroblastoma, a melanoma, skin cancer, breast cancer, colon cancer, rectal cancer, prostate cancer, liver cancer, renal cancer, pancreatic cancer, lung cancer, biliary cancer, cervical cancer, endometrial cancer, esophageal cancer, gastric cancer, head and neck cancer, medullary thyroid carcinoma, ovarian cancer, glioma, lymphoma, leukemia, myeloma, acute lymphoblastic leukemia, acute myelogenous leukemia, chronic lymphocytic leukemia, chronic myelogenous leukemia, Hodgkin's lymphoma, non-Hodgkin's lymphoma, or urinary bladder cancer.

63. The method of any one of claims 60-62, which is an in vitro method, an in vivo method, or an ex vivo method.

64. A cell or a progeny thereof, obtained by the method of any one of claims 48-59, wherein the cell and the progeny comprises a non-naturally existing modification (e.g., a non-naturally existing modification in a transcribed RNA of the cell/progeny).

65. A method to detect the presence of a target RNA, the method comprising contacting the target RNA with a composition comprising a fusion protein of any one of claims 25-27, or a conjugate of any one of claims 28-30, or a polynucleotide encoding the fusion protein, wherein the fusion protein or the conjugate comprises a detectable label (e.g., one that can be detected by fluorescence, Northern blot, or FISH) and a complexed spacer sequence capable of binding to the target RNA.

66. A eukaryotic cell comprising a Clustered Regularly Interspaced Short Palindromic Repeat (CRISPR)-Cas complex, said CRISPR-Cas complex comprising:
(1) an RNA guide sequence comprising a spacer sequence capable of hybridizing to a target RNA, and a direct repeat (DR) sequence 3′ to the spacer sequence; and,
(2) a CRISPR-associated protein (Cas) having an amino acid sequence of any one of SEQ ID NOs: 2-7 and 9-17, or a derivative or functional fragment of said Cas;
wherein the Cas, the derivative, and the functional fragment of said Cas, are capable of (i) binding to the RNA guide sequence and (ii) targeting the target RNA.


SEQUENCE LISTING (120 sequences)

SEQ ID NO: 1 - protein, 775 aa, Artificial Sequence
  Description of Artificial Sequence: Synthetic polypeptide
MAQVSKQTSKKRELSIDEYQGARKWCFTIAFNKALVNRDKNDGLFVESLLRHEKYSKHDWYDEDTRALIKCSTQAANAKAEALRNYFSHYRHSPGCLTFTAEDELRTIMERAYERAIFECRRRETEVIIEFPSLFEGDRITTAGVVFFVSFFVERRVLDRLYGAVSGLKKNEGQYKLTRKALSMYCLKDSRFTKAWDKRVLLFRDILAQLGRIPAEAYEYYHGEQGDKKRANDNEGTNPKRHKDKFIEFALHYLEAQHSEICFGRRHIVREEAGAGDEHKKHRTKGKVVVDFSKKDEDQSYYISKNNVIVRIDKNAGPRSYRMGLNELKYLVLLSLQGKGDDAIAKLYRYRQHVENILDVVKVTDKDNHVFLPRFVLEQHGIGRKAFKQRIDGRVKHVRGVWEKKKAATNEMTLHEKARDILQYVNENCTRSFNPGEYNRLLVCLVGKDVENFQAGLKRLQLAERIDGRVYSIFAQTSTINEMHQVVCDQILNRLCRIGDQKLYDYVGLGKKDEIDYKQKVAWFKEHISIRRGFLRKKFWYDSKKGFAKLVEEHLESGGGQRDVGLDKKYYHIDAIGRFEGANPALYETLARDRLCLMMAQYFLGSVRKELGNKIVWSNDSIELPVEGSVGNEKSIVFSVSDYGKLYVLDDAEFLGRICEYFMPHEKGKIRYHTVYEKGFRAYNDLQKKCVEAVLAFEEKVVKAKKMSEKEGAHYIDFREILAQTMCKEAEKTAVNKVRRAFFHHHLKFVIDEFGLFSDVMKKYGIEKEWKFPVK

SEQ ID NO: 2 - protein, 866 aa, Artificial Sequence
  Description of Artificial Sequence: Synthetic polypeptide
MAVDYSLKNEWYREINKSCFTVALNVAYDNCKAKGHENLLREAQRSKGGITNEQIKNVQTEIKTRLEDIRSHFSHFYHDEKSLIFEKDNIVKDFLESAYEKAQSSVIGSTRQSDYKGVVPPLFEPHDGMITAAGVVFLASFFCHRSNVYRMLGAVKGFKHTGKEELSDGAKRDYGFTRRLMAHYSLRDSYVIKAEETKSFRDLLGYLSRVPQQAVDWLNEHNQLSEDEKKEFLNQKPSDEESQEQSKTENTDRQADRMPRRSLRKTDKFILFAAKFIEDWAQKEKMDVTFARYQKTVTEDENKNQDGKQVRDVQLKYEKDTKKLNPDFDYKWTYYIRNNHAIIQIKPDEYKQAVSARISENELKYLVLLIFQGKGWEAIKKIGDYIFHIGNKIKIGRFDHNEERRMPSFLKNPPADIIGEMVENRLKYIRDELNKVIETIKKEEPQNNKWLLYKGKKISIILKFISDSISDIKKRPDVNEYNTLRDMLQKLDFDNFYERLKSYVSEGRIEQTLYDEIKGIKDISTLCIKICELRLAALEELEKEGGDDLNKYIGLAVQEKHKNYDDSNTPQKKAERFLESQFSVGKNFLRETFYDEYIKNRKSLYEIIKEKITGITPLNENRWYLMDKNPKEFESKDSKIIRGLCNIYIQDILCMKIALWYYENLSPSYKNKLKWDFIGQGFGYDRYKLSYKTDCGITIEFKLADLNRLDIIEKPKMIENICHSFILEKDVKKQTISWHEFRQDGIAKYRKLQKEVVEAVFEFENSLKIPDKNWLTQGYVPFNKNKRFEDKGFSTFILEEAVRKGKIKSDDKEPLRKVRTDFFHEQFDSTDAERRIFDKYMPAKHDGKNKGGKMQEKQEKSYTRRI

SEQ ID NO: 3 - protein, 798 aa, Artificial Sequence
  Description of Artificial Sequence: Synthetic polypeptide
MEKEQGLYSIDRYQGAGKWCFAIGANRAWDNYNERPKLFSESLLRYEKATRRDWFDEETRGLIKKSDVRQRLRKIRCYFSHYCHDNTCLGFDPDDDLRKIMEKAYERAIFEQRKHLSTETDIETPALFEPHGRITAAGVVFFCSFFVERRILNRLMGRIPGFKKTEGEYGATRQMFSKYCLRDSYSIRASDSNAVLFRDILGYLSRAPSQYYRHNKDQCDKDGHPERKKDKFINLALRYLESFVPARLRNHTLSVGRKEVVRMETNAVAEGEGEYRPYPPKAKVKVVFTEDDPERPYYITHNTVILQTAKKEEDIHHCKVGVNELKYLVLLCLQGKAEKAVAGIEGYVRRIQGRFADHTNKVARDDDERLVRGLPEFVRVASGIETPDEVRELKSRLDHIRKKWQTKKAESAEAQLHRKARDVLRHINWESQRPLGIEQYNRLLELLVNRDLESFAAEMKELKRRGLISEELLKSVEGIRNLNTLHVKVCNLVLTRLEHLVENDPEELKRHIGIVPREEKEGPSYEEKVRAFVQQPMMYRGFLRNSFFKGSGKSFAKLVEEELHKKGCPDVPLGTDYYLVRDLERDERKNRFHNDNAALYETLALDRLCVLMARDCLVRLNRNLEKHATRISWEATDAGDTICLELPRRDRDHESFRLSFGVRDYPKLYVMDDPVFLCGLMKHFFPDNQAIQYHELYSEGINKYTAMQAEGIAATLKLEEKTIKEKNMQIPATGYIRFCEIVSQSDFAPGEKRVLKNVRNGLLHYHLEFEPTEWAEFREIMKREGFDTAKKRKSTRKK

SEQ ID NO: 4 - protein, 804 aa, Artificial Sequence
  Description of Artificial Sequence: Synthetic polypeptide
MKIKNENTDKKTELYSIDKYKGRDKWCFAIVLNKAQTNLTENPDLFEQTITKYDRIRKEGWFDEETKKLIYIQENEHKIKGEIKTLAREVLKNLRNYFSHHFYKQDCLIFPKDNIVRIIMGRAYERSEYEIKKNLKEDISIELPALFEPEGKITTAGIVFFTSFFVERRFLHRLMGSVQGFTKTEGEYKITRDVFAKYCLRDSYSVKAQDNDAVMFRDIIGYLSRVPTESFQHIKNPKKQNESQLSERKTDKFISFALKYLKDYGFEDLKGHYTAFFARSEIKKEKEDIEIKDDKKHKPHRMKSKIEIHFDKTKEDRFYIERNNVILKIQRKGGRANILRMGIYELKYLVLLCLSGKAREAINRIDDYLNDLRNKIPHIENMNKEGIGEQIRSLPGFVRSQLGFVQIDDEKKKENRLDYVEKKWEKKRAESKELKLNRKGRDILRYINERCKKPLTIDRYNRILELLVEKNIEGFYHELEELRKTGRIEKNITQALVGEKNINALHIKICKLVQDELKSLEKEDLKKYIGLTPKEEKVVSFEEKLGRILDKPVIYKGFLRYQFFKNDKKSFARLVEEIIKEKTGGLDVPIETEYYSISTLGRFDKANKTLYETLAMDRLCMMMARRYFLSLNKILAKRAQNIEWKKESGKEFIVFKFNMPQDTGKSISIRFSPKDYTKLYVKNDSEFLARLCQYFFPNEKAIDYHKLYSHGINKYTNFQKEGIEAILELEEKIIKKRKINSPENYLSFEEILNQSIYNDEEKNTLIQIRHSLLHYQILFSKNDLTKFYNVMKREGIEKIWSLVI

SEQ ID NO: 5 - protein, 878 aa, Artificial Sequence
  Description of Artificial Sequence: Synthetic polypeptide
MAVNYSLREKWYRGVNKCCFTVALNIAVDNCKSKGCETLLKEAEHSKGGITDEQIQQSYTEVEKRLNDIRNYFSHFYHGDECLIFKKDDIVKRFMESVFATAVSNVVGGTKESDYKGVVPPLFEQSNEDYMITAAGVIFLASFFCHRSNVYRMLGAVKGFKHTGKEELSDGQKRDHGFTHRLLAHYSLRDSYSVKIEETKSFRDLLGYLSRVPQQAVDWLNERNELSEDEKKEFLNQKSSEEESPEQPEPENAEWRTEKTSRRSLRKTEKFILFAAKFIEDRAEKEKQDVTFARYQKTVTKEENKNQDGKQARVVRLKYEEDKKDDEKPREHFNLEWMYYIRNEHAIIQIKPKDKEAVAARISENELKYLVLLIFEGKGGDAFNKLSDYIFRMTQKIKSGQINPNEARLPSFLKNPVKNITDKMVRNRLDYIRGQIKDVLEKINMEEPQNNKWLIYKGKKISLVLKFISDGISDIKKRPNVKEYDTLRDTLQKLDFNRFYERLKSYVSDGRLAAALYDKIKGIDDISELCKKVCELMFARLAELEKKGGFELYRYIGMEVQEKDEKYDEWNSPQKKAERFLESQFSIGENFLRESFYSEYCQKQECIDKEISLNTSVKNRKSLVYIVKEKLKDIMPLHNDRWYLIDRNPKDFERKDSKVIKGLCNTYVQDVLCMKMARWYYGQLNPALKNNIKWDETGQGHGYDRYKLSYRTNFGITIEFKLADFTRLDIIEKSDMIENICRSFIKPNRTISWYDFKQDGVEEYRKRQYKAVRAVFAFEESLIIPGRDWLSQGFVPFIKNEEYVKKGFSLFVLDEAVRQLKIKGSDKDAMRQVRNDFFHEQFQAKDEQWKVFEGYLSCFMIDRPKGEKNKKRYNGNKK

SEQ ID NO: 6 - protein, 871 aa, Artificial Sequence
  Description of Artificial Sequence: Synthetic polypeptide
MEKYLIKNFEGINKSKFTVALNIANDNCKNKGIQELLKEAQRSKGGITDTQITEVQEHIKERLNSVRNYFSHCYHEKKPLYFEANDPVKIFLEETFAKAVENLQGRFLSDKYKLTVPPLFEPNQNNTITAAGVIFLASFFCHRSYVYRMLGGIPGFKRSDKKKWGDGQKIDYGFTRKLMSFYSLRDSYSVNVQENKELTAFRDILGYLARVPGQAIDWLIEKGKLTKEEGKQFYLGEQSEEREEKAKKEEIKYALRKTDKFMLFAVRFIEDWAEQERIKVEFARYEKMTIVNENKKQDEKEERKVKFVSDEPTAAGWTYYIRNNHAIIKIIPDDKKKKAVSARISENELKYLVLTIIDGNGKNAIAYIGDYIFRTARQIENKSYNAESEKYAPAFVRGGQKKSVDKRIKYIRDEIQQVINDIEAEQEKQKNEQDAPAENRTWLIYKGKKISIILRYVNDNIAEYKKRLSVTEYNELRGYLQQLDFINFHRKLAEYQHHGRLPNGFAESINKFQDLSKLCIEVCERQKKKLQEMAAKGGIELEQYIGLAPKEENQEQNKYATKANNFIKVWLSIPENFLRQKFYDKFCKQQECKNKGSDKPDNTSVPQRKYFIAIIREKNIRPIHADKYYLLGQNPKDYERPDGKIIRQLCDVYCKDGLCMAMAKWYYENRLGKFKDLIEWQTGDDKQQHGYAGHTLEYQATEKIKIRFKLADFTRLDIIEPPERVKNICRQWETELLKKTRDGTISWYDFKLNGLEPYRQWQGYAVADIFWFEESLKINETQWQGRTHMPFNFEKDKPLWCNILDEAVKQNKIEKQDTQALRRVRHDCFHEEFLANYEQLKIFKNLISDKAKDAKPKDKKSRKNEQKYGKR

SEQ ID NO: 7 - protein, 813 aa, Artificial Sequence
  Description of Artificial Sequence: Synthetic polypeptide
METTEEKKEIYSIDKYQGAGKWCFAIILNRARDNYEGNPHLLSESLLRFEAVNRRDWFDEETRELIIQMDAETKTDSNLKPSTPAYKILIGIRNYFSHFYHTEECLYFKEDDPIRIILENAYKKAKQHHIERLEKETDIEFPALFESNDRITSAGIIFFSSLFVERRILNRLMGYVGGFKKTKGEYNITREIFSTYCLRDSYSIMAADSNAVIFRDILGYLSRVPSEYYQHNKEKCEKENKPKRKTKKFIYFALRYLEKFVLKEIKNQKVSIARMEVVREKSKEAEGEDEQHKPYPDKAKVKIVFDSIGKELPYYINHKTVIMKIQRNGEAVNFCKIGINELKYLVLLCLQGKSVDAIAKINGYIDRIKKRLENPKTRIDIDSERDNEFIKGLPEFVKIQSGRTPDEEREKKSRIDYIRKKWEKKKEESPKTELHRKGRDILRYINWHCEPPLGSEEYNHLLSLLVNKDLDGFENELKELKRTEQISKKLLEMLKGFSNLNELHLKVCSIVPVELVYLEKNDPEKLAEYIGLIPQKTDKEPPSYEEKVKNFIKQPMIYKGFLRDTFFSSGKTFAKLVEETFLHKYPHSDVPLGRDYYHVTTLDRFHKDNSILYETLALDRLCVVMARKFHETLNQELAKESKQIVWENNTIILELPRSKTSSSDTFQICFDIKHYMKLYVMDDVEFLGGLMRHFFQKEKTIEYYNLYSFGINKYTEMQRNGIEAILRLEEKIIREKNIKRKPDKNYISFYEIMEASNYPDNDKKTLNKVRRALLHYHLKFEPADYNKFVAIMKREGLERKKKTGKATRRWVKK

SEQ ID NO: 8 - protein, 790 aa, Artificial Sequence
  Description of Artificial Sequence: Synthetic polypeptide
MNGIELKKEEAAFYFNQAELNLKAIEDNIFDKERRKTLLNNPQILAKMENFIFNFRDVTKNAKGEIDCLLLKLRELRNFYSHYVHKRDVRELSKGEKPILEKYYQFAIESTGSENVKLEIIENDAWLADAGVLFFLCIFLKKSQANKLISGISGFKRNDDTGQPRRNLFTYFSIREGYKVVPEMQKHFLLFSLVNHLSNQDDYIEKAHQPYDIGEGLFFHRIASTFLNISGILRNMKFYTYQSKRLVEQRGELKREKDIFAWEEPFQGNSYFEINGHKGVIGEDELKELCYAFLIGNQDANKVEGRITQFLEKFRNANSVQQVKDDEMLKPEYFPANYFAESGVGRIKDRVLNRLNKAIKSNKAKKGEIIAYDKMREVMAFINNSLPVDEKLKPKDYKRYLGMVRFWDREKDNIKREFETKEWSKYLPSNFWTAKNLERVYGLAREKNAELFNKLKADVEKMDERELEKYQKINDAKDLANLRRLASDFGVKWEEKDWDEYSGQIKKQITDSQKLTIMKQRITAGLKKKHGIENLNLRITIDINKSRKAVLNRIAIPRGFVKRHILGWQESEKVSKKIREAECEILLSKEYEELSKQFFQSKDYDKMTRINGLYEKNKLIALMAVYLMGQLRILFKEHTKLDDITKTTVDFKISDKVTVKIPFSNYPSLVYTMSSKYVDNIGNYGFSNKDKDKPILGKIDVIEKQRMEFIKEVLGFEKYLFDDKIIDKSKFADTATHISFAEIVEELVEKGWDKDRLTKLKDARNKALHGEILTGTSFDETKSLINELKK

SEQ ID NO: 9 - protein, 789 aa, Artificial Sequence
  Description of Artificial Sequence: Synthetic polypeptide
MENIKLEKQKAAFYFNQAELNLKAIEGNIFDKGRRKTLFDNPKILSKVENFIFNFKDVTKNAKGEIDCLLSKLMELRNFYSHYVHKPDVKELSKGEKPLLERYYQIAIEATGSENVKLEIIENDKWLTDAGVLLFLCMFLKKSQANKLISGISGFKRNDTFGQPRRNLFNYFSVRERYKVVPDMQKHFLLFVLVNHLSEQDDYIEKAQQPYNIGEGLFFHRIASTFLNVSGILRNMEFYTYQSKRLKEQRGELKREKDIFTWEEPFQGNSYFEINGHKGVIGEDELKELCYALLSYNKSKYAVEQIEKFLKGFGEVKSEQEIRDSDILNESYFPTNYFAESNIGSIKEKILNRLGKTDDSYKKTGTKIKPYDMMKEVMEFINNSLPADEKLKRKDYRRYLKMVRIWDSEKDNIKREFESKEWSKYFSSNFWMAKNLERVYGLAREKNAELFNKLKAVVEKMDEREFEKYRQINSAEDLASLRRLANDYGVKWEEKDWQEYSGQIKKQISDRQKLTIMKQRITAELKKKHGIENLNLRITIDSNKSRKAVLNRIAVPRGFVKEHILGWQGSEKVSKKTREAKCKILLSKEYEELSKQFFQTRNYDKMTQVNSLYEKNKLIAFMAVYLMGQLNIRFDKPTRLNELEKAEVDFKISDKVTAKIPFSQYPSLVYAMSSKYADSVGSYKFENDEKNKPFLGKIDIIEKQRMEFIKEVLGFEEYLFEKKIIDKSKFADTATHISFREICDELIQKGWDENKLTNLKDARNAALHGEIPAETSFREAKPLINGLKK

SEQ ID NO: 10 - protein, 799 aa, Artificial Sequence
  Description of Artificial Sequence: Synthetic polypeptide
MINIELKKEEAAFYFNQANLNISGLDEVIEKQLPHIGSKKENAKKAIDKIFDNITVLKKVENFVFYFKDVAKNERVELDALLLKLIDLRNFYSHYVHNDNVKILSDGEETLLEKYYQIAIEATGSKDVKLEIIDNEKKLTDAGILFLLCMFLKKSQANKLISSISGFKRNDKEWQPRRNLFTYYSLREGYKVVPDMEKHFLLFTLVNHLSTQDENIENTQPSDDIGRGLFFHRIASTFLNISGIFNNMEFYPYQSNRLKERRGDIAPDKDSFAWIEPFQGNSYFKINGYKGVIGENELKELCFAVLLHNKSKYAVEQIEKFLKCFKEVQSKQEIIECDILDECYFPANYLNQPETKSLKEKLLSRITGKINYSFDTAEKAFDKMKDVMEFINGCLPSDEKLKRKDYSRYLKMVRFWGGEKDNIKREFEGKKWTRFFPSELWHKRTLEDVYKFALKKNKKRLEELKVKIEGLNEDDLLKYQKVNNIKNLENLRLLAHDLDLSWREKDWGEYSGQIKKQISDNQKLTIMKQRVIAELKKKHGIENINLRISLDSNKSIQAVLNRIAIPKGFIKRHVLHLQENEKTSRKIREAKCKILLSKKYEYLSRKFLDEKNFDKLTQINGLYEKNRLIAFMVIYLLKQLGLELKNETKLIELKKTRVKYKISDKVAEDIPLSHYPSLVYAMSRKYVDNIDNYEFPDEYAKKAILDKVDIIENQRMEFIKQVLGFEKYLFDNNIIDKSKFTDVETHISFVKIHDELIEKGWDTEKLSKLKHARNKALHGEIPGGTSFEKAKLLINELKK

SEQ ID NO: 11 - protein, 877 aa, Artificial Sequence
  Description of Artificial Sequence: Synthetic polypeptide
MKKQKSKKTVSKTSGLKEALSVQGTVIMTSFGKGNMANLSYKIPSSQKPQNLNSSAGLKNVEVSGKKIKFQGRHPKIATTDNPLFKPQPGMDLLCLKDKLEMHYFGKTFDDNIHIQLIYQILDIEKILAVHVNNIVFTLDNVLHPQKEELTEDFIGAGGWRINLDYQTLRGQTNKYDRFKNYIKRKELLYFGEAFYHENERRYEEDIFAILTLLSALRQFCFHSDLSSDESDHVNSFWLYQLEDQLSDEFKETLSILWEEVTERIDSEFLKTNTVNLHILCHVFPKESKETIVRAYYEFLIKKSFKNMGFSIKKLREIMLEQSDLKSFKEDKYNSVRAKLYKLFDFIITYYYDHHAFEKEALVSSLRSSLTEENKEEIYIKTARTLASALGADFKKAAADVNAKNIRDYQKKANDYRISFEDIKIGNTGIGYFSELIYMLTLLLDGKEINDLLTTLINKFDNIISFIDILKKLNLEFKFKPEYADFFNMTNCRYTLEELRVINSIARMQKPSADARKIMYRDALRILGMDNRPDEEIDRELERTMPVGADGKFIKGKQGFRNFIASNVIESSRFHYLVRYNNPHKTRTLVKNPNVVKFVLEGIPETQIKRYFDVCKGQEIPPTSDKSAQIDVLARIISSVDYKIFEDVPQSAKINKDDPSRNFSDALKKQRYQAIVSLYLTVMYLITKNLVYVNSRYVIAFHCLERDAFLHGVTLPKMNKKIVYSQLTTHLLTDKNYTTYGHLKNQKGHRKWYVLVKNNLQNSDITAVSSFRNIVAHISVVRNSNEYISGIGELHSYFELYHYLVQSMIAKNNWYDTSHQPKTAEYLNNLKKHHTYCKDFVKAYCIPFGYVVPRYKNLTINELFDRNNPNPEPKEEV

SEQ ID NO: 12 - protein, 864 aa, Artificial Sequence
  Description of Artificial Sequence: Synthetic polypeptide
MKKSKVKLNGVKAVYHISPDVRVIAAFGRGNNSVLDKHIENGSVEELQNHSDIEVNISRKTYSFRKKSLKKAAGQFSVPDNTNDQLGIREELEKEIFGRKFDDNIHIQAAYAVNDIIKMLAVAANLAETAINGLDRENTENDMIGFCIIPHITYQTYADDKRSKFEGFIEKVKTQGTFSYFPDILPKFKKEESEEESDNEKLYYIMCIISLIRNSATHSKSSNSDTTDYIFGEFNSVNKEALTTTADNLIKSKIDFINKEGFSKNQKNNIYRLLKAKADTPENTARLIRRLYAFTIRKQDKNLGFSLKKLRECAIRSIDRSIEYMKYLPSKKYDTVRSKLYTLMDFVVYSYLKYHKDGKKFSKEMVEQLRAAESDKVKDDIYRDEAEKLYNIEIISRTINALISDIKSDFDQPKHGNECYQPINDGMKEAEKDFITTDQLSLFTKFIYVLCQFLDGKNINILLSSLISKFQQIEAFNGDIRKLNLNIRDDGKIGYDSKKYSIFEKSGQIADDLDKLRGVIKMDINDLNAYETMIKDALRVIGVDESDIESIYQTHFKTQDKKDSVSGFFRNNIINSRRFRYIIKYINPSDAYRIIQNENVRNYVLGRMNDAIIDRYAHSVGIEDKVHDKRKVLSDILSKVKFDNFTKLTYINPKDKNKGEKAKEREKPKAILGLYLTIVYLVVKSLVRINSQYVMAVYHLERDSRLCPGVSSNNLLSMTNHYCDRDNHLLKEKHIVKLERYKKTQKTQKTICTAYRNAIAHLSAVRKGVKYIGDIQKADSYFGIYHYCMQKLIYSANGQPFAEFVRSIFGDEKELDKLRNGSYSQAILRALNYPFGYNPARYKNLSYEKIFIRAWQDEDTNKKT

SEQ ID NO: 13 - protein, 880 aa, Artificial Sequence
  Description of Artificial Sequence: Synthetic polypeptide
MNKIHKKQGKTTAKSLGLKSVLKIENDLVVTTFGKKDNPMVVEQSINKASGEKELYVDEDQVKFDSSLIKEKNILSLDSIQHSNHQIIVNIDQKDASEIGMDYLRLKPELEKEFFGKTFYDNVHIQIAYNLLDLKKIIGLHIGNAIQALENLGRDGSDLVGICDATKPLNYLDDVKQKADIGFMNRLKPYFMYFDGVLKLDNSKNKNGELNQLDIENWDVIRILSLIRQGCAHAGAYSSLLYTAQNNKVYADLINKALSIFSDDLDKFNKSFLKQSKMNLFILFDLYNCRFDRSLQEKIIKEYYRYVLYKDNKNLGFSLKNVRNLIIEGKYDEQERSGKLQTIRSKLNTLLDFYLYGYYQKNPTFVENIVAKLRESKNDEDKEKVYEEEYHRLLSENNYLVDKKCSDIVYRINEAVKNRKIFVNANINAVVEKVSCSCFPSLIYVLCKFLDGKEVNELTTAIINKLENIASLINALVTLKSYGGFSEQYKIFDYPNINGLIDDFRMVKNLTSTKRKLKKASGGEDRIGRQLYADAINIFKEDSFVSANDEKGTGLDQYVNKFFSKDDLGARKVRNLLLNNIIKNRRFVYLIKYIDPKDCYKLVHNEKIVRFALGQYDESQMPLNQLQKYYDAVIENREGFRKCNDRKKIIDTLVSEINRVSIDGILDIGNRLVNRGNNDYINHQKQIISLYLTIAYLIVKGVVHTNSLYFIAWHAYERDNNFKFGNDGKDYLALTKEYLTNKKKRVKQLLDHNIEEANNSLDSKYFSAYRNKVVHLNFCNIFVNYLDGIGDIHSYYDIYQYVIQKWSIAERSKDFIDPQYLTKLSNDLKQYRTYQRNFLKIINLPFAYNLARYKNLTIGDLFNDKYPLPKETVKEFYNEE

SEQ ID NO: 14 - protein, 880 aa, Artificial Sequence
  Description of Artificial Sequence: Synthetic polypeptide
MAKKKRMSAKERKQQQINLRIKKATEDSTKKVNTTVAVNNKPISKEIKKSKAKLAGVKWVIKANDDVAYISSFGKGNNSVLEKRIIGDVSSDVNKDSHMYVNPKYTKKNYEIKNGFSSGSSLTTHPNKPDKNSGMDALCLKTYFEKEIFKDKFNDNMHIQAIYNIFDIEKTLAKHITNIIYAVNSLDRSYIQSGNDTIGFGLNFNIPYAEYGGGKDSNGKPENKSAWEKRESFIKFYNNAKDRFGYFESVFYQNGKQISEEKFYIYLNILNFVRNSTFHYNNTSSHLYKERYCKINPKNNLKTDFEFVSYLNEFVKNKFKNVNKNFISNEKNNLYIILNAYGEDIEDVEVVKKYSKELYKLSVLKTNKNLGVNVKKLRESAIEYGYCPLPYDKEKEVAKLSSIKHKLYKTYDFVITHYLNSNDKLLLEIVEALRLSKNDDKKENVYKIYAEKIFKAEYVINPIKTISNLFAEKGDKLFNEKVSISEEYVEDIRIDKNIHNFTKVIFFLTCFLDGKEINDLLTNIISKLQVIEDHNNVIKAIANNNDAVYKDYSDKYAVFKNSGKIATELEAIKSIARMENKINKAFKEPLLKDAMLALGVSPNDLDEKYEKYFKTDVDADKDHQKVSTFLMNNVINNSRFKYVVKYINPADINRLAKNKHLVKFVLDQIPHKQIDSYYNSVSTVEEPSYKGKIQLLTKKITGLNFYSLFENCKIPNVEKEKKKAVITLYFTIIYILVKNLVNINGLYTLALYFVERDGFFYKKICEKKDKKKTNKDVDYLLLPEIFSGSKYREETKNLKLPKEKDREIMKKYLPNDEDRKEYNKFFKQYRNNIVHLNIIANLSKLTSTIDKEINSYFEIFHYCAQRVMFDYCKNNNKVVL

SEQ ID NO: 15 - protein, 895 aa, Artificial Sequence
  Description of Artificial Sequence: Synthetic polypeptide
MKKNSNDKTNAKRMGIKSFIKNGDERFITTSIKNEFPVELKLDVIKKTCEPAHEPVSFDYDPKKIDFEKPVLKEKLTSGQSGQKLSTRLFIQKDRDICGIRRKYLEKIFNSNFIEEKKDSNLPMQIVAKVLSTEKVFSNALNKIISQFLSMPRGGVTDNHGEYEIIGNIINHKSLQELNKEKKTKRIKKYLQSVIKNQSYLYNKQFLLSLDESKGSRNDIDENELYDYIRFLAILRNGIAHVFYEKNEPETAKESLFRLVDFIKNDKKLEGAFAKIKIQVNTLYKCRKEEYIKKSGKNFEIIRKIYQNDKPDEKVKDWIRYDFDKSYKYIGLSVAKLGNYTSWAKDIDNLRDKSNPDSGYAGIMHRLNEFSVYLKVKALSTEEKDKYLKNLISKENCEEKDKYYKNIAQFFCSSDLKFANVLQMVKEIKKNKGCTSEDKNCKLCVDERKFNDLSVIVYFISCFLDNKDQNIFLSDLINRFGALSDLLRIQNKILGAGNKYNENYSFLKNERYVTEIKMELETIFALVKVSYKKEDKAFNRLLEDGLVMFGFSKDEAGMKVAGLKEIKEKKEGHYKNKSRSFLINSIVNSRKFAYLAKSIDPQKVPAIIKNEHIVRYILGRINKTNPGQIGRYWRYIMSQNHAGTDKVDDLTNEIIKINIKNILNDAGGWQKSKLNDNNNKKKLKYQQLIGLYLTVAYIFVKNMLECNARYFSAFAQIEKDYLIYTNSDEFYYIDKNKKNLVTERYLKLVKDIIEKNKNTVRKDKIFRKKRQRKHLADISKSIIEFEKLPCCIFTLLRNITEHLNVASNIDIIEGYGKRAGKYHKNAPASYFIFYHYIIQKILADKICTRNLLNIINTYGEPSISFIKIIYVPFAYNLPRYLNLTDARIFCNMDDK

SEQ ID NO: 16 - protein, 857 aa, Artificial Sequence
  Description of Artificial Sequence: Synthetic polypeptide
MKKLKNPSNRNSLPSIIISKFDSSKIYEIKVKYEKLARLDRLEIGDMSLDENLNILFKKVNFNGIDLEILNPLLLDFDSYTISGKLQKNSTNKTILTLKKDGKIIKYNVLEKDNKYFKNGKEFVIPKDVKEEGKRLVNDKFLLTIEDKKREENSLPKKRKKETQRDILKDETIEIYKRISSNSNIKSEDIYRIKRYMLFRSDMMFFYTFIDNFFYCLYKNKNEQLWNTNFKEKENLGKFIEFTLNDTLKNPRNGILKSYSKDLKVVQEDFVKIKDIFEKIRHALAHFDFTFIDNLLSNNIEFDFNIKLLNIVIEDSQDLYYEAKKEFIEDEKMDILDEKDISIKKLYTFYSKIDIKKPAFNKLINSFLIKDGVENSKLKEYIKEKYNCHYFIDIHDNKEYKKIYNEHKKLISENQNLQLNSKENGQKIKINNDRLEELKGKMNELTKANSLKRLEFKLRLAFGFIKVEYNIFKDFKNNFSEDIKKDMNIDLEKIKSYLDTSYSNNQFFNYKVYNKKTKQKDIDKDIFDDIEKETLKELVENDSLLKIILLFYIFTPKELKGEFLGFIKKFYHDTKNIDKDTKDKEEPLEQIKQEVPLKLKILEKNLTILTIFNYSISLNIEYDKNNNSFYERGNKFKKIYKDLKISHNQEEFDKSLLAPLLKYYMNLYKLLNDFEIYLLLKYKNKDNLNKESLNKLINDEQLKHNDHYNFTTLLSEYFNFDPKKNKKYETLTILRNSISHQKIDNLIYNLDKNKILEQRVKIVELIKEQRDIKETLKFDPINDFTMKTVQLLKSLENQSEKRDKIEEILKQQDLSANDFYNIYKLKGVESIKKELFIRLGKTKIEEKIQEDIAKGSI

SEQ ID NO: 17 - protein, 839 aa, Artificial Sequence
  Description of Artificial Sequence: Synthetic polypeptide
MNSIEKIKKPSNRNSIPSIIISDYDENKIKEIKVKYLKLARLDKITIQDMEIRDNIVEFKKILLNGIEHTIKDNQKIEFDNYEITAYVRASKQRRDGKITQAKYVVTITDKYLRDNEKEKRFKSTERELPNDTLLMRYKQISGFDTLTSKDIYKIKRYIDFKNEMLFYFQFIEEFFSPLLPKGTNFYSLNIEQNKDKVVKYIVYRLNDDFKNQSLNQFIKKTDTIKYDFLKIQKILSDFRHALAHFDFDFIQKFFDDELDKNRFDISTISLIKTMLQEKEEKYYQEKNNYIEDSDTLTLFDEKESNFSKIHNFYIKISQKKPAFNKLINSFLSKDGVPNEELKSYLATKKIDFFEDIHSNKEYKKIYIKHKNLVVEKQKEESQEKPNGQKLKNYNDELQKLKDEMNKITKQNSLNRLEVKLRLAFGFIANEYNYNFKNFNDKFTLDVKKEQKIKVFKNSSNEKLKEYFESTFIEKRFFHFCVKFFNKKTKKEETKQKNIFNLIENETLEELVKESPLLQIITLLYLFIPKELQGEFVGFILKIYHHTKNITNDTKEDEKSIEDTQNSFSLKLKILAKNLRGLQLFNYSLSHNTLYNTKEHFFYEKGNRWQSVYKSLEISHNQDEFDIHLVIPVIKYYINLNKLIGDFEIYALLTYADKNSITEKLSDITKRDDLKFRGYYNFSTLLFKTFMINTNYEQNQKSTQYIKQTRNDIAHQNIENMLKAFENNEIFAQREEIVNYLQKEHKMQEILHYNPINDFTMKTVQYLKSLNIHSQKESKIADIHKKESLVPNDYYLIYKLKVIELLKQKVIEAIGETKDEEKIKNAIAKEEQIKKGYNK

SEQ ID NO: 18 - DNA, 36 bp, Artificial Sequence
  Description of Artificial Sequence: Synthetic oligonucleotide
gctggagcagcccccgatttgtggggtgattacagc

SEQ ID NO: 19 - DNA, 36 bp, Artificial Sequence
  Description of Artificial Sequence: Synthetic oligonucleotide
gctggagcagccctcgatttgctgggtaatcacagc

SEQ ID NO: 20 - DNA, 36 bp, Artificial Sequence
  Description of Artificial Sequence: Synthetic oligonucleotide
gctgaagcaaccctggttttgcggggtgattacagc

SEQ ID NO: 21 - DNA, 36 bp, Artificial Sequence
  Description of Artificial Sequence: Synthetic oligonucleotide
gctgtagaagcctccgatttgtgaggtgatgacagc

SEQ ID NO: 22 - DNA, 36 bp, Artificial Sequence
  Description of Artificial Sequence: Synthetic oligonucleotide
gctggagcagccctcgatttgcagggtaatcacagc

SEQ ID NO: 23 - DNA, 36 bp, Artificial Sequence
  Description of Artificial Sequence: Synthetic oligonucleotide
gctggagcagccctcgatttgcagggttatcacagc

SEQ ID NO: 24 - DNA, 36 bp, Artificial Sequence
  Description of Artificial Sequence: Synthetic oligonucleotide
gttggagtagccccggatttgcggggtgattacagc

SEQ ID NO: 25 - DNA, 36 bp, Artificial Sequence
  Description of Artificial Sequence: Synthetic oligonucleotide
gctgtgatagacctcgatttgtggggtagtaacagc

SEQ ID NO: 26 - DNA, 36 bp, Artificial Sequence; identical to SEQ ID NO: 25
  Description of Artificial Sequence: Synthetic oligonucleotide

SEQ ID NO: 27 - DNA, 36 bp, Artificial Sequence
  Description of Artificial Sequence: Synthetic oligonucleotide
gctgtgatggacctcgatttgtggggtagtaacagc

SEQ ID NO: 28 - DNA, 36 bp, Artificial Sequence
  Description of Artificial Sequence: Synthetic oligonucleotide
caactacaaccccgtaaaaatacggggttctgaaac

SEQ ID NO: 29 - DNA, 36 bp, Artificial Sequence
  Description of Artificial Sequence: Synthetic oligonucleotide
gttaaataccacctaagaatgaggaggttctataac

SEQ ID NO: 30 - DNA, 36 bp, Artificial Sequence
  Description of Artificial Sequence: Synthetic oligonucleotide
gaacgatagcctgctgaaatatgcaggttctaagac

SEQ ID NO: 31 - DNA, 36 bp, Artificial Sequence
  Description of Artificial Sequence: Synthetic oligonucleotide
gattgaaagctatgcgaatttgcacagtcttaaaac

SEQ ID NO: 32 - DNA, 36 bp, Artificial Sequence
  Description of Artificial Sequence: Synthetic oligonucleotide
gagatagacccttgttaactcgtaaggttctgtgac

SEQ ID NO: 33 - DNA, 36 bp, Artificial Sequence
  Description of Artificial Sequence: Synthetic oligonucleotide
attggatatacccctaatttgagaggggaataaaac

SEQ ID NO: 34 - DNA, 36 bp, Artificial Sequence
  Description of Artificial Sequence: Synthetic oligonucleotide
gttggactataccctcgtttgtagggggaataaaac

SEQ ID NO: 35 - protein, 7 aa, Artificial Sequence
  Description of Artificial Sequence: Synthetic peptide
GSGGGGS

SEQ ID NO: 36 - protein, 15 aa, Artificial Sequence
  Description of Artificial Sequence: Synthetic peptide
GGGGSGGGGSGGGGS

SEQ ID NO: 37 - protein, 336 aa, Unknown
  Description of Unknown: ADAR1DD sequence
SLGTGNRCVKGDSLSLKGETVNDCHAEIISRRGFIRFLYSELMKYNSQTAKDSIFEPAKGGEKLQIKKTVSFHLYISTAPCGDGALFDKSCSDRAMESTESRHYPVFENPKQGKLRTKVENGEGTIPVESSDIVPTWDGIRLGERLRTMSCSDKILRWNVLGLQGALLTHFLQPIYLKSVTLGYLFSQGHLTRAICCRVTRDGSAFEDGLRHPFIVNHPKVGRVSIYDSKRQSGKTKETSVNWCLADGYDLEILDGTRGTVDGPRNELSRVSKKNIFLLFKKLCSFRYRRDLLRLSYGEAKKAARDYETAKNYFKKGLKDMGYGNWISKPQEEKNF

SEQ ID NO: 38 - protein, 336 aa, Artificial Sequence
  Description of Artificial Sequence: Synthetic polypeptide
SLGTGNRCVKGDSLSLKGETVNDCHAEIISRRGFIRFLYSELMKYNSQTAKDSIFEPAKGGEKLQIKKTVSFHLYISTAPCGDGALFDKSCSDRAMESTESRHYPVFENPKQGKLRTKVENGQGTIPVESSDIVPTWDGIRLGERLRTMSCSDKILRWNVLGLQGALLTHFLQPIYLKSVTLGYLFSQGHLTRAICCRVTRDGSAFEDGLRHPFIVNHPKVGRVSIYDSKRQSGKTKETSVNWCLADGYDLEILDGTRGTVDGPRNELSRVSKKNIFLLFKKLCSFRYRRDLLRLSYGEAKKAARDYETAKNYFKKGLKDMGYGNWISKPQEEKNF

SEQ ID NO: 39 - protein, 385 aa, Unknown
  Description of Unknown: ADAR2DD sequence
QLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPADRHPNRKARGQLRTKIESGEGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLT

SEQ ID NO: 40 - protein, 385 aa, Artificial Sequence
  Description of Artificial Sequence: Synthetic polypeptide
QLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPADRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLT

SEQ ID NO: 41 - protein, 198 aa, Artificial Sequence [sequence omitted]

SEQ ID NO: 42 - protein, 208 aa, Artificial Sequence
  Description of Artificial Sequence: Synthetic polypeptide
MTDAEYVRIHEKLDIYTFKKQFFNNKKSVSHRCYVLFELKRRGERRACFWGYAVNKPQSGTERGIHAEIFSIRKVEEYLRDNPGQFTINWYSSWSPCADCAEKILEWYNQELRGNGHTLKIWACKLYYEKNARNQIGLWNLRDNGVGLNVMVSEHYQCCRKIFIQSSHNQLNENRWLEKTLKRAEKRRSELSIMIQVKILHTTKSPAV

SEQ ID NO: 43 - protein, 229 aa, Artificial Sequence [sequence omitted]

SEQ ID NO: 44 - protein, 7 aa, Simian virus 40
PKKKRKV

SEQ ID NO: 45 - protein, 16 aa, Unknown
  Description of Unknown: Nucleoplasmin bipartite NLS sequence
KRPAATKKAGQAKKKK

SEQ ID NO: 46 - protein, 9 aa, Unknown
  Description of Unknown: C-myc NLS sequence
PAAKRVKLD

SEQ ID NO: 47 - protein, 11 aa, Unknown
  Description of Unknown: C-myc NLS sequence
RQRRNELKRSP

SEQ ID NO: 48 - protein, 38 aa, Homo sapiens
NQSSNFGPMKGGNFGGRSSGPYGGGGQYFAKPRNQGGY

SEQ ID NO: 49 - protein, 42 aa, Unknown
  Description of Unknown: IBB domain from importin-alpha sequence
RMRIZFKNKGKDTAELRRRRVEVSVELRKAKKDEQILKRRNV

SEQ ID NO: 50 - protein, 8 aa, Unknown
  Description of Unknown: Myoma T protein sequence
VSRKRPRP

SEQ ID NO: 51 - protein, 8 aa, Unknown
  Description of Unknown: Myoma T protein sequence
PPKKARED

SEQ ID NO: 52 - protein, 12 aa, Mus musculus
SALIKKKKKMAP

SEQ ID NO: 53 - protein, 5 aa, Influenza virus
DRLRR

SEQ ID NO: 54 - protein, 7 aa, Influenza virus
PKQKKRK

SEQ ID NO: 55 - protein, 10 aa, Hepatitis delta virus
RKLKKKIKKL

SEQ ID NO: 56 - protein, 10 aa, Mus musculus
REKKKFLKRR

SEQ ID NO: 57 - protein, 20 aa, Homo sapiens
KRKGDEVDGVDEVAKKKSKK

SEQ ID NO: 58 - protein, 17 aa, Homo sapiens
RKCLQAGMNLEARKTKK

SEQ ID NO: 59 - protein, 8 aa, Homo sapiens
PQPKKKPL

SEQ ID NO: 60 - protein, 130 aa, Artificial Sequence
  Description of Artificial Sequence: Synthetic polypeptide
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIY

SEQ ID NO: 61 - protein, 133 aa, Artificial Sequence
  Description of Artificial Sequence: Synthetic polypeptide
MAKLETVTLGNIGKDGKQTLVLNPRGVNPTNGVASLSQAGAVPALEKRVTVSVSQPSRNRKNYKVQVKIQNPTACTANGSCDPSVTRQAYADVTFSFTQYSTDEERAFVRTELAALLASPLLIDAIDQLNPAY

SEQ ID NO: 62 - protein, 128 aa, Artificial Sequence
  Description of Artificial Sequence: Synthetic polypeptide
MSKTIVLSVGEATRTLTEIQSTADRQIFEEKVGPLVGRLRLTASLRQNGAKTAYRVNLKLDQADVVDCSTSVCGELPKVRYTQVWSHDVTIVANSTEASRKSLYDLTKSLVVQATSEDLVVNLVPLGR

SEQ ID NO: 63 - DNA, 10 bp, Artificial Sequence
  Description of Artificial Sequence: Synthetic oligonucleotide
tcgatttgct

SEQ ID NO: 64-66 - skipped (3 entries)

SEQ ID NO: 67 - RNA, 36 nt, Artificial Sequence
  Description of Artificial Sequence: Synthetic oligonucleotide
ggcccaacaugaggaucacccaugucugcaggggcc

SEQ ID NO: 68 - RNA, 29 nt, Artificial Sequence
  Description of Artificial Sequence: Synthetic oligonucleotide
ggcccaugcugucuaagacagcaugggcc

SEQ ID NO: 69 - RNA, 34 nt, Artificial Sequence
  Description of Artificial Sequence: Synthetic oligonucleotide
ggcccuaaggguuuauauggaaacccuuagggcc

SEQ ID NO: 70 - skipped

SEQ ID NO: 71 - DNA, 30 bp, Artificial Sequence
  Description of Artificial Sequence: Synthetic oligonucleotide
gcagcttcaccttgtagatgaactcgccgt

SEQ ID NO: 72 - DNA, 30 bp, Artificial Sequence
  Description of Artificial Sequence: Synthetic oligonucleotide
gttcatcacgcgctcccacttgaagccctc

SEQ ID NO: 73 - DNA, 30 bp, Artificial Sequence
  Description of Artificial Sequence: Synthetic oligonucleotide
tgcttcacgtaggccttggagccgtacatg

SEQ ID NO: 74 - DNA, 30 bp, Artificial Sequence
  Description of Artificial Sequence: Synthetic oligonucleotide
cgtctggccttcctgtagccagctttcatc

SEQ ID NO: 75 - DNA, 2601 bp, Unknown
  Description of Unknown: Cas13 coding sequence
atggctgtagattactcactaaaaaatgaatggtatcgggaaataaataaatcctgttttacggttgcgcttaatgttgcatatgacaattgtaaggcaaaaggtcatgaaaatcttctacgtgaagcccagcggagcaaaggcggtatcacaaatgaacagattaaaaatgttcaaaccgaaataaaaactcgtcttgaagatatacgcagccacttctcgcatttttatcatgacgaaaagagcctgatatttgaaaaagacaacatagttaaggatttcctggagtctgcatacgaaaaagctcagtcaagcgttataggcagtacgagacagagtgattataaaggtgtggtaccgccattattcgagccgcatgacggcatgataacagcggccggggtggttttcttggcatcgtttttctgtcatagaagcaatgtatatagaatgttaggggcagttaagggctttaagcataccggtaaagaagagcttagcgacggagcaaaacgcgattacggttttacacgcaggctgatggcacattattcactacgggatagctacgttattaaggcagaggaaacaaaatccttccgagacctgctcggatacctgtctcgcgtgccgcaacaggccgtggactggctgaacgagcacaatcaattaagcgaagatgagaaaaaggaatttcttaatcaaaagccttccgatgaagaatcgcaggaacaatccaaaactgaaaatactgacaggcaggccgatagaatgccaagacgttcacttcgcaaaacggataagtttatcttgttcgcagcaaaatttatcgaagattgggcgcagaaagaaaaaatggatgttacgtttgcaagataccaaaaaaccgtaactgaggatgagaataaaaaccaggacggcaagcaggtaagagatgtgcaattaaagtatgagaaagacacgaaaaaactaaacccggattttgattacaaatggacttactatatacgcaacaatcacgcgattatccaaattaaaccggacgaatataaacaagcggtatccgcaagaatctctgaaaacgagctgaaataccttgtgctattgatttttcagggcaaaggttgggaagcaatcaagaaaataggagactatatctttcacataggaaacaagataaaaatcggcaggttcgaccataatgaggaacgccgaatgccgtcgtttctgaaaaatccgcctgcagacatcataggcgaaatggttgaaaaccggttaaagtatattcgtgatgagcttaataaagtgattgaaacgataaaaaaggaagagccacaaaacaataaatggctgttatataaaggaaaaaagatttcaatcattctgaaatttatctccgacagcataagcgacattaaaaaacggcctgatgtaaatgaatacaacactttacgtgatatgctgcaaaagcttgatttcgataatttctatgaaagactaaaaagctacgtaagcgaaggcagaattgagcagacgttgtatgacgagattaaaggaattaaggacatcagtacactctgtataaaaatttgtgagctcagactggctgcccttgaagagcttgagaaagaaggcggcgacgatctgaacaaatatattggactcgctgtgcaggaaaaacacaaaaattacgatgactcgaatactccccaaaaaaaggctgaaagatttttggaatcgcagttttctgttggcaaaaattttctgcgcgagacattctacgacgaatatatcaaaaacagaaaaagcctgtatgaaattatcaaagagaaaataacaggcatcacgccgctgaacgaaaaccgctggtatctcatggataaaaatccgaaggaatttgagagcaaagacagtaaaattatcagagggctttgcaatatttatattcaggatattctatgcatgaagattgctttgtggtactatgaaaacctgagcccctcttataaaaataagctgaaatgggatttcattgggcagggattcggctacgaccggtataagctttcttataaaactgactgcggtattacgatcgagtttaaactcgccgatttgaaccggctcgatattatcgaaaaaccaaaaatgatagaaaatatctgccatagctttattctggaaaaagacgtcaaaaagcaaacgataagctggcatgagttcagacaagatggtatcgcgaaatatagaaaactacagaaagaagtagttgaggctgtatttgaattcgagaatagtctgaagatcccggataaaaattggctgacgcagggatatgtcccgtttaataaaaataagaggttcgaagataaaggcttctctacttttattcttgaggaggctgtaagaaaaggtaaaataaaatcggacgacaaagagcctttacgcaaggttcgcactgatttctttcatgagcaatttgactcgaccgatgctgaaagaaggatttttgataaatatatgccggccaaacatgacggtaaaaataaaggtggtaaaatgcaggaaaaacaggaaaaaagttatacaagaagaatttga

SEQ ID NO: 76 - DNA, 2375 bp, Unknown
  Description of Unknown: Cas13 coding sequence
atggcagcgggttctttttgtaaattccgaaggcagaggctgcgactaaaatcagcgcggcaaaataaagacctcttctacgtgcagaagtaataagaacgaccagcaaagctatggcagcggaaacgaaacaggctgctatagcaccggtatagtttataagaataagcacaagcaggcagccagccgctgcgccagtaaggtctgcgaaatatacacggttaatgttccgggcggcggtgctaataatataagcgatgcaaacaccggcgaaaaagaacggagaagcagtcagtattattatgataaacagcgacagcatattgctgtagtctttgttgattaatatatcgagagggtagaaatggattctcggtatgaaaattattgtagcaatggttgatagtgcaaacagtcctgccattttgttgaggaattcatggccgtttgatgaacccggcttttgctttctggttgctaaataagcaccggcagcaccaaaaccgagcatagctatgctgattaccaggtaagtaaaatggtggtacataatgaatgaaaagactcgagtcagggcaacttccaggctcaacacggccatagctaatagaaaaaccagaagtgaacgaagtggaaggtcgcttgaagtgctcatatataaattccttaaatatggaacaaatgattaaattgagtttttgtcaaagctggatatctataatatataaaattttataagtgacaagcgaattctatattttctgaaggtaaactgatggattgtcggaacggatgtttttagttagaataataaccagtggaattggggcgggacaaattagttgacagggtagttgctcggctcgaagccgcaaaacaaggggggcgggttttggtcgaagggacatgggggtcctttgcgcctttattagtcaggcacatatcgcagcaaatcaaaaagccgattctatatatccgcccgcatatcgatgacgccgacaatgctgccgatgacctgctcgcattcggcgctgaaaatgtgcagcaattaccagcctgggaaggcgccgaagatattgccgatgccaccgatgaaatccgtgcagaaagggctaagctcgttacctgtctctcatctctcgcgaccggaaaagacagcaatttaattattcccgcttcgattcaggccttattgcagccggtaccgtctccggattcgctacgcgaaaattcgctcgaattgaaaaccggcttcaatttgccgcaggaaaaactaatcgagtggctcgttgaagggcaattcgagcgcgtcgataaagtcgatatgccaggccagttcgcccgccgtggcggcattgtcgatatctatccggttctggttgaaatgaccgagcagactcaaacgacttctccgctgcgtatagagttttttggtgatgagatagaaagtatccgcggcatcgacctggatacacaaaggtcgggcggtgacataaaaaacataaagataatctcatcagtagcaggcggacaggcggatgagctttttataaatgtcctgcctgaaaatacgctaatcgtaatcgaagaggtaaacgatgttcaggaggtcgcgacagtttaccttgaaagattgtctttgcctgaaaagttattcaactgggaacaggtttataaatcgttccaaaacttcggtcagcttcacataagcagattcgccggtactgatagcggtgactggttgagaatcaacgtcaaaagcattcagcagttccagccgagtacgacaaccccgatgtatcgggggtcgggccataaagaggcgctcgaacaattagtccggcaggcacgaggcggtaagaaagtatatctgtattgcgaagccgccgcggaaattgaccgtgttaccgaaatcatagagcaaagcgataaatccctgccgaaaaatctaaatcttgttaaggggtttatcaatcaaggctttgtaatcgagtcccgatacaatcgggatacgattgtcatcagccatcacgaattgttcgggcagttcgcgattcgcagaagataccggccaatccgcccgagcgccccggtcgatacgctggaggatttacaggccggtgattatgtggtgcacgcctcctatggagtcggcaaattcctcggcgttaccacgatagaagataaaacaggcgccggtgaatttttaacgctggagtatgccgacaaggtcaaggttcaggtctcggtggacaatattcacctcgtacagaaatatatcggcaccagcccgcgcagaccggtgttaagcaaggtcggctcgaaaaagtggctcaaacaaaagaaaaaggtggaagaatcagttcacgatttag

SEQ ID NO: 77 - DNA, 2397 bp, Unknown
  Description of Unknown: Cas13 coding sequence
atggagaaagaacaaggtctatattccatcgaccgctatcagggagccggcaaatggtgttttgccatcggggccaacagggcctgggataattacaacgaaaggccgaagctgttttctgaatcgctcctacgatacgagaaggccactcggcgagactggtttgatgaagagactcgcggcctcataaagaaaagtgacgttaggcagcggctgcggaaaatccggtgctacttctctcattattgccatgacaacacatgccttggttttgatcctgacgatgacctcagaaagataatggaaaaggcgtatgagagggccatcttcgagcagagaaagcacctgagcacagaaacggacattgagactcccgcgctgttcgaaccgcacgggcggattaccgcagccggggtggtgttcttctgttccttctttgtcgaacggcgcattctcaatcgactcatgggccgtattccagggttcaaaaaaaccgagggtgaatacggcgccacgcgacagatgttttcgaagtattgccttcgggacagctactccatacgcgcgagcgattcgaacgcagttctgttccgggatatcctcggatatctgtcgcgggcaccgtcgcaatactatcgtcataacaaagaccagtgcgataaagacgggcatccggaacgcaagaaagataagttcattaatctcgccttgagatatcttgagagctttgtgcctgcacgactgcggaaccacacgttgtctgtcgggcgaaaagaggtcgtccgaatggaaacaaacgcggtggcggagggcgagggagaataccgcccgtatccaccgaaggccaaggttaaggtcgtattcacagaggacgacccagagcggccctactacatcactcataacacggtgattctgcaaaccgcaaagaaggaagaggatattcaccactgcaaggttggtgtcaatgaattgaaatacctcgttctcttgtgtctccagggaaaggcggagaaggccgtcgcgggaatcgaggggtacgttcggcgaatccagggacgctttgcagaccacacaaacaaggtggcacgcgacgacgacgaacggcttgttcgagggcttcccgagtttgtgcgggtagcgagcgggatagaaacaccggacgaagtcagagagctgaagagcagacttgaccatataaggaagaagtggcagacgaagaaagcggaatcagccgaggcacagctccatcgcaaggcccgggacgtacttcgacacatcaactgggaaagccagcggcctcttggaattgagcaatacaatcgattgttggagcttcttgtcaatagggatttggagtcattcgcagcagagatgaaagagttgaagcggagaggactgatctcagaagaacttctgaaatcggtggaaggtatccgcaatctcaatacgcttcatgtgaaagtgtgcaaccttgttctgactcggttggagcacctggtggagaacgacccggaggagctgaaaaggcacatcggaattgtgccgagggaagagaaagaaggcccttcttacgaagagaaagtgcgagcgtttgtccaacagccgatgatgtacagaggattcctgcgcaattcattcttcaaaggatcgggaaagagtttcgcaaaactggttgaggaggagttgcacaagaaaggatgtcccgacgttccgctcgggacagactactatcttgtgcgcgatctggagcgagacgagagaaagaaccgcttccacaacgataatgccgcattgtatgaaacgctcgcgctcgatcgattgtgcgtattgatggcgcgagactgccttgtccgcctgaacagaaatctggaaaaacacgcaacgagaatatcgtgggaggcgacggacgccggagacacgatttgccttgagcttccgaggcgagatagagaccacgagtccttccggctgagttttggcgtcagggattaccccaaattatacgtcatggacgatccagtgtttctctgcggattgatgaagcatttcttccccgataaccaggcgatacagtatcatgaactatacagtgaaggcattaacaagtacactgccatgcaagcagagggaattgcggccactctgaaactggaagaaaagaccatcaaagagaagaacatgcaaatcccggctacgggatatattcgattctgtgaaatcgtgagccaaagcgactttgcacctggggaaaagagggtcttgaagaacgttcgcaacggcttgctgcattatcatctggagtttgagcctaccgagtgggctgaatttcgcgagattatgaaaagggagggattcgatactgcaaagaaaaggaaaagcacaaggaaaaaatga

SEQ ID NO: 78 - DNA, 2415 bp, Unknown
  Description of Unknown: Cas13 coding sequence
atgaaaattaaaaatgaaaatacggataagaaaacagaactatattcgatagataaatacaaaggccgcgataaatggtgttttgctatcgttctaaataaagcacaaacaaatttaacggaaaatcctgatttattcgagcaaaccataacgaaatatgaccggatcagaaaagaaggttggtttgacgaggaaacaaaaaaattaatctatatacaagaaaatgaacacaaaataaaaggagaaataaaaactttagcacgtgaggtattaaaaaatctgaggaattacttttcacatcatttttataaacaggactgcctaatatttcctaaagataatatagtcaggattataatgggaagagcatatgaaaggtctgaatatgaaataaagaaaaatctgaaggaagatatttcgatcgaattacccgctttattcgagccagaaggcaaaattacaacggcgggtatagtattttttacttcattttttgtcgaacgaagatttttacacaggctaatgggtagtgttcaagggtttacaaaaacagaaggagagtacaaaataacaagggatgtttttgctaaatattgtcttagagacagctattcggttaaagcacaggataacgacgcagttatgtttcgcgatattattggttatttgtcacgagttccaacggagtcattccaacatattaaaaatcctaagaaacaaaatgagagtcaattaagcgaaagaaagacagacaagtttatatctttcgccttaaaatatcttaaagattatggatttgaagatttgaaaggtcactacacggctttttttgcgagaagtgagattaagaaggaaaaagaagatatcgaaataaaagatgacaaaaaacacaaacctcacaggatgaaatcaaagattgagattcatttcgataaaacaaaagaagatcgcttttatattgagcgcaataatgtcattttgaagattcaaagaaaaggcggacgagccaatatattaagaatgggtatttatgaacttaaataccttgttctcttgtgtttgtccgggaaagcaagagaggcgatcaatagaatcgacgattatcttaatgatttacggaacaagatacctcatattgaaaatatgaacaaagagggtattggagaacaaataagatcccttccgggatttgtaagatcacaacttggctttgttcagattgatgatgagaaaaaaaaagaaaacagactggattatgttgaaaaaaaatgggaaaaaaagagagccgaatcaaaggagcttaagttgaatcgaaaagggcgggatatactcaggtatattaatgaaagatgtaagaagcctcttacaatcgatagatataatcgcattttagaacttctggtcgagaaaaatatagagggattttatcatgaactcgaagaactgagaaagacaggtcgtatagagaaaaatattactcaggcacttgtcggagaaaaaaacattaatgcacttcatattaagatttgtaaattagtgcaggatgaacttaaaagtttagaaaaggaagacctaaagaaatatataggcttgacacctaaagaagaaaaggtcgtgtcgtttgaagaaaaactcggtaggatcctcgataagccggttatttacaagggatttcttagataccagttcttcaaaaatgacaaaaagagtttcgcacgacttgttgaggaaataatcaaagaaaaaacaggaggtttagatgtacccattgaaacagagtattatagtatttcaacgcttggcagatttgataaagctaacaagacgttatatgaaacgcttgcgatggacaggctttgcatgatgatggcaaggcgatattttcttagtttgaataaaatacttgcaaaaagagcccaaaatatcgaatggaagaaagagagtggtaaagaatttattgttttcaagtttaatatgcctcaggatacaggaaaaagtatttcgatacggttttctccaaaagattatacaaaactttatgtgaagaatgattctgaatttttagcaagactttgccagtatttttttccgaatgaaaaagcgatcgattatcataagttatattctcatggaataaataaatatacaaactttcagaaagaagggatcgaggcaatacttgagcttgaagaaaagataatcaaaaaaaggaaaattaactctccagaaaattacttatcatttgaagaaatacttaatcaaagcatttataatgatgaagaaaagaatactttgattcaaatacggcattctcttttgcattatcaaatactttttagcaagaatgatctaacaaaattctacaacgtaatgaaaagagaggggattgaaaaaatttggtctttagtgatttaa

SEQ ID NO: 79 - DNA, 2616 bp, Unknown
  Description of Unknown: Cas13 coding sequence
atggaaaaatatttgataaaaaacttcgagggcattaacaaatcgaaattcacagttgctcttaatatcgcaaatgacaattgcaaaaataaaggcatccaagaattattaaaagaggcgcaacgcagcaaaggcggaataaccgacacccaaatcacagaggttcaggaacacattaaagaacgcctcaatagcgtaagaaattacttttcgcattgttatcatgaaaaaaagcctttgtattttgaggcaaacgaccctgtaaaaatattccttgaggaaacatttgcaaaagccgtagaaaatctgcaaggaagatttctttctgacaaatacaaattaacggttccgcccttgtttgaaccaaatcaaaataacactattaccgcggcaggagttatctttcttgcgtcatttttttgccacaggagttatgtatatagaatgcttggcggcataccaggttttaagcgcagcgacaagaaaaaatggggcgacggacagaaaattgattatggttttacgcgcaagctcatgtcgttttattcgcttcgagacagctattcggtaaatgtacaggaaaataaagaacttaccgcttttagagatatactcggttatctggcaagagtgccggggcaagcaatagactggctgatcgaaaaaggcaaattaacgaaagaagaaggaaaacaattttatctcggagaacaaagcgaagaacgagaagaaaaagcgaaaaaagaagaaataaaatacgccctgcggaaaacagataagtttatgctgttcgcagtgcggtttatagaggactgggcagagcaggaaaggataaaagtcgaatttgcccgttatgaaaaaatgacaatagtgaatgaaaacaaaaaacaggacgaaaaagaagaaagaaaagttaaatttgtatccgatgaaccgaccgcagcgggctggacttattatatacggaataaccacgcaattattaaaataataccagacgacaaaaagaaaaaggctgtttctgcgagaatttctgaaaatgaattgaaatatctggttttaacgataattgacgggaatggtaaaaatgcaattgcatatatcggcgattacattttcagaacagcgaggcaaatagaaaacaaatcttataatgccgaatctgaaaaatatgcgccagctttcgtcaggggcggtcaaaagaaatctgtcgataaaaggatcaaatatattagagacgaaattcaacaagtaatcaacgatattgaagcagagcaggaaaagcaaaaaaacgaacaagatgcgcctgctgaaaacagaacgtggcttatatataagggcaaaaaaatttcgataatacttcgatacgttaatgacaatatcgccgaatacaaaaaaaggttaagcgttacagagtataacgaactgagaggttacttgcagcaacttgactttataaattttcacaggaaacttgcggaatatcagcatcacgggagactgccaaacggttttgctgaatcgataaataaatttcaggatttgagcaagctatgcatagaagtatgtgaaaggcaaaaaaagaaacttcaagagatggcggcaaagggcggcatcgaacttgagcaatatataggtcttgcaccaaaagaagaaaatcaagagcagaataaatacgcaaccaaagcaaataactttataaaggtgtggctttcgatacccgaaaactttttgcggcagaaattttacgataaattttgtaagcaacaggaatgtaaaaataaagggagcgataaacccgataatacttcagtgccgcagaggaaatattttatagctataataagagaaaaaaatatcagaccgatacacgcagacaaatattatctgcttgggcaaaatccgaaagattacgaacgacccgacgggaaaatcataagacagctttgcgatgtttactgcaaagacgggctttgtatggcaatggccaaatggtattatgaaaacaggctcggcaagtttaaagatttaattgaatggcaaaccggcgacgataagcaacaacacggttatgccggtcatactcttgaatatcaggcaacggagaagataaaaatacgttttaaactggcagattttacaaggcttgacataatagaaccgccggagagggtaaaaaatatctgccggcaatgggaaacagaattactcaagaaaacacgggatggaacaataagctggtatgactttaaattgaatggccttgagccttacagacaatggcagggttatgcggtagcagatatattttggttcgaggaaagtttgaagataaacgaaactcaatggcagggacgaacacacatgccgtttaattttgaaaaagacaaacctttatggtgcaatattcttgatgaggcggtaaaacaaaataagatagaaaaacaggatacacaggctttaagacgagtcaggcatgattgttttcatgaagaatttttggcaaattatgaacaattgaagatttttaagaatttgatttcggataaggcaaaagacgcgaagcctaaagacaaaaagtctcgcaaaaacgaacaaaagtatggcaaaagataa

SEQ ID NO: 80 - DNA, 2442 bp, Unknown
  Description of Unknown: Cas13 coding sequence
atggaaacaacagaagaaaaaaaagagatttattcaatcgacaaataccaaggagcagggaagtggtgttttgccatcattctgaacagggcgcgtgataattacgaaggcaatccacatttactttccgagtctctgctgaggtttgaagcagttaatcgaagggactggtttgacgaggagacaagagaacttattatacaaatggatgcagaaaccaagacggattctaacctcaaaccaagtacacctgcgtacaaaatactgattggtattagaaattacttttcccatttttatcatacagaagagtgcttatatttcaaagaggatgatccaattcgcataattttggaaaatgcatataaaaaggccaagcagcatcacatagaaagactcgaaaaagaaacggatatcgaatttcctgctctgtttgaaagtaacgatagaattacatcggcggggataatattcttttcgtctttatttgtcgagcgtcgcatccttaaccggctgatgggttatgtcggtggttttaagaaaacaaagggtgaatataatatcacgcgggagatattttccacttattgcctgagagacagttattcaattatggccgcagattcaaacgcagtgatattcagagatatcttagggtatctgtcccgtgttccttcggaatattatcaacataacaaggaaaagtgcgaaaaggaaaacaaaccaaagcgtaagacaaagaaatttatttattttgcgttgagatatctcgaaaaatttgtcctgaaagagataaaaaaccaaaaagtgtctatcgcccgaatggaagttgttcgagagaagtcaaaagaagctgagggcgaagacgaacagcataagccctatccagacaaggcaaaagtcaaaatcgtttttgattcgattggtaaggagctgccgtattatattaaccacaaaacggttattatgaaaatccaaagaaatggcgaggcggtgaatttctgcaagattgggatcaatgaattgaaatatcttgttcttctgtgtcttcagggaaaatcggttgatgctatagcaaaaatcaacggctatatcgatcgtattaaaaaacgtcttgaaaacccaaagacaagaatcgacattgacagtgaaagagataacgaatttataaagggtcttcctgaatttgtaaaaatacaaagtggaagaacacctgatgaagaaagagaaaaaaagagtcggatcgactatataagaaaaaaatgggagaaaaagaaagaggaatcgcctaagacggaactacaccgcaaaggacgggatatacttcgatatataaattggcattgcgaaccaccactgggatcggaggaatataaccatcttttatcgctcctagtaaataaggacttagatggttttgaaaatgagctaaaggaattgaaacgaacagagcaaatttcaaagaagttattagaaatgctgaaaggtttttccaatcttaacgaacttcatttaaaagtgtgcagtattgttccggtggagttggtgtatctggagaagaatgatccggaaaaactggcagaatatatcggccttatcccccagaagacagacaaagaacctccatcctacgaagaaaaggttaagaattttatcaaacagccaatgatttataaaggatttttgagagatacttttttcagttccgggaaaacatttgcaaagctggttgaagaaacctttttgcataaatatccgcattccgatgttccgctgggaagggactattatcatgtaactactctggaccgattccataaagataattccatcctttatgagacgcttgcattagacagattgtgtgtggtgatggcgagaaagtttcatgaaacattgaatcaggagcttgctaaggaatcaaaacaaattgtctgggaaaacaatacaatcattctggaactaccaaggtcaaagacaagctcgtcagatacattccagatatgttttgatattaagcattatatgaaattgtatgtgatggatgatgtggagtttctgggcgggctgatgaggcattttttccaaaaagaaaaaacaattgagtactataatctctatagcttcggaatcaataaatacaccgaaatgcaaagaaatgggattgaagcgattctaagacttgaggaaaaaataattcgtgaaaaaaacatcaaacggaaacctgacaaaaactatatcagtttttatgaaatcatggaggcaagcaactatcctgacaatgataaaaagacattgaacaaggttcgcagggctctgttgcattatcacttgaaatttgagccggctgattataataagtttgtcgcgattatgaaaagagaaggtttggaaagaaaaaagaaaacaggaaaagcaaccagaagatgggtcaaaaaatga

SEQ ID NO: 81 - DNA, 2373 bp, Unknown
  Description of Unknown: Cas13 coding sequence
atggaaaatattaaattagaaaaacagaaagcagctttttactttaatcaagcagagcttaatttaaaagccatagaaggtaatatttttgataaaggaagacgaaagactctgtttgataatccaaagatactttccaaagtagaaaatttcattttcaatttcaaagatgtaacaaaaaatgcaaaaggtgaaatagactgcctgctgtcgaaactaatggaacttagaaacttctactcgcattatgtacacaaacccgatgtaaaagaattaagcaagggcgaaaaacctctactggaaaggtattatcaaatcgcgattgaagcaaccggaagcgaaaatgttaaacttgagataatagaaaatgacaagtggctcacagatgctggtgttctgcttttcttatgtatgtttttgaaaaaatctcaggcaaacaagcttataagcggtatcagcggttttaaaagaaacgatacctttggccagccgagaaggaatttatttaactacttcagtgtaagggaaaggtataaggttgttccggatatgcagaaacatttccttttgtttgttcttgtcaatcatctctctgagcaggatgattatattgaaaaggcgcaacagccatacaatataggcgagggtttatttttccatcgaattgcttctacatttcttaatgtcagcgggattttaagaaatatggaattctatacctaccagagcaaaaggctaaaagagcagcgaggagagctcaaacgtgaaaaggatatttttacttgggaagagcctttccagggaaatagttattttgaaataaatggtcataaaggggtaatcggtgaagatgaattgaaagaactttgttatgccttgctgtcatacaataaaagtaaatatgccgtggaacagattgaaaaatttttaaagggttttggtgaagtaaaaagtgaacaggagataagagatagtgatatattgaatgagagttattttccaactaattatttcgccgaatcgaacatcggtagtataaaggaaaaaatacttaatcgtttgggaaaaactgatgatagttataaaaagacgggaacaaagattaaaccatacgacatgatgaaagaggtaatggagtttataaataattctcttccggcagatgaaaaattgaaacgcaaagattacagaagatatctaaagatggttcgtatctgggacagtgagaaagataatataaagcgggagtttgaaagcaaagaatggtcaaaatatttttcatctaatttctggatggcaaaaaatcttgaaagggtctatgggttggcaagagagaaaaacgccgaattattcaataagctaaaagcggttgtggagaaaatggacgagcgggaatttgagaagtatcggcagataaatagcgcagaggatttggcaagtttacgccggcttgcgaacgattatggtgtgaagtgggaagaaaaagactggcaggagtattctgggcagataaaaaaacaaatttctgacaggcagaaactgacaataatgaaacaaaggattacggctgaactaaagaaaaagcacggcatagaaaatctcaatcttagaataaccatcgacagcaataaaagcagaaaggcagttttgaacagaatcgcagttccaagaggttttgtgaaagagcatattttaggatggcaggggtctgagaaggtatcgaaaaagacaagagaagcaaagtgcaaaattctgctctcgaaagaatatgaagaattatcaaagcaatttttccaaaccagaaattacgacaagatgacgcaggtaaacagcctttacgaaaaaaataaactcatagcatttatggccgtttatcttatggggcaattgaacatccggtttgataagcccacgagacttaatgaacttgaaaaagcagaggtggatttcaagatatctgataaggtgacggccaaaatcccgttttcacagtatccttcgcttgtgtacgcgatgtccagcaaatatgctgatagtgtaggtagttataaatttgagaatgatgaaaaaaacaagccgtttttaggcaagatcgatataatagaaaaacaacgaatggagtttataaaagaagtccttggttttgaagagtatctttttgaaaagaagataatagataaaagcaaatttgccgacacagcgactcatataagctttagggaaatatgtgacgaactgattcaaaaaggctgggacgaaaacaaactaaccaaccttaaagatgcaaggaatgcggccctgcatggtgaaataccggcggagacctcttttcgtgaagcaaaaccgttgataaatggattgaaaaaatgataa

SEQ ID NO: 82 - DNA, 2403 bp, Unknown
  Description of Unknown: Cas13 coding sequence
atgatcaatattgaattaaaaaaagaggaagcggcattttacttcaatcaggctaacctcaacatttcagggcttgatgaagttattgaaaagcagttaccacatataggcagcaaaaaggaaaacgctaaaaaagcgattgataagattttcgataacatcacggttttgaagaaagtggaaaattttgtcttttacttcaaagatgtagctaaaaatgaaagagttgaactcgatgctttattgctgaaattaatagatttgcgaaatttttattcacattatgttcataatgataatgtcaagattttaagtgatggcgaagaaaccttactggaaaaatattatcaaattgcgatagaagcaaccggaagtaaagatgttaaacttgaaataatagataacgaaaaaaagcttacagacgccggaatattgtttttgctgtgtatgttcctaaaaaagtcacaggcaaataagcttataagctctatcagcggttttaaaagaaacgataaagagtggcaaccaagaaggaatcttttcacttattatagtttaagggagggatataaggttgtacctgatatggagaaacattttcttttattcacactcgtaaatcatctatcgacacaggatgaaaatattgaaaatactcaaccttctgatgatatcggcagaggcttattcttccatcgaatagcttcaacttttttgaatataagcggaatattcaataatatggaattctatccttatcaaagcaacagactcaaagagcgaaggggagatattgcccctgataaggatagttttgcatggattgaaccttttcagggaaacagttattttaaaataaatggatataaaggagtaatcggagaaaatgaattaaaggaattgtgctttgctgtacttttacataacaaaagtaaatatgccgtagaacaaatagagaaattccttaaatgttttaaagaagtacaaagcaagcaggagataatagagtgtgatatattggatgaatgttattttccagccaattacctgaaccaaccggaaacaaaaagtctaaaggaaaagttactaagcagaataaccgggaaaattaattattcatttgatactgctgaaaaagcttttgataagatgaaagatgttatggagtttataaatggatgccttccgtcagatgagaaattgaagcgaaaagattacagtcgttatctcaagatggttcgtttctggggcggggaaaaggataatataaaaagggagtttgagggtaaaaaatggacgcgattttttccaagtgaactttggcataaaagaactcttgaagatgtgtataagtttgcactaaagaagaacaagaaacgactcgaagaattgaaagttaaaatagaaggccttaacgaagatgaccttttaaaataccaaaaggttaacaacataaaaaatttggagaatttaaggcttcttgcgcatgatcttgatttgagttggcgggaaaaagactggggggaatattcgggacagataaaaaaacaaatttctgataatcagaagcttacaataatgaaacagagggtcatcgccgaattaaagaaaaagcacggcattgaaaatattaatcttagaataagtttagatagcaataaaagcatacaggcagttttaaataggatagcaattcccaaaggttttataaaaaggcatgttttgcacttgcaggaaaatgaaaagacctcaagaaaaattagagaggcaaagtgcaaaattctgctttccaaaaaatatgaatatttatcgagaaaattcttagatgaaaagaacttcgacaagttgacacagataaacggtctttatgaaaagaacaggcttatagctttcatggtcatttatcttttgaagcagcttggccttgaattgaaaaatgaaacgaaattaattgaactcaaaaaaaccagggtaaaatataaaatatcggataaggtggcagaggatatcccgctttcacattatccatcgcttgtttatgcaatgagtcgtaaatatgttgacaatattgataattacgaatttccagatgaatatgccaaaaaagctatcttggataaagttgatataatagaaaatcagcgtatggaatttataaaacaggttctcggttttgaaaagtatctttttgataataatataatagacaaaagcaaatttactgatgtggagactcatataagttttgtaaaaatacatgatgagcttattgaaaaaggatgggatacagaaaaactgtccaaactcaaacatgcgaggaataaagctctgcacggcgagataccgggtgggacttcttttgaaaaagcaaagctcttgataaatgaactcaaaaaatgatag

SEQ ID NO: 83 - DNA, 2634 bp, Unknown
  Description of Unknown: Cas13 coding sequence
atgaaaaagcaaaaaagcaaaaaaacagtttcaaagacttcgggattaaaagaagcgctttctgtacagggcactgtgattatgacctctttcgggaaaggaaacatggctaatctgtcttataaaattccttcttctcagaaaccacagaatttgaattcttcagcagggctgaaaaatgtggaggtatccgggaaaaaaatcaaattccaggggcggcatcctaaaattgcgactacggataatcctcttttcaaacctcagccaggcatggatctgctttgtttgaaggacaagctggaaatgcattatttcggcaaaacatttgatgataatattcatattcagttgatttatcagattctggatattgaaaagattctcgctgtacatgtcaataatattgtatttacgctagataatgtgctacatccccaaaaggaagaattgacagaggatttcattggcgcaggcggctggcggatcaatctggattatcaaacactgcgagggcagacgaataaatatgatcgtttcaaaaattatatcaaaagaaaagaattgctgtattttggggaagcattttatcatgaaaacgaacgtcgttatgaagaagatatttttgcaattctgacattgctttctgctctccggcaattctgttttcacagtgatctcagcagtgatgaatcagatcatgtaaatagcttttggctttatcagctagaggatcagctttctgatgagttcaaggaaacgctgagcatcctctgggaagaagtcacagagcgcattgatagtgaatttctcaagaccaatactgttaatttacatattctgtgtcatgttttcccaaaagaaagcaaagaaaccattgtccgggcttactatgaatttctgatcaagaaaagtttcaagaacatgggcttttcgatcaagaaattaagagaaatcatgctggaacaatctgatttaaaatcattcaaagaagataaatataattccgtccgtgcaaaattatataaattatttgatttcatcattacttattattatgatcatcatgcattcgagaaagaagccctagtttcttccctccgcagttccctgacagaagaaaacaaagaagaaatctatatcaaaacagccagaacattggcatctgctctcggtgcggatttcaaaaaggccgctgcggatgtcaatgccaaaaatatcagagattatcagaaaaaagcaaatgattaccgcatctcttttgaagatattaaaattggcaataccggaattggctatttttcagaattgatttatatgctgacattattactggatggcaaagaaatcaatgatctgctcacaactctgattaataaatttgataatattatttcttttattgatattctcaaaaaattaaatcttgaattcaaattcaaacccgaatatgcggatttcttcaacatgacaaattgtagatatacccttgaagaactccgtgtgatcaatagcattgccagaatgcagaaaccctctgcggatgctagaaaaataatgtatcgggatgccctccgtattctgggcatggataaccggccagatgaagaaatcgacagagaactagaaagaactatgcccgttggtgcagatggtaaattcattaaaggaaaacaaggtttccgtaattttatcgccagcaatgtgattgaaagctctcgctttcattatctggtcagatataataacccccataaaacaagaacccttgtgaaaaatcctaatgttgtcaagttcgtcctcgaaggtatcccagaaacacagataaaacgctatttcgatgtctgcaaaggacaggaaattccccctacttcagataaatccgctcaaattgatgtactcgccagaattatctcctctgttgattataaaatctttgaggatgttccccaaagtgcaaaaatcaataaagatgacccttcccggaatttttccgatgctctcaaaaaacaacgctatcaggcaatcgtgagcctgtatctaactgtcatgtatctgattactaaaaatctggtctatgtcaattcccgttatgtgatcgccttccattgtctggaacgtgatgccttcctgcatggtgtcacactccccaaaatgaataaaaaaatcgtttacagccaattgaccacgcatctcctaactgataaaaattatactacttatggccatctgaaaaatcagaaaggtcatagaaaatggtatgtgcttgtgaaaaataatctccagaattcagatattaccgctgtctcctcattcaggaatatcgttgcccatatcagcgttgtacgcaatagcaatgaatatatcagcggaatcggtgaattgcattcctattttgaactttatcattatttagtacaaagcatgattgctaaaaataactggtatgataccagtcatcaacctaaaacagccgaatatctcaataacctgaaaaaacatcatacttattgtaaagattttgtcaaggcttattgcattcctttcgggtatgtcgtgcccagatataaaaatctcacaatcaatgaactgtttgacagaaataatcctaatccagaaccaaaagaagaggtctaa

SEQ ID NO: 84 - DNA, 2595 bp, Unknown
  Description of Unknown: Cas13 coding sequence
atgaaaaaatcaaaggtcaaacttaacggtgtgaaagcggtatatcatatcagtccggatgtaagagttatagctgctttcggcagaggaaacaattccgtacttgacaaacacatagaaaacggctccgttgaagaattacaaaatcattcggatatagaggtcaatatcagccgcaaaacatactctttccgcaagaaaagcctgaaaaaagctgccggacagttcagtgttccggataatacaaatgatcagctgggaataagagaagaacttgaaaaagaaatattcggcaggaaatttgatgataatatacatatacaggcggcttatgcggttaatgatatcataaaaatgctggcggttgccgcaaatcttgccgaaacagccataaacggacttgacagagaaaatactgaaaatgatatgattggtttttgtataatccctcacatcacatatcaaacatacgcagacgataaacgaagcaaattcgaaggcttcatagagaaagtaaaaacacagggaacattttcttattttccggatatattgccgaaatttaaaaaagaagaatctgaagaagagagtgacaatgaaaaactgtattatatcatgtgcattatcagcctgatacgcaattcagccacccattcaaaaagcagtaattccgatactacagactatattttcggagaattcaattccgtcaataaggaggccttgacaacaacagccgataatctgataaaaagcaagatagatttcatcaacaaagaaggattcagcaaaaatcagaaaaacaatatttacagactcctgaaagccaaagcagatacaccggaaaatactgccagacttatccgcaggctttatgccttcaccatacggaagcaggataaaaacctcggtttcagtctgaaaaagctgagagaatgtgccatccgttccatagaccgttccatagagtatatgaagtatttaccaagtaagaaatatgatactgtcaggagcaagctttatacactgatggattttgttgtttactcgtatctgaaataccataaggatggaaaaaagttctccaaagaaatggtagaacagctcagagccgccgaaagtgacaaggtcaaagatgacatctatcgtgacgaggctgaaaagctgtataacatcgaaatcatcagccggacaataaatgcactgataagtgatattaaatcagactttgatcagcctaagcatggcaatgaatgctatcagcctatcaatgacggtatgaaagaggcggaaaaagattttattacaaccgatcaattaagcctttttacaaaattcatttatgtactgtgtcagtttcttgacggcaaaaacatcaatatccttttgtcctcacttatcagcaaattccagcagatagaagctttcaacggtgatatcagaaagctgaatctgaatatcagggatgacggaaaaatcggctatgacagcaaaaaatattccatttttgaaaaaagcggacagatagctgatgatcttgataagctccggggtgtcatcaaaatggacatcaacgacctgaacgcatacgaaaccatgataaaagatgcccttcgtgttatcggtgtcgatgaaagtgacatcgaaagtatttatcagacacatttcaaaacacaagacaaaaaagacagcgtatcgggctttttcaggaataatatcatcaattccagacgtttccgctatatcatcaaatatatcaatccttcggacgcataccgtatcattcagaatgaaaatgtcagaaactatgttttaggcagaatgaatgacgcaattatagaccgctatgcccattctgtcggcatcgaagacaaagtacacgacaaaagaaaagtactttcagatatcttgtcaaaggtaaaattcgataatttcacaaaactgacatatatcaatccaaaggataaaaataagggcgaaaaggcaaaggaaagagaaaagcccaaagccattctcggattgtatctgacaatcgtttatcttgttgtcaaaagccttgtgcgtatcaattcacagtatgttatggcggtatatcatcttgaaagagacagccggctgtgtcccggtgtcagttcaaataatcttttatccatgacgaaccattactgtgaccgtgataaccatctgctgaaagaaaagcatattgtaaagctggaaagatacaaaaaaacccaaaaaacccaaaaaacaatttgtacagcctaccgcaacgcaatagcacacctttccgcagtaagaaagggcgtaaaatatatcggagatatacaaaaagcagactcttattttggaatttatcactactgtatgcaaaaactcatttattcagctaacggacagccgtttgcagaatttgtgcgttccatattcggcgatgaaaaggagcttgataagctcagaaacggcagttattcacaggcaatactccgtgcattgaattatcctttcggatataatccggcaaggtataagaatctcagctatgagaaaatattcataagggcatggcaggacgaggacaccaataaaaaaacgtga

SEQ ID NO: 85 - DNA, 2643 bp, Unknown
  Description of Unknown: Cas13 coding sequence
atgaataaaatccacaagaagcagggtaaaaccaccgcaaagtctttaggtttaaaaagtgttttaaaaattgaaaatgatttagttgtcacgacatttggtaagaaagacaatccaatggtagtggaacaatctatcaataaagcaagcggcgagaaagaattatacgttgacgaggatcaagttaagtttgattcttctttaattaaagagaaaaacatcctatcgcttgattccattcagcatagtaaccatcaaataatagttaatattgatcaaaaagatgcttctgaaattgggatggattatcttcgtttaaaaccagaattagaaaaagagttttttggtaagaccttttatgataacgtgcacattcaaatcgcatacaatttgctcgatctcaaaaaaataattggcctccatatcggtaacgcaattcaagctttagagaatttaggaagagatggcagcgacttggttggcatttgtgacgcgaccaaaccattaaattacctcgatgatgtcaaacaaaaagcggatataggttttatgaatagattaaagccatattttatgtactttgatggcgtcttgaaacttgataatagtaaaaataaaaacggcgagctaaaccagttggacatcgaaaactgggatgtgataagaatattatcattaataagacaaggttgtgctcacgcaggtgcatattcttcactattatatacggctcaaaataataaggtttatgctgatttgatcaataaagcgttatcgatatttagtgatgatttggacaaattcaataaatcatttctaaaacaaagtaaaatgaatttgttcattctttttgatttatataattgtcgttttgataggagccttcaagaaaagattattaaggagtattatcgctatgtattgtataaagataataagaatcttggatttagcttaaaaaatgtcagaaaccttatcatcgaaggaaaatatgatgaacaagagagaagtgggaagctgcagacgattagatctaaactcaacacattgttagatttctacctatatgggtattatcaaaagaacccaacctttgttgaaaacatagttgcaaaactacgcgagtctaaaaacgacgaggataaagaaaaagtctatgaagaggaataccatagattattatccgaaaacaattatctagtagataagaagtgttccgatattgtttatagaatcaatgaagcagtaaaaaataggaaaatatttgttaacgcgaacatcaatgccgttgttgaaaaggtttcttgttcttgcttcccatcattgatatatgttttatgtaagtttttagatggcaaagaagtaaatgagttaacaaccgccatcataaacaaacttgaaaatatagcctctttaataaatgcattagtaactttgaaatcttatggtggcttcagtgaacaatataagatttttgactacccaaacatcaatggattaattgatgattttagaatggtaaaaaatctcacttctacgaaaagaaaattaaaaaaagcaagcggtggagaagataggattggcagacaattatatgctgatgcaattaatatatttaaagaagacagctttgtttctgctaacgatgagaaagggaccggtttagatcaatatgtcaataagttttttagcaaagatgatctaggagcaagaaaagtaagaaaccttttgttaaacaacattattaaaaatcgtcgtttcgtatatttaataaaatacattgaccctaaagattgttataaattagttcacaatgaaaagattgtgaggtttgcattaggccaatatgatgaaagtcaaatgccactcaatcaacttcaaaagtattatgatgctgtcattgagaacagagaaggatttagaaaatgtaatgatcgaaagaagattatcgatacattggtttctgaaatcaacagggtttcaattgatggaattcttgatatcgggaacaggctagtaaatagaggaaataatgactatataaatcaccagaaacaaatcataagtttatatttaacaattgcttacttaatcgttaaaggtgttgttcatacaaactcgttgtatttcatcgcatggcatgcttatgaaagagataataatttcaaatttggtaatgatggtaaggactatctcgcattaaccaaagagtatctaacaaataaaaagaagagagttaaacaattgctagatcataatatagaagaagcgaataactctcttgattcaaaatacttttcggcataccgaaacaaagtggttcaccttaatttctgcaacatatttgtcaattatttagatggtataggggatattcattcttattatgatatataccaatatgttattcaaaagtggtcaatagcggaacgttcaaaagacttcattgatccgcaatacttaacgaaattatctaatgatttaaaacaatacagaacttatcaacgcaacttcttgaaaatcatcaatcttccgtttgcttataacttagcaagatataagaatcttaccattggtgacctcttcaacgacaaatacccactcccgaaagaaactgttaaagagttttacaacgaggaataa

SEQ ID NO: 86 - DNA, 2643 bp, Unknown
  Description of Unknown: Cas13 coding sequence
atggcaaaaaagaaaagaatgtctgcaaaagaaagaaagcaacaacagataaatcttcgcataaaaaaggctactgaagattcaactaaaaaagtcaatacaacagtagcagtgaataataagcctatcagtaaagaaataaaaaaatcaaaagcaaaacttgccggtgttaagtgggttattaaggctaatgatgatgttgcttatatttcttctttcggtaaaggtaataattctgtactggaaaaaagaattataggggatgtatccagtgatgtaaacaaagacagccatatgtatgttaaccctaaatacaccaagaaaaattatgaaataaaaaatggcttttcctcaggcagtagccttaccacccatcctaataaacctgacaaaaattccggtatggatgcattgtgcctaaagacatattttgaaaaagaaattttcaaagacaagtttaatgataatatgcacattcaagctatctataatatttttgatattgagaaaacacttgcaaagcatattacaaatattatttatgcagtcaattcccttgaccgtagttatattcagtcaggtaacgatacaataggatttggacttaacttcaatattccttatgcagaatacggtgggggaaaagactctaacggtaagccggagaataaatctgcttgggaaaaaagagagagctttattaagttttataataatgctaaggaccgttttggttattttgaaagtgtattttatcaaaatggtaagcaaatttctgaagaaaaattctatatttaccttaatattctaaactttgttcgaaacagtactttccattacaataatacaagctcccatctttataaggaaaggtattgtaaaattaatcctaagaataatttgaaaaccgattttgaatttgttagttatttaaatgagttcgttaagaataagtttaagaacgtaaataagaatttcatttctaatgaaaaaaataatctttacattatcctaaatgcttatggtgaagatattgaagatgtagaagttgttaaaaagtattctaaggaactatacaaactttctgtactaaaaactaataagaacctaggtgtcaatgttaagaaattaagggaaagtgctattgaatacggttactgtcctttgccttatgataaagaaaaagaggttgcaaagttaagctcgattaagcataaactatacaagacttatgactttgttattactcattatctaaatagcaatgataagctattgttggaaattgttgaggctttgcgtttatctaaaaatgatgataaaaaagaaaatgtatataaaatatatgcagaaaaaatatttaaagcagaatatgttattaatcctataaagactatcagtaacttgtttgctgaaaaaggcgataagttatttaatgaaaaggtaagcattagtgaagaatatgtagaagatattagaattgataagaatattcataacttcactaaggtaatattcttcttaacatgtttccttgacggtaaagagattaatgacctactgactaacattatcagtaagttacaggttattgaagaccacaacaatgtaataaaggctattgccaataataatgatgcagtttataaagattacagtgataagtatgctgtatttaaaaattccggtaaaattgcaaccgaattagaagcaataaagagtattgcaagaatggaaaacaagataaacaaggcattcaaggaacctttacttaaagatgctatgttggcacttggtgtcagtccaaatgatcttgatgaaaaatacgaaaaatattttaaaactgatgttgatgccgataaggaccatcaaaaggttagtacattcttaatgaataatgttattaacaacagtagatttaagtatgttgttaagtacataaaccctgctgatattaaccgtttagcaaagaataaacaccttgtaaagtttgttcttgatcagattccacataagcaaattgactcttattacaattcagtttctactgtagaagagcctagttacaaaggaaaaatacaattactgacaaagaaaattacaggacttaatttttattctttgtttgagaattgtaaaattcctaatgtagagaaagagaagaagaaggcagttatcaccctttatttcacaattatttatattcttgtgaagaatttagttaacattaacggtttatatactttggctttatattttgttgagagggatggatttttctacaaaaaaatttgtgaaaagaaagataaaaagaaaacaaataaagatgttgattatctgttgttaccggagattttctcaggcagtaaatatagagaagaaacaaaaaatctaaaactgcctaaagaaaaagatagagaaataatgaaaaagtatctacctaatgatgaagataggaaagaatacaataaatttttcaaacaatatcgtaacaacattgttcacctaaatataattgcaaatctttctaaattgacaagtactattgataaggagattaactcttattttgagattttccattattgtgcacagagagtaatgtttgattactgtaaaaataacaataaagttgtactgtaa

SEQ ID NO: 87 - DNA, 2688 bp, Unknown
  Description of Unknown: Cas13 coding sequence
atgaagaaaaattctaatgataaaactaatgctaagaggatggggattaaatcttttataaagaatggggacgaacgttttattactacttctattaagaatgaatttccggtagaacttaaattagatgtgattaaaaaaacgtgtgaacccgcgcatgagcccgtttcttttgattatgatcctaaaaaaatagattttgaaaagcccgtccttaaggaaaaattgacttcaggacagagtgggcagaaactaagcacccggttatttatacaaaaagaccgtgatatctgcggaatcaggcgaaaatatttagaaaagatatttaatagtaattttattgaagaaaaaaaagatagtaatttgcccatgcagatcgtcgctaaagtgcttagtactgaaaaagttttttccaatgctttgaataaaataatctctcaatttctttcgatgccaaggggaggcgttacagataatcacggtgagtatgaaataattggtaatattataaatcataaatctttgcaggaacttaataaagaaaagaagaccaagagaataaaaaaatacctgcagagtgtaattaaaaatcaatcatatctgtataataagcagtttcttttatctcttgatgagtctaaaggttcaagaaacgatatcgatgagaatgagctttatgattatataagattcctggcaattctgcggaatggtatagctcacgtcttttatgaaaaaaatgaaccggagaccgcaaaagaaagtttgttcagattagttgattttattaaaaatgataaaaaacttgaaggagcttttgcgaaaataaaaattcaagtgaatacattatataaatgtagaaaagaagaatatataaaaaaaagcggtaaaaattttgagataatacgaaagatttatcaaaatgataagccggatgagaaagtaaaagactggataaggtatgatttcgataaatcgtataaatatataggccttagcgtagccaagttaggcaattatacttcgtgggcaaaagatatagataacttaagagataaaagtaatcctgacagcggatacgcaggtattatgcataggctcaatgaattttcggtatatcttaaagttaaagctctatcaactgaggaaaaggataagtatttaaaaaacttaatttccaaagaaaactgtgaagaaaaggataagtattacaaaaatattgcccaatttttttgcagttctgacttaaagtttgctaatgttctacaaatggtcaaggaaataaaaaagaataaaggatgtacgtcagaagataaaaattgtaagttatgtgtggatgaaagaaaattcaatgacttatcggtaatagtgtatttcatttcttgctttttagacaataaagaccaaaatatatttttaagcgacttaattaacaggttcggagcattatccgatttgctcaggattcagaataaaattttaggtgcgggtaataagtataatgaaaattacagttttttaaaaaacgagcgatatgttacagaaataaaaatggaacttgaaactattttcgcccttgtaaaggtaagttataaaaaagaagataaggcttttaacagattgcttgaagacggtctggttatgtttggtttttctaaagatgaagctgggatgaaagtagcaggattaaaggaaataaaggaaaaaaaggaagggcattataagaataaatctagaagttttttgattaattctatagtaaattcgcgaaaatttgcatatctcgcaaagagcatagatccgcagaaagtccctgcaatcataaagaatgagcatattgtcagatatattttaggcaggatcaacaagactaatcctgggcagatcgggcgctattggcggtatataatgtcccagaatcatgccggaacggacaaggttgatgatcttacaaatgaaattataaaaattaatataaaaaatatattaaacgacgccgggggttggcagaaatctaaattaaacgataacaataataaaaagaaattgaagtaccaacagttgattggtctgtatttgactgtcgcttatatattcgtaaagaatatgcttgaatgtaatgcgcgctatttctctgctttcgctcagatagaaaaagattatctaatttatactaactcagatgaattttattacattgataaaaataaaaaaaatcttgtcacggagagatatttaaaattagtaaaagatattattgagaaaaataaaaatacagtaagaaaagataaaatattcaggaagaagagacaaagaaaacatctggctgatatatctaaaagtattatagaatttgaaaaattgccttgctgtatattcactttactgaggaatattacagagcatctgaatgtcgcttcaaatatagatattatagaaggctatgggaagagagctggaaaatatcataagaatgctcctgcttcttactttattttttatcattatattattcaaaagatcctagcggataaaatatgcaccagaaacctgttaaatataattaatacttacggagaaccttccatatcttttataaaaattatttatgttccgtttgcttataatcttcccaggtatttgaatcttacggatgctaggatattttgtaatatggacgataaataa

SEQ ID NO: 88 - DNA, 2574 bp, Unknown
  Description of Unknown: Cas13 coding sequence
atgaaaaaactcaaaaacccctcaaatagaaactctctaccaagtattataatctcaaaatttgactcatctaaaatatatgaaataaaagtaaaatatgaaaaactagctagacttgatagactagaaataggagatatgagtttagatgaaaacttaaatatcctttttaaaaaagtaaatttcaatggaattgacttagagattttaaaccctttgctcctagattttgactcttacactataagtgggaaacttcaaaaaaacagtacaaataaaactatattaactttaaaaaaagatggcaaaatcatcaaatacaatgtactagaaaaagataataaatattttaaaaatggtaaagagtttgtaatacctaaagatgtaaaagaagaagggaaaaggcttgtaaatgataaatttcttttaacaattgaagataagaaaagagaggaaaatagtttacctaaaaagagaaaaaaagagactcaaagggatattttaaaagatgaaactatagaaatatacaaaagaatctcttcaaatagtaatataaaaagtgaagatatatatagaataaaaagatatatgctttttaggtctgatatgatgtttttttacacttttatagataactttttctattgcctttataaaaataaaaatgaacagttatggaatacaaacttcaaagagaaagaaaatttaggtaaatttattgaattcaccctaaatgatacccttaaaaaccctagaaatggtattttaaaaagttatagtaaagatttaaaagtagtacaagaagactttgtaaagataaaagatatatttgaaaaaattagacacgctctagcccattttgattttacttttatagataatcttctctcaaacaatattgaatttgattttaatataaaacttttaaatatagttattgaagatagccaagatttatattatgaggcaaaaaaagagtttatagaagatgaaaagatggatattttagatgaaaaagatatttcaataaaaaaactctataccttctattcaaaaattgatataaaaaaaccagcttttaacaaactcatcaactcttttttaatcaaagatggagtagaaaatagcaagttaaaagagtatataaaagagaaatacaactgccactattttatagatattcacgacaacaaagagtataaaaaaatctataatgaacataaaaagctaatatcagagaatcaaaacttacaattaaactcaaaagagaatggtcaaaaaataaaaataaataatgatagattagaagaattaaaagggaaaatgaatgaactaacaaaagccaattctctaaaaaggcttgagtttaaattgcgtcttgcttttggatttataaaagttgagtataacatatttaaagatttcaaaaacaattttagtgaagatattaaaaaagatatgaatatagatttagaaaagataaaaagctatcttgatacaagctactctaataatcaattttttaattataaggtttataataagaaaactaaacaaaaagatatagataaagatatatttgatgatattgaaaaagagactctaaaagagcttgtagagaatgactccctactaaagataatactacttttttatatctttactccaaaagagttaaaaggggagtttttaggctttattaaaaaattctatcacgatacaaaaaatattgacaaagatacaaaagacaaagaggagccactagagcagattaaacaagaagttcctctaaaattaaaaatattagaaaaaaatctaactattttgacaatatttaactactctatctctttaaatatagagtatgacaaaaataataacagcttttatgaaaggggaaataaatttaaaaaaatatataaagatttaaaaatatcccacaatcaagaggagtttgataaatcccttttagctccacttttaaaatactatatgaatctatataaacttttaaatgattttgaaatatacctacttctaaaatataaaaataaagataatttaaataaagagagtttaaataaattgataaatgatgaacaattaaaacacaatgaccactacaactttacaactcttttatcagaatattttaattttgaccctaaaaagaataaaaaatatgaaactctaacaatacttagaaatagtatttcccatcaaaaaatagataacttaatatataacctagataaaaataaaatcttagaacaaagggtaaaaatagttgaactaattaaagaacaaagggatataaaagagactctaaaatttgacccaataaatgactttaccatgaaaacagtacaacttctaaaatctttagaaaatcaaagtgaaaaaagagataaaatagaagagatactaaaacagcaagatttatctgcaaatgatttttataatatttataaactaaaaggcgtagaatcaataaaaaaagagctttttataaggcttggaaagacaaaaattgaggaaaaaattcaagaggatatagctaaaggctctatttaa

SEQ ID NO: 89 - DNA, 2527 bp, Unknown
  Description of Unknown: Cas13 coding sequence
atgttaattgaatagtatagaaaaaatcaaaaaaccctcaaatagaaactcaattcctagtatcatcataagtgattatgatgaaaacaaaatcaaagagataaaagtaaaatacctcaaattagcacgacttgataagataactattcaagatatggagataagggataatattgtagagtttaaaaaaatacttttaaatggtatagaacatactatcaaagataatcaaaaaatagagtttgacaattatgagataactgcttatgtaagagcctcaaaacaaaggcgagatggaaaaattacacaagcaaaatatgtggtaactattacggataaatatctaagagataatgagaaagaaaaaagatttaaaagtacagagagagaacttccaaatgatacacttttaatgagatataaacaaatatcgggatttgatactcttacctcaaaagatatatataaaataaaacgctacatcgatttcaaaaatgagatgttgttttattttcagtttatagaagaattttttagccctcttttaccaaaaggaacaaacttttatagtttaaatatcgaacaaaataaagataaagtagttaaatatattgtttatcgcctcaatgatgattttaaaaatcaaagtttaaatcagtttattaaaaaaactgatacgataaagtatgattttttaaagattcaaaaaatattaagtgattttcgccatgctttggcacattttgattttgattttatccaaaagttttttgatgatgaacttgataaaaatagatttgatataagtactatttctcttataaaaactatgcttcaagaaaaagaggagaaatactatcaagaaaaaaacaactatatagaagatagtgacactcttactctttttgatgaaaaagagagtaatttttctaagattcataatttttatataaaaatatctcaaaagaaaccagcttttaataaacttataaatagttttttatcaaaagatggagtgccaaatgaagagttaaaaagctatttagctactaaaaaaattgatttttttgaagatatacactcaaacaaagagtataagaaaatctatatcaaacataaaaatctagtggtagaaaaacaaaaagaggaatcgcaagagaaaccaaatggacaaaaacttaaaaactataatgatgagctccaaaaactaaaagatgagatgaataagattaccaaacaaaactctcttaatagacttgaagtaaaattgcgacttgcttttggatttatagcaaatgagtataactataattttaaaaactttaatgataaatttacacttgatgtaaaaaaagaacagaagataaaagtatttaaaaatagttcgaatgagaagttaaaagagtattttgaatctacttttattgaaaaaagatttttccacttttgtgtaaagtttttcaataaaaaaactaaaaaagaggaaacaaaacaaaaaaatattttcaatttaatagaaaatgaaactttagaagaacttgtaaaagagagtcctcttttacaaattatcactctcttatatctttttattccaaaagagctccaaggtgagtttgttgggtttattttaaaaatatatcatcatactaaaaatatcacaaacgatacaaaagaggatgagaaaagcatagaagatacacaaaactctttttctttgaagctaaaaatattagccaaaaacttaagaggtttacaactttttaactacagcttaagtcataatacactttacaatactaaagagcacttcttttatgagaaaggaaatcgttggcaaagtgtttataaaagtcttgaaatctcccacaaccaagatgagtttgatatacatttggtaatacctgttattaagtattatataaatttaaataaacttataggggattttgaaatatatgctctcttaacatatgcagataaaaatagtatcactgagaagttatcagatataacaaaaagagatgatttgaaatttagagggtattataatttttcaacacttttatttaaaacttttatgattaatacaaactacgaacaaaatcaaaaatctacacaatacatcaaacaaactagaaatgatattgcccatcaaaatattgaaaatatgttaaaagcttttgaaaataatgagatatttgcacaaagagaagagatagtaaactatcttcaaaaagagcataaaatgcaagagattcttcactacaatcctatcaatgattttactatgaaaacagtacaatatctaaagtctttaaatatacatagtcaaaaagaaagtaaaatagctgacatacacaaaaaagagagcttagttccaaatgattattatcttatttataaactcaaagtgatagagcttttaaaacaaaaagttatcgaagctataggggaaacaaaagatgaggaaaaaataaaaaatgcaatagcaaaagaagaacaaataaaaaagggttataataagtaa

SEQ ID NO: 90 - DNA, 2601 bp, Artificial Sequence
  Description of Artificial Sequence: Synthetic polynucleotide
atggccgtggactacagcctgaagaacgaatggtaccgggaaatcaacaaatcatgcttcaccgtggccctcaatgtggcctacgacaactgcaaggccaagggccacgagaacctgctgagagaggcacaacgtagcaagggaggcatcaccaacgagcagatcaagaacgtgcagaccgaaattaagacccggctcgaggacataagaagccatttcagccatttctatcacgatgagaagagcctgatcttcgagaaggacaacatcgtgaaggacttcctggaatccgcctacgagaaagctcagagcagcgtgatcggatctaccagacagagcgactacaaaggcgtggtgcctcctctgttcgaaccccacgacggcatgatcaccgccgctggcgttgtgttcctggccagcttcttctgccacagaagcaacgtgtacagaatgctgggcgccgtcaagggctttaagcacaccggcaaagaagaactgagcgacggagccaagcgggattacggctttaccagacggctgatggcccactacagcctgagagactcttatgtgatcaaggccgaggagaccaagtcctttcgggacctgctgggctacctgagtagagtgccccagcaggctgtggactggctgaacgagcacaaccagctgagcgaggacgagaagaaagaatttctgaatcagaaaccaagcgatgaagagagccaggagcagtctaagaccgagaacaccgacaggcaggccgacagaatgcctcggcggtctctgagaaagaccgacaagttcatcctgttcgccgccaagtttatcgaggactgggcccagaaggagaaaatggacgtgacctttgctagataccagaaaaccgtgacagaggacgagaacaagaaccaggatggcaagcaggtgagagacgtgcagctgaagtacgagaaggatacaaaaaaactgaaccctgatttcgactacaagtggacatactacatccgcaataaccacgccatcattcagatcaagcccgacgaatacaaacaagccgtttctgccagaatcagcgagaacgagctgaagtacctggtgctgctgatcttccagggcaagggctgggaggccatcaaaaagatcggagattacatcttccacatcggcaacaagattaagattggcagattcgaccacaacgaggaacggcggatgcctagctttttaaagaatcctccagccgacatcatcggcgagatggtggaaaataggctcaagtacatccgggacgagctgaataaggtgatcgaaaccatcaaaaaggaggaacctcagaataataagtggctgctgtacaagggaaaaaaaatctccatcatcctgaagttcatctctgattccatcagcgacattaagaagagacctgacgtgaacgaatacaacaccctccgggatatgctgcagaaactggactttgacaatttctacgaaagactgaagagctacgtgagcgaaggcagaatcgagcaaacactgtatgacgagatcaagggcatcaaagatatctccaccctgtgcatcaagatttgtgaactgcggctggccgctctggaagagctggaaaaagagggcggagatgacctgaacaaatacatcggcctggccgtgcaggagaagcacaagaactacgatgacagcaacacccctcagaagaaggccgagagattcctggaaagtcagttctccgttggcaaaaacttcctgagagagacattctacgacgagtacatcaagaatcggaaatctctgtacgagatcatcaaggaaaagatcacaggcatcacacctctgaacgaaaatagatggtacctgatggataagaaccccaaagagttcgaaagcaaggatagcaagattatcagaggcctgtgtaacatctacatccaagacatcctgtgtatgaagatcgccctgtggtattacgagaacctgagcccttcctacaagaacaagctgaaatgggacttcatcggccagggattcggctacgaccggtacaagctgtcttacaagacagactgcggcatcaccatcgagttcaaactggctgatctgaatagactggacatcatcgagaagcctaagatgatcgaaaacatctgccacagcttcatcctggagaaggacgtgaagaagcagacaatcagctggcacgaattcagacaggacggcatcgccaaatatcggaaactgcagaaggaagtggtggaagccgtgttcgaattcgagaacagcctgaagatccctgataagaactggcttacacaaggctacgtgccattcaacaagaacaagcgcttcgaggataagggtttcagcaccttcatcctggaggaggctgtccgcaagggaaagatcaagtctgatgacaaggagccccttcggaaggtgagaaccgattttttccacgagcagttcgacagcaccgatgccgagagaagaatctttgataagtacatgcccgccaagcacgacgggaagaacaagggcggcaagatgcaggaaaagcaggagaagagctacaccagaagaatctga

SEQ ID NO: 91 - DNA, 2397 bp, Artificial Sequence
  Description of Artificial Sequence: Synthetic polynucleotide
atggaaaaggaacagggcctctacagcatcgaccgctaccagggcgccggcaaatggtgcttcgccattggcgccaacagagcctgggacaattacaatgagcgccccaagctgttctcagaatccctgctgagatacgagaaagccacaaggagagactggttcgacgaggaaacccggggcctgattaagaagtctgatgtgcggcagagactgagaaagatccgttgctacttcagccactactgccacgacaacacctgtctgggctttgaccccgacgatgatctgagaaaaatcatggaaaaggcttatgaaagagccatcttcgagcaaagaaagcacctgtctacagagacagacatcgagacccctgccctgttcgagccccacggcagaatcaccgccgctggagtggtgtttttttgcagcttcttcgtcgaacggagaatcctgaatagactgatgggccgcatccccggcttcaaaaagaccgagggagaatacggcgctaccaggcagatgttcagcaagtattgtctgagagatagctacagcatccgggcctccgacagcaacgccgtgctttttagagatatcctgggctacctgtctagagctcccagccagtactacagacacaacaaggaccagtgcgacaaggacggccaccctgagcggaagaaagataagttcatcaacctggctctgagatacctggagtctttcgtgccagctagactgaggaaccacactctgagcgtcggcagaaaggaagtggtccggatggaaaccaatgccgtggccgagggcgagggcgagtacagaccctacccacctaaggccaaggtgaaggtggtgttcaccgaagatgatccagagagaccttactacatcactcacaacacagtgatcctgcaaacagccaagaaggaagaggacatccaccactgtaaagtgggagttaacgagctgaagtacctggtgctgctgtgcctgcagggaaaggccgagaaggcagtggctggcatcgagggctatgtgcggagaatccaaggcagattcgccgaccacacaaacaaggtggccagagatgacgacgagaggctcgtgcggggcctgcctgagttcgtgcgggtggccagcggaatcgagacacctgatgaggtgcgcgagctgaagagcagactggaccacatcagaaagaagtggcagaccaagaaggccgagtccgccgaggcccagctgcacagaaaggctagagatgtgctgagacacatcaactgggagtcccagcggcctctgggcatcgagcagtacaacagactgctggagctgctggtcaacagagacctggaaagcttcgccgctgaaatgaaagagctgaagagacggggactgatcagcgaagagctgctgaagagcgtggaaggaataagaaacctgaacaccctccatgtgaaagtgtgcaacctggtgctgaccagactggagcacctggtcgagaacgaccctgaagaactgaagagacacatcggcatcgtgcctagagaggaaaaggagggccctagctacgaagagaaggtgcgagccttcgtgcagcagcctatgatgtaccggggcttcctgagaaacagcttctttaagggcagcggcaagagctttgccaagctggtcgaggaggaactgcacaagaagggttgtcctgacgtgcccctggggaccgactactacctggttcgggatctggaacgggacgagcggaagaaccggttccacaatgacaacgccgccctgtacgaaacactggccctggatagactttgtgtgctgatggccagagattgcctggtgagactgaatcggaacctggagaagcacgccacaagaatcagctgggaagccaccgacgccggcgacaccatctgcctggagctccccagaagagaccgggatcacgagagcttcagactgagtttcggcgtacgggattaccccaagctgtacgtgatggacgaccccgtgttcctgtgcggcctgatgaagcatttctttcctgacaaccaggccatccagtaccacgaactctatagcgagggaatcaacaagtacaccgccatgcaggccgaaggaattgccgccaccctgaaactggaagaaaaaaccatcaaggagaagaacatgcagatccctgctaccggctacatcagattctgcgagatcgtgtcccagtccgacttcgcccctggcgagaagcgggtgctgaagaatgtgcggaacggcctgctgcattatcacctggagttcgagcctacagagtgggccgaattccgggaaatcatgaaaagagagggattcgacaccgccaagaaaagaaagtctaccagaaaaaagtga

SEQ ID NO: 92 - DNA, 2415 bp, Artificial Sequence
  Description of Artificial Sequence: Synthetic polynucleotide
atgaagatcaagaacgagaacacagacaagaaaaccgaactgtacagcatcgacaagtacaagggcagggacaagtggtgcttcgccatcgtcctgaacaaagctcagaccaatctgaccgagaaccccgacctgttcgaacaaaccatcacaaagtacgacagaatccggaaagagggctggttcgatgaggaaaccaagaaactgatctacatccaggagaacgagcacaagatcaagggcgagatcaagaccctggccagagaagtgctgaaaaacctgagaaactacttctcccaccacttctacaagcaggattgcctgatcttccccaaggacaacattgtgcggatcatcatgggccgggcctacgaaagaagcgaatacgaaatcaagaagaatctgaaggaggacataagcatcgagctgcctgccctgttcgagcctgagggcaagatcaccaccgctggcatcgtgttcttcaccagcttcttcgtggaacgtcggtttctgcacagactgatgggctccgtgcagggctttaccaagaccgagggcgagtacaaaattaccagagatgtgttcgccaagtactgcctgagagacagctatagcgttaaggcccaagacaatgacgccgtgatgttcagagatatcatcggctacctgagcagagtgcctacagaaagctttcagcacatcaagaatcctaagaaacagaacgagtcacagctgagcgagcgcaagacagataagttcatcagctttgccctgaagtacctgaaggactacggcttcgaggatctgaagggtcactacaccgccttcttcgcaaggtctgagatcaaaaaggagaaagaggacatcgaaatcaaggatgacaagaagcacaagccccaccgaatgaaaagcaagatcgagatccactttgataagaccaaggaggacagattctacatcgagagaaacaacgtgatcctgaagatccagagaaagggaggccgggccaacatcctgcggatggggatctacgagctgaagtatctggtgctgctctgtctgtccggcaaggcccgagaagccatcaaccgcatcgacgactacctgaacgacctgcggaacaagattcctcacatcgaaaatatgaacaaggaaggcatcggcgagcagataaggtctcttcctggcttcgtgcggagtcagctgggattcgtgcagatcgacgatgagaagaagaaggaaaaccggctggactacgttgaaaagaagtgggagaagaagagagccgagtctaaagagctgaagctgaatagaaaaggcagagatattctgcggtacattaacgagagatgcaagaaacctctgaccatcgaccggtacaatagaatcctcgagctgctggtggagaagaacatcgaaggattttaccacgagctcgaggaactgagaaagaccggccggatcgagaaaaacatcacacaggcccttgtgggagagaagaacatcaacgccctgcatatcaagatctgcaagctggtgcaggacgagctgaagagcctggagaaggaagatctgaaaaagtatatcggcctgacacctaaggaggagaaggtggtgtcctttgaggagaaactcggccggatcctggacaaacccgtgatctacaagggcttcctgagataccagttcttcaagaacgataagaagagcttcgccagactggtggaagagattattaaggaaaagaccggcggactggatgtcccaatcgagacagaatactactctatatctacactgggccggttcgacaaggctaacaagacactgtatgaaaccctggccatggacagactgtgcatgatgatggcccggagatacttcctgagcctgaataagatcctggctaagagagctcagaatatcgagtggaagaaagagagcggcaaagaattcatcgtgtttaagttcaacatgcctcaggatacaggcaaatctatcagcatcagattcagccctaaagactacacaaaactgtacgtgaagaacgacagcgaattcctggctagactgtgtcagtacttttttccaaacgagaaggccatcgactatcacaaactgtacagccacggcatcaacaagtacaccaacttccagaaggaaggaatcgaggccatcctggaactggaagaaaagatcatcaagaaaagaaagatcaacagccccgagaactacctgagcttcgaagagatcctgaatcagagcatctacaacgacgaagaaaagaacaccctgatccaaatcagacacagcctgctgcattaccagatcctcttctctaagaatgacctgaccaagttctataacgtgatgaagagagagggcatcgaaaagatctggtccctcgtcatctga

SEQ ID NO: 93 - DNA, 2637 bp, Artificial Sequence
  Description of Artificial Sequence: Synthetic polynucleotide
atggccgtgaactactctctgagagaaaaatggtatcgcggcgtcaacaagtgctgtttcaccgtggccctgaacatcgccgtggacaactgcaagagcaagggttgtgaaaccctgctgaaagaggccgagcattctaaaggcggaatcaccgacgagcagatccagcagtcttatacagaggtggagaagaggctgaacgacatcagaaattacttcagccacttctaccacggcgatgagtgcctgatcttcaagaaggatgacatcgtgaagcggttcatggagagcgtgttcgccaccgccgtcagcaacgtggtgggcggcaccaaggagagcgactacaagggggtggtccctccactgttcgagcagagcaacgaggactatatgatcaccgccgccggcgtgattttcctggccagcttcttctgccacagatctaacgtgtaccggatgctgggagccgtgaagggcttcaagcacacaggcaaggaagaactgtccgacggccaaaagcgcgaccacggattcacacaccggctgctggcccactacagcctgcgggacagctacagcgtgaagatcgaggagacaaagtcttttagagatctgctgggctatctgagcagagtgccccagcaggccgtggactggctgaacgagcggaacgagctatctgaggacgagaaaaaggaattcctcaaccagaaaagttctgaagaagaatctcctgagcaacctgagcctgagaacgccgaatggcggaccgaaaagaccagccggagaagcctgagaaagaccgagaagttcatcctgttcgccgccaagtttatcgaggatcgggccgagaaggaaaagcaagacgtgaccttcgccagataccagaaaaccgtgacaaaggaagagaacaaaaaccaagacggaaagcaggcccgggtggtgcggctcaagtacgaggaggacaagaaagacgacgagaaacctagagagcatttcaacctggagtggatgtactacataaggaatgaacacgccatcatccagatcaagcccaaggacaaggaagccgtggctgccagaatcagcgagaacgagctgaagtacctggtgctgctgatcttcgagggaaaaggaggcgacgcctttaacaagctgagcgactacatctttagaatgacccagaagatcaagtccggccagatcaaccctaacgaggccagactgccttcctttctgaagaaccccgtgaagaacatcaccgacaagatggtgcgtaatagactggactacattagaggccagattaaagatgtgctggaaaagatcaacatggaagaaccccagaacaacaagtggctgatctacaagggcaagaaaatcagcctggttctgaaattcatctctgacggcatcagcgacatcaaaaagcggcctaatgtgaaagagtacgacacactgagagacaccctgcagaagctggacttcaatcggttttacgaaagactcaagtcgtacgtgtccgatggcagactggctgccgctctgtacgataagatcaagggcatcgacgacatcagcgagctgtgcaagaaggtgtgcgagctcatgttcgccagactcgccgaactggaaaagaaaggcggctttgagctgtacagatacatcggcatggaagtgcaggagaaagatgagaaatacgatgagtggaacagccctcagaagaaggccgagagattcctggagagccagttcagcatcggcgaaaacttcctgagagagagcttctacagcgagtactgccagaagcaggagtgtatcgacaaggaaatcagcctgaataccagcgtcaagaacagaaagtctctggtgtacatcgtgaaagagaagctgaaggatatcatgcctctgcacaacgaccgctggtatctgatcgacagaaaccccaaggatttcgaaagaaaggacagcaaggtgatcaagggactttgcaacacctacgtgcaggacgtgctgtgtatgaagatggccaggtggtattacggccagctgaatcccgccctgaagaacaacatcaagtgggacgaaaccggacaaggccacggctacgacagatacaagctgagctaccggactaacttcggcatcacaatcgagttcaagctggctgattttacaagactggatattatcgagaagtcagatatgatcgaaaacatctgcagaagcttcatcaagcctaacagaacaatctcctggtacgacttcaaacaggacggcgtggaagaatacagaaagcggcaatacaaggccgttcgggctgtgttcgcctttgaagagagccttatcatccccggcagagattggctgagccagggctttgtgccattcatcaagaacgaggaatacgtgaagaagggattcagcctgttcgtgctggatgaagctgtgcggcagctgaagatcaagggcagcgataaggacgccatgagacaggtgcgcaatgacttcttccacgagcagtttcaggctaaggatgagcagtggaaggtgttcgaaggctacctgagctgcttcatgattgatagacctaagggcgagaagaacaagaaacgttacaacggcaacaagaaatga

SEQ ID NO: 94 - DNA, 2616 bp, Artificial Sequence
  Description of Artificial Sequence: Synthetic polynucleotide
atggaaaagtacctcatcaagaatttcgagggcatcaacaagagcaagttcaccgtggctctgaacatcgccaacgacaactgcaagaacaagggaatccaggagctgctgaaggaagcccagcgaagcaagggaggcatcaccgacacccagatcaccgaggtgcaggagcacatcaaggaacggctgaatagcgtgagaaactactttagccactgctaccacgagaaaaaacctctgtacttcgaggctaatgatcctgtgaaaatcttcctggaagaaaccttcgccaaggcagtcgagaacctgcagggaagattcctgagcgacaaatacaagctgacagtgcctccactgttcgagcccaaccagaacaacaccatcacagctgccggcgtgatctttctggccagctttttctgccacagaagctacgtgtacagaatgctgggaggcatccctggcttcaagcggtccgataagaaaaagtggggcgacggccagaaaatcgactacggctttacaagaaagctcatgagcttctatagcctgcgggactcctatagcgtgaacgtgcaggagaacaaggagctgaccgcttttagagacatcctcggctacctggctcgggtgcccggccaagccatcgattggctgatagagaagggcaagctgacaaaggaagagggcaaacagttctacctgggcgagcagagcgaggaacgggaagagaaggctaagaaggaagagatcaagtacgccctgagaaagacagacaagtttatgctgttcgccgtgagattcatcgaggactgggcggagcaggagcggatcaaggtggaattcgccagatacgaaaaaatgaccatcgtgaatgagaataagaaacaagacgaaaaagaggagagaaaagtgaagttcgtgtctgacgaaccaaccgccgccggatggacctactacatccgcaacaaccacgccatcatcaagatcatccccgatgataaaaagaagaaggccgtttctgccaggatctcagagaacgaactgaagtacctcgtgctgaccatcattgacggcaacggcaagaacgccatcgcctacatcggcgattatatcttcagaacagcccggcagatcgaaaacaaatcctacaacgccgaaagcgagaaatacgccccagccttcgtgcgcggcggacagaagaagtccgtcgacaagagaatcaagtacatccgggacgagatccagcaggtgatcaacgacatcgaggccgagcaggagaagcagaagaacgagcaggacgcccctgccgagaacagaacctggctgatctacaagggaaagaagatctctatcatcctgagatacgtgaacgataacatcgccgagtacaagaaaagactgagcgttacagagtacaacgagctgagaggctacctgcagcaactggacttcattaacttccatagaaagctcgccgaataccagcaccacggcagactgcctaatggctttgctgaaagcatcaacaagtttcaggacctgtccaagctctgtatcgaggtgtgtgaaagacagaagaagaagctgcaagagatggccgccaagggcggaattgagctggaacaatacatcggcctggcccctaaggaagaaaatcaggaacagaacaagtacgccaccaaggccaacaacttcatcaaggtgtggctgtctatccccgagaatttcctgagacagaagttctacgacaagttctgcaagcagcaggaatgtaaaaacaagggcagcgataagcctgataataccagcgtcccccagagaaagtacttcatagctatcattagagaaaagaatatcaggcccatccatgcagataagtattacctgctgggccagaatcctaaggattacgagaggcctgatggcaagatcatccggcagctgtgcgatgtgtactgcaaggacggcctctgcatggctatggccaagtggtactacgagaaccggctgggcaaattcaaagacctgatcgagtggcagaccggcgacgacaaacagcagcacggatacgccggccacacactggaataccaggctacagagaagatcaagatcagatttaagctggctgattttaccagactggacatcatcgagcctcctgaaagagtgaagaacatctgtagacaatgggaaaccgaactgctgaaaaaaaccagagacggcacaatcagctggtatgatttcaagctgaacggacttgagccttacagacaatggcagggctatgccgtggccgacatcttctggttcgaagaaagcctgaagatcaacgagacacagtggcaggggcggacccacatgcctttcaacttcgagaaggacaaacccctgtggtgcaacatcctggacgaggccgtgaagcagaacaagattgaaaaacaggacactcaggccctgcgccgggtgcggcacgactgcttccacgaggagttcctggctaattacgagcagctgaaaattttcaaaaacctgatttctgataaggccaaggacgccaagcctaaggacaaaaaatctagaaagaacgagcagaagtatggcaagcggtga

SEQ ID NO: 95 - DNA, 2442 bp, Artificial Sequence
  Description of Artificial Sequence: Synthetic polynucleotide
atggaaaccaccgaggaaaaaaaagaaatctacagcatcgacaagtatcagggcgccggcaaatggtgcttcgccatcatcctgaatcgggctagagacaactacgagggcaaccctcacctgctcagcgagagcctgctgcggttcgaggctgtgaacagaagagattggttcgatgaggaaaccagagagctcattatccagatggatgccgaaaccaagacagacagcaatctgaagccttctacacctgcctacaagatcctgatcggcatcaggaattacttcagccacttttaccacactgaagagtgcctgtacttcaaggaggatgaccccatcagaatcattctggagaacgcctacaagaaggccaaacaacaccacatcgagagactggagaaggagaccgacattgagtttcctgctctgttcgagagcaacgacagaatcacctccgccggcatcatctttttcagctctctgttcgtggaacggcggatcctgaacagactgatgggctacgtgggcggatttaagaaaaccaagggcgaatacaacatcaccagagaaatcttcagcacctactgcctgagagacagctacagcatcatggccgccgacagcaacgctgtgatcttcagagatatcctgggctatctgagcagagtgcctagcgagtactaccagcataataaggagaagtgcgagaaagagaacaagcctaagcgcaagacaaagaagttcatctattttgccctgcggtacctggaaaaattcgtgctgaaagaaatcaagaaccagaaggtgtccatcgccagaatggaagtggtgcgggagaaaagcaaggaagccgagggagaagatgagcagcacaagccataccctgacaaggccaaggtgaagatcgttttcgactccatcggcaaggaacttccttactacatcaaccacaaaacagtgatcatgaagatccaacggaacggcgaagccgtgaacttctgcaagattggaatcaatgagctgaagtacctggtgctgctgtgtctgcagggcaagagtgtggacgctatcgccaagatcaacggctacatcgaccggattaagaagcggctggaaaatcccaagacaagaatcgacatcgacagcgagcgcgacaatgagttcatcaagggcctgcccgagttcgtcaagatccagtccggtagaacccctgacgaggagagagagaagaaatctagaatcgactacatcagaaagaagtgggagaagaaaaaggaggagagtcctaagaccgagctgcatagaaaaggcagagatatcctgcggtacatcaactggcactgtgaacctccactgggctctgaagagtataaccacctgctgagcctgctggtgaacaaggacctggacggcttcgagaacgagctgaaggagctgaagagaaccgaacagatcagcaagaagctgctggagatgctgaagggatttagcaacctgaacgagctccacctgaaagtgtgcagcatcgttcctgtggaactggtgtacctggaaaagaatgacccagagaagctggccgagtacatcggtctgatcccccagaaaaccgacaaggaacccccctcttacgaagaaaaggtgaagaacttcatcaagcagcctatgatctacaagggcttcctgcgggatacattcttttcttccggcaagacattcgccaagctggtggaagagaccttcctgcacaagtacccccacagcgacgtgcccctgggccgggattactaccacgtgacaacactggacagatttcacaaagacaacagcatcctgtacgaaaccctggctctcgatagactgtgcgtggtgatggccagaaagttccacgagaccctgaaccaggagctggccaaggagagcaagcagatcgtgtgggaaaacaacaccatcatcctggaactgcctagaagcaagaccagcagctctgacaccttccagatctgtttcgacatcaagcactacatgaagctgtatgtgatggacgacgtggaattcctgggaggcctgatgcggcacttcttccaaaaggagaaaaccatcgagtactataacctgtacagcttcggcatcaacaaatacaccgagatgcagcggaacggcatcgaggccatcctgcgcctggaagaaaagatcattcgggaaaagaatatcaaaagaaagcctgataagaactacatctctttctacgagatcatggaagccagcaactaccctgataacgacaagaaaacactgaacaaggtgcggagagccctgctccattaccacctgaagtttgagcccgctgattacaacaagttcgtcgccattatgaagagagagggcctggagagaaagaagaaaactggcaaggcaacacggagatgggtcaagaaatga

SEQ ID NO: 96 - DNA, 2370 bp, Artificial Sequence
  Description of Artificial Sequence: Synthetic polynucleotide
atggaaaacatcaagctggagaagcagaaggccgccttttacttcaaccaggccgagctgaacctgaaggccatcgagggcaatatcttcgacaaaggcagaagaaagaccctgtttgataaccccaagattctgagcaaagtggaaaacttcatctttaacttcaaggacgtgacaaagaacgccaagggagaaatcgactgcctgctgagcaaactaatggaactaagaaacttctacagccactacgtgcacaagccagacgtgaaagagctgtctaagggcgaaaagcctctgcttgagagatactaccagattgctatcgaagccaccggcagcgagaacgtgaagctggagatcatcgagaatgacaaatggctgaccgatgccggcgtgctgctgttcctgtgtatgttcctgaaaaagagccaggccaacaagctgatcagtggaatcagcggcttcaagcggaatgatacattcggccaaccaagaaggaatctgttcaactacttcagcgtgcgggaacggtacaaggtggtgcccgacatgcagaaacatttcctgctgtttgtgctggtgaaccacctgtcagagcaggacgactacatcgagaaggctcagcagccttacaacatcggcgaaggcctgttcttccacagaatcgccagcaccttcctgaacgtgtctggcatcctcagaaacatggaattctacacctaccagagcaagagactgaaagaacagaggggcgagctgaagagagagaaggacatcttcacctgggaggaacctttccagggcaacagctacttcgagatcaacggccacaagggcgtgatcggcgaggacgagctgaaagagctgtgctacgccctgctgagctataacaagagcaaatatgccgtcgagcagatcgagaagttcctgaaaggcttcggcgaggtgaagagcgagcaggagatcagagatagcgacatcctcaatgagagctacttccccaccaactacttcgccgagagcaacatcggcagcatcaaggaaaaaatcctgaacagactgggaaagaccgacgatagctacaagaaaaccggaacaaagatcaaaccctacgacatgatgaaagaagtgatggaatttatcaacaacagcttgcctgccgatgagaagctgaagcggaaggactacagaaggtacctgaagatggtgcggatctgggatagcgagaaggacaacatcaaacgggaattcgaatctaaagaatggtccaaatacttttctagcaacttctggatggccaagaacctggaaagagtgtacggcctggccagagagaagaatgccgaactgttcaacaagctgaaggctgttgtggaaaagatggacgaacgcgagttcgaaaagtaccggcaaatcaacagcgccgaagatctggcctctttgcggcggctggcaaatgactacggcgtgaagtgggaagagaaggattggcaggagtacagcggccagatcaagaagcagatctccgatagacaaaagctgacaatcatgaagcagagaattacagccgagctcaaaaagaagcacggcatcgagaatctgaacctgagaatcaccatcgacagcaacaagagcagaaaggccgtgctgaatagaatcgccgtgcctagaggattcgtgaaggagcacatcctgggctggcagggatctgagaaagtgtccaagaaaacccgggaagccaagtgcaagatcctgctgtccaaggaatacgaggaactttctaagcaatttttccagaccagaaactacgacaagatgacgcaggtgaacagcctgtacgagaaaaacaagctgatcgctttcatggccgtgtatctgatggggcagctgaacatccggttcgacaagcctaccagactgaacgagctggaaaaggccgaggtggacttcaaaatcagcgacaaggtcacagccaaaattcctttcagccagtaccccagcctggtctatgccatgagcagcaagtacgccgactccgtcggctcttacaagttcgagaacgatgagaagaacaagccttttctgggcaagatcgatatcattgagaaacaacggatggaattcatcaaggaagtgctgggctttgaagagtacctgttcgagaagaagatcatcgacaagtccaagttcgctgacaccgctacccacatttcttttagagagatctgcgacgagctgatccagaagggctgggatgagaacaaactgaccaatttaaaggacgccagaaacgccgctctgcacggcgagatccctgctgagacaagcttcagagaggccaagcctctgatcaatggcctgaagaagtga

SEQ ID NO: 97 - DNA, 2400 bp, Artificial Sequence
  Description of Artificial Sequence: Synthetic polynucleotide
atgatcaacatcgagctgaagaaggaagaggccgccttctacttcaaccaggctaacctgaacattagcggactggatgaggtgatcgagaagcagctgccccacataggctctaaaaaggagaacgccaagaaggctatcgacaagatcttcgacaacattacagtgctgaagaaagtcgagaacttcgtgttctactttaaggacgtggccaaaaacgagagagtggaactggacgccctgctgctgaaactgatcgacctgcgcaacttttacagccactacgtgcacaacgacaatgtgaagatcctgtcagacggcgaggaaacactgctggaaaaatattaccagatcgccatcgaagctacaggcagcaaagacgtgaagctggagatcatcgacaatgagaagaaactgacagacgccggcatcctgttcctgctgtgcatgttcctgaaaaagagccaggccaacaagctcatttccagcatcagcggcttcaaacggaacgacaaagaatggcagcccagaaggaacctgtttacctactactccctgagagaaggctacaaggtcgtgcctgacatggagaagcacttcctgcttttcaccctggtcaaccacctgagtacccaggatgagaacatcgagaacacccagcctagcgatgacatcgggagaggcctgtttttccacagaatcgccagcaccttcctgaatattagcggcatctttaacaacatggaattctacccctaccaatctaatagactgaaagaaagacggggcgacatcgctcctgacaaggatagcttcgcctggatcgagccttttcagggcaacagctacttcaagatcaacggctacaagggcgtgatcggcgagaacgagctgaaggaactgtgcttcgctgttctgctccataacaaaagcaagtacgccgtggaacagatcgaaaagttcctgaaatgttttaaggaagtgcagtctaagcaggagatcattgaatgcgacatcctggacgagtgctactttccagccaattatctgaaccagcctgagaccaagtccctgaaggaaaagttactgtctagaatcacaggcaagatcaattactccttcgacaccgccgaaaaggcctttgataagatgaaggacgtgatggaattcatcaacggatgtctgcctagcgacgagaagctgaagcggaaggactactctcggtacctcaagatggtgcggttctggggtggcgagaaagataacatcaagagagagttcgagggaaagaaatggacccgctttttcccctccgagctgtggcacaagagaacactggaagatgtgtacaagttcgccctgaagaagaacaaaaaacgactggaggaactgaaggtgaagatcgagggccttaacgaggacgatctgctgaagtaccagaaggttaacaacatcaagaaccttgaaaacctgagactcctggcccacgatctggacctgagctggagagagaaggactggggcgaatacagcggacagatcaagaaacagatcagcgacaatcaaaagctgacaatcatgaagcagagagtgatcgccgagctgaagaagaagcacggcatcgaaaatatcaatctgcggatctctctggatagcaacaagagcatccaggccgtgctgaacagaatcgctatccccaagggctttatcaagcggcacgtgctacatctgcaggagaacgagaaaaccagcaggaagatcagagaggccaagtgcaagatcctgctgagcaaaaaatatgagtacctgtctagaaagttcctggatgaaaagaacttcgacaagctgacccaaatcaacggcctgtacgagaagaatcggctgatcgcattcatggtgatttacctgctgaagcagctgggactggaattaaagaacgagaccaagcttatcgaactgaaaaagaccagagtgaagtacaagatttctgataaggtggccgaggacatccctctgtcccactaccctagcctggtgtacgccatgagcagaaagtatgtggacaacatcgataattacgagttccctgatgaatacgccaagaaggccatcctggacaaggtggacatcatcgagaatcagcggatggaattcatcaaacaagtgctgggcttcgagaagtacctgttcgacaacaacatcatcgacaaaagcaagttcacagatgttgagactcacatctccttcgtgaaaatccacgacgagctgatcgaaaaaggctgggacaccgagaagctgtctaagctgaagcacgccagaaacaaggccctgcacggagaaatcccaggcggcaccagcttcgagaaggctaagctgctgatcaacgaactgaagaagtga

SEQ ID NO: 98 - DNA, 2634 bp, Artificial Sequence
  Description of Artificial Sequence: Synthetic polynucleotide
atgaagaagcagaagagcaagaaaaccgtgtccaagactagcggcttgaaagaagccctgagcgtgcagggcaccgttatcatgaccagcttcggaaagggcaacatggccaacctgagctacaaaatccccagctcgcagaaaccacaaaacctgaactcatccgctggcctgaagaacgtggaagtgagcggcaagaagatcaagtttcagggcagacaccctaagatcgccacaaccgacaatccactgttcaagcctcagccaggcatggacctgctgtgtctgaaggataagctggaaatgcactacttcggcaaaaccttcgacgacaacatccacattcagctgatctaccagatcctggacatcgagaagatcctggccgtgcacgtgaacaacatcgtgttcaccctcgacaacgtgctgcaccctcaaaaggaagagctgaccgaggatttcatcggcgccggcggatggcggatcaacctggattaccaaacactgagaggacagacaaacaagtacgaccggttcaagaactacatcaaaagaaaagagctgctgtacttcggagaagccttctaccacgagaacgagaggcggtacgaggaggacatcttcgccatcctgaccctgctgtccgctctgcgccagttctgctttcacagcgacctgtctagcgacgagagcgaccacgtgaactccttctggctgtaccaactggaagatcagctgagtgatgagtttaaagagacactcagcatcctgtgggaggaggtgacagagagaattgacagcgagttcctgaaaaccaacaccgtgaatctgcatatcctgtgccacgtgttccccaaggaatccaaggagacaatcgtgcgggcttactatgagttcctgatcaagaaaagcttcaagaacatgggcttcagcatcaagaaactgcgggaaatcatgctagaacagagcgatctgaagtctttcaaggaggacaagtacaacagcgtgcgggccaagctgtataagctgtttgatttcatcatcacatactactacgaccaccacgccttcgagaaggaagccctggtctcctctctgagaagctctctgaccgaggaaaacaaagaagagatctacatcaagaccgccagaacactggcttctgcccttggagctgactttaagaaggccgccgctgacgtgaacgccaagaatatcagagactaccaaaaaaaggccaatgactaccggatctcttttgaggacatcaagattggcaataccggcatcggctacttttctgagctgatctacatgctgaccctgctgctggacggcaaggaaatcaacgacttattgaccacactgatcaacaagttcgacaatatcatcagctttatcgacatcctgaagaagctgaacctggagtttaagttcaaacccgaatacgccgacttcttcaacatgaccaactgcagatataccctggaagaactgagggtgatcaacagcatcgcccggatgcagaagcctagcgctgacgccaggaaaatcatgtacagagatgccttacggatcctgggaatggacaatagacctgatgaagaaatcgatagagagctggaaagaacaatgcccgtaggcgccgatggcaagttcatcaaaggcaaacagggctttagaaatttcatcgctagcaacgtgatcgagagcagccggttccactacctcgttagatataacaacccccacaagacaagaaccctggttaagaaccccaacgtggtgaaattcgtgttggaagggatccccgagacacagatcaagcggtacttcgatgtgtgcaagggccaggagatcccacctaccagcgataagagcgcccagatcgacgtgctggcccggataatttcttccgtcgactacaagatcttcgaggatgtgccccagtccgctaagatcaacaaggatgaccctagcagaaacttcagcgacgccctgaagaagcagagataccaggccattgtgtctctctacctgacagtcatgtacctgatcaccaagaatctggtgtacgtgaacagtagatacgtgatcgccttccactgcctggagcgggacgcctttctgcacggcgtgaccctgcctaagatgaacaagaaaatcgtgtacagccagctgaccacccacctgctgacagataaaaattacaccacctacggccacctgaagaaccagaagggccacagaaagtggtacgtgctggtgaagaacaacctgcagaacagcgacatcaccgccgtgagcagcttcagaaatatcgtggcccacatcagcgtggtgagaaactccaatgagtacatcagcggcatcggagaactgcattcttacttcgagctgtaccattacctggtgcagagcatgatcgccaaaaacaactggtatgacaccagccaccagcctaagaccgccgagtacctgaacaatctgaagaagcaccacacctactgtaaagatttcgtgaaggcctactgcatccctttcggctacgtggttcctagatataagaacctgaccatcaacgagctgtttgacagaaacaaccctaatcctgagcctaaagaggaagtgtga

SEQ ID NO: 99 - DNA, 2595 bp, Artificial Sequence
  Description of Artificial Sequence: Synthetic polynucleotide
atgaaaaaatctaaggtgaagctcaatggcgtgaaagctgtctaccacatcagtcccgacgtgagagtcatcgccgctttcggccgtggaaacaacagcgtgctggacaagcacatcgagaacggatctgttgaggagctccagaaccacagcgacatcgaggtgaacatcagcagaaagacctacagcttcagaaagaagtctctgaagaaggccgccggccaattttctgtgcctgacaacaccaatgaccagctgggcatccgggaagagctggaaaaggaaatcttcgggagaaagtttgacgacaacatccacattcaagccgcctacgccgtcaacgacatcatcaagatgctggctgtggccgccaatctggccgagacagcaattaacggcctggacagagagaataccgagaatgacatgatcggcttctgcatcataccccacatcacctaccaaacatacgccgatgacaagagaagcaaattcgagggcttcatcgaaaaggtgaaaacccagggcaccttcagctacttccctgatatcctgcctaaatttaagaaggaagaatcagaggaagagtccgacaacgaaaagctgtactacatcatgtgcatcatcagcctgatcagaaactctgctacacacagcaagagcagcaacagcgataccaccgactacatcttcggcgaattcaactctgtgaacaaggaagccttgacaaccacagctgataatctgatcaagtcaaaaatcgatttcatcaacaaggaaggctttagcaaaaaccagaaaaacaacatctaccggctgctgaaagctaaggctgacacccccgagaacaccgccagactgatccggagactgtatgctttcaccattagaaaacaggacaaaaatctggggtttagcctgaagaaactgagggagtgcgccatcagatccatcgaccgaagcatcgaatacatgaagtacctgccatctaagaaatacgacaccgtgcggagcaagctgtacaccctgatggacttcgtggtgtatagctacctgaaatatcacaaggatggcaaaaagttctctaaggaaatggtcgagcagctgcgggccgctgaaagcgacaaggtgaaggatgacatctaccgggacgaggccgagaaactgtacaatatcgagatcatcagcagaacaatcaacgccctgatcagcgacatcaagtccgacttcgaccagcctaagcacggaaatgagtgttaccagccaatcaacgatggcatgaaggaggccgagaaagatttcatcaccaccgatcagctgagcctgttcacaaagttcatctacgtgctgtgccagttcctcgatggcaagaacatcaacatcctgctgtctagcctgatcagcaagtttcagcagatcgaggcttttaacggcgatatcagaaaactgaacctgaacattagagatgatggaaagatcggctacgacagcaagaagtacagcatcttcgagaagagcggccagatcgccgacgatctggacaagctgagaggcgtgatcaagatggacatcaacgatctgaacgcctatgagacaatgatcaaggacgccctgagggtgatcggcgtggacgagtccgacatcgagagcatctaccagacccacttcaagacacaggataagaaggacagcgtttctggcttcttcagaaacaacatcatcaacagtagaagattccggtacatcattaagtacatcaaccctagcgacgcctacagaattatccagaacgagaacgtgcggaactacgtgttgggccggatgaacgacgccatcatcgacagatacgcccactccgtgggcatcgaagataaggtgcacgacaaaagaaaggtgctgagcgatatcctgtccaaggtgaagttcgacaacttcaccaagctgacctacatcaatcctaaggacaagaacaagggcgagaaagcaaaggagagagagaagcctaaggccattctgggcctgtacctgacaatcgtgtacctggtcgtgaagagcctggtgcggatcaacagccagtacgtgatggccgtgtaccacctggaaagagacagcagactgtgtcctggagtgtccagcaacaacctgctgagcatgacaaaccattactgcgacagagacaatcacctgctgaaagagaagcatatcgtgaagctggaacggtacaagaaaacacagaaaacccaaaagaccatctgcaccgcctacagaaatgccatcgcccacctgtctgccgtgcgcaagggcgtgaagtacatcggtgacattcagaaggccgattcttacttcggaatctaccactactgtatgcagaaactgatctacagcgccaacggccagcctttcgccgagttcgtgcggtccatttttggcgatgaaaaagaactggataagctgagaaacggctcttacagccaggccatcctgagagccctgaattaccccttcggatacaaccccgcccggtataagaacctgagctatgagaagatctttatcagagcctggcaggacgaggacaccaacaaaaagacctga

SEQ ID NO: 100 - DNA, 2643 bp, Artificial Sequence
  Description of Artificial Sequence: Synthetic polynucleotide
atgaacaagattcataagaagcagggcaagaccaccgctaaaagcctgggcctgaagagcgtgctgaagatcgagaacgacctggtggtgaccaccttcggcaagaaagacaaccccatggtcgtggaacagagcatcaacaaggccagtggcgagaaggaactgtatgtggacgaggaccaagtgaaattcgactctagcctgatcaaggaaaagaacatcctgtctctggattcgatccagcacagcaatcaccagatcatcgtgaacatcgatcagaaggacgcctccgagatcggcatggattacctgagactgaagccagagctggaaaaagagttcttcggcaagacattttacgataatgtgcacatacagatcgcttacaacctgctggatctgaagaagatcatcggactgcacatcggcaacgctatccaggccctggaaaacctgggacgggacggcagcgacctggtcggaatctgcgacgctacaaagcctctgaattacctggatgatgtgaagcaaaaggcagacatcggcttcatgaaccggctgaagccttacttcatgtacttcgatggtgttctgaagctggacaatagcaagaacaagaacggagagctgaaccagctggacatcgagaactgggatgtgatccggatcctgtccctgatcaggcagggctgtgcccacgccggcgcttacagcagccttctgtacaccgcccagaacaacaaggtgtacgccgacctgatcaacaaggctctgtctatctttagcgacgacctggacaagttcaacaaatcattcctgaagcagagcaaaatgaacctgttcatcctgtttgatctgtataactgcagattcgaccgaagtctgcaggagaagattataaaagagtactacagatacgtgctgtacaaagacaacaaaaacctgggcttcagcctgaagaacgtgcggaacctgatcatcgagggcaaatacgacgagcaggagcggagcggaaagctccagaccatcagatccaagctgaacaccctgctggacttctacctgtacgggtactaccagaaaaaccctaccttcgtggaaaatatcgtggccaagctgcgggaaagcaagaatgacgaagataaggaaaaggtgtacgaggaagaatatcacagactgctgtctgaaaacaactatctagtcgacaagaaatgtagcgacattgtctaccggatcaacgaggccgtgaagaacagaaaaatctttgtgaatgccaacattaatgccgtggttgaaaaggtgtcttgcagctgcttccccagcctgatctacgtgctgtgcaagttcctggacggcaaggaagtgaacgagcttacaactgccatcattaacaagctggaaaacatcgcctctcttatcaacgccctggtgacactgaaaagctacggcggcttttctgagcagtacaagatcttcgactaccctaacatcaatggcctgatcgacgacttccggatggtgaagaacctgacaagcaccaaaagaaagctgaagaaggccagcggcggagaggatagaatcggcagacaactgtacgccgatgccatcaacatttttaaggaggacagcttcgtgagcgccaacgacgagaagggcacaggcctggaccagtacgtgaacaaatttttctccaaggacgatctgggcgccagaaaggtgcgcaacttattgctgaacaacatcatcaaaaaccgcagattcgtgtacctcattaagtacatcgaccccaaggattgctacaagctggtccacaacgagaaaatcgtgcggttcgccctgggacagtacgatgagagccagatgcctctgaatcaactgcagaaatactatgacgccgtgatcgagaatagagagggcttcagaaagtgtaacgaccggaagaaaattatcgacaccctcgtgtccgaaatcaacagagtgtctatcgatggcatcctagatatcggcaacagactggtcaataggggcaacaacgattacatcaaccaccagaagcagatcatcagcctgtacctgaccatcgcctatctgatcgtgaagggcgtggttcacaccaacagcctgtactttatcgcatggcacgcctatgagagagataacaacttcaaattcggcaatgacggcaaggactacctggccctgacaaaggagtacctgacaaacaagaagaagcgggtgaagcagctgctggaccacaacatcgaggaagccaataatagcctggactccaaatacttcagcgcctatagaaacaaggtggtgcacctgaacttttgcaacatcttcgtgaactacctggacggaatcggcgacatccattcttactacgacatctaccagtacgtgatccagaagtggtccatcgctgaaagaagcaaggatttcatcgaccctcaatacctgaccaagctgtccaatgacctaaaacagtacagaacctaccaaagaaatttcctgaagatcatcaacctccctttcgcctacaatctggccagatacaagaacctgaccatcggcgacctgttcaacgacaagtacccccttccaaaggaaaccgtgaaggaattctacaacgaggagtga

SEQ ID NO: 101 - DNA, 2643 bp, Artificial Sequence
  Description of Artificial Sequence: Synthetic polynucleotide
atggccaagaagaaacggatgagcgccaaagagagaaagcaacagcagatcaacctgagaatcaagaaagctaccgaggatagcaccaagaaggtgaataccaccgtcgctgtaaacaacaagcctatcagcaaggaaatcaagaagagcaaagccaagctggcaggcgttaagtgggtgatcaaggccaatgatgacgtggcctacatcagcagcttcggcaagggcaacaacagcgtgctggaaaagagaatcattggagatgtgagctctgacgtgaacaaggactctcacatgtacgtgaatccaaagtacaccaagaaaaactacgagatcaagaacggcttcagctccggatcttctctgacaacccaccccaacaagcctgacaagaacagcggaatggacgccctgtgcctgaaaacctactttgagaaggagatcttcaaggacaagtttaacgacaatatgcacatccaggccatctacaacatcttcgacatcgaaaagaccctggctaagcatatcaccaacatcatttacgctgttaacagcctggacaggtcctatatccagagcggcaacgacacaatcggctttggactgaatttcaacatcccctacgccgagtacggcggcggaaaggacagcaatggcaagcccgagaacaagtccgcctgggagaagcgggaaagcttcatcaagttctataacaacgccaaggatcggttcggctacttcgagtccgtgttctaccagaacggcaagcagatcagtgaagaaaagttctacatctacctgaacatcctgaacttcgtgcggaacagtaccttccactacaacaacaccagctcacatctgtacaaggagcgctactgcaagatcaaccccaagaataacctgaaaaccgatttcgagttcgtgagctacctgaacgagttcgttaagaataagttcaagaatgttaacaaaaacttcatcagcaacgagaagaacaacctgtacattatcctgaacgcctacggcgaggatatcgaggatgtcgaggtggtgaaaaagtacagcaaagagctgtataagctgagcgtgctgaaaaccaacaagaacctgggcgtgaacgtgaagaaactgagagagagcgccatcgagtacggctactgtcctctgccttacgacaaagaaaaggaagtggccaagctgagctccatcaagcacaagctctacaagacctatgatttcgtgatcacccactatctgaactcgaacgacaaactgctgctcgagatcgtggaggccctgagactgagcaagaacgacgacaagaaggagaacgtttacaagatctatgctgaaaagatcttcaaggccgaatacgtgatcaaccctatcaagacaatctccaacctgttcgccgagaagggcgacaagctgttcaacgagaaggtgagcatcagcgaagagtacgtggaagatattagaatcgataagaatatccacaacttcacaaaggtgatctttttcctgacatgcttcctggatggcaaggagataaacgatctgctgaccaacatcatctctaagctgcaggtgatcgaggaccacaacaatgtgattaaggccatcgccaacaacaacgatgccgtgtacaaggattacagcgacaaatacgccgtgtttaaaaatagcggcaaaatcgccaccgaactggaagcgatcaagagcatcgctagaatggaaaacaagatcaataaggccttcaaggaacctctgctgaaggacgccatgctggccctgggcgtgagccctaatgacctggacgagaagtatgaaaaatacttcaagacagatgtggacgccgataaggaccaccagaaggtgtccaccttcctgatgaacaacgtgatcaacaactctagattcaagtacgtggtgaagtacatcaatccagccgacatcaatagactggctaagaacaaacacctcgtgaaattcgtgctggaccagatccctcacaagcagatcgacagttactacaacagcgtgtctacagtggaagaacccagctacaagggcaagattcaactgctgaccaagaagatcaccggcctgaacttctacagcctgtttgagaactgcaagattcctaatgtggaaaaggagaagaagaaggccgtgatcacactgtacttcaccatcatctacatcctggtgaaaaacctggtgaacatcaatggcctgtacacactggccctgtacttcgtcgaacgggacggctttttctacaagaaaatctgcgagaagaaagataagaagaaaacaaacaaagacgtcgactacctgcttctgcctgagatctttagcgggagcaagtaccgggaagagacaaaaaacttaaaacttcctaaggagaaggacagagagatcatgaagaaatacctgccaaacgatgaggacagaaaggaatacaacaagttctttaaacagtacagaaacaacatcgtgcacctcaatataatcgccaatctgtctaagctgaccagcacaatcgacaaagaaatcaacagctacttcgaaatctttcactactgtgcccagagagtgatgttcgattactgcaagaacaataacaaggtggtgctgtga

SEQ ID NO: 102 - DNA, 2688 bp, Artificial Sequence
  Description of Artificial Sequence: Synthetic polynucleotide
atgaaaaagaacagcaacgacaaaaccaacgccaagcggatgggcatcaaaagctttatcaagaacggtgatgagcgatttatcacaacgtccattaagaacgaattccctgtcgagctgaaactggacgtgatcaagaaaacctgcgaacctgcccacgagcctgtgagcttcgattacgaccctaaaaagatcgacttcgagaagcctgtgctgaaggagaagctgacatccggccaaagcggccagaagctgtctacaagactgttcatccagaaagatagagacatctgcggcatccggagaaagtacctggaaaagatcttcaactctaacttcatcgaagaaaaaaaggacagcaacctgcctatgcagatcgtggctaaggtgctgtctaccgagaaggtgttctctaacgccctgaacaagatcatctctcagttcctgagcatgcctagaggcggcgtgaccgacaaccacggcgagtacgagatcattggcaacatcatcaaccataagagcctgcaggagctgaacaaggagaagaaaaccaagagaatcaagaagtacctgcagtccgtgatcaagaaccagagctacctgtacaacaagcagtttctgctgagcctggatgaatctaaaggaagcagaaatgacatcgacgagaatgagctgtacgactacatcaggttcctggctatcctgcgcaacggcatcgcccatgtgttctacgagaagaacgagcctgagacagccaaggagtctctgttcagactggtggacttcatcaagaacgacaagaaactagagggcgcgttcgccaagatcaagatccaagtgaacaccctgtacaagtgccggaaggaagaatacatcaagaaatccggcaagaacttcgagatcatccggaagatctaccaaaatgataaacccgacgagaaggtgaaggactggatcagatatgactttgacaagagctataagtacatcggactgagcgtggccaagctgggcaattacaccagctgggccaaagacatcgacaacctgagagataagtccaaccctgatagcggctacgccggcatcatgcacagactgaacgaattcagcgtatacctgaaggtgaaggccctgagcacagaggaaaaagataagtacctgaagaacctgatcagcaaggaaaactgcgaggaaaaggacaaatactacaagaacatcgcccagttcttttgcagtagcgaccttaagttcgccaacgtgctgcaaatggtgaaggaaatcaagaagaacaagggctgcaccagcgaggataagaactgtaaactgtgcgtggacgagagaaagttcaacgatctgagcgtgatcgtgtacttcatcagctgcttccttgataataaagatcagaacatcttcctgagcgacctcatcaacagatttggcgctctgagcgacctgctgcggatccagaacaagatcctgggcgccggaaacaagtacaatgagaactacagcttcctgaagaatgagagatacgtgacagaaataaagatggaactggaaaccatctttgccctggtgaaggtttcttataagaaagaggacaaggcctttaatcggctgcttgaggacggcctggtgatgttcggcttcagcaaggacgaggccggaatgaaggtggcaggcttgaaagaaatcaaagagaagaaggaaggccactacaaaaataagtccagaagcttcctgatcaacagcatcgtcaacagccggaagttcgcctacctggccaagtctatagacccccagaaagtgcccgccatcatcaagaacgagcacatcgttagatacatcctgggcagaatcaacaagaccaaccccggccagatcggaagatattggcggtatatcatgagccagaaccacgccgggaccgataaagtcgatgacctgacaaacgagatcatcaagatcaatatcaagaacatcctgaacgacgccggaggctggcagaagagcaagctgaacgacaacaacaacaaaaagaaactgaagtaccagcagctgatcggcctgtacctgaccgtggcctatatctttgtgaagaatatgctggaatgtaatgccagatacttcagcgccttcgctcagatcgagaaggattacctgatctacaccaattctgacgagttctactacatcgataagaacaagaagaacctggtgaccgaacggtacctgaagctagtgaaagacatcatcgaaaagaacaagaatacagtgcggaaggacaagatcttccggaagaagcgccagagaaagcacctggccgacatcagcaaatccatcattgagttcgagaagctgccatgttgcatcttcaccctgctgagaaacatcaccgaacacctgaacgtggccagcaatattgatatcattgagggatatggaaagagagccggcaagtaccacaagaacgcccctgctagctactttatcttttaccactacatcatccagaagatcctggctgacaaaatatgtacaagaaacctgctgaacattataaacacctacggcgagccctccatcagcttcatcaaaatcatttacgtgccatttgcctacaacctgcctagatacctgaatctgaccgatgccagaatcttctgcaacatggacgacaagtga

SEQ ID NO: 103 - DNA, 2574 bp, Artificial Sequence
  Description of Artificial Sequence: Synthetic polynucleotide
atgaaaaagctgaagaaccccagtaatagaaattctctgcctagcatcattatctccaagttcgactctagcaaaatctacgagatcaaggtgaagtacgaaaagctggctagactggatagactggaaatcggcgacatgagcctggacgagaacctgaacatcctattcaagaaggtgaacttcaacggcatcgacctggaaatcctgaaccctctgctgctggacttcgatagctacaccatctcaggcaaactgcagaagaattcaacaaacaagacaatcctgacactgaagaaggacggcaagatcatcaagtacaacgtgctggaaaaggataacaagtatttcaagaacggcaaggaattcgtgatccccaaggatgtgaaggaagagggcaagcgcctggttaatgacaagttcctgctgaccattgaggacaagaaaagggaagagaattccctgcctaagaagagaaagaaggagacacagcgggacattctgaaggacgaaaccatcgagatctacaagcggatctccagcaacagcaacattaaaagcgaagatatctatagaatcaagcggtacatgctgttcagaagcgacatgatgttcttctacaccttcatcgacaacttcttctactgcctgtacaagaataagaacgagcaactgtggaacaccaactttaaggaaaaggagaacctgggcaagttcatcgaattcaccctgaatgacacactgaagaacccacggaacggcatcctcaaaagctacagcaaggacctgaaggtggtgcaggaggacttcgtgaagatcaaggacatcttcgagaagatcagacacgccctggcccacttcgacttcacattcatcgacaatctgttgagcaacaacatcgagttcgattttaacatcaagctgctgaatatcgtgatcgaggattcccaggatctgtactacgaggccaagaaagaattcattgaggacgagaagatggacattctggatgagaaggacatctccatcaagaaactgtataccttctattctaaaatcgacatcaagaagcctgcttttaacaaactgatcaacagcttcctgatcaaagatggagtggaaaactctaaactgaaggagtacatcaaggagaagtacaactgtcactacttcatcgatatccacgacaacaaggagtacaagaagatctacaatgagcacaagaaactgatctctgagaatcagaacctgcaactgaacagcaaggaaaatggacagaaaatcaagatcaacaacgacagactggaagagctgaaaggcaagatgaacgagctcacaaaggccaacagcctgaagagactggaattcaaactgagactggccttcggcttcatcaaggtcgagtacaacatcttcaaggacttcaagaacaacttcagcgaggacatcaagaaagacatgaacatcgatctcgagaagatcaaaagctacctggacaccagctacagcaacaatcagttcttcaactacaaggtctacaacaagaaaacgaaacagaaggatatcgacaaggatatctttgacgacattgagaaagaaaccctgaaggagctggtggagaacgacagcctgcttaagatcatcctgctgttttatatctttacccctaaggagctgaagggagaattcctgggattcatcaagaagttctaccacgataccaagaacatcgacaaggatacaaaggataaggaagaacccctggagcaaatcaagcaagaggtgccactgaagctcaagatcctcgagaagaacctgaccatcctgaccatcttcaactacagcatcagcctgaatatcgagtacgacaagaacaacaattctttttacgagagaggcaacaagttcaagaagatctacaaggatctgaagatctctcataatcaggaggagttcgacaagagcttactggcccctctgctaaagtactacatgaatctgtacaagctgctgaacgactttgagatctacctgctgctgaaatacaaaaacaaagataacctgaacaaggaaagcctgaacaagctgatcaatgatgagcagctgaagcacaacgaccactacaacttcaccaccctgctgagcgagtacttcaactttgatcctaaaaagaacaagaaatacgagacactgacaatccttagaaactctatcagccaccagaagatcgataacctgatatacaacctggacaaaaacaagattctcgagcagcgggtgaagatcgtggaactgattaaagagcagagagatatcaaagaaaccctgaaattcgaccccatcaacgattttaccatgaaaaccgtgcagctgctgaagagcctggaaaaccagagcgagaagcgggacaagatcgaagagatcctgaaacagcaggacctgtctgccaatgatttctacaacatctacaagttgaagggcgtggaatctataaaaaaggaactcttcatccggttgggcaagaccaaaatcgaggaaaagatccaggaggacatcgccaagggcagcatctga

SEQ ID NO: 104 - DNA, 2520 bp, Artificial Sequence
  Description of Artificial Sequence: Synthetic polynucleotide
atgaacagcatcgagaagatcaaaaagcctagcaaccggaactcaatcccctccatcatcatctctgattacgatgagaataagatcaaggaaatcaaagtgaaatacctgaagctggccagactggacaagatcacaatccaggacatggaaatcagagacaacatcgtggaattcaagaagattctgctgaacggcatcgagcacacaatcaaggacaaccagaagatcgagttcgacaactacgagatcaccgcctacgtgcgggcctctaagcaaagaagggacggcaagatcacccaggccaagtatgtggtgaccatcacagataagtacctgagagataacgaaaaagagaagcggttcaagtccaccgagcgggaactgcctaacgacacactgctgatgcggtacaagcaaatcagcggattcgacactctgacaagcaaggacatctacaagattaaacggtacatcgacttcaagaacgagatgctgttctactttcagttcatcgaggaattcttctctccactgttgcctaaaggcaccaatttttatagcctgaatatcgaacagaacaaggataaggtggtcaagtacatcgtgtacagactgaatgatgatttcaagaaccagtccctgaaccaatttatcaaaaagacagacaccatcaagtatgacttcctgaagattcagaaaatcctgagcgacttcagacacgccctggcccacttcgacttcgatttcatccagaagttctttgacgacgagctggacaagaatagattcgacatcagcaccatcagtctgatcaagaccatgctacaagagaaggaggaaaagtactaccaggagaagaacaactatatcgaggacagcgacaccctgaccctgtttgacgagaaggagagcaattttagcaagatccacaacttttacatcaagatcagccagaagaaacccgccttcaacaaactgatcaacagcttcctgagcaaagacggcgtgcctaatgaagagctgaagagctacttagcaaccaagaagatcgacttcttcgaggatatccattctaacaaagaatacaagaagatctacatcaagcacaagaatctggtggtggaaaagcagaaggaggaaagccaggagaagcccaatggccagaagctgaagaactacaacgacgagctgcagaaactgaaagatgagatgaacaaaatcacaaagcagaatagcctgaacagactcgaggtgaagctgcggctggctttcggcttcatcgccaacgagtacaactacaacttcaagaatttcaacgataagtttaccctggacgtcaagaaggaacagaaaatcaaggtgttcaaaaatagcagcaacgaaaagctgaaggaatacttcgagagcaccttcatcgaaaagagattcttccacttctgcgtgaagttcttcaataaaaagaccaagaaggaagagacaaagcagaaaaacattttcaatctaatcgaaaatgaaaccctggaagagctggttaaggagtccccactgctgcagatcattaccctcctgtacctgttcatccccaaggaactgcaaggcgagttcgtgggctttatcctgaagatctaccaccacaccaagaatatcacaaacgacaccaaagaagatgagaagagcatcgaggacacccagaactctttttctctgaagctgaagatcctggctaagaacctgagaggcctgcagctgttcaactacagcctgtctcacaacaccctgtacaacacaaaagagcacttcttctacgagaagggcaaccgctggcagagcgtgtacaagtccctggaaatcagccacaaccaggatgagttcgatatccacctggtgatccctgtgatcaagtactacatcaatctgaacaagctgatcggagattttgagatctacgccctgctgacctacgccgataagaacagcattactgagaaactgagcgacatcacaaagcgtgacgacctgaaatttagaggatattacaacttctctaccctgcttttcaaaacattcatgatcaacacaaactacgagcagaaccagaaatctacccagtatatcaaacagaccagaaacgatatcgctcaccagaacatcgagaacatgctgaaggccttcgagaacaacgagatcttcgcccagagagaagaaatcgtcaactatctgcagaaggagcacaagatgcaggagatcctgcactacaatcctatcaacgacttcaccatgaaaaccgtgcagtacctgaagagcctgaacatccacagccagaaagagagcaagatcgccgacatccataagaaggaaagcctggtgcctaacgactactacctgatctacaagctgaaggtgatcgaactcctgaagcaaaaagttatcgaggccatcggcgaaaccaaggacgaggagaagattaagaacgccatcgctaaggaagaacaaataaagaagggctacaacaagtga

SEQ ID NO: 105 - RNA, 10 nt, Artificial Sequence
  Description of Artificial Sequence: Synthetic oligonucleotide
ucgauuugcu

SEQ ID NO: 106 - RNA, 36 nt, Artificial Sequence
  Description of Artificial Sequence: Synthetic oligonucleotide
gcuggagcagcccucgauuugcuggguaaucacagc

SEQ ID NO: 107 - RNA, 36 nt, Artificial Sequence
  Description of Artificial Sequence: Synthetic oligonucleotide
gcugaagcaacccugguuuugcggggugauuacagc

SEQ ID NO: 108 - RNA, 36 nt, Artificial Sequence
  Description of Artificial Sequence: Synthetic oligonucleotide
gcuguagaagccuccgauuugugaggugaugacagc

SEQ ID NO: 109 - RNA, 36 nt, Artificial Sequence
  Description of Artificial Sequence: Synthetic oligonucleotide
gcuggagcagcccucgauuugcaggguaaucacagc

SEQ ID NO: 110 - RNA, 36 nt, Artificial Sequence
  Description of Artificial Sequence: Synthetic oligonucleotide
gcuggagcagcccucgauuugcaggguuaucacagc

SEQ ID NO: 111 - RNA, 36 nt, Artificial Sequence
  Description of Artificial Sequence: Synthetic oligonucleotide
guuggaguagccccggauuugcggggugauuacagc

SEQ ID NO: 112 - RNA, 36 nt, Artificial Sequence
  Description of Artificial Sequence: Synthetic oligonucleotide
gcugugauagaccucgauuugugggguaguaacagc

SEQ ID NO: 113 - RNA, 36 nt, Artificial Sequence
  Description of Artificial Sequence: Synthetic oligonucleotide
gcugugauggaccucgauuugugggguaguaacagc

SEQ ID NO: 114 - RNA, 36 nt, Artificial Sequence
  Description of Artificial Sequence: Synthetic oligonucleotide
caacuacaaccccguaaaaauacgggguucugaaac

SEQ ID NO: 115 - RNA, 36 nt, Artificial Sequence
  Description of Artificial Sequence: Synthetic oligonucleotide
guuaaauaccaccuaagaaugaggagguucuauaac

SEQ ID NO: 116 - RNA, 36 nt, Artificial Sequence
  Description of Artificial Sequence: Synthetic oligonucleotide
gaacgauagccugcugaaauaugcagguucuaagac

SEQ ID NO: 117 - RNA, 36 nt, Artificial Sequence
  Description of Artificial Sequence: Synthetic oligonucleotide
gauugaaagcuaugcgaauuugcacagucuuaaaac

SEQ ID NO: 118 - RNA, 36 nt, Artificial Sequence
  Description of Artificial Sequence: Synthetic oligonucleotide
gagauagacccuuguuaacucguaagguucugugac

SEQ ID NO: 119 - RNA, 36 nt, Artificial Sequence
  Description of Artificial Sequence: Synthetic oligonucleotide
auuggauauaccccuaauuugagaggggaauaaaac

SEQ ID NO: 120 - RNA, 36 nt, Artificial Sequence
  Description of Artificial Sequence: Synthetic oligonucleotide
guuggacuauacccucguuuguagggggaauaaaac